SEQUENCE LISTING (450 sequences)

SEQ ID NO: 1 - DNA, 5600 bp, Ralstonia solanacearum
tcagaacttcccatcaacccggatgctgctggcgatttcctttgccagctggtgcccagcctccacaggccggtcaaagactgaaacgctgagcaggtacatgccgcccatcgttgggatatagaacaggtcttgagcacccggctggtttcgcaaatggacagtcgcgaggttgtgcagcgtttgtcgccccgagatatcgactgtctcgtaaggcgtccattcgatgatgacgctgcgctttgcgggcagccccgttgagcgctcgacaaactcttgcaactgacgagcaaggcggtttttcgtttggtccggcattgcggagaacgtatctggtttgaagccctcttggacaacaaacgacagagacgcaacagcggtatcgtcacctggtcccgagacggccaacagcgtgcggcttcgcctggtgtgagcggcatcagcctgtgctctatccttttcgaattcagccgtgtctacgcgccaggccgtaggcacggacagtgacacaccgtcaacgcgctgtgtagacatctcgtcggcgacagcgtgagcagcgaatccaatgctcgcgaaaacagcagcaagaagggcaagaatgcgcatggggtgacccggattttgaatcgagtgaagcatagcgcaatgccctcaagctttcgtttgtgaacagatcgtagtggcaacatcggacagctgctcttgctggaggtgtgcctacaacccgcatctggtcgatatcagttctagccaaggtctggtcaaccggtgaatagcttaaaccgccggatatcagcgcctgagttgacgttgtatcggtggttgctgaagtgtcctggcaattggtgctcactactgctccagcgaaacgacgtcgcgaatcgattcgaactcgcgcacgaggtcaggctggtgttggtgtagataggcttgcaccggctcacactcgagaagcctcacgaggtaccctttagcgagcacaagattgagtacgtcttggccgtaggtctgttccacgattttgtactgctcctgcaggttgctcatctcccgctccatcttcgccatctgctcgtgcgtcacgcctgttagcttggccggcttctttccatcaacaagacgagaggcaggcgtggcaacaagcaacgcttctgcataggcgacggtgacattgttcgcggcgatcatcaattccacgcactcgacctgtcgcgttggccgcatcttgcgaatcgcgcgggctacctctggcgaaaactgccggtctctcaacaggtcgacggcctcagggcaaatgccttccaacaggttcaattttttcacgatctgggagatgtctacggacagcaccttggcaaggcgttccggtgaaacgccacgctcaatggcacgccggatcatgaagtgctcctggacgctcgacaggcggttgatgcggttgttgtaggtgtatgcctcatcgtcagtagcgacaagacaggccgcctcttcatgcccaaggtcgtgcagcaccagcagccgtatatgcccatccagcaacgtgtggtttcccgactgctgatcaaccgcggtcacggaaagtggctcgatcaggcccacctcttcgatcgaggagcggatctgcttgaactttctggtgcccgtgatgccggagggagtcctgcgagaggggagaatgcacgtcagaggaacagcaagtggctcaggcacaaagcccagagtaacgcccgtcatggcgaactcccagtggaccaaacacgctcagccagatatttgggaagtgtcgccaaagattccgctcgcagaaggttgacgaaattctcgtcggcaaatagctgccgcaacgctccgacgacgaacagcaaccgttgctgagtgaactccgccttgcggaccatggccttctggcgctccacctctccctcatacgtacgcaccaagcttgacgtggtcacgtctgcgcgactcccggtcatatagcgggaggcagatctccctaaggtatgtcgccgctcgatgagccgtctcgcgcttatcagctgcttgccgcgcagcttccccgactcatatgcctcctggagggcggcttggatagccttgtcgtcatcgccagccccaacgatcgcgagcgccgccgtcagcggaatcgtgcccttttcgacggcaatcagcagacgctcctccccatgatggagcaaggtcagaacaccctgcacataggcggcggtcaagcccgtcttttctgcgatgatcttcggcgagtacccgctatcacgcaactggcggatgccggctaacagctccagaggacggcattgccggcgcgcgatgttctcggcaaggctcatgatgaacgcatcctcgtcgctgacgtgaaccactattgccggtatcgtggtctcgcccagcgagcggaatgccttgagccgtccttcgccgcagacgagaagaaacttctcactgccatccgctgcggccctcggcgtgacggtgattggcttctttagcccaatggtcttgatgtttccaacgatctcgtcaaagaccttgctgttccgctcacgtgggttgatcacgtcgatacgatcgatcggaatcatccggatctcaccaggttgtatccgcgtattcatgctgcccttctcagctttgcacgctcggccatgtgatacaggtagtccagcgtgtcgaagcgatagctctcatactcgatggcattgcgctcggccaagcgaatgccagggcgtgcgaagtcgagacgcgggagcaggtagtagtccaaaggcgcgtcgttcgccttgttcagacgaatggccacagtgacgtccggcaagaggcttgtatcgaaccgaaccttccagtgatacccgccgttcggtcgcggctgacagcgcgacaagacaagcgacacggtgaactcctgattaacgtccaggatgtccgtggccgggtctcgaagaaccgtaccgccgagatcggcaatagcttgctccgcttgcgacacgatcttcggatgcagttgccgcaggaatcgattgatctccaggtaacgatagtcccgatccggcgtaaagcccaccgcctggtaggcgcgtatcaggctgccgaagcgataggcgtacgccgcagaggagggcatcccctcggtctcgtcgatgatgaggccggacagaaagccgcgattccgatagaggttgcgtagccgctcgataagctcttcactgctgtaccgacgcgcgcgagcccggatgatgccctgagcggtgtagaacatgtccggcgctacgatgggctcgaaggcaccatccttgcggatccacatttcaggtgcgttcacgacgcgagcccgcgcgagcttgtacgacacacggttatagatgttgttgccgatgtatttctcgttggtgagcacctccctcacggttgcgcgagtccagtcccggtccaagtccgtgcgcgcatgcatgctgttcagtctcgcggcgatttcgaactcgtttagggactcatcgataaaccagctgtagaccagattgacaatacgcacctcgctctcgggacccggcaccaggacaacgcggtcggtctgcaggctcttgcgctggccgcggcagagctcggctttcacgaggccatgctcgtcgaccagcatgcgacgcaggccgtagccggcgggaccgccttgccggaaccccagttcgatcagccggcactggcctgcgaagaccttcgctgagagctcgcggctgtattcaccggccattgcgcgcttgacgcctttgacgatcgttgacaccggcgagccatcgttgtcgaactgctcggcacagtagatgacctggatgccggcacggcggcagatgtactcgtagtacgcgctctcgtcggcatcctggaagcgcccccagcggctgacgtcatagaccaggatcgcctggaagtccgcgcgaccgttggtcacatctcgaatgaggctttgcagtgcctctcgcccgtcgatccgcaggccactcttgccctcatcggcataggtgcggacgacttcgatgcctcgtgacgctgcgtactcgcggatcttgtctcgttggttctgggtcgagtactgctggtgctcagtcgacatgcgtacgtattccgccgcccgaagcgcagaaggcgcgatcggcgtagcgcaggtgtactgatcgattgccatggtggtcaccggccaacgtgtggcgccgctctcccgcgtcaggaggtgccgttctgctccccggggcagcacacaggatcacgttagcaggtggccgcgcaccgtgcctcacatcatctctttgcaatcagccctggatggtgcgcgcaccggcggcagcattacgatctgcaccctccagacgtccatctttgcaaccccgtcatcggacgcgtgctggagcatgtaagtgcctgaaggcagaggcgttttcggagctgacgcgtacatctttgcaatcgtccttctccttaggttcctggcacgctgtagatctcggttgcgcgccgtataggccgggtgagcctcgcgataccgtcgccagtagtcgctatggcactgactccatgcagcgcgggaccgcgcctggttgtcgcgatagtcgtcatcagagcgcaagtgctgctgttgccaccgtcggcgacgttggcgctggcactcggcagcggagcaatagcgttggcctggagtttgtgggcggggaagaaagagggtgccgcatgctgcgcaccgccgccgaatttgattcatcgcgagctccgggcaaggtcactgcgcggggccagcaagtagatgtggcgttcggggcattcaacccgaaatggacagctcgcacatgagtcgagcactcttccaccgggcgcgagccgctacggattgcaaatgatgtatgcacatacatcaggaaaacagtgcccgacgcgctcgattttgccacggctcaagggttgctctaccatgggccaaaaagtaggagctcgacggccgctcgtcttgcaagtgattgatttttaaggtatcttttggccacttgaggtggcgtcaagattttgcacttttcttggcacgtgtgccgagtcttttcagtaattgcggcaagggacgcgcgactcagtgtctcaatttatgaaaaaacgcttttaaatcatagtgcgatttttcaggaattgtgacactcaacaggttgttgcagaccccagaaaagttgatgtaattcacaaataagtgaatcgatccaaggcggccgcattgccgtggcgcaccagatctacccattaacatttcgtgcagccccacaaaaagaccgggcctctcaagaggtccggtctttttttaggtgtcggtagccacctcgccagtaggaatcgctctgacagagaaaggtgatgtcggtcgagctgcgcgcaatcgctacaggggtatagctgggacttgccagtcaaccccgctcacgagaaagcgtcctccgaaattctagcaatgtttgcgggcccgacggtaacacgccagaaactagggaaccgctgatcaatgaagtcggcgaatggaatcgcgagtcatgccatcttggtttttcggattgagg

SEQ ID NO: 2 - DNA, 5269 bp, Ralstonia solanacearum
agggggctggtggagtcgctacatgcttgaagttactaaaattgtggattttttgacggcacaaccctagacccagcttggctctgactgacgatgacaaaaaaatctgagttatggatttttgggacaaatcaggactgagccggctggtccggtccatgggtaggggcgtactttgagcggtcagagcggccgtacgtggcaggcctgtttcccgcgtgcgttgcgtcgaggccgcgtgaatggcgtactggggcgcccgctaccgggccgaattgctctgccaccgttcccgatgacgggatgttgtggtgtgcgtcagtcagcatgattctcgttggtagaatatgagcaataatcacaagaaaccaaccgaagtcgtttgactgcctccgctgcgcggggccaatggggcgagatacgcataatgacggcagaacgtgccactccttcgcgaattgggactttccggttctgataatggcaatttcgctcgctttcgagaatgagcaaattcgccagacctgcgaaagtagccgttctgctaagcgccgccttggtgtaacggtagcggctcaactccaggtgcggctcgctgacctcaaggccttggatgccgcgagtgacgtaattgacatgggctttgctcggctcgacccgtccgaagatacccggcttgtcattccgctgggctgtgagtatgttctgagggtagccgcgcatcatcgtcccgaaccacgttcgtcaactggtaagttggactggaagaacgtgactcgcatcaaaattctggctatcgagtgcaccggtgaaagctgaccatttgttttctcccacttgggctgtgccgccagggcggaccgtagttgacttgctcgctgcgaggggttggtcgcgggatgagctggccaactccattgagctggtgcgagaagatctcgatgacctgattgacggaaccgcacagctatcggaaagcgttgcttcccggttggggggcgtctttggtacgagcgccgccttttggattcgtcgagaggagcagtaccgcgagcagctcgcgagtttgcatgtcaaagctgatgtggcggatgcgggctacaacgagtggctgaaggcactgcctcttgcggacatggctcgctttggctggttcgctccaggcgcgacctccgctgacaaactggcggcgtgtcttgatttctttgatgtcccgaacttggacgtgtggagtcgggactacgacgatgtgaagcgtgcggttgccttccgggcatcagccgcccacccagtgaatgtacctgcgaccaccgcgtggcttcgacaaggcgagcgtgaaagtgagcgtattgagtgctcagcgtgggacccggccaagttctcatcggcgctgaccaaagcgcgtgcgctaacccgtaccaaggacccgcaggtttttgtgccagctctacaagcgctatgcgctacgtcgggcgttgcagtggtaatcgtgcgagcaccgtccggttgcagagccagtggtgcgacgttcttctcggctccagataaggcggtcctgctactgtcgttcagatatctcacagacgaccagttttggttttccttttttcatgaggctggtcatctactgcttcatacggacagggatagtctcatcctggagacgtcggagcgaacgacgtccaaaatggaagctgaggcgaacgagttcgctgcgaaaactcttttgccagtcgagctcaaccgccggctacctgaggtggctggcgacgttcgagcgatacttcgtcttgctcgagaggccggcattgccccaggcatcgtcgtcggccaactgcagcatcgcggtttgatcaaacaccagcacctcaacaagctcaaggttcgctatagctggtagtagttctagccacggaaagccacgaacttgtttgggctcttctgccagttgcatagcgagtcttccagcacttgcattccaacgttcagatggttatcaagcacaaccaggagttggtcagcttgcgcgcacgagatgtgcattgcctcggtcgttccggcgaatagcaagcgggcacccgacagcggtccagtagcaccaaccatgtaagccgaaggtgcacgaaggctcgctaggccgagctttgcgaccatcgtgaggtagtcgaacttggcagttctgccgaaccggtggacgactttcatgctcttgtatagatacccaaacagcgcccttgggttgtcgccgacggctgccgtggcacgttcgagcattgccgcatgcgaacgttcggggcctacccattccgcgtagctttcaagcaccacatccactttctcgatactttcgtacttgcggtggttgccgaattggagctgctttcgattttccagcaactccggaaggcggtcgttgaaccagattgtgaaaatattcggggcgctgttgaactgctcccacgtccaaataaactggtcattccatgcgccatagatcgcgcggataaggttccatctggttcgcaggcttttcccgaaatggacgaagaggaacgcgagccagaacgcctcatcaagattttcctgctgccggtagagagtcgcagcccgcaaggggtcgaacaagtctgacttaggatcccgtctctgtgggcagatgccgcgccgtccgatagtcgttacgaagtcgacgcggcggatactctcgacaagctgctcaacataggcaatgcgattgtcgtcgttcgcgatgcccggcagcggcttgttgtccgccttaaactcaagcagttttgcttcgaggtcagctgccaaggcttgattcctaagagtcgctaacacaagtcatccacgaatcgagcgcagatgatggcggcagcaagcgagaggagcgcgcaatggatatcgagccgtcgttcgaaacgaacgcgtatctttccaaagccggcaaaccaagcgtgcgtgcgctcaaccacccaccggtaacggcccaggcgctccctgctctcgatgcccttgcgggcgatccgggcccttatgccgtgccgcttaagatactgccgacatcggcggcagtcgtaagccttgtcggcatgcagcttgtcggggcgcttacgcggccggccatccaggccacgtatcgcgggaatcgcatccaaggtggattcgaatgcgatcgagtcatgtcgattcgcccctgttacggtgatggccaggggaacgcctcgggcatcgactacgaggtgccgcttgctcccgagtttgccgcgatccgtcgggctcggcccggtttgttcgccccccgggggctgggcacgcttgccccgtcgatactggcccgactccaatcaatttggtcgtgctcgcgcagtcgaaccagcatggcccgatgcagcttgtcccacacgccgcctgcctgccagtctcgcaggcggcgccagcaggtcatgccgctgccaaagccaagttcctggggaaggtcttcccacggaatacccgtatgcaacacatacaggatgccgctcaacgctgcacgatcattgaccgagcgccgccggccacctttgggcgaggcgacaaactccgggatcagcggctccagcgccaaccacaactctttgctgacttttcttctcgacatgcctcgtacgataacccgcttcttgccgtagggccagttgtgttagcggctcaccgctaagtctcatatcttcatccttctgattggctaaaccaacaggggcgtggctattcgtcggcgggggtgtccaatgaggccattccggcatccagttcagcaggcgtgaacagcagaccaataggctcggcgagctccggcgcaggctggctcttgcgaacgcgttgctgtatgcacgcgtcatagttcgccgtgctcatcttcttggctaacgcacgaattgaccgcgaactgttaggagtcttatttttcagatgccgctgaataagcaacaagcggacgaggtctgcccagtagggttcgaactggttgacgttcacggcgtgccccaagcgaatttcccgttccgcatctaccagcgcgttaagggccggctgagggtcaccgaccggcatcgatggcatggcgatgcgctcctgcaagccctcgtcgacatactttgtcgcctggtccgtgttcttcgagtacaaatgtaggcttccgacggcgtgcttgtatgtgcctagttccgcgccgacggcccgtgcaactatctcctgcagcatcgtgaacgcgaatatgtcatgaggcaggccgagataggcgtcgttcgaccgcatcgtcacgaacatgtggagctgacgcgctctgagcgagaactgcatggtgcacgtgcaggggatatcttttctgcctaccgccacatcgcaaccaaggtcttcggcgttgaacagttggatgaccgcacggcgtgtatcgttgttgccgctcaacttctcgatgacgttgctgacttggttgacaccacgcatgttgaacaagcgagggccgtatgccccgtgaacggtgtttccgtcgtccgagaactcgtcgtaccggctgatgtagtactggatgaagctgagctcgttgctgccggccaagtaccacaggaactcaccaaggcagctgaacaatgtactgcgctgttcagtgcggctcacacgggcacgtgggttcctgatttggagcaagaccccgtgcaggtcactgcattcgccttttgatggcgtgatggacgatttcccgcgcaaaagacgtctgtagaccttgagcaataggtcatcaagcgtttctgcagtcatgaacatgctgggcaccccaattcatgtcacgcgctccctctgtggctcagcgttcgctggcctccaaagcgaggtcaatcaaagagagccagttgcgctgtctcgcgggcttgctcgctgagtagctcttgcttcagtcgccggccgctcgttatcaatgtgcgtcggtctaccaactttgctcccttgctcagtgcaatttcccgagtttcgagggtgatgtcgtagtgggggagggacgcattggcttggaaccatccgcggtgcaatccgattgagcgagcgaatgagtggagctcgtcgaggttgtcagctactagatggcaccacttgcgcccgcgccagggaattcgggcgttgtcggtatacacagccatcgtcagccccaaaataattctgattttactgattctgcattcttgcgagtgctgaccaagcataggccagcacaatgcattctagagcgtgt

SEQ ID NO: 3 - DNA, 18546 bp, Ralstonia solanacearum
  misc_feature (9530)..(9679): n is a, c, g, or t
atttgttcgcacaagagccatctgcaccactcgctgaagcccttcgccccaagaccttggacgaggtcattgggcagtcccatctgctgggtgacggcaagcctttgaggctggccttccagtcaggcaagccccactccatgatcttctgggggccgcccggtgttggtaaaaccaccttggcacggctcacagccacggccttcaagtgtgagttcatagccctctctgcggtgttttcaggtgtcaaagacatccgtgctgctatggagcaggcacagcagaatctatctatgggcaagagcaccatcttgttcgtagacgaaattcacaggttcaataagtcccaacaagatgctcttcttccttatgcagaaagtggcctcgtaacctttattggtgccaccactgagaacccttcttttgaggtgaactcggctctgctgtcacgtgctcaggtctatgtgctgaagtcactgacagaagaagaactgaagctgctcttgaagcgagttcaagaagaagatgctcttggtgacttggagtttgaagataaagcagtagataccatcattggctatgcagacggtgatgcaagaaggttcttgaacctgcttgagcaatgcaaaacggctgctggtgctgctggtgttcagcatgttaatagtgacttcattcagaatgccctaactctcaatagcaggcgttttgacaagggtggagacaacttctatgaccagatttcagccttgcacaagtctgttcgtggctccaaccctgatgctgccctttattggctgacacgcatgttggacggtggtgctgatccacgctatctatccagaagaatcgttcgcatggcttgggaagacattggcttggctgatccacgagccatgcagatagccaacgatgcagctttaacctatgagcggctgggtagtccagaaggtgagctggccctgggtcaagcggtcatctatctggctgtagctgccaagagcaatgccggttacaacgcctacaaccaagctagagccttcgtgaagcaggataagagccgtgaggttcctgttcatctgcgtaatgcaccaacaaagctcatgaaggaactcggctacgggcatgaataccgttatgctcatgacgaacccaatgcctatgctgctggtgaaacctaccttcctgaaggcatagaagagccgggttggtatcagcctgttcctagagggttggagatcaagattggtgagaagctgtccctgctgaagaagtgggatgaagaagccaaaaagaactgaataaggggatcagatggagcaaagaacacttctctacgatgagcggttccttgaaagctatgctggagccatcatcactgatcctgctacggcgattgtggagcttgtcgccaactgctgggacgcttatgccacagaagtgaagatcacatggcccgatgttcaacttgaaaagcagttcaagattgcggacaacggtcatggcatgacccgtgaagagtttcaatacatctggcggaccatagcctacaaccggttgtcgtcaggtggaaacacaacggctcccccagcagatgttcaaggcttgccacggcttgtttttggcaagaacggtaaaggccgttttgccagcttttgcttcgcttctgaatacttgattacttcaaggaagaacgggcaagagttcgtctgccgtgttcacagaacctcaactgatccgctcgttctagaagaaatatcgttcaaggaaaagggtgttgagggtcatggaacggagattgtgggtaacggcatcattccccggctcatgttctcggaagagaaagctcgtgaattgatcggtagccgtttccttgccaaccctgctttcaaggtgaagttgaacggtagtgaaatcacgttcaatgacatccctgacttgttgtccaagagcgaagtggatgttgaagggtacggcaaggcagttattcttcacattgacaccaagaaggctgacaagacaacgaagcaacatggactggcatggtgggttcaaggtcgtgctgtcggtgaatgcaagtggagtcgtagtgactacgaaagaattcttgacggccgaacttcagaggcgaagcgattcacattcattgttgaggctgactatctaaatgcccaagatgcagtgctgggtgattggagtggcttcaaggacgaaaaccaggcgtgggtgaagacccgcgaggccgttcaagatcgcatccgccaaatcatttttgattccagcaaggcagagcgtgaaagccgccgcagtgcagtcattgaaaagattggcacaacgatgaacaccctgtcccctgttagcaaggaccgggtggaaagcttcgtcaatgaggtggtagatacctgcccgaactttggtgaaaacgagatcgtccagttgtccagcatccttgcaaagctggagcagtccaagtctcgctatgggttgcttgatctgctgcacaagtgcgagccaaacgactacgacacccttcatgacatcctgaaggaatggaccatagggatggcaaagctggttttggacgaaattcagaaccgcttgaaattgattggggagcttcgcaccaaactgaaggaagttggcgttgatgaagtccatgagttgcagccgttgtttgagcgtggcctgtggatgttcggggctcaatttgaatccattgagttcacgtccaacaagggcatgaccaatgtaatcaagaccatcttcaaggatgaggatggtaagggcactcgtaaccgtcctgacttcgtagccctgcctgacagcagtgttggcttctatgctcgtgcttcatataacgaagattacgatgaagacggtgttgaacatctagttattattgacttgaaaaccactggtcttgctctcggtagcaaggagaaagatcaagtctggaagtatgtgaaggagctacgagaaaagggctacctgaagaagcatacccgtgttgatggatttgtcttgggagacaagattgaatcgggtgagaatgaccctacaacgcatggtgaagaagtcaaaatttcacccatgctgtatgacaccatcctcattagggctgagaagcgccttctgaacctgcacggtaaggtcaaggatgctccgttcctgatagagcagcaggaagtcctgaagaagtttttggagcctattgaagtcattcaggcggaattggtggaagctgacccagtggtggcgaaatgacctcgtccgtgaattggatcgcagcctacaactacctgtttgcttctttcaacagtgagaacaaggatctctatgttggcggttctgtattttgtcgcatggtgcagcaggtcgatcctggctcaccaagctaccaacagttgctgccgctgagacaaagccaaggaaagtccaattcgcgcaaggacttttattgggaccttatccaaggtcttccagaggcccagcgatttcaactctatcgtgtcttcatcaaccacattgaagttcacgacaagccagcggcagacaacatcaggaacattgtgttcggcggtggttatgcggtgcccactacggttgttcccgttgacctctggaactctgagaagctgaacaacagtctcaatgacattgaccatgccattgatgctcaccattacaaccgggcaacgacactgagctacacgtgtcttgaagggctctacaagacatacgttcgcaagcacgttcctggacaaatggcgctgacggatctcatgccattgtgcaaggtggtgaaggaggacatcagcaagaagcttcaggcacaaggcccgtttcctgtcgagattgtcaatgccatgccgaccttgaccaatgccattgcgaactcaagaaacggcttcagtgaatctcactttggtgacgactcccagcggtggcttgctttgtttgctcgtgatctgaccaactcaatcggtcgtctcatgctcaacttcatgtgaccaccggcgagaggaagatgactacttctagaacatgcgcactttgcccaaacatactgtcagccaaaaataaaacaaaagagcacatcatccccaatgcgattggcggcaggaagaagaccactgggtttatctgcaacgattgcaataacaagttgggtcagagctgggatgcggagcttgcaaggcaacttaactggttttccttggcagtcggcatcagtagagagcgaggtgagccacctaaacaggttgtgcaaacaatcgatggggacaggtattggcttctcagcgatggctctttcacgccagaaaaatcatcttattcagaaaaggatctgggtggggcaatcgagatcaacctgaccgccaagacggttgaagaggcaaagcagcggattcaaggtatcgccagaaaatacccgaagtttgacgttgaaaaagctctcaatgagttggagataaaaaccagctatttggattcgcctcttcatgtcagtctttctttgggtgggccagaggctggaaggtctgtagtcaaaactgcgttttcttttgcatctgaatgtggtgttccacatgctcaatgcgagaaggctcttgaatatcttctagatcctactgctgaaaatttcccctttggatttgcctacttgtctgacctggttgaaaaccgaccaaaggacaagatttttcattgtgtgtctttgcatggtgatccaaagaagaagcggctatggtcttacattgaatacttcggaatcttcaggcttgttgttctcctaagtgatgtgtatgtgggtccctacaagaatgaggtttatgccatcaatccagttgatggagaacggcttgccgtgaaggtgactacccagatcagcgaagaagaatttacttcgttgatgaggggcgaaggtttcgaccaagaaaagcatagagcagcggcggactatgccctgcctatcatcctggatagagggcgaagccgaaccttggagcgcacagtcaaggaaggctttgaacatgctgccaagcagcttggaatcagtgaaggcgaaatcattcccaaggaaaaggccactgagttcactgcctttatgatggagaaaatctctccatacatagagcatctagttcgtagtgggcacagaaatgaccaaccataagccagcaaacggcaccaagaagctcctgcttattgaccttgagaacaagcacaaggtagacctgtctcccttggatgagagttacagagctatcatttttgtgggtgccaaccagaacccgccaaaagcttccaagaagccagcaacagcccataggttcaagagggtggacttcctgaagatttcaggtgctggcaagaatgccttggacttccacatagcctttgaactcggcaggaccttcgagactgcacctgatacccaatgcttcgtgctcagtggtgacaaaggctttgacccgctgctgaaccacttgaatagcaacggtatgagttgccgaagggtggaaagcattgatgagcttgtgccagctacggtggtgcctgtagcagttccatctgatgtggttgtctgccctcactgtaagaaggccagcaccattgagcatcacggcggccactggtgttctaactgtggaaggtttgctactccaccagatcccaagctgctgccgtccaatcaacccggctacaggcagtctcaaagagacaggtctgaagaagactatgccatctattcggtatgtggctggtgccatcaaagaactgatatgactggcggtatctatgacgatggggaatggatgtgtggaggctgcattgccaggtgcgccagatgaaagcaccttcgggtgctatttttttgcctcctgaagcgtcattgctttcttgatcgccttggctgtcgccggtccccggacctcacaaacaagaagaacgtcggagcgttccagcctcacggcatcctgcaagggagcatggacttgaaacctgcatatcaaccctcatgttgtggtggtttatcgttgccagagaggaaggccgtgccaagaaccattgaacagggtttcaacgattttcttgtaaatcttagagccactgctgctgagactgccgcaaccgtgtctcatcgggcatccatcaaggcatgcctggaaaataactttggcttgaaacgcttcacccgaatcggttcgtttggcaatggaacaaatgtgtcgggctacagcgatgttgactacttggcttgtctgccgactacctcattgaaacaatcatcaacctattctctcaggttggtgaaagaggcattagaaaaccgcttccccagtacgggtgttgtggttcgttgtccaacagtggtttgcccctttggtcaatacaaatctcagcatacagaagttgtcgttgcagactatgtgaaagaacaaaatggctacaaggtctatgaaatagctaatggcaatgatgggtggatgcagatttgccctgatgctcacaatgcttacgttgaacatgtggatggtaagcacggaggtaaagtcaggcagttgattagactgatcaaggcatggaagttctatcagaatgtacctatcaaatcgttctatctagagatgaaggtggcgaaatatgcgagcaatgagcaagccatcatccatcacctagatgtgaaatattttcttgaccgtctcaataatgaaggtctgtccgccatgcaagaccctatggggatcgctggctatatctatccttgcaacagtgacgttcagaagattgacgcactctcaaaactaaacacggcagctacacgggccaacaaggctgaagctgctagagcattggggaatataaaagaagcttttgattggtggcgactgctttataacgacaaattccccacctactacctatgagtatcattcaaagacaggacctgccagagaacattgagcttctggctgctcaaaggaacctctattcaagggccaagaacatcattggacttcaaatggtgctcagtggcccggttgccatttgtgctgcgatcacaacgattgtgcgaccagatttaaaggggtatgtggcactctggggcatcttggctgtggtgtttgatctctttgtcttcacgccttgggtcaagaagctaagggatagtgcagctagggttcaagagctttttgacaccagggttttggggttagactggaatgaaatttccgtgggcaaaaaacctgagcctgaactgattcatgaggaagccaaaaaatacggagtgggtgaagaaaaaagggccagcctaaagaattggtatccactcatcatagacaaagttcctgaaatttttggggtaatcattagccaaagatccaatgtgtggtgggattgcaagatgcgaaggaaatactcctttttcgtaagagtagtccttgtctccatcgcactaggattgatcggttatggcctctatgaaaagaaggatatgtttgaatttttggcctacatcgtggcacctcttgcctctacctatgtctttggctaccggcaaatgatggagcatagtgatgccgcagataggctagataagctgagggacttgtctgagaaagtgtggtctgacgcggtttctggaaaagatataccgacgctgaagattaagtgcaggacattgcaggaccagatttttgatcatagaaagagaaatccaccagtctttgatttcttgtttaagtggttcagggatggaaatgaagcactgatgaacaaaggggctgaagctcttgtggcagaaatcaagaagtaaagaagtgcctatggaatattttttattggctgaattagttagttaaattcatatgaatttaactaaccgtcaaccaatcaagaaaacagtacgttagagtcatactttctatgaagttgggagagagtatgaggaaaactttgttgtcgttggccgtggtgttgggtggttgtgcctcggcacccaaagagcctccattggtgctttatgggcaggtgctggctgtacagcctcatcaagtggtagagcagaaggtcaacatgactggtgcggtcgtgggtggtgtagctggtggggtcgtcggcaaccagtttggcaaaggaaatggcagaaaggccatgactgcccttggtgtggtcgctggtgctgtggtgggctcccaagtgagtaagagcagtgaagtgaagaatgtcactgacttgcaagtgaggatgcctgatagcaccgtcattgatgtgactaccaatgatgttgggttcagggtgggtcagaaggtgaagatcacccagcaaggtaagcaggccaccattcaagccatgaactgaaggcaccatgctcaaaaagaaagcaccctgaatgggtgcttttttgtttatctatttaacttcctccatctacacacacgttataccagtatgtatagctattccactgactcaagcttatgacccagtaggcaagaccattcaagttgtatgtcctgctaggtaatattctttcttgggatgctgtttgccactcatatctgcatctcccccaaggtccttggctttctctagaagatatgtagccttcacttgcagataatggaacataagtagaagtgaatgagccaccttcaccgggtaacatactatcgtagtaacgatagatggttttgctctgagatggtggaattggtggagcataggcagcagagcacattgctgggcttccataattgccagcacaaccccatgtgtattgattgttggcgaagttgaagccgtaggactgaccagtagcacaaacattagtgggattgctggacagagttttgccattgtcagcaccacatgcaccaacttctctctggtttgccgtacataattgactacctaaactattgctacaagtccaagagtaagttgtttctccatttgcggtggtagtagaaactgcactgggtgttcctgcatcgcacaaaaagctgttaggagcacctggcaagatagatccagcagaagggccgcatttaggtttaggggtggggctggctacatatgttggtgcaacaaatgtgtttgctttaggtattaaagcgagcattgactgctgctgtaaaacacaagttgaatcgcaatttaatgatctgtccgattgctgatattgagcatgtgcctgtggtgtgaatatggctgctgccaataatattccgaatgctaatttggttatacttctcatgaatttcccttcctttaaataatcataagaaatatattcaattagtcaaatttcacgcataaaaaacatccatgtagggtgcttttttatttcctcttgcccatctggtcgcgttttctggattttgccaaattcaagatatggttgttctcataaatcttggcgacctcaatcacctcatggtgcaaaccggtcaggctcaggcatttctctcggaggggcaccgtgaaatgcgctacgatttccttttttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctctgagatggtggaataggtggagcataggcagcagagcacatgctggggcttcataaattgccagcacaaccccattgtgtatgatgtggcgaaaagttgaagccgtaggactgaccagtagcacaaacattagtgggattgctggacagagttttgccattgtcagcaccacatgcaccaacttctctctggtttgccgtacataattgactacctaaactattgctacaagtccaagagtaagttgtttctccatttgcggtggtagtagaaactgcactgggtgttcctgcatcgcacaaaaagctgttaggagcacctggcaagatagatccagcagaagggccgcatttaggtttaggggtggggctggctacatatgttggtgcaacaaatgtgtttgctttaggtattaaagcgagcattgactgctgctgtaaaacacaagttgaatcgcaatttaatgatctgtccgattgctgatattgagcatgtgcctgtggtgtgaatatggctgctgccaataatattccgaatgctaatttggttatacttctcatgaatttcccttcctttaaataatcataagaaatatattcaattagtcaaatttcacgcataaaaaacatccatgtagggtgcttttttatttcctcttgcccatctggtcgcgttttctggattttgccaaattcaagatatggttgttctcataaatcttggcgacctcaatcacctcatccaatgaggctccttgcttgacctggtggagcacaatcacttcttggttgtgtttgacagcagccttgataaaacctctgtctgtacaattgacaccaaggttctcaagctctatgactcggccaaggttgccgtcaacacaggcaaaggccatcttgtcaaaggtgttgccatgctcatccacaagatccatgaccttgccaatgttgccttcctttgaatgcttttccatttcatcttctatttcttttagggttctttctttcatattgcctccggggattgtttttttcgaaatttacttataaagtcaaatcttgttgatcttaggtttggctgctacctgttgttctttgagctgaagcttctccacaagcctttggttagtaataagcttgctgatttcgtgccccatgtccggttcattgagtagatttgctacatacaactcaatcatgttgacggctgtggtaactcgctcaatcatgaagttgggtgctgactgaaggtagtcatagttggtgatgtcaccaatccagtgctgggtagaaatcttccccagcttctgattcctgatagccctttcatgaggggtcatgcctggagtgattgggaagacctggatgaaggcttcacgaacgaattttttcacactggcttcttctgagtagtaggtgttcctcagttcattgaagtcctgcccaaagtaatccttcttcaagtatctttccttaaagaaatccatcgtgaggtcgtattggggattggcttcaaccttcttcaagtaggcagcactagccttcttgaagtatttgtaggcactctcatcagaaatagtgtcccaagcccagtattcatcctttgtcttcttcttgaggcgtaaatcatcttttgggatgttcttcaaaaagatggctactaatctggcaggagagaagcctgtggcttgatgaataatggatagcgtgcctatgttggcctttatttgtttgtatgcattagattttttcttgatcattgtttattcctgtttcgtgtacatctagcagaaatgaaaatgatgtcaagtcaaagtcatatgaatttaactaatcaatgggaaaaataaaaaaagcacctcatgggtgcttgacaatttatgaaaacatgatttaatgatttttgtggtacatgaataaacccttttgtttatcacgacattgttttaaggcacttacttcggtaggtgcctttttatttatatttgagttcattgtctttggcttccttgagcttcctcttctcaagctcaaggttcaatgcgacaagtttttgctcaatggcaatgatctgagcctgaagcttcgtggctcgtgtgccggtggcaccagccagcagcaacagcaggttgtgatactgcatggacaaaccaccgatctgagcctccaaacctccaacagcagaacccacattgggcaggttggtggggttctcacgctcgtgctgcatgcgggccactctgtggggggcagagtggttgatgccttgctggccaagctgaacctccttggcatgttccttgatctccttgagcttcttttcaagcttgctgcggatctcggtggtgtcacgttcaagagtagttggcaccttctctttggtgggtgtcttgccatccttcctattgccaccagaaggccctttcttggccgcatatgcacgttttgcaccaggtctaggggtgaggtagcgtttggtgttgaaggctgttctggctgcaatgaagtccaccagcttggaacgttggtccgcggactcggccggggtgaggaacttgggtatcttggactggtggtgttccttcaccaaggccgcatagtcactgtccttgctgaatagcgagcccttcaatctggtgttccttccagcaccaggtaagcgaacggtgatgaactcgtcacctaccaaggcaatgccaatgcctcgcttcttcatgtagccaatgagctggttacggttggctatggagccactgacgatgcgagtgtgaagctcatcagcaaagtcattcttggcttccttctcgcgtttaccgttgggggacttggcttcaaatggcttgaggctgatcttcaagggatcgggcaccacctgtgcaaaaccgaagttgtcattcatcacggcactgaaggcattgaagaactctatgttcttctccccaggcggatggatgttcagttgctggccggtcttcaattcggtattggcaacaaggaagtgaagttcaaggttgcctttgtctctgtgcatcacccagaagtcagcgaaattctcatcaaccttcaagcctggcatgaaggtggaacggaaggcttcgatcaatgtggcgatctgctctggtgtgacagactgggattcaagtccatcacgaaaagcgatgacaccagaggtgtacttgtgctgccttttcgtggcattggcaatctctgtgaaggtggttggatcgccaaaaagaatttcaggcatgatagatcgcttcttgccttcatgatcggtgtcactgagcaggtatctaactgctgattcggaacctcctttgccagtggggaaaatggtgacgttcatatcttccctcgttttgaagatcctttctcaccaaggttggactcaagtttttctttgagtgctttcttctctaaaaagtggccatactcttcttctgaatattgatgtccattgacccaccattggtgtggcttgccatcaagaaggacagcaggcccgtcttctctatgaagttcgccattgaaaaagtgcttgatgtagacggcagatacggtttctactccggtggcaaaatcagttgaaaccatcttgaattctgctaccccagaaaaatcatctggtaacgcttttgttatgctcatttcttccccttgaataattctgcgatagtgctcactccttcctgaatcttggtgagttcctggaacatccctttggtggtgtcgttccacatcttcatgcgattcaaagaacgagcaatctgatttatgttgctgccgatctttccaagagaagcaagaagctgaacacgcaacttgtgttctgccttggtttcctggttggcttgctggtgtgatggaagcaggattgaacgggcataaatcgctgtggaagatgcacctgcttctacggctcgctgttggagagccatccattcttcaggtgtgaacctgatctcaaaacgcttggttcgctttttattgttggcttctgtgccagcttgacctgcccttattttgagagtcgggtctttttcttttttgtggtatgccattggggcctcctttgttcttgtttttatcgagcctcgcgcagcagcgggtgtgggtgaaacccacgatattcaaaccaagaatagcccgaaagggatatacgtaggattgattgaccttgcttaaactacttttcccccattttttgaatttannnnnngntagagtaacggtgctttattaaatccnnnnnnnnnnnnnnnntagggaaacgctgatttatcagtctcagccgtcatcgctgacgcagctgaggccgttgtagcaggtagctcacggaacggagccatagtgatgaagcggcagatgagctttgcagaagcggaaagcgcaggtaagaagcgcgtgaccaaacgccagcgcttcctggcagagatggagaaggtcgtgccgtggcagcgcttgctgtcggcgattgggccccactatccgagaggcgagcgaggtcgcccgccgattggtctggagaggatgctgcggatctacttcctgcagcagtggtacgggctgtcggacgaaggtctggaagacgcgctgcacgacagtatggcgatgcgagccttcgccggcatcgatctggcggtcgaggacgtgccggatgcgaccacgctgttgaagttccgacgcctgctcaacgaacacgacttgacgcgaaagctgttcgacgagatcggcatcatgctgtgcgagcgggggctgatgatgaaggaaggcacgatcgtggatgccaccatcattgaagcgccgccgtcgacgaagaacgccgacaagagccgcgacccggagatgcatcagaccaagaaaggcaacgagtggcacttcggaatgaaggcgcatatcggagtcgatgcgtcgtcgggcttggtgcacagcgtggttggcacggcagccaatgcgtccgatgtgtcgcaggcccacgcgctgctgcatggccacgagacggatgcgtttggcgatgcgggctacaccggcgttgagaagcgcgacgcgatgcaaggcaagcgagcgacgtggcatgtcgcgatcaagcgcggcaagatcaaagcgatgcgcgaaggcccaatcaaggacttgctgatcgcggtggagcgaaccaaggcacagattcgggctcgggtcgaacatccgttccatgtcatcaagaatctgtttggtcatcgcaaggttcgttacaagggcttggccaagaacacggcgcaactgttcagcctgtttggtttggccaatctggtgctcgccaggaggcagttgctggccagtcctgggagcattgcgtcctgagcgcgcaaagcgcgccaagataggtgcgaaagcagcgaaaaaccgtgctgaatcgaagcatcccttcctcaatccgaaaaaccaagatggcatgactcgcgattccattcgccgacttcattgatcagcggttccctagcgcattcaaaatctaagtcaacgatcatcagcagtatgagcagatgttttatgactcaaaatttttatgaataaaaaacggactgtgtcatcgtcaataaaatcaatagaagatgatgtcgccatgagacatgggacagattcatcatatatgtgttcatgcaacataagacaaattcatcatatacgtagccatgcgacataagtgcaatttgtcattgtggttgacttcgataaaaaatacgtttcaatgagtcatgagcgatttggagaaaattttgtcatgactaagaagagcataaagaatgaaagaacaacagtaaggattgggcctcgaatgtccagaggcctttatcagcttgcgaagtatgtttatggcgtgaaaggcgtcaactttgaggatcaacttgaagagttggtggaaaaagatctggactatcagatgaggcaaaagtggtttctcaattccacttctggaaaagagggtcagtattttgagaaattaaaaaaagcaattggtgactcgaaagatgatctttcatctgaggatgtcagtgactttgattcaaacaacccaaaaaaggatggaggcgagaatgagtgatgaatacaaagattgcttccagccgaaagaagtgaagatcagtgccaagatgccttttgaactccatttggctgcgaagcaactttactcaatgcaaggcacaagcattggggaaagggttcaaaaattaatcaggactgacctgaaaaaaatgatgagtcgtgattggttcaaggagttgccagaggaagaagtagccatctataagatgttgcttgaacttagcgagcgagaaagagtgaaatgaaatttcaatacgataaaaccataatcaccactcaatctgatttgcaagtgtcagtggcagacttgagggatctggttcaagctttcactattccaaatgaagcccaaagggttcaagaaattcaattgattcttgaatcagttgtgaggaagaatggtctgcaaccaggtgctttaggtgtggagtaatcaacctgctgcgcacacgccttgtggcggccccggcctcacgtggttaaggaacgacgaagcgttctcactagggaaacgctgatttatcagtctcagccgtcatcgctgacgcagctgaggccgttgtagcaggtagctcacggaacggagccatagtgatgaagcggcagatgagctttgcagaagcggaaagcgcaggtaagaagcgcgtgaccaaacgccagcgcttcctggcagagatggagaaggtcgtgccgtggcagcgcttgctgtcggcgattgggccccactatccgagaggcgagcgaggtcgcccgccgattggtctggagaggatgctgcggatctacttcctgcagcagtggtacgggctgtcggacgaaggtctggaagacgcgctgcacgacagtatggcgatgcgagccttcgccggcatcgatctggcggtcgaggacgtgccggatgcgaccacgctgttgaagttccgacgcctgctcaacgaacacgacttgacgcgaaagctgttcgacgagatcggcatcatgctgtgcgagcgggggctgatgatgaaggaaggcacgatcgtggatgccaccatcattgaagcgccgccgtcgacgaagaacgccgacaagagccgcgacccggagatgcatcagaccaagaaaggcaacgagtggcacttcggaatgaaggcgcatatcggagtcgatgcgtcgtcgggcttggtgcacagcgtggttggcacggcagccaatgcgtccgatgtgtcgcaggcccacgcgctgctgcatggccacgagacggatgcgtttggcgatgcgggctacaccggcgttgagaagcgcgacgcgatgcaaggcaagcgagcgacgtggcatgtcgcgatcaagcgcggcaagatcaaagcgatgcgcgaaggcccaatcaaggacttgctgatcgcggtggagcgaaccaaggcacagattcgggctcgggtcgaacatccgttccatgtcatcaagaatctgtttggtcatcgcaaggttcgttacaagggcttggccaagaacacggcgcaactgttcagcctgtttggtttggccaatctggtgctcgccaggaggcagttgctggccagtcctgggagcattgcgtcctgagcgcgcaaagcgcgccaagataggtgcgaaagcagcgaaaaaccgtgctgaatcgaagcatcccttcctcaatccgaaaaaccaagatggcatgactcgcgattccattcgccgacttcattgagcagcgcttccctagcgtgatagaaatgaaggagtagaacgaatgaagaattgggtgggaatatttcttatgttgtggaatgtctgccacttcaaacatgaagaatggcatttaatgagacagcgattgaaattaaaaaagcacctctagggtgcttttgtttttttacatggcctgctttgttggaatgaatgcgtgtattcgtttcaatgaagtgggtgctccttagctgtgaaaaatatctagccacctctttctacgcttgatgctatctttatgtgccagcaggaaagggaaaaagtcctcatagtgtggaatcaccattgaggaaattccattttctgcttcatattcactcgccttgtgggccattcctgtaagtaagaagtggatgggtcgcagcatctcgatcagtcgccagagaaccagtggggcactcttctcaatgacatattgacggtgtaggcagtcaaacaccatcatggggtgtccatgctcatcaaggtggctggtgaagccgttgggtggcccttctctatccatgcggtacatgaggttcagtgctgacctctcacgcacctggtgcggtgtgtagtcttcataggtagctctggcaagatcgagccacttgtagagagggcttctgtccatcttggcttcgcagatttcattcagattgaagtagcgggcaccatcaccgtattcactaatgaagttcaatagaacttcgtcattggcggtcagagtcggtgctacagcattggggtggctgtagatgggcttcaacctgatgcactcttcatagagcacatcaatcttgtgtccaaagcctttaagctgcttggtggtcggtgtctggtagttgttcgtcagcatgtggtgtgtcaccacagccaacttcagaagcctctccataccaatggagagatgaaagaacgccgaataaaagtagccgtccttgtcctggaagaagttggccttaagcagaaggtcgaaacctgacagcaacgtgttctttgttaggtgagcttcttgtgcaagaaggctgaattttttatcaaaacctatctgcttca

SEQ ID NO: 4 - DNA, 4868 bp, Ralstonia solanacearum
aggggtttgtagacatggagccatcgcaatcgagcaatgccgaagggctgagcggagacgatatggaggttgtctctgcagtatcccggcgatacaggcgttatcacaagatagccgcaaggcatctgccggaatgggcgccgtctggggttacgctaagcctcatacaagcgatgttgaacctccgaaacgttcgttgcgcaaagagaaacattcacgagaacgatgacggcacccagtcaacccatgcggagcatctgagatatgccgaggccgaaatagctcgtctcgtgcgtctgtgtgcctatagcaatgaaatgcagaaggcttggcggacaatatcaactatgccagctcgtggtggtggcgatccggcattctccatatgttcaaaaatctcctccacgctggggcaatttggtgacctccccaagaggcttccagcacaacgcaagaaatcgctttctcgtgtggcgaaggcagtggacgatctgcttgaggcaatcgaatcggagagcgaggcgcaggactctcttgagattgcactgattgaatacctgggaggtcagaacctcaagatgcgctctgacgacggagaggaaataggagtcgatgaatctctcgatcccttccgagcgcttagtccggccatcattccatgcgacgatacagaaggcttggaggactcccctcattgctccaagccctggcacgagtggtcatctttcgatagatacggttggcttcacgatgaagtatccatgacgggaatggccgagttgctgatcttttgtcgcgacagaataaatgccttggctcaggagccgcccaaaattctgcagcctggaagaccagatggtgggctacagccgtttctgattcggagtttgtccaactggatgaagcgcatatacggacagcccttggacgactctgtagcactgcttgtctctgccacgctagatctgccagagcctctgacaagagaagcgatccgcccgtatctgaaaggcaccgggaagatttcgagcaggaatcgatagatttcttcccgtttcaaaatatcagagtccacgcaagctagggaaacgctgatttatcagtctcagccgtcatcgctgacgcagctgaggccgttgtagcaggtagctcacggaacggagccatagtgatgaagcggcagatgagctttgcagaagcggaaagcgcaggtaagaagcgcgtgaccaaacgccagcgcttcctggcagagatggagaaggtcgtgccgtggcagcgcttgctgtcggcgattgggccccactatccgagaggcgagcgaggtcgcccgccgattggtctggagaggatgctgcggatctacttcctgcagcagtggtacgggctgtcggacgaaggtctggaagacgcgctgcacgacagtatggcgatgcgagccttcgccggcatcgatctggcggtcgaggacgtgccggatgcgaccacgctgttgaagttccgacgcctgctcaacgaacacgacttgacgcgaaagctgttcgacgagatcggcatcatgctgtgcgagcgggggctgatgatgaaggaaggcacgatcgtggatgccaccatcattgaagcgccgccgtcgacgaagaacgccgacaagagccgcgacccggagatgcatcagaccaagaaaggcaacgagtggcacttcggaatgaaggcgcatatcggagtcgatgcgtcgtcgggcttggtgcacagcgtggttggcacggcagccaatgcgtccgatgtgtcgcaggcccacgcgctgctgcatggccacgagacggatgcgtttggcgatgcgggctacaccggcgttgagaagcgcgacgcgatgcaaggcaagcgagcgacgtggcatgtcgcgatcaagcgcggcaagatcaaagcgatgcgcgaaggcccaatcaaggacttgctgatcgcggtggagcgaaccaaggcacagattcgggctcgggtcgaacatccgttccatgtcatcaagaatctgtttggtcatcgcaaggttcgttacaagggcttgcgggaaaaacgcgccaacgggtggccccggttggggtgggcgaacccttggcccgccaggaggcaatgctggccagtcctgggagcattgcgtcctgagcgcgcaaagcgcgccaagataggtgcgaaagcagcgaaaaaccgtgctgaatcgaagcatcccttcctcaatccgaaaaaccaagatggcatgactcgcgattccattcgccgacttcattgatcagcggttccctagatgaatcgtcgactgctacgccggcggtggggcttcgtgaatgacccccatgcgcaccatctcgtcgatttcggcacgagcaactgccacaatgtcttccagtgtgcggaaacgagggctttgagggcccatgacgtcttttgtgactggatggcccagcgcgtcgcggtaccgaatccggccagtgcaagtcgcttcgctaagctcttggactaggacgatctgcgcgcctttgtacgcttcaacgggtagttgagccatggctggcctccggttttgatttggagcttctgcgtcggccgttgccaagcctgtcgcccatgtcgataacgatgagctcggttgcggcaatacgctggatctcagccatgatgggcagttcgcgcgggctactgggctcgcttgcagccacaccggtggcgtgcttcgcttttgggccaggcgggtagaaaacgaagagggcggtggacggcgcatccggctcggcacgcaggcgcttgttgagaatcttcaagtacttcttcttggtgaccttcttggttgccacagagcctcctcagagggtttcaggtacctcatgcagctgacagtacgtgttgaccagttccttcacctcggccgcttcattgggcgttccgaggcgctcaacgaacgcattgagcgcgaccagaagcgagcagatggccagcaccacggtgtcggcgtactgcgtgtagtcggggccgatctgtaagccctcaattttccctgatgcgttcaggatcagatgatgttgcaacgacgtggccgaagggtgtgcatcttgagacagttgccggtagatcaccgtgtagagatctcccaggttgagatcttcggccaatccacgaaggtctatgtcctggtgcttgcccggcgcgccaatttcatcgagcaatgcttcggccgctgcactctgttcggtcgttaccccaggcatctcgcgcaggtcggtggccacttgtactcggaaccgttggtggccgccctcgaaggcatcgaagaaactggtggtcggttcgtcgcccttcttgggtttgaggcgagccccttgcgaaagcagtaggtcgcgcgaggccgcggcgcagtacagcgctgtctcgaatgcggatcgcgccatcgatcgcgattgcgcccgcaagccccgtgccgccagaatcaccgccgcctcgaagtcctgcaccgcacggctcagcgaggtcacggccaaaagatcggcgtcgcgttggaagatgtcgcggggttgaattagcagtgcaacagcaaagcggttgactcgctccatcagtgttgtatggcatggaaaaccactcggtacttggtttgcgagggtatcaacgtcaggcgacaggaacccatctctgctcagcatacgggagtgccctccgcactacacgctagcagttgcgtggctgccagcggcctttcgagccttcgacggctagaaattgtgtgatccgagcttgaccacatctagcgtcgaattaacccgtcgcatttcgaatgcggcccggcggatgcgctcagagtgagcctcgaagaccgcaagatcgttgtgcggctttccctgcgctagcgtggcaagagctgttcgtgagatcgacgcgggaatctgcttctcgccgtccgtgatcgtaaaggccagatagtcgccagttatgactgccttcggggctgaaatctgaaagctctcgtccatatcgcctcccactctcagtctcggggcatcctagcatgacccatctgacggaagggaccgtgccggcgaggccggtgccactaaacccgccatgaagatggggcaaatccactgtccgtcgctcgggaaatcaatcagcgacaccgatgcctgtggtgtgagccacccgaggtggtggagttcaggagagccggaaggtggggcggctgctcttcaaagccagccggacacgccaccgtggcgtgagcacgtcccgcggtgcgactcgctgaagctgtacgacccgtcccggcacttggcggtcgcgccgttcggcacgctcccatccttgctgtgcgctggcgagtggacgtcatgcccctgccggttggtgtagtgtccatgctcaacgagcgcggattcgttcggagttggccggtagttggggtgacgtggcgctctggcgaatgagggctgcgcgaccatcgcggcgacggccataacgacggccgccatcgtgggtatggatttcatgtgtctccccctgatggtgtttgtgcccaccatcctgatccatccccggggaaagaaagcccccacagaagtggggctcggctgtcggtggccacgggttccacggattcaaccatctgtcggttgatgcaaccgcggtgcatgagtggccggctactccagagaattcctgccgccgaagcgcgatcagagccggcgtatcgggatgtaggcccgggcgagaggcaggctgcattgagctcgagccagctgcgtgcgggctctttaaatttctgagattcgcagcttgtaggcggtgccgcttgactcgccactaatagctagggaaccgctgatcaatga

SEQ ID NO: 5 - DNA, 3825 bp, Ralstonia solanacearum
atatcgccgcctggatgataggaaaaaagatcaatggcgtttcgcattcgtcactatccacacaatgacttgctgaacctcgcgtcgcatcactgcgagacgatcaagatgaaggtgaaaaacaaccagctcgaaggtataacgctcgattgcatgagctgtttgatcgcacttgcctttgcggtggaggcgctagtcaattttgttgggtctcggaaagtgaccgactggaacgagatggccaaggctccgagcaaggtggcaaagatcgcgaaagcagtgggactcgacatccagccagagtttgagcccttttccgcaatctatacgctccggatcattcggaacggattggctcatgggaagccgcttcacaaaagcgccagcgccaaatcttctgacgagttggcgattgccatgcaggccccgtgggcaccatattgcacccctgaacaagttatagccctgtatgagcaggtacgcgaacttcgtagggtgttgttcaaaagcgcaaacatccgatgggttgacagcctgacctctgccattggctcgcacggataggaaaaccatggcaaacgttgtgaccttgctcgatcactggcagacgctcgtcggcgcgttcataggcggcctcatgggcgtagttggtgcgctaatcgttgcagtactgaccgtgcgccgccagcgctgggtgatggcctctgcgttgctccccgacatgcaacaactaagagccgccgatgatgcgctcgaacgagcgcttcactccattgatccgccgctcggagagtgggttcgatctcagtggtgtgcagagcgtcttgcacaaacgcaccccacgttgtcggcgcttcatgacggtgtagccgtgacccaggtgtctgaccttgatgcgcgactaagcgcacacttgatgctctgtcggatgaggcacaaggacttggacgcacttctgcagcaatttgtcgccgcgctccatggctcgcgggcgccgatgcctgcggccaatgttcccgcagcgatgaccgtggtgcggcgaagtgcaaaacgagtacagcaggcgtgggatctatgcgtcgaacacgggacgcttgcggaatacctgctggatcgattgattttcaataggtggccaaacatttggcaccggctgcgcatgcgttggtggccgaacgaccttgaccatcgctcgatgcatttgctgaattcggggtcgatcctgggcacacaggactcccatcaggacgacgtggccggataggaaaaatcaattctcgtactaatgacgagcttggagattcgacatgcacgacatcgaaacagacggcttcttggcacccgaggccctggcgtttcgggatgcagtgcgagtccaatttgtcgagcaaatccgtgagtgcgaggatgtttcgcgccgcgccacgacgcaagtatttgagcaacacatcgcgaacgtgacggttgcaaatgcgacggcagcctcgctgtgggcgagatgtttgagtgcaagccaggccgccatcttgctggctgagcgcggcatgggtgtggaggcgctggcacttttgcgtacggcttatgagaacttgttcttctgcgctgcgctggtcaagcagccggccgtgatagtgcgcctcgccggtgaagatacgggtcagcgtctcaagcaggcgcgtgaaatgctcaaagacaaacagatcttcgcggcaatcacggatgcagatcgggcgctgcttgaggcgttcgtccgcgatacaccggctgccacgaaagccatcagtgcgtacgaagccgctgagatagctggcatgaccggcatctatcaaggtgcgtggcggacattctcattgattgcgacccatgccacgctgaccggggccgggcacgcatttggcaatgacctgtacgatctacagttcggcccctcgttcgcaaacgtcgactcggcattaggcttggcccgtgactgcattcggctcgggcttgatgcggttgccccgatttttactgcgtgaaagacgtgggaagacaagcatggcggaattccacctcgaactttaggttccaatcggtcctttgctcgaacgaaggagccggtagcgattttgtcctacagtggggtaggaacaaattcgtgcgaaaacagggaacaaatttatgcacatgaacggttgctgctaatcgtgcgtctgcggcgaccattttatttaacataagataaattatgcgaagtccaacttgtaggggccaaaccagaatgtccgcttttagccagatcagaatgtcctctggcgagtcctatactggtagcgcctgagtcggaactggagcgcaccatgaaggatcgagggttgatcaccatgagtctgcgcgaagttgaccgcttcaaggtcattcaagccgccgccgaaggtcttttgccgcaatggcgcgcggctgaacggctgaatctgacgacgcgtcaagtccggcggttggtccaacgctggcgtgtagacggcccgacggggctgctatcgcgtcagcgcggccagcctggccatcgacagctgccccgcatgctggaagcgcaggcgcgcgccctgatccaggctcaatacgctgattacggcccgaccctggcggccgagaagctacgcgagcgccatggcgtcgacctggccaaggaaacggtacgacggagtcgacgtttgcttactttgccgcgacccgcgcttatctggaaaggcacggcaagcctgtggccttctacagcgacaagtccagcgtgttccgagtcaatgccaggggcgccaccgcgggtcgcgactatacgcagttcggccgagcgctttatgaactcaatatcgagagcatctgtgccaattccagccaggccaagggtcgtgtcgaacgtatgaatggcacgctgcaggatcgcctggtcaaggcgttgcgcctgcacaatatcagcacgctggaggccgccaatgcctttgcgccggtcttcctggcggacttcaacttgcgctttgccaagattccccgcagcgatttcaacgcgcatcgcccggtacaacctgaagaggatctcgaacgcatttttacctggcgggaatggcgcaaggtctcgaatcgactgaccctgcaatacgatcgtacgctgtacctcatcaccgatcagcccgagcatcgtccgctggtccaccgctatgtcgaggtggcggaatatcccgacggccacatcgaactgtgggccgacggcatctccctgccctatgtcctctatgaccggctttgcgagatcgatcagggggcgatcgtcgagaacaagcggctcggacacgtactgcagattgcccaacgcgtgcaagcgctgcgtgataatcggcgcaacctcgaccggccgtcacgcaccctcattggccagccaccacggcaccctgagttgcgtgcgccaggcaccaagtcaccgcgacagttgagttcagaagacttgcggcgcgccatcgaggagctggcaccaccgccccaccctgcggatgccgcaccgcctcaggccaactcgactgccctacggaaacatcccggaaaggcaccaagaaagaagagcaccacactgcactaaccggacattctgatctggacagagataggacattctgatttagccacaacaaaaatttgtcagggccaaatagaattgtcgtatctgagccaaatagaaatgtcgcgcctctggccaagccaggcatgctgccggtttcccgtcagtcgtgactggaggctgagactacggagctgtatgaggacaaaccggtccggcaagcaacccggaccgct

SEQ ID NO: 6 - DNA, 7057 bp, Ralstonia solanacearum
gtcttggcccaggaggcagttgctggccatttcttgggagcatatggttcttgggccgccaaagcgcgccaatgataggtgcgaaagcagcgaaaaaccgtgctgaatcgaagcatcccttcctcaatccgaaaaaccaagatggcatgactcgcgattccattcgccgacttcattgatcagcggttccatagcaatgccctccgaatcctgcagtagaccgcatcgatctttaaaccatcctgtgcagcggctacggtggtcgaaaactgtcgctcaatcatcacaagctccatcaaagtttttttctgtgacgcagtcagctatggttcttgcagacaggacaagtccactggccgaagtgtgcgtgtatgccgacggagagaggcaagcgaatctttcttcccgaagtctcgtagcaacttgcgcaataagagccgctaacataactggccctacggcaagaagcgggttatcgtacgaggcatgtcgagaagaaaagtcagcaaagagttgtggttggcgctggagccgctgatcccggagtttgtcgcctcgcccaaaggtggccggcggcgctcggtcgatgatcgtgcagcgttgagcggcatcctgtatgtgttgcatacgggtattccgtgggaagaccttccccaggaacttggctttggcagcggcatgacctgtcacctgaaacgcgttcactgcagccgatacttcgattgcctgttcttgaatgcgggctcggtgccttcgagaatctggacaactcggcccttgatctccggttgctcaatcgcacccgcttcatactcaaatcgaacaccctcggtgttacgagacgcgtagacaatttgctcggcgtcacacaccaccgccaagttcggattgtgtgtaaccatgatgacctgccgcctctccttcgccttcttgatgcacttgactaggatcttgtagatggtctggttgtcgaggttctcctccggctggtcgatcacgatcggaatgtcgtccttgtcgaccagaaggtagaaaaccaagagcaataatccgcgctcgccaggcgagagttggccaatgtcttgccccccgaaggtcaacgagtagcgcggcgaaaggtattcaaggccaaagatgtagtcgagaacttcggtagcttcgactcccttgcgaagctgatcggccaaccgcgtctcgcggctgtcctgcccttcgcgctgatcggagcgcatggtttggtcaatccgctgtgcgaaggcaacggcgtcttcgggcgccgcgaagctagcctcttggagcatgcgcctaacgagctgattgctttcgtcgacgccagaaaaagttccccgcacttggcgattgagcttgccgagaaactgatcctggaatccagtctcctcaatgcggacgtgaaattcaagaggaagattcatgtccatctgcccagccgactgcacgaacgcctggacaggcaggtagagccgttggtattcgtcgaccgtgccgcgaatcaacccatggatctctcgtgatatcccggagcgttgctcaatgagcttctggagctcatcaggaagcgcggcaagcgccgcaatctcggattccaactgcgcgatagttcccggcttgtcggcaggccccatcacgtcggcctttgatttttcccacttgaccaactcctccttgtagagcacgaagagacgctgacgctcgccgagcttgctcttggcctcagcgagggaagcgaaaagctcctcgcgtcgcttcaagatgctgtcgggctctgcactttgaagctttgcgtcgattgaatcgatctccccttgcatcgctttggcgattccctcaacaggcccggcattgatccgggtctcgaacaatgagtcgatgggaatctcaaccccgagctctgctaagagctgtttcaactcgctttcgaaagcttcgctctgcttcttctggttagccaaagcctgggcgatgcggcgcgcgactgcttgcccctttgcagtagtggacttcgcctcccgaagccgtgcttcctccttgccgacatcctgcagttgctgctcaagaccgtcgatcttggttgcggccgctttggactcttccaatgcttcaggggaagcattcggatcctcaacctgtctaggcttggcttcttcgagggaggcgagctcagccttctttgccgcaagctgcttgaccaagctctccttgaattccgggaaaagcctgcgctccgtaccaagaatctcgacgttgagcgcagcaatctggccccgcagcccccccatggccttgttgatctcggtcaccttgaaattgagcagcgcgtccatcgatgactggccaagttggtcctccagcgggacgtgtgagtagatgatttttcgcaactcggcatcgaaagtcgccgatccgcccgcacccagctcattgcagagcgtctcaaggtaactctgcggcagatacttcacgcgctccacactactgggctcgggatcttggtccaaatcccgcgtagtcggcgagccgtcgcgccagaccaaggttcccacgaagttttgtgcaagccgtgttcgcggatcccggaagcgcaccgcgttcaaaaacgagaagctacgatagtttttggtgttccccgccaacgcgataatgtcagcaagagcgctctttccgctccccttgttcccgatgattgccaccaaatctgaattcagcggcagttccatgtcgaaccatgtgtcagcgagctttgagccttccttcttggcgaccctaactgagcggatgaacttcgttcgattgagctcaacgtactggcgcttggccgggttatcgccaacgcagacgcgatcatcgaactcttcgatagcctgctgcagacccctgaaggtgcagtccgccttgatccaggtaaagcagttaccgatgcggtccttgtcggtcgatgcgctcagccaatgtgcatcggaacagtcaagcaagttggagttaacaccggactccttcaacttggcgcgcgccttggcgtgatcctctggggattccgcagcggtaaagacaagatctgcactgttaatgagcgtctttttctccgcgatcgagtggtcatcacatttgaggttctcccactcggtcttgccgactgcaaggatgaacctccccttgaggacctcgttctccagggccttgcgcaagccatccgcgctgacgttgaggttgttgaaaccctcaatgagagggctcccataccccacccgcttgtctgctggggccgagtcaatgatggctgcaccaagagcttcaatgctttgccgggagatcactgagttccattttccttcgccagtcgacccaggaagaagacggtagctcggggcgattgcggaaataaactgctggcggatgaattcaggatccacctgatcgaagatgatgtggatgttgatcctgctccagcttgatggtgccttgccgctttttcccccttggacaacgccgccgaacttgtccaaccgcagttccacgaccggcagaatcagatcgatgttcttcagccttccctgctcgcgcttcgccttcagcaccttttcgtagccatcgacgagcacatagtcgttgataccgattactttgaactcctctggcagcttttcaacgtcagcgagaaacgcttcccaggcttcatcttggctccccgggtagttgtgatagaaggattcgggcgtgtggacgtgtaaatcccatttcttccaagtggagccggccagggcaaggggcatgttcttgtctcgctgaagttgttgttcacaagcgggcattgaatcaaataacccggctcacgatggaggccaaacgcacgggatcactctctaccagtctcttaacggcggagaggctcgacaccccaatcagctcttggatcagcttggtcgatcggccttgccgatgaagcctcaccgcgaatgtccggcgcgcggaactggcattgccgttggcgatgccagcctgctggtgtagccgtctgaatatctctgccagggactcgcacgaatagctagtggcaccggccgcagtcacgcgacgcgtaaagccgaaaggctcaccctcgccagtcagaaagaggggactccgacaatccaatccacggtacgcggctggacttgtggtgatgccatgtcgaaacgccagccggtgttcgagatagacgtcgatcgcagcgcgcacctttgcactggcccatagcagcggtcgcactctgccgttgaaggcaatctccgctctcacgtgcgattcgatgcgtgtctccccattcgcctgcagatagtcggacaggcgcagcttggcgatttcgttgggcgtaaggccagtgccataggcaaccaataacagggcgacatcgcgcttgctgttctgcccagtgacggctgcaaccttcagtacgtggcgaacctggcgctcttcgatgactgatggctttggcatcccgtaatgctcccgcttgtggttgcgttacgagacagtatggaaaaatttacatccgagtgcaaaacgaggccgccccatccggtcaaccctcggaaaattgtgcgcagtggcagcgcgccgatctccggcaaaatcgggcgatgagtcccctactcagcctcgggtcgccgggcagccgttcattttctggtaccaaaggcgtccccgagctgccgcgcgcgcttggcctcatcgctgtgcaggtagatcgaggtggtcgagactgatgcgtgtcgcaggttgtcgcgcacggtggtcaattccgcaccgcgtgcgagcgcgtgggtggcatgcgtatggcgcatccaatgtggactggccttgcgcagcttctcagccagcaaagggtgatcgctgtcgataatttcggcggccgaccagaaaaaacgccgcaccacactccacaaccgcgttcccgtaatgctgccattgggatcgagccccaacccaccaatcatcggcgtcttcggatcccaacgcgcgcgcgtaatcggtagcccgcgttcaaccaggtagcggtcgattgccgcccgagccagcggcggcagcgcaaccttaccagcgcgactgcccttccccaccaggttcagccagtgatcgtcctgccggtccgtttcgatatcaccgagcttcgcagaaacgagttcgcaggccggtcgcatagccgaagtccagcacgaagcgcagtcgctgcgcggcggacacctcccagccgtacgaccactcaaggccatcggcgatggtgcggactagcgcccattccccctcgctgaaagcatgcgaggcatcgagcgcagtcgttcgcccacctccgcgcaccttgattccagcaaaggggttagccagcacgtagcgctgctggatcagccagcggaacatggcccccaatacggacaacgcataggcgatcgagcgcgctgacaatccgtctgcaaacggccgccattcgacggacgtgcgcggccgcgccgggccgacccaccgctcgcgtggcgtcggtcggcgcaggaagctccggtaggcaatcgcgtcctcggtggtgagcgacgacagtggccgaccgcgttcgacgatcgcccacaagatcaggcgttcggcctctttgcggtacgcccgctgggtggcgggcgattcgtgcagcgatagccacgcttggaccgcctcatagtcgttgtcggcagtgaggacgcaggtctcgcgcggcgcacggaattgcccccgcgagccgtcgacctcgttcggcaggtgcaattgctcccacggcaccaccacgccgcggacctgttcagcaatcagggcccgggcgcgctcggtcaatcgaggatgttcggcgaagaacggccggcgcggcgcgcggacggtcagatccgccagggtggtgatgccggcggcccgcagcgcggcgactgtacgcgcatccaaccacagcgccacgtcatcactgatgaccggctctggcagtcgcaacgttcggagcaactcgatcgcctggtcggctgccctgccctgtttggcgcgttcggccgctggtatctcgaacagcaccgcgaggtcttcgcgctgacgctgccgtgccagtaccgcgagctggcgccggatgcgaccaagcacgccgcgtgaggactgccccggcgtaaggcgctcgcccagatagcgctcaacggcatcgtgggtcgacatgcctgcatgccaggcgcgcaatgccgccagttcggcggcatcggggaactccggcgaagtgctgacagcgggattgacgggaagcagacgcgttttcattgaagccagcttagcgcttcaacgttgctatttcgataagtattcttatcgcaatagatcaattagacagcgaaacttccttccgtaaaaacgcatcagacatggcgacaccagcttcgcttgatgctccggacaacaacggccgaccagcgctccacaaggcgccttttcgcgtcagccaggatcggcaaggttttctgccgtgagagcgatggcgcatcacgtcacgtcgttcgcgaagtacgccgctgccctttttagaaccgggctcaccggcttcgcacccgcatcggtcaaccgcctctcccggtcagttttgaccccgttgcatgttgtcgggtccgctacgcccggcgtggcccctgctgccgccatgccccgcccggcttgaccgcttgccgcttgccgcttgccacttgccacttgccacttgccacttgccgcttgccacttgccacttgccgcttgaatccgagcatgtgccgtgccgctttgccttgcttgtctttgttcttctcatcgctcgggctgatcggctctttggtcagcaccctggtttcaataaattatttcaatcaatccaattaaacattcctatgcgcatcccggtccggtgttttgtagcatgcaattgattcatgttgatgaccatcaaaccgtccgattttggatttaggtagagtcatcggaatttcattgcagtcattgcttttcaacttctccatcttgaaaccgggatccgtta

SEQ ID NO: 7 - DNA, 6751 bp, Ralstonia solanacearum
tgagtttgaacccgcattggcggtggacgggttctgctgctgcaactggcgctcacggttgattgacggtcgccagtgaaggtctcgtctacaagtgaagtactccccaggactcagcgggaaactgagctgttcgagagcgagcttgcgaatgtgatcgccttcgggttcgcgtgattgctctcgatacctcagcgtgatttcctgcttaacaggatcaatgacgaaccaccgggcccccctccgctcataaaatgccgcgcgcggcccttcatccgccccacctcctgcataaatattgtccccgaccacgacaacgtcgccactaccgctaacctgtgggcgaagtgacttgaacgttcgcccttggtcgaaggatacgtagactgcggcatcatcccgagcgaccacatagggcgaattgaccaccttgaacatccccgtcggattgcgcttgccattaccgccaaactgcgtccggatgcccaactgctcgtctacatagaacaattccgcctgacacggcaggttatatcccggccccggatcgttctcgatgtagcggtgatcgtcgaagcggtacacaacctgtttaggtggacgcgccatattactcagcgtcttctcgctgatcgtgtcgccgaaaccgaccgacgctgcagcccggtccgcctgggaaagaccgcagccagtcagcgatagcagcgtgacggttgcggcaaccagcaacatgcggcgcatggcgacgggatgtttctcctgtttcaacgagagctccttaagtagttctgaatggatgcgcacgctctcagcctcgacgggggcgccagcggcggatcatatggtggtcaagcgacacgtcggtgccaacctgcccacccctgatgaccgtacgcgtttcgttctgaccgggccgcacaatggcgtagctgtcaggcatggtgttgcctagcttgtccaccacgggcaggggttcatcgggtttcagtgctgacgtgaccgcgtgcggcaccaccggccggctatccggccccgcgaagatatgcccgctggcatgcacttgatattggccgttggtgccactcgtaatccccgactggctcacgtcgatatagcttccgcccccgttgatgcgcaccccgtccaccccggtgatatccacccgcttgccctccatcttgatatccagattcgccaggatgttgatgccgttcttgagcgcctgcatgtcgatatccccgctggcggccaccagcttcaggccgagccggtaggcaaacaaacgaatcgcgtccttggcgctggcgaggaagctgcgccccgtggcaatgctggcgtgcccgccgctggtgatcgcatggtgcgcgttgctctgctggtgcgtgctgcccgccgtactgctctggatgccggccgggctggccagcatcaggtgcggttgctggaactcggggaattgcccctgcgccgggtcgccgccgttgcctttcaggtcggcattctgctggtgcaattgcttggcgacctcgccctggtcgtccgcttgctgcacccctgccgtgcgtgccgcttccgacaatcgttcgtgcagttcgtgggccaccgtcagccgcgcgaccggcgcgcccgcgtccatcatgtgcccccgcccaccgggcagggcttcggtggtgagctgcatcccggcagcggcacggttcacgccgtggccctcggtatccagtgcgaaaccttggccgcgcgggttctgccggccggtcgtgccgtcgatccgcgtgatgtgccccaggctcaatgcactatccagatgatcgctcttgagttgcacctgaatcttgcccgtggtgtcgtccaagaccaaggcgttgctgcggcctccgggatcgttgccgccgtccggggtcagttcccggctgcggatgcccatcagcgccttctgcgtcggcaactgccacggcggcatgcggtgggcgttatggacgacgctgattacgaggaaatgatcgggattgccatccagtgcgaccaccacctcgctgcccacgcgcggccacgacaccatgccggtttcgccgcccgcccaattgctcgccacacgcacgcgtgcactgaattttccttcccggtcccagtggaaccggatcaggatgcggccgtattcgtccacatccagagacccgaggccgtcgcggccgaccaccagcgccgtctggtgtccggccaccttccattcgatgctgtttcgtccccggccggggcgccagggcaccgtgcgcttgatcgcggtaaagcggttgcggtattcgcccttgcggccggcaagcggctgcgccttctgccccgcctgctgaccaagttcagcaaggtagttgttgctcgcgctatggctggcctccacgatcagatattggctgtcctcgtcgcgctccgtgttgctgaagtgcccggacaactcgaaccataagccgctcgccacgctggcgttgttgccctcggcttggtactgctgcgcgtgtgcttcgatttcttcacggcgcaagttgagctgcgcaacgcccctggggtcctctttcgggaaaccgtagtgccccgcgtattcgtgcacctccagctcgggaacgcgcccctgcttgcgctgcgtcgattccgtatggtgctgcgctctcgggcgcttgaaatcgaaggcgctccccgcgaacaacgcggccaccgctgtctgtatcggtgtccagcgctggatggcgttttcttcctgcggcccgccctcggcctggaagggtatccggggagcgctctcctccaccggcgggcattggctattgctgtcgaagatgacgagccggtggccggtcgcatcgtgttcatcgcggcaggtcttgccctgcgcttccaaccgtcgatagatgtagttgtgatcgctttcgccctggccgccgccctgcacggccatggtgaacggtgcatcctctcccgccacgtcccagcgccattgcgcccaagccccgtagtcttgcaggatttcgctgatctgctggtacaggctctggtcccggaatacgcggttgttcacccgatgccgcgcgaaccacagccacggccggagtacggcccgataggtcgctatgccggggtcactgcccaccaacgagaattccgccacatgtccggtgaaccagcgcggcgtgccgtccggcttgaccagcgacactgccagcagtttgcccaacatgtccgccaacacgagacccgcatggtctgccaacagcgtcaactcgaatcggaagtcttggcccaggccctcctgggcttccaggcgctcgacaaccatctgcacgggaaaggagcggccccggtggtcggcggccgaagggggatcgtcgtccgggaacgacaggcccatcaagcgctggttctgctggaaaaagaagaggtcttgcaaatgtgccgttcgatcgaattcggccatgctctcacttcttggaatgttggcgacaaacagaaacagccacggccaaaacgggcgcggctggatacgctgagctcagaggaagcgaagaaaggaacgggctgatattcagcgaatgaagatcagacacccaacaagccgaatatcggccctgactatgaagtgaacatcctttctttattatcggaaactctccttcccttgttatcggcgggcagattaccgatacaggaaaacccgcgcaagggcactttagtgccgattttaaggatcaaaaaccggcacttttgagttttgttaacaaatcataacaaatgtgccgccatgaggtgcaggtaggcgattcgtcgggcagggagggaagcagcgtgcgcgtagtgccaccgcacatcatgagcccggggctgcgggtacgccgggggcgtttcgaccctgtgcgcgcggttgggaccccgcctcacctgcgcgcttcattgagttgtttttatgcaggtgcataagccgctgttgaccgcctgtgacaaaggattgcccgcgccaaacacagtagcaaatgcatgcaaatctatgcaccaagcctatgcaacaccggtcagcgggctgctatgccgttgacaaggtatatgtccagcgaacgtccctgtccgggtgcgggcctgcatagagcaccaccacgtatgtcagcccttgatgcgtgaaaacttggtagtagagcctggccgctccctgcggggttgtcttcctcccctggctcaaatgcgtgttggacgtcccccggtggatagtgtttgtgccgtccagcacctccagggtgatggtgataccaccagtctgggcatgatcgaatcctcgagcgcggaagaatgcatcgatccgcccgccagctccgtccatcgcaaattcaaccagcgcatcaactgcactcctcaaaaagtcgtagcggtggtctgcgacggtatcgcaagtggcaagcactccagtggcaaaaataatctgaggattcgcttcagcgacaaggcggaagtactccgacggaggcggcgtcccttgctgttggctgacaggcagctcttgccgaaggcgcgcgtacgcgtcattgatgatcgtggtgtcaggcttctgcttctttcctaagagcggccatccgtcctgaccggcggcgacacgaataatgactcctcccatttccgcaaccgtccccagttcaacctctaggtcagccgggatgggcgcatacggaaggacaatcacatgggcgtcagtgtagtggtcaaggctgtcgatgtatgcacgaaacgcatacacagcatccttttcaatcggcagaagtgtgagcgtaccctccgctccgatgccgaccagaatgctccgcccttcaatgccattacgtccgcagtcggcatcgaattttcgctgaagcgacttaaacagaccgatgacgaacactgcaggcattctatgggcccatccagtatcacagacgtgatctgaccaagatggtgctcaacagggtcggcaaaacgaggcgggcaatatcctcgtcgctcagcaactccaagagcttcaacgcttgctgccgcgccggaatctgactcgctgcggcacgcggtgcagtagggatagatgggccggccaagtcaggcactggagacgtgggtacgaacgcggctggctctttggctgccgcgtgtagccggttcaatgcaatgccatttctttcagcccactcgctgacgacctcaacgacttttgctgtacggtgtcggcgccatccccaggcatagttcccaagtgcatccgtgaatgccttattcactgtgctcgagtatgcctcagcagccaacggcccgagcgcctccttttgcgccgctggaagtggcgcttcctgaagatactctcgcatccacgcaccctgctgatccctgtcgatcggcaaaatagcaatgaaggaatctggcgcggattcgacttcggcgcgctcccgactctgtcgttcgcgcagatagtgcaccaccttatcagtggccttcttgaagtagcgcaagcgatcttcgtctgggttggcaaatgcctgccagatatcgctccgaagcaccggcgtcgccacaagcctcggctgaggcaacgcaggctcgggggtagcgcgggtagcgccacgcaaggaaactaagatatcgcccttaccctgggggcgatcgacactcacgagatcgccgtggacgcgcagaaggtagtcagaaaacctcttgaaccctagagacttttcatcgaactcaccaaagccgctcacggcaaatgcccgtttgagccgttgcttaaggcgtgcgccaagttcgggggattgttcggaaagaagctgcgtaagcagttgtgtggacgcagcgtgcgaatcggacatagcggtctcagcagggtcagaggcaaaaggagatagcccgccacagagtgtggcgggtatttccaatactgaaatcatcaattgcttctgagttccctgacttgaagccacgtcgtctaaccggcagaactcggcacccggtagcacatcggcgctgctcaagctttcaagatggccctggatactttgatccggcgaatctcgaaatacttccaaggcggaaggttctggaattctactgccggtcgaccggttgtcaagcaggcctcaactcgtcacaacgaccagcgcacaccttgcacgccacaattatgactcatagagcactttatgtgatgtactacagcgtagctagacttcaccctgcgtagccgtgatttgcattggatgaccttgttcgaccttagttttgcagtttgtgagttgcacaaggtcttgatttatgaaaggtactgagtcatcccaaaaactccgaaagcaggtatcgccgcgaccgccctcgacgatagcgcagataggacaggaggcagtttggcgctcggtccagcatggtcgggttggagcaacctagactctggccgccccccctgatcgtggcaaatagccagccttccgttgctttggcacaacgtccgaatggccacgtggcactacttttgagaaggccgaaaaaccgggggcatcaatgggggcatcaaccccacccacccagaacaaatccgtttataaacaaccagctatagttcgtatgtgatacctgtcggcggaccaccggatacgacaaaaagccaggcaaccgcctagcttttccgtttccgcagcagcataacaacgaggcccgccatccatgcgcgggcctatggctgcggatgcgccggggcctcctt

SEQ ID NO: 8 - DNA, 11740 bp, Ralstonia solanacearum
  misc_feature (5055)..(5154): n is a, c, g, or t
tccgacaggctcttgagcacatacaccgccgcccgcgacagcaatgcgccgttcacctcgaacgacgggttctcggtggtcgcgccgatgaaggtgaacaggccgctttccacgtgcggcaggaaggcatcctgctggctcttgttgaagacctgtacgatgcaatgcacctgttgaaaatcaataggttggcgtgcatgcatccggatttttcgcacaaacaacccgttttcatgcacaattttttgcaccaacgattttgattgtgcaatggatcggccccttggcttgtgccggtccccaaggacctcactgaacaagcgcccccttggcctgcacactgccccaaagttaggtgcttgagactcaaaaaaacaggaaggagtcactgtgtcggtctacccaatcaagtacaaggttctgaacaaggagactggccgcaaggtcagcgtcaccaggtggcgtgttcagatccgcaaaggtggcgagagcatcagcaagctctttgatgatgaaagcaccgccaagtcttgggaagaacaagaacttcaccgcatcaacactggtgttcctcagaatctcatttttgatatgaaatatcaacttgagatgcccgacatgcgcaggcttctcaaggactactttgatcaatacatgagcaagcaggcagcttcttcactgaagacgaaccagaaccgttgcctgagtgccattccctcaatccctatctaccttaaagatttgggtccaaagattgacaactatcgttataaaagcacgattgttgatgccatgatgggcaggaccttcaacaaagaccacgttgaatttggtaacttcaaaattgatacggtagatttctggctcttgattgcctacatggattctcgtaggaaatctggcatcaaggacaacaccatccttcgtgaaatctccactatcagttctgcctttgagaaggtctacaagctctatccagaacaattccccaacagcatcatgaacccagtcaagatgcttcctaaaggtgaaaagcctaaagcctatcttggcaggaaacgggttctctctgatgatgaagctgttcagattgcagaatggatgaagcttaaagccaatcaggagccttactacctgttcgttacttgcttggagtctggagcacggaaaagtgaagtgctgggtgctcagtgggaaaacgtcaatcttaagctttggtctatccacattccgaaaaccaagaatggcaagccaagggacatcatgattcctgaagatgatgatttccgcgaatggttgaaggccaataggcttgctaaaggacctatctttaaactgacagcttggaacttccgtcaatactgggtggatgctttgaaggcacttggtctttatgatgatccagacacaagacttcacttccatgacacccgccgcactgcattgaccaagctcattcgtcagaagcgatctaacaactttcagattgccaaagagatgggagtgtcgcctcagactgttgagcagaccatccagaacatgcctgataggttgtcagaagtgtttgcgaagcttcgttctggtcaggcactcaatgaagaagaaatcatgctgctggctggtcacagttcttcatccatgaccaacgcctactttggcgacaggaactaacaagcagcaagggataaaaacggatagaaatgacagataaaataatcatttccaatgaagagaaaatggctgatgttcagcttgaaattctcttctctggcccattgagccttcttgatcctagacatccagcaatcagtgaagagcttcagaagcagattctgtatgacaggtaccacaacaaactgattgcagatggcctggaaacaggccattttttctacgattacaacaacgatttaaggtttcaaaccaagcaaccgctctatacctttgcagggttcatctttgaagcctttgccgtcaatgttttcaataacaacatgagaaccattggcaagaaggctttttcttggtgtacaaaccgagagcagtgcaaggacgattacattgatcaattcaaggtaattggaactggattcattacaacaaagtcactgtttccgaacttctacgggccacaaagcacttttgacctcatttttatcaagaaaaatatcaggcaagatgtgcatgagccagcaaccgttttgggaacaacaaaccaagctggaattcaaatcaaagccatcactggctcagaagaaactgaaataattgagccattaagaactggcagatactcccatgttctgacttttcttcgtcactcagatggagtgcatagctacacccattgcatggaagttttaaactccatgcatagaagaggtgttttggggctgccagaaaaatatgcccttgagcgacgtatcagccacccagaaatgtttgggatcgaccaacgtgaggttgatgattactaccaatacatacttcattggtacacaggtcatgccaatcccgatcaatttattactgaaggtgctggtttggaaatcaaaggctataaatattcaaacggcttgcttgtgccaaccgattaagttcaaccgttttacacccaacctaaaatgctagttctagcgactagctttttcatattgagaaacgggatcaacatagatacaactacccagttcttgaagtaaaaaatattcaagaggatagttgacgcaccttgttcttgcacatagagtacagtcatcaacaagcaagaagaactgtagaagcttgttgagtgtgttatccttcaagtatccatagttctatcttttttaacggagaatttttcatggccgaaaccaagaagctgacccttgctgagatccttgccaagcgcaaagagtttgatgacatggtggcggctcagttggaagcggaacgtcccgctgctctggcagccatgctggaacaaatcaagatgttcaacttcacggctgatgaacttggtttccatgctgctcctgatgcacgtgccgctgctggtggtgccaaggccggtgctacccgcaacatgagcaagccgctgaagtccaccaagaccgacgaaaccagtgtgtggctggctcacccgcccaagttcttggaagctgaaggtgctttcaccacctacaagagcggcaaacccgttgatgcatggctggtgactcccaccgacaagaaggccaagaccaacttcctgaagaagctggcaagccgtgaaggcaaggttcccaccaaggaacaactgggtgaaatcaccgaggcagaattcaaggcagcctaagctatagcagcttaagataaaagcacccttcggggtgctttttgccgttgacgattaagaatattgagtgaagtgacgtcttatgatcattgcccaagtagaagaaaaagttaaagcccttgtgggcaatattgataaggctgaatttctctatggccttttggaatgctatagcaagcctaaagcctccattactcgcctgaaaatggttggcaaaggtagctataacctctccaagaacgaaaatgaagttttatggaagaagcaggtctacttcaaagccactgacagcgatcagcttctatccgtcatcgatgaaatgaagaatgcagaggtggttacaaaacaccaacctcgcttcattattgccatcaatgatactgacctcttggcgattgataccaagacggcagacacgcttgatacatctcttgccgaactgaataagaagtttgactttttcctgccttgggcaaatatggaaaaagcccagtttcagcctgaaagtgctgctgatgttaaagcagccgaaaaaatggcgaagctgtttgaccttctccgagataacaacccggcaaccactgcacaagaaattcactggcagaacgtctttctgtccagaatcttgttctgctactttgcagaagacacaggcatctttgaccccaagctgtttaccaaccacatagcctcacacacggctgctgacggatcagacttggacgactacttgagtcgtgcttttgatgtcatgaaccaggaagaccgtaatggtgttcctgactatcttgcagacttcccttatgtgaatggtggtttgttcgctgacaagctgcctgttccgaaattcaatcggaagtctcgtgccatgctcattgagtgtggggccgatctggactggtctgaaatcaatcctgacatcttcggctccatgattcaggccgtagttgaccccagccaacggggcaacatgggcatgcactacacctccgtgcagaacatcatgaaggtggttgaacctctcttcctcaacgaattgaaggaagagtttgaaaagcactctgctagcaagaccaagctggaacaactgcttcttcgcttggaacacctgaagatttttgaccctgcttgcggatcaggcaacttcctcatcattgcatacaaagaacttcgccagcttgaaatggaaattttcaagcgaattcaggaactgacacctgaaggaattttcccgctttccagaatcaagctctcccagttctacggcattgaattggacgatttcgctcatgagatcgccatcctttctctgtggctggcagaacaccaaatgaaccaaaagttcaaagagacttttggacataccaatccagcccttcctttgaagagcagcggcaacgtcgtcagcgataacgcaatccatgtcgattgggacgagtttcttgctcccaaagcagagcatgagacataccttcttgggaatccaccctaccttggagcacgtaaccaggaaccagagcagaaagcagaccttgaactgatttttgaaggccacgaggaatacaaggactcagactatgtctgctgctggttcatcaaggccgcaaagttcattgctggcaaaaatgccaagtatgcgttcgtggcgaccaactcaatctgccaaggtgagcaggttgcctacctatggccggtggaagtttggtcaattcccgcgtcatgctatctacgaatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaactggtagtgcgcggcacttcgcacatgccgcgcaaaagagtgtcacggcaataaaaaaagcacccgaaggtgcttttttcttaatcggtagcgatcctacgtccgcctgtagtagccaacaatctcttccctcacggcctcagcaagctccagcttcatatgacctgtcagcttcatttgctgacccagtgcatcggcgcaaacataggcagtttggacatatccattggcagaaccactaactgattctgtaacaacagagaatttcttaatgtcagagcgagcaagttgcttcttgcccccaccaaactgaaccccatcaggaagaagacggatgcggtatttacgacctgcaaacacaaacttcaagagtagcaacggagcagccaatgcgaccagtgccgcctttcccatgtgattctcaatgaatccaatggaaatgtattcaccgagaaaaaccagaccgatccaagccgccacatagataaacagcagaatcacggtcagccctactggattagatgcggctccgaatgcgcctgcacggctacctttgaccacaacttcagccgagccatcagattgctgagtcacctggtattgcttcacaaacgagcttttcatatgtaggggatcaatcatgttcaaggttgttctggtaagtcgctggcatcaacaagctgtttttccagctactcatcctcaacccgacctttcgggttgatactcaactctaacgcatgttcatctttttgggttgacaggcaactatttgggttgtcaaagatcgtggagagaacatgccgccagagaacaaccaacacggagaactattcaagagcattgggcgcacgttggcccagcggcgggaggctaagggcctcacccaggatcaggtggctgaagcccttcacatcggcacagaggctgtatcccgcatggagcgaggcatcaccatgcctaccgtccaacgcctggcagagcttgccgaggtgtacgagtgcggcatcgatgagctactgatagcaagtagcaccaggaccggcgaccaggccgaactgatctctcagatcctccacacgcttcccgaagcagacagagccatgatcgttgaggtggtgcagcggattgccgcacgtttgaaggatcggctttaaatctaagaaggctatggatttatggacgatatgcgttgcctaacggcatgcttgccgtggacaactcttacgagttccccaccgcacataccttcgcccacaagctccacatcctgcgatcactttttttgattgatctttcttccatgaagccaaacaaagaagccgcagagcaacaagaagcttgtcgctcatacttacgatctttttcttgatttttatgatgtgatgcgcaaaagcttgatgttgtcagagatctgcttatagtgtacgtatcgattggagaaatcatgccccctcacctcgaaccaggagcatcacaaagcagcatcgcccatgactcagaacaacaagttctgtctcactggattgaaggtcaaccagctttcaaggaagtcctctccaagttgcccgacacggatcaatgtttctcccccaaccagattgatgccatccttgccattcggcgcaggatcacagaagcgaagcacgcccatgactgagcagtcacccttgccaaccgacgaggatatttgggaagaacttttgcactcggaagccagcaagcagttcttagataagctgatcaaaaaagtccaagagtttgtagagcaacaaacgcattgaagcaccttcgggcgcctttttattgcctaccatcttggcttgactccctttgacgtactaccttcttcaaagaacttcttcctgattgccaatattcgattctcaacggctttcacaggtttgtcgtagacacttctcacctcgaccaccttcttcttgttgaaagaaggatgttggcggttgaattcttcctctgagagcttgatggcacgaagatgccactctctagtgccgtctgagccttcaacggcagggccgtcttctctgtgtagctggcctttctgataccacgccgttgcaccactggcagcttctactgccggaccatctgtgcggtgcagcttgcctctgagataccaagagcaactgccatcggcattgagaacagcgggaccgtcttcgcggtgcctcgcaccatcaatccaccactcctgacttccgtctccccagataactgctgggccatcggtgcggtgaagttttccgtggatataccaggactgggttccatctggctcaacacaagccgggccgtcttcacgatgtaggtgatcgttgaagtaccaaacgtctatgccgtcctcttggtagcgtccgtttttcatgtacgcacctcccccagtctattgattctcaatctagcaagatcaatcttaaaaacaagagtcgtaagttatcgactatggagcactttcgggtgctttttttacgaccctacatgcctagtaggaccaccacccataaggagtggccctttcttctttacaggcgtacagttcgttctattgattgatcgacattaattaatcaataaatcaactaacaaaatggagaattttatgaaaaaagaaaacaacggccaagcacgaataatagaaacaaacataggtcacttccaatactgcgactttgcaatcaaggtcatatgagttgtcaagaagtgattctttctgactctagcacctgaatatcaggtgcatttatttgaatttaatttagaacacccacttcatacctaccaagtgaatgactcacatggaaaggagggttctaacaatgacttaactacaaggatatttattatgaaaacttttatcgcggttccaaatggcgtgtttcctcttaaagaagttcttgctgcacctaccaccaagatcatcatcaaaaatcttagtgctgatccacgtacccacggtaggaagttgaggattttcctttccttctatcagacctttgacatggttgcccagcttcaagaccaaggtgttgatccttgcacagtggatgtggaagacattgccacggctgcttcatttctgattgaaaagggctttgacaagattattgaaacctttgaaccagaagaccgtgccttgctggattctttgtttcaagaaagtgaggtgagccatgtctaagacagaaaccacaccagaacttgatcttccaggcattccatttggcattctgactttggaacagttcattgcttcttctgaaggtcaggaagtcattcagaccatgaagactatgagcagtgacccatattttcttatggtgccttttcttacctcatatcaatactatgacatgctggcacaattgaatgacagggctattccaatgaaggaagaacgagagaatgatgaagttctcttgaatgttgcaacaaggtatgtggttgaacatcttccaaaaatcgttattgatattggtcaagagaaaatgaaaagtctgattaagatgctttcaccagagccacgccagtgagaaggaggctgcatcgtcagcctgccaggtgattcaaaaagactgtattgagcaatagagcacccttgaggtgctttttgcattcttgatttccctcctggtctattgacatagtttttttatttttatatctcccacataaacacaacattttttaggagataactatgtccaatgacaacaaaaaagaacaatcagtagagagccatccacttgatatgctttatcattccttccatgatagaaccaatgaagatgaatattttcttgttgtgattgctccagaaaaaccattgcaacaaaccgttcaaatcgatttaaaaactttcatttcaaacttaaatttaggatataaagaacaaaagatagatattttaaaaattgaaatttcagacacttcttgcgtggtatatttagatccgaaaagtgaatttatagacatgattgaaaaaagaataaccggagccactgtttatggtgcattgcatatactagccatgaagatttgttattttttgagtgaacatggacatgagaaatattacaatattgatttgggctataaaacaaaaagaaagaaccccaccatcttcttacaagatttggtcgtaatggactagaagataaataaaccaaagcaccttcgggtgcttttttgttgccccaacaaaaactactccaccctacgtgcctagtaggaccacttgattcccagtcctgtacctttttcttcaaaaagcgcaaactacgtttattgattgaagaccagaagaaatcagtcaataactacagcgatcaactatttagcgactacccacttaacacctaccagtgaaatggttcatctggaaaggagggattaaatatgacttaaaacacaaaaggaattcatcatgaaagaacaaaaatattttgaaggtgttgccagcaactacttcagcaacgtggatggacaggctggccttgccgcagtcccttccaacttcgttgacttggtactgaccgacccaccctatggcattgcagacggtgccaaactcaccaagaagggcagcaagatcgtcaccactcagcaggcatggggtagtgactttaaggatagctgggcgaccattgatcagtattaccagtggctcaagcccttcattgctgaattcgttcgggtgatgaaggacaacggctccatgattctcttcttggatcgcaagtacaccgggttgattgcccactacttggaacgagactttgacttgaatttcaagaacaaggtctacttcaagaagaagaaccctgttcccagcattcgcaagaacaactaccgcagcaccattgaagaggctgtttggttcaccaagggaaaggagtacaccttcaacttcggtgcccagagcgacatgcttcaggtctatgagggtcccattgggaagaagaagacccagcaccctaccgaaaagtacacatggatggtggagcctttgatcaggaaccacagcaagcccggtgacgtggtgttggacgcctttgcaggctctgcaaccaccctcgtagttgcgaagcagcaggatcggcacgccattggctttgagaagagcaccgccctgtatgagatggccaaggcccgcatcgaggcagagcagcttgagtttgcattccatgcaccatgtgacgaagcagccatcgtgcgcgagctggatcagctcatggacgccgagcttgcagacatctccagcagccgcagcagtgccgcggatctggctgacctggtcacagcgtaaggagggcagcagccatgaaagaaatgaactgcctccaatactgcatcaacggcatgagtgaccgattgttcagcttcgctaagaccaaggaaggcaaggcactgcttgctgccttcaagaagctcatcgtcattcgtgagaaccagatcaaagagcttctgattgcttacaacagctacttcatggtggctgctgccatgcagctcaagggcatgcctcagcacccacgggccatgattgagttcatggcatcagaggaattcagtgccctgcatgctgaactggtgaagaccgttgaggacaactaccccttgctcatgtcctgcttggataggaagcagaagcgaaaacttgattccctctttgaataaacaaaagcaccctacgcggtgcttttttgttggcatcgactcaatccgcgagatttacaccaacaagaaagagaataggcacaagcctactctctgagcagaaaaaaacaccctgtggggtgatttttatttacgaacacaatctatcttgatgaccttcctgctcttcagaatttcatcaacttcaggtgtcagtgttttgtgaagctcaagcatggctgctgacaccatgcagccttcaaaccaatactccttcacttcacctttgacgactgctgggccatcttcacggtgcagcagcccgtgacgataccaagcagcatagtttctacccacgaccgcaggcccatcttcacggtgcagcacaccgttcttgacccattgcttcacttggttgtctacaacacattgcagcttggtcttcatttcagtcggcaatagataacgagatttttcattctagcgtgctttttgttgacttccatttatcatctgcaagaataaaaaaaaggagacaccatgaagaggtcatcaattccaatattaaccgcaatgcttatgtcattaatgacagtccaatcatttgcaggaactacagacaccgccacaaaaaccacggcaacacttgccgccacttgcactatttcagcaagttcaattggttttggaatgcttatgcttcctttaactactcaaagtgcaaacagcaccatgaatgtgttatgcaataaaggagccccatacaaaattgatttggcttatggtggagtttatggaggcggtagcaattcttatatgaactactcagcctgggagagcggtagttatgttaatgtctacaatttttatattacaaaacggatgcgccccgtcgctttgacg

SEQ ID NO: 9 - DNA, 27297 bp, Ralstonia solanacearum
  misc_feature (11955)..(12104): n is a, c, g, or t
cgcacagcatgatgccgatctcgtcgaacagctttcgcgtcaagtcgtgttcgttgagcaggcgtcggaacttcaacagcgtggtcgcatccggcacgtcctcgaccgccagatcgatgccggcgaaggctcgcatcgccatactgtcgtgcagcgcgtcttccagaccttcgtccgacagcccgtaccactgctgcaggaagtagatccgcagcatcctctccagaccaatcggcgggcgacctcgctcgcctctcggatagtggggcccaatcgccgacagcaagcgctgccacggcacgaccttctccatctctgccaggaagcgctggcgtttggtcacgcgcttcttacctgcgctttccgcttctgcaaagctcatctgccgcttcatcactatggctccgttccgtgagctacctgctacaacggcctcagctgcgtcagcgatgacggctgagactgataaatcagcgtttccctagcggctgagttgagctgaccgatcaggccagcatacttctgccgcagtgtgtcgcggaaagcaacccaatccacgttgacctgccgcacctgggcagcgtcatgcgacagaaaaagccattgcccttcgtcactcaggcaccacagcggcacggcccagtcgggatcacgccactgcgcagcaatcgcagagcccaggaggttgcgctggtctgtttccgacgagggatacgtatgaacggcgcccagcgctgaggaggcgaagcctgccacgatggatgcctcgcacgcggctttgatggcggcgagcttcgccgccatcacctgcgcgagggtttgagaagggaatggcgccgccgagttcccgttttccacccatgccaggtacgcgtgatagtccacattcctggcatcctgcggaatgattgcgccgtcctcctcacggaggatggcgccattgatccattgcttgtatcccatgacttacagcctcgcattcaggatgtagttaacccggcaccatgttccgccgtacacgccccaagttgcatccatatcgatcgtggatgcgctgcacccgatgccgccgctcgtcatcccctggttgtggattgtcgcgccgttgtagacggtgtacgccccagaattgccagccatatccttaaacgtcatcgatggccccgcgcgcatttgaaccggaagagtctttgtcccctgcaaactggtcgagttgacagcgattgctgcactcccactgccacttgcgattggcgcgccgccgcagtggaatgcatccgcctggtagtagcgctggcacgccaccagctcaattggaatgggccgaaactccacggccgttgcggtagctccttcttccaggctccactcggtgaaccagaaatctttggccacgacagcgccacacgtagcgtccaccccgatctccaggccgttcgagcagtcaccgagcgccactgtgaacgtcagtaactgcgacgttccggacgccacagccaccgtcttgctgttgattgcgttgaccgatgcgaaattgtcggctgccgtgggcgttctcagtaccaccgtgtagttgatggccgcgccaacggtgtgccgcactttaatctggaacgtaacggtctgattcttgaggtttgctgcgtcagtagcctccatgcggtaacgccatgaaagttgccccgccccggtgagcgtgcacccctgaaagcggacggccttgccgctgcggcccacaggggccgccgtgtcctgcactagcgtccctgccgtaatcgcgccaccagaagcccagccagcaatcatctcaaccggcccatactgaggcgacgtagaaagcgacggcgatgtcgcctgaatgacaacctgcgccccgctgttgatgagcttgttgcggtacgacgtggagcccgcagagccggtagcggcctgcaatagccccacaatcgcttggcgaaactgggtgttatcagccttgttcagcacaaggccgcaccactcgatgaacgacgccacttcctcttggatggcgttcatgaacggtgccgtaatctcagtcggcggccggttggtcccggcgtcttccgccacccacgtgccgttgacgtttccggccccatcaatccggtgcatagttacctccaactcccgtaatgtcttcaaaccaaagaaacacgtgcgcttgctggaatgccgccagggcttgccagagcgcagccggatcgaccaccgagcggtagtagcgcacgcgcagcacgtagcgggatcgggtgccccataggcggttgccaatgtggctgcccacacgcgcaggccccaccaggtggtccacctgcaccagatcaatggggaacatcagcgttggcgcgtcatggctccacaaccggttgccgaccgtgctgccaatggcggctatccggcgcggcatcggctcgcccacgccggcaatggcctcggccgcaacgcggcggtactcgctgatgtgccagcttccgaacttgggccggtgcagctcaatggcggcgtccagaaccgcttgcgtgtcggtgcccaggcgctccagctcggccgcagtgcccgccagcatggcgtcgcccatgccgccgtccggccattcccacgcggcgccaggcggcaacagcgccttgagcgcgtcggtgtagtcgcgtgtggtgtgcgccgtgagcttcatgcccacaccacaggattcatcaccagcacttcgcccgctgcgacggcgatatcggccagcggcgcgaggcgggtgtattgggtggtgacggtggcaacggcagcatccacctcggccatggccagcaccgatgtttcgctgacctcggccagcacggcggccatcagcgccgaagacacggcggtgcggttggcggcggtgtcactgccggccagcagatgaatagacgggttcacgtcgtgccgcagtggtgccaccgcgcgccaatccgccgtggcgggggcaatgtcttgcagatgcgcagctactgcggccagtacggcagcagtcggcatgcggtcggccagcgtgttgcagatcggccggacgatgattgttcccatgcccagcacgtggcgctgaacgagggccgtgctaacggatgggtgggcgcttctggcccaaaacctgtagtcgtcgtccttgccggaacgagcgccacgggtggtgatggtggtccactcgtcggccacccgtgcgcgccaatcgtccacgctctcgtcttcagcgccgcccgtgattcccgcgtcgcctaccgtcaaggtgccattgacgccaggaatcgggtccaccaccgtgagtgccagacccgcagccaggttgctggccaggccggtggtggtgcagcgaatggaaacaggggtagtggggccagcgccgatatcgactgccgccgccacaatgtaatccagcccgttggggccgcgcagcggcgtgccagcaaggatcggcatgcctgccacaccggtggccagcgcgttgcccgtgctagcggtggccatgaggcggtcaacaccgtagagcgccgcccagtcgtagaggcgttccagctcgcacgtgagcggcgagcactgggcatgaagccattcgagatggccatgcatgccgtgcgcggcacgtgcccacatggcagccaggggaccacgcaggaccgcaggcattgcagccagatcggcggcaatgcgtgctttcaggtcggtataaagcggtcgaacgtaggcggtcacaaaggggctcgcacaatgaactttcgtccattgtgaaagccgctgacctctagaactacgctggaaacgtttcctgccgcctcggtggtgacctcttccacggtcacgtctgtgagggcgggcgcctgggctgtaaatgcccgacgcaccatgttgatagcttcgcggctggcgacggacgtaccaaaggccggttccggcctcggtatcttcataccagccgcgccggtcgtaggtgtctgccacgcggccggcgggcgcctctacgtccgtgaacaacacggcgtagaccaacgtggccgccgcggcatccgcgtcgcccagcgccgggtcatcaagcgccacgtcgaacttcccccagtcgctttgaatgagcttcagcatgtcaattcacgtccgaagtgggcgtgccaggggccgtgctcgtgtgggtgtgcttgtcgctcacgtccttaccattcaccctgagttctccggtcacatcagcaccgcccttcatctgcgcagcgccgccgccgtcgccgaagtagccacccaccgagctggtttggcccgccactgtaaggttaccgccgatcttggcgtctttcgtcgtctcgaacatgggcgtatcggccagcaccttcgtgctcgccttgacctcgatgatgccgccgcgcttgatgtgcacccagttctgttcgtcgtcgtgaatggcgatttcgccctccaccagctccatttggtagcgcttgtccccgatgatgagcgccacgccataggagcggtcaccagacgggaaagccaggtacgtttggcagccggccttcggccggtaggaaaagccatagggctccacacggttgatgttgtccaacgtctcttcgtccagcacgcgcacctgcaccttgctcgcgccgactacaaggccaacgccctgcgcgaacagcaactggagccgtgcccaaacctgttgcccgatcatttcttgtgccccttgccgcgcacgcctgcgccgcgcttggatttcttcttttcttcgcccaggaacgcaccccggttcatcacctgtagcagcgtgacgcggccgtgcttgtcatccatacggaatgtccgttcaccgatcaggaacacaccatcaatttcttcttccgggatgatgacgcgcacctgggtgttgagtgcccacgggtgccacttgccgtcggtgtcctgatagcgccagcctggcacctccagttcgatgcggtgggcacgcgccaggcgccggttccgctccaggagtgcgcgccgctcgcagccgcccaggccgttgctgtgtcggtccgccacaacgtgcatgggccggaaatacgtgatgccattgtccttcacggcacccttgagtgcggaatcactgctgtagtcgaacgtcttcacaacgtagtccgaaaaccgcagcttgtattcgtcgatgacctcgtagaccttgaagtgcatgccgtattgcagcgtggccacgggcgcagcgctggtaggcgacgtgagcaccaggccaccgtccggcaacggatacagcagcaggtttgccgcccgcacagcgttgatgagcgcgttggcaggcacctcgcactgcatcgaaaaactgggcaccacgggcgtttcagcgtcgatcttcacagacactttgaacgtggcgcaaaggcgcttcacgatctcgctcagtttcaggccggatagcgtcttggaatactggcagtccacaagctcacgaccaagcgaccgggcgtgaaagttgattgtgtggctggtgggatcgacgcggcggcggttgacatccggtcgcaccttcgtcaccagctcgtcgccgatcagcacgtcgatgacggtgttcgccgtcatgcccatgccgctgccgccgttcgcgccgctgcccggccgcgcgatggcgagctgtacggcggcgcacagatcgtccaccgactcccgaatttccaccttctgccagtagccgtagcgcgtgccgtcaaagcggatttccgccaatgcctcatccatacacacgccccttcacaaacagcggatggcgcgccgcattgcgcgccaggaacaccgattcatccacgcccagccggtgcgcaagcacgatggccggcagcgggttggtcacgtcgcgcaacgcggtcggcttcaggtcttgggccagcagcgcttcaatgaccgccgcccgcgcggccacggccgcgtcaaacacagggtcgggcatgctgggcagcagcgcatccagggcagtcaccacggcgcccagggctgcatctctgtctgcctcggcgcggtagtccgccatggcgatttcgatggcggccgtcaccagcagccggccgcgcaacgcttcttcgcgcagcagattgcgacgcacggcaccatccgttgaggcaacaccagacagcgccagcggcgcagccgtggtggccacggatgcgatgcgagccgccacacgcacgcgcgacgtatccgggtagtccgtcccatcggcaccaccgcccaggctgttggtgaggctgcgcaatgcgttggaatacgcgctgggtatggccatcagcgtggccagatcgcccttggccccggcaatcaggttcatgacctggctcgaccaggtgagcggcagcgtggccagggaaatgacctggcgcagcgcttcgagccgctgactcacggccgcaacgaatgcagtcatgccgtcaaagctcatcgactccagcgtgaagtcgtccatggcggcgtcggccaagctgctgttgcggtcataggccacgtccaccgcatcggtttccccagcggctaccgtgccgccgcccggcacgaagtcaatcgtgatggagcaatagccgcccttgtcgttgctctcgtgaacagaccaatcatgtgcacggacccacttgtcacccagccacgggtgcgtcagccaatcggcgcccggctggttgagcagttccaagaacgcatcgcgctccagatcgtagtccggcccgatgaaataggcgttcagcttccagtcccacgccttcgcgcccaggtcttccacctcgggctcttccgcgcccggcagctcgtgcacagcgaggcgacggccgccgcgtgcttcgtggctctcggtcagaaaatcgacgccgcgaaagcttgcgccgaccaaacggtcttcccagctcatcagggcgctccgttccaaacattaccggtgttcatctgcacgttgccgcccgtggcctgcacagactggctttgcagcacaaggccgggggcaaggcccactgtcatccgggcgttcacgtccaccggcttctgctcggctggcttcagcaggtctttgagaccttcccaggctaagcccagaccgccaccgacagcggcaccaacaccggtgcctaggacgggcaccacgctacccaccatggcgcccatagcagcgccattgagcgcactggagccgtagcgggaaatagccgactcttcaccaaaagctttctcaagggcgtagtcacccaccagagcgcccacgccagcgatgccgccgaccttgcccattttcatcgcgccccggccgaccgcgctacctgtcgcccaagcagcggcattggtgatggcctgtccgcctggcagacctttcccgcccatcgcaatcgatgccaggccagccacacccgccagtgcgccgagtgcggccgtggcgagcgtggtagtgccgaccagaagcggatgcttcttcgccagatcgccaaaggcttcagctgccttgccaattgtacgtagaaggttgtccatggcggacttttgcgccacggcggcatcttccttggcctgctccatcttgaagtcggccgtatctgcaatgcccttgaaggaggtggcgatttccgagcgctcgggggccaagccatactggccccgcaccttgcccaacacgtctttggtgtattcctgattgttcatcacgcccaggaacgccatcagcgactgctggttgtggaacaccttgccgaccgccgtaccctgcacctgggcgctcatggcttccagaatctgccgctggccttccttgtcgttcttgtcggtggcacgcagcttggcttgcagcgcttgaaacttggcatccttggagaaaatcgactgcatcatgtcgatggtggcgcccaccttgtcaatgcctcggctctggtagtccaggaacacgtcatcaacaccctttaggcgcttttccttctcgccgcgtttcagcttctggccgttggccaggtactgctccgccatgtacttgcggaagtgcggtgtgttcagctcgttcagcaggtcgcgcaggttgttgcctgcctcgtcgcgggtgcctgcggtaatgaccgatgcctgattccaggcggccagcttcgcaaagccttccttgcctgataagcccaggttgctggccattgccatctgctgcggcagccacttcgccatatctcgcagctcgaagccgcccgcttggccggccaccatggcagcgctcaggatttgcggcatgtcgtccgcgctgatcttgaatgactgtttcgcgcggatggcgatggtggccagtgcgttggcgtcggtgccggatgcagtggcggccttcatgatgcccggcagcatcttcattgcatccgtatcagacacggtgccggacgcgatcatggtgtcaagtgcttccgctgcctgctcgcgcgtgccaccgccttcgcgacgcgccttgttcacagcatcctccagcgacttggtgccgaccttgcggcccgcggcgtcgcgttcggaataggcggtgttggccatgttggccagacgccggtcatagctcatggagcgttccgcaggccccctgagcgtgtaggcggcagcaccgatgccggcgatggcagcaccagtgaacttcaggcccgcagcggtcttctgcgcagcagtcagccgccccatttcgttggtcagctcagtgaccttctggcgcatctggccgaccgctgcggcttgctcgcgccatgacatggtgccggagcgcgccagccggttgtatgccgcttcggtttgctggatttcgcgctgaatggcgcgctcagagcgcacgcccaaggtttcgcgcgcctgggcaagccgttcataggagctgcgttggcgcgcggcgcttttctcggctgcatcggcagccttctgcgccgccttctcggtggccgtggccgtcttggcagcagtctgctctgccgttttggcagtcttctgaatcgcggcattcgcgctgccatcatcaacaatcttgatcttgacggcgacatcaacagcaggcgacgacatgaaaaaagctccagtaccggaaggtatctggagcttatcgggggtgcttctttcctggcaggcaggaaatgtttcctaccagtctgtgcgcgctactcgaatgtcttgccgtccatctgtgctgcaatctttgcccaggtgaacagctcgggcaacggcatggctttaacgacgggcagcggctggtgcaaaccgcgcgccaccaatgccgcagcgagcagcacgcgcgttacttttttcgcgctgcggcggcggcttcatcctcgggcggcgcttcgccgctttcggctgccggttcctcggcttcgtcttcgtcggcttcgatgatagcgtcggccatcttctcggcccggcgatagtcaatgccgtgcagcttcttcaccagctcttcgtcggtgcccgtcaggctggcgatcagcgcaatgcgttgggctacgccgccgcgcttgtcgaacgacagatagtcttcggcagtggcgtagtcacggaacgtcaggtggctgaccgtgactttgccgaacgccagcgggcgtttcaggttcagtttctgtgccatgttgttggtctcttcagtggcgggtaaaggtcaagttgcggcgtgcagtgcgtcgcgcaaggcgtagcccatcagcggccacaccttttgcgcagcgttctggcgcgcaatcttgcggccgatttcggcattgaagttctcagggcttgcgcaggccgattcaccggtcacggtgaaaccattgcgcaataccagcacacagaaggtcagcaggtgcagggacgggtggcgatcccgaaagtggccgcggccgtcccgcccggccatcatgccgtcttcggcagtgaagtaatgctcgcttgcgatgttcgcttcgatgtcggccggcgtaacacgcggcgcagtcaggcccatggactgaatttccttctcgatgttgtccatagcctgctcttaggaaatccgctcggacgtgttggcgatgatggtcaccttgccatcaccatcgcccacgggcacgggttccgacgtgaatgcctggctcatgaggtacacagtgccatcagccaggcgcacagtcacatcttcgtccttgatgtcgttgatcgcgtcaacgtccagccctggcgtcagattcaggttcagctccagcttggccggcgcgccggactcggtgtagccaccgtcttgcggcaggcggcctggcttgtgctcgcgcttcttgccactcggcgtgaacgtaccgggcttgtcggccagcggcagcttgccgatggacggcaccgacacggtacggatgttgttgagttgggacacgttgaactcccttcaaattgcgttggaaaccaagacttcgtgcgcgtcgatcagaccaccggtacagccttccggaagcgcgagcggcccgcgaggatgtagaacggcgagttgaccaccgggtcatctaggaagttgaagcgactggggttgtccgggtcttgctccaccaccagagtgctcttgtagtagtcatacgcctgcacccagccgtatcgcgacatcagcacgttcttgtagagcgacagcaggaacgctttcacgccatcttccgtggtgatgggcaggcccggccggtagccttcgtccgtcttggcggcaacggtgccccggaacttcttgatggcgcccgtgcgctgttcgtagcggatgcgctccgtaacctcggccacgttgatatccagataggcgtcgtcggtgctgccatcggagcgatgcaggtacatggaaatcagccgcttgatgtagcaggaaccgtccgtgcctacctccatgaggctcatgcccttgaagagcaggctgttcccgctggtccactcgtgatagctcgtggccacaagcccaggcagcgatgtaccttccagcgagataaccgggttgttgtacagcttcggcgcagcggcggcggccagcgtcgcggcggcttcccaggtagtgggcgggttgatgcccagcgacaggtccgtgatgtgctcgtagttcttcgtctcgccaaagctggctgctgctgcgtagtccccacgataggcagcgaatgcgcggaagccggcctgcacgggcacccggtagcggcgttggctctctgcatgccatgccgccagcgtggcggcatcgttgatgcccagcacgacgtagcggaaccacttctgaccgatcagcgttgccaggtcgcccggcagcgggtcgcccgtaccgcctgccatggcggtcagcgcgagcgcgagccctgcgggcatgtcttcgccgtacaggttcaaccgcaggtcgatggcgttgccgcaggtgcctttatggcgcgcgctgagcgtcaccgcagcaccagcggcaacggcagtcaccggaatgtccgcacctgcgtccgtgatggcttgcgcaatggccgtcgcaatctgcgcagccgtcttgcccgcagccacgcccacggtgatggcgcgttgtgcgatgtacagcgcgagcgtgccgtcaccggtggccgcactgttgacgttgatggaaccggttgctgctacgccggccgcgttgtccgcgtagggcagcatgtacaggtcgagcgtctgatcgacattgcgatagcgcgcggccatctgcgccagcatcgaaccatcgcctgccagcgtcttggcatccgctacgctggcgacacgcaccagttcgccggtcggtgccgtgccgctggccaatttctggcctaccagcagtacgacaggcaaatcactgctcaggcccgcctggctcccatcaatctcgatgtaagcgcccggatagcgcagcgccgaaggcacttcactgaaggtaatggtcacagcttttctcccgtcaacagctcccggtctcttgtggtagggcccttgtcatcgtcagcctgaaatctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaggggggggggggtttacccgcaagcccccccagggggaaaagggcagtttcccccggaaatgttccggcaccggggttcccgggaggggttgggcaaaagggcggttcccattttggcccagcgtttttgcccggcacccacgccccccgggggagggggcgttgtgcgatgtccagcgcgagcgggccgtccccggtgggccccactgtggacgttgatgaaaccggttgctgttacgcccggccgcgttgtccgcgtagggcagcatgtacaggtcgagggtctgatcgacattgcgatagcgcgcggccatttgcgccagcatcgaaccatcgcctgccagcgtcttggcatccgctacgctggcgacacgcaccagttcgccggtcggtgccgtgccgctggccaatttctggcctaccagcagtacgacaggcaaatcactgctcaggcccgcctggctcccatcaatctcgatgtaagcgcccggatagcgcagcgccgaaggcacttcactgaaggtaatggtcacagcttttctcccgtgtaaatgagcctgtcaaatgcaggcgggtccggtaggtagttggtaatcaaggcgtcgaactcgtagcgatcgcgccagtacaaatcgccgtcggtgtactccaggacttggccaccgacgaacttgatggggcgcacgccaggctgaagctgccagcccagcagcagcgttttcacggcaatgcggtacttcagcaactcatcgtccgtatcgccttgcttgtgcgtgcggcggttttcaatggcaatcaccacatcgaaggccagcgtcacgttctcagcccgctcgcctgcgtgctgcaccttgtctgctgcccggacgatccacgcggccggcagcggcaacgcttcagggcgaacctgggcgaactcggcagcaccagctacctgccgaaaccagatgcctgcgaactcggcaggcttcaacttcacgtgttcaatcagtggcgtcagcgaaatcaccgcccgacctcatccacataggtgcggccaaagcggcgcggtgcgctttcgatgatggccagatcatcagaaggaaccggatcatcggatgcgggcgggacgaggctgatatcgccacgggcgtgcgccttgagcgtgtccagcacaccttcataggcgcggcgcacgtcgtcggtcatggcctccgcaccttgcaggtagtacagcgccacggtggatgccagccgcgccagcagcgtggtccgcacccgctcgggaatgctgtacgacagcagcagcgcatcggcgtctgccaatgcgcggtcgatagcatccagggcaagcgccaccgcctcttgctcgcgcggcgtgaggtcgcccaggtcgcctccctcaatggccgtgcgcagcgcgtcttccggcaccatgtccatgtcggcgggaaccgccaactgggctagccgcctagcgttgctgcgcgcaagcagatcagagcgggaggcgaagcccattacttgcccgccttcttggcggccgccttggtctcgggcgagtcgtcttgcgattgcgtggtcgagtcttcagcctcttcgggcatatctgccgggcgcgtttccaacacctccagcatctgctcttcgtccaggcgcttggcggttgctttgtccacttccactttttgccatgccttgctgaacttgaagccgcagcggtggaacgtctcgctgctcttcttcggttgcacgcgcacaaagagtgtcgtcacgttgattctcctaaatcggttcctgtacccaacccggcggaacaattcccgccggggacgcacctgctctcgctacggggtatgtgttgttacagccagggcgtaacgaccagttccaccttgttgtagttggtgttgctggagccgttggcgttctgcgcggccttcagcagcgcttcggcatcggccatgttgtccgggccgcagagcagcgtgtccggcacgatgcccagcttgcgctgaccgtcacccctgaacttcatcatggcctcgtaggccgcgttgaagttggcagcgttgagcgcagccttggagccgaacgcgcattgccagaagccgtaagcggcctcgccgcgccaacggccgccgaagctgtacacgtccaggtcgaagacgttggtgttctgcgtggaggtgatgctgttgaactccgcgggcatgcgctgttgcaggtagatcggagacgctgcgcgcttcgtgcacagcagcacccaaggtgcgcccgcaccatcctgcatgttgctcacgttggtgacggcaccggtgccgtcttcgttctcgtacaccgaatgatcggtgtcgaagaaatactggccgtcgtagcagacggaggcgaagccagcgcccagcgcttgaaacaccagatcgttcttcagatcggtggccgactggccggccgactcggcgatggtgccgtactggccgatttcgtcgtcttcgatgtccgtacgctgcacgtccaccgtgcactcgaacttgcggttgaccacctgataggcggtttccttgaacttcttgtgcaggcgtgcgccgacccattcccggaaggccgggaactggctcagccattcgtaggtgttggatttgccggtactggtgactagcttggcgatcttcttccagtcttccggcgtcagcaccaagccagcattccagcgggcaatcagcgtggtcttcagttggtccaactgggcttgcgtgagtgcacccatcgttttctcctgttgggttttgggatgattttgtcgtgtggactagcggcgcgagctaggccgcttttcagcgtttggcggcgacgtactgctcgggggtgacccccatcttcttgcacatcgcaagctcttcgtgggtcaggccatgggcaccttcgctcttgccatcggcctgcttgtccaggatggccagcggcttgctggcgtccaggtactctgtcagcgcggccagcgattgcttttcagcccacggcttctgcgccggggtcaggcggccatcggtcagcgcggctttcagcagatcgccgtgttgtttgcgctctgcggccagcgcggcttcggcttcttccttctcgatgccttccaccttggccttcagcgtgtcgcgctctgccgtaagggcagcaacgttggtcttcaggccatcgcgctcagtggtcagcgctgcgagctgcgatttcaggccgtcacgctcggtcgtgagcgcggcgatttcttgctgcgacatgtcgaactccccttctcgaaaattgaattgcttgcgggccagagcggccacggcttctagcccatccagggcgggtgtgttggtgagtgcgaccgaaaggatttcgagcacttcacctgtgacggggctgtagaagaagacagtgctgatgtagcggtacttcttctgcgcgatgagctgggcggcgtcgccgacccatgcgatgttggtggcgtagagccctttgccttcgcgccattcgagcgtgcggggaatccagccagccgcatcggcacgctggccattccgttccttgttcaggctctggtggtcgtagtcgaccaggatgtcgttcttctgctgctcggccagcgcgatcacgcgcgcggcgatggcggcgtcgagctgccaagccgcgcagtcgaacgggcgaccatccgttgcccggaacgggccgagcggcagcaaatgggcttcagtcggaacggcaccatcgctgccgggcaccaactcgaacgctagcgcggctattgcggtttgtgcgatggggggattctgcgtcacagcgatgctcctaatcgatgaggagcattcttgacttcagcgagactttggtcaggtagtgaaatgtttcatgcgcgcggtaatctgggttaccgtgtttgttatgctttcgcgaaacccttgataatccaggagatttgatagatatgccgactgcatcctgcacatttacgctcaacgggcaggcaaccagcacgctaacgtgcgacggccgcgtttatgtggctttctctggcaataaagggcatgagaacaagcccggcgataccggcatcgctggacaagggccattaccaaccgggcgttattacatcgtggaccgtgagagcggcgggcgcctggggccgatgatggactcgatccggtcagcggtcagcaactccaatcacgccgagtggttcgcgctctaccgcgatgatggcgcgattgacgactggacgtttgtgagtggcgtgcggcgcggtaatttccggctgcacccagttggcccgcgaggcattagcgagggctgcgtgacattggtcagcaaacagcaattcgcggctctgcgcacctacctcaagtctcgtcagaccggggttgtccccggcacgaacacgaagtattacggtgtgctggaggtcaaatagccatgcggtttgccctcaagaccgtcgctgtcgtgttactggcgttcgcatatacgcgggccatttatctctggtcttggcttgacccattctttgccagttcggcatggcagcgaatctatggcttcctcgcgcacctgtttggcgtgagcggtgcggagcaaggcgaagacttgcagctcgccatgttccttgtggtggcactgctgctggcgatcttgtcagtttggctgttcgaccgctacgtgtgtcggccggcctgccacacattagcgcggcagagaaccgcgaaaaggccctaacgatgccgcctccgggcggcattcttgtatgtatggggcggaattcaggcgaggtctggtgggcgaccgtctaaaacgcgctcagagccgttttaggctgctgatggaccgtgggaggtccacgccccggaaaggcgtttttaaacggctttcgaacgcgaatggaggcatcccgcaaggcggcgctccccaaaggggcaccgtgcggggtcagaagaggtctagctgtcgatcgtcgtcctcacggcagatgttgacaatctgacgggacgaaaggtgatggcgccgagccaaagcgttgatgctggcgttgtgccgatctttgcgaatctgcgcgtcacgcacgcggataaacagcttgtcggccttgggcagccaacaacggccatccgaatccagatgcggagccagcgtcgtccgcaaacgggccagttcgtcggcctccaagcccagtgcgtgagtacggtactgcgggatgctgacattgacgccaccatggtgggtcagccagtcccgcgcccgaccgtaaccaagcgcgcggaccaccgcacgcagcacgggtggcaaggttctcagcaaatcttcatcgacagccgggtaggcgtctaccacgcgctcgatttccgacgcggcatgcgcagcgggatggtgatagtgcggaatgaccatggcttacccgcgcgccagccaactcttgagagcttcggcaatcgtttggcattcagctacggccaagctgtccaagtccggcacgtgacggcccacttgacgcgcacagaactcaagcagcgccgggcgcgttgccttagccaccttgcccgcctgcccgagcttgaaccacatgcgcacgaggtgagcgatgcggggtgggacttccttggtcacgccagcctgacggaacacgcgccgatagcgcgaccacccccgacgctcgtagtcgtccagcacagcgccgagctgcgcgaggttgagggtactggcggacacgcggccgtcaaccacggctgcgccatggcgtgtaagcaggtcgcggtgggtctcttcagaccagccaggcaggctgaccatggcccagcctttggcgatgccgacaagctgccgatgatgcttgatgaggaacgtagacattttcatgcccccagcatttggggtgcatccacccctgttccaaccccacgatggagggtggcgccgcgcccagcgcgaacgccttgcacgcagtcgcccacctgggaaacatggcgcccaggttcgcggcaccgcgtctcaacttgctcggtcacggcatagttcgcgcgcatgtaggcatccaccgcgctgcggtgggcttcgggcggcacggtggcacccaccagctcgcgcacggcaaacacccatccttcgcagaacatgtctgcgcgggccaccttgttcttggagcggcagcgcttgagcgcggtcttgatgtagtcggcccgcgcgcggcgaagctggcggaacagaacccgatagctgtaaccggcgaccgtcgaagcgctggccataccgataaatacgtagccgccttggatggtggttcgaccggacaggcggttggaaaacaccagctcgcaaccaaaggcggatgcgatcatgctggccagccacacctcatagcgggccggcgtccgggtcgcaccgctcttcacccacagctcggccacgccggccgccagcatgtcgtcttcgctgacgccataggcggtcatcatcttctgcgcctgacgcatggcggcagcggcctcatgcgggttgctggattgcccaagcgccaaacacttcttgatcttgtcgattgcggtttcgcggtccataactcccctctggctgctcatcagtgtccggccaccacgccgggcagacacccgcttgcgcgggcgtttcgcgggtgggccggctaggccgctgctgcatccttcagggccttggcagccgagaacttcggcgcgcgcttggccgcaatggcaataacgtcgccagtcttcgggttgcggccggtcttggcggcacgctccacggagccgaacttgcccaggtcggtgatcgtcagctcgttaccggcgcgcacggtgtcaagaatggtttcggtcagggcgttgagcaggtcttcagccttgcccttggttacttcggccttgtcggccagatgcttgatgagttcttgcttggtcattacaaaagttctcctatggtttgccgcgatacgctcgcggcgggcgttactgaaatcgaaccgccagggcgttacagagcggcaaagtccagggtgacggctgtgggcttcttggcaccctcggcgcgctcgtagaagcggatgtaggccgtggtgcccaccacatccaccgactcgccgatggcctgcatggcgcacgtccacttgtcgtcgctgatggccaggcggcgcagcgcgagcacgcggccggtgttgatctttccctccttgtccgtttcgaaggcttgctgcaccagcacctgaatcttcgggtcgctgcccttggcccattccttgatgcactcgtcgatgagcaccttggcggcttgcaggcgttcatcgaaagcgatgttctcggcctgggacacgactacctgctggcgaccgtcgaacgtgctcaaggtgacgttgccctttgcaccgcccagcttcacttcgtagcgctcggccgagagcgatacaaatgcgtcggtgtcgttaaaggcttggcgtttgaaatcggccatggctgcttgcagtgccttggctttggccaccagctcgcgcaccagcttgtcgcgctggaggtcgatggggtggatggtcgattcgggcaccaagtggccccgcgcgttggcgcgatagccagccggaatctcggtggggtgttgcgttactgtggtcattggcgtcctttcttcaggttgttgagggcttcgcgcacttcttgaggcatctcggttttcggcagttgctcgcttaccgctgccggccctacggtggtcgtggccgcttggcggcgggcttccggtgtgccagccccagcaatgccgcgccgctgcgcctcttggtcggcttccgcttgactcgctgcgcgggcggcctgccccgcgattacagagcgcagccacccgtgcgacttcagggggagttctaggcggccgtggttcgcttggtccactgcgtagctgaaagccgacgcccacagcgccatgggcgccgcgtacacggcaccgtcgcgcttaacctgggccgctcgcatctccggctccagctcgcggatgaggcttgccatgcgtccgttggccatctgcgtcttcttcggtccaaataggccgatgtagcgcaggagcggcttgatgaacgagtcgcctgccgggtgcacgtccaccagcgccagcagcatgtcgcgcacgccgtcatcggctagcaccacatccaggctcatccgcacgcggcagttcgggcagacgacttcaggcagcggcattgccacctccaaccagatcggggttcgtttcgcgcagacggcggtacagcacgcggcggcgcccggcgtacttcgcttgaaggcgcgtgaggttgtcccgagcacgggccatttcttcctgccgccactccacatcgcgcaccgcgtggatctcttcgcggtcgatggcgcgaagctggcgctgcacgcccacccgcacgcgcaaccggcgccactccgcgcagattcggcgcaagcccgccagcagttcggctgcaacaggtccctgggtgatgcctgctagctgtttcatgtcttcaccttccatttcacttcgtcggggatttggcatgtgccttcgatccaggcgccaaacgcggctgcctctgctgaaggtgcgccgttggcgatggcatccaagaagcgcacggccgctggaccggtggcatagatgcgcttccggcgccgtgtatgggtaatcgtgatgcccggtgggtcaaactgttccgccatggctgcctccgctcaggccagcgattgcaggcacagcgcctgtttggccaccgcatccaccagcttcacgtccagcggacgcccacggcggaactccttgatgccagcgaccaacccctccaccagcatccgcgcgctgcccttgcagtattggtaaaggcgttgcaccacctcttcaggtacgtcttccgtgccgaagccggcttgcaccagggcggcggcgtcttccaggttgatggcgcgcaccgtctcgggccagaagcccgtgcgcgagcggatttggtcaaactggccatgcagcggcttgatgaggcccgacaggtgttcggtgccgcagagcacgatgccgacgttggctagatcacggaggcggcgcagcgtgtggagctggtgcggcgtgagcgtttcggcctcatccacgatgagcagactgtcggtgttgcgaagcgccgtcaccacggagcggaacttgtcatcaatgctgcccttgccgtcatagccggccaccacgcgggccagcagacgcacaaggctctgcggcgtcatggtgggcgtggcctcaatcaggtgcgtgttcggatgcgatgccacgtattgcttgatggcaaaggtcttgcccgtgcccacgtaaccggtgaacaccgcgaagttgcgatagcggcgggccatgctgcacgcggcgtgcgcgagcttgaagacgctggtttcgaccggggccaccacatcggccttcgtctcttcagcgtggcgcatggccgagtccaccgacgccagcagcttgcccggactggtggcatagctgcctttgagaatctggttcaagctgcttgcgctgatgcgcgccagccgcgccagagcggcttgggtgtaaccacgctcttccatccaggggccaatgcggccgataagcgcaacgtctgcggcggcgtaatgggcgggccaggtcgtttgcttgtccatggggttctcctgttattcgtcgttcatcgtcaggtcgagcagcagcggcgcgtcgtcgctgtcgtccaccaggcgggcttccacctcgattgcaggcagcacgccgtcggccacgctgtccacatcgagcaccatgcccgcacgggccttctgctcgtccatcttttgttgcaggcgcttgatggcatcggcggcgcgcgcctggcgcttttcttccaggcggttgggcgcgatggcgtcaatggccgtgacgaggtgtgcgtcgcaaatccaccggccttcctgcgtgcgcatgacggcgacacggtcatccatcaggtcgtattccatgaccaccctgtggccgttgaaggcgtgcagctcggggtgcgtgtaggcacgcttgccgtgcttgatggctgcgcgattcaccgtcagcaccaccgcctggcgcttcagttcggtcacgttggcgtgcggcggaagcggtgccagttgcgaccacagcgcggcgcgtgtcgtgttctggtcttctgggtgcgggcggttggcgtagcggtccacccaggcgtgaaggcgtcggcaaactggggcaatgtgggcagcacaagccggcccgccttgacctcgcgcacggtgcggttcagcacttccggcgccatgtcctcgccgcagtagaaagccggctgccacagcttcaggaagtcgtctttgacggtgcggaagaagcgctccacccagcccttgccgtgcgggttacctgggatggcgtggatgatttgctgcacgccagcgcgggcgtagaaacctgtcatctcgtcgctcatcagcttgttcttgtagccggacccgttgtcgatgtagagcatcggcggcacgtggttccagcgggcgaagcactctgcccacatgttctgcacggcatacgtgccttcatgttcgtcggcccgccatcccacggggaagcggctgcgcatgtcgattgccaccgtcagctccgggcgccagatatcgcccgtgacggggtgcgccaggtacacgtccgcgcggtagccgtcggccacatacacatcgccgggcaaggcgttttcggtcgagcggcggatgtatgccttctcggtcaggcggtacaggttgcggccaatgcgtgcggggctgttgcggccgagcatggcaggaacccccgtcaggtagttgcgtacttggtcgtagctgaccgcgaagccgtccacttccgccaggcggcgatgtacagcggccatatctggtttgccgggcgcgttgaaatactccagcgccggcccccaccagccggcggcctccaccacgcggcccttgtggtcgggcagcagcgcagttgcgccgccttcgcgatactgagcgcaccattcgcagatggcggagcgcgatggcgtcttccggcctgccttggcggcgccagccagcgccatggcgaagtggctgggcaggctgccggcctcgccgcgctccagcagcagcgccaccgcgttgttctgcgtcacaccgtcttccgtcatggcgcgcacgtacgccaccaccgtctcacgccacgtcgccacttgccgggccctgtcggttgcctcgcgccacgggtcgcgagcacgaagcgccagcacctttgctgtgggcatgaccgctaccgcgccacttgcgccgcctgcgtccacgcgctgaaccagttgtgcccgtgccatgacttactcccccttagccttgttggtggaacccttgggacggccggcgccgcgcggtttggctgcatcacgcttctcttgccggtttgctgcttcggcggcgtggcggttttcgatcatcgggtagtcgatcagccattggcgggcctcgtcagggctgaggatgtgggtgccttggatgcgttcgggcatgtcgtcttcgcgcacgaggctggccatgtggtcgatcaaatccaggccgcgcgaagccatgacgtgtgccgtcacccaaatctgttccatctggaggcgccattcgggcgactcgtcgccgttgacctcatcgaacagctttttcaggctggaagtgttgagttcaacgccaagttgcagggccatgcactcttcgcggatttcctctgtgcgcaagaggaattcggtggtgcgatttacttgcgacaggcgtttgacctgcgagtgcgcacgctccagctcagcttcgtagttcttttcacgccgcttcagcgcgcgaatctcttccttgagttcggccaccgtcatcaggtcggctttgccgatcatgtcgttgccggactcggcggcttggtcgatgacttcctgcggcagcgaggccaagagcatcaccttgaccttgccgagcgccagcacgccttccagttcgtcttgcggcagttgagccacaaacttcgcgatgcgcatggcttcgtacacgcgctggtttgcaagtccatgcgcgttgatccaggtcagaaagccgtccttttcaactcccgaacgttcgggagtcgatctgcgatccccaccatgcccaagcgaaatcaactcttgggcgcgcagaaatgccatcccagcacgcgtaatttcgaacatggcacggccgatagagtcagcgccgatgcgggccagctcgccgaggtcatcggtggtcacgttgaaatgcgacgcgactgcggcgagattcgccactggtgcgagcgcgatgggcagcaccgccgcagcggttgcaatatccactggctgcgcgtcgtccgtctgcgtggcgtgaactagagcgccttccgtgatgcccaggtcgatatcttcggttgcttgtacggctttggttttcttcgtcatggtcggcttcacgcagctttcttgagacccagtgccacggcgactttgtgagcctcgccgtaacgccccttgttcacgccgttgatgaccgcgataaccgtgcggtattcaaagcccttctcctttgcccactttgcaaaggtcttgccctcgcgctcaaagcgcgcctttacttgctccggggtcattgctacgctccatccgttacgattaagaatgactcattatgggaaagaaaactttcccagtcaatacattccagggaaattttttatgaccatcggagagcgtttgaaggaagagcgcgagcgattgcgcctcacccagccggccattgcctcggccgccgacacgacgaagcaaacacagcacgcatacgaaaccgaccgcacgccgccgaaagccagctatctggccgcaatagcgatcctgggcgttgatgtggcttatgtgataacgggggagcgggcgcttaatacggcgcgcacgccgatggaagttgcactgctggagaactaccggcacagcccagcagaggtgcagcggggggttagcatgcttctcgcacaaacctccggagccgcagattcagtcgccggaaagggaaagaaaaatcgatcctcggaggaagtgaagaagtgaagcgcacaatgctcattcttgcgatagcaactattgctagcgcaccagccatggctaaaggaccgatttcgccagaggtcttcagagacagctataacgcgcatgcgaaagacctacttcccgcgttgcagacaacacagtgcaggaccatcagcgctgcgaaggagccgggcaaggcgatcttggaatgtcagaccgtggcaaacaacacgctgttgagcatcaacggcatgaacaagcgtttcgacggtgcttggctgatgctggacgcgagtgcattgccacatccttccgacttgacgcgggcgggcgggttgttgctccgcgttgcgaagggctaccactacggcaaccatctggcccttgctcaggaggcgattattggtgcgcaaagaagcaaggggaagcaatcctgcattgacgatcccgcatcaggtagccgcctctgcgtctcgaccgataacggcacgatctacaacatgatcttggagcaagccgagcccaagaagggctgatacatgcactaagtggatacccgcccccaggaaacgtttcacgcctaatcgcgctccgcgcgcgtgcgtaacctctggtcattccaatgacccggaggttacgcatgaaactccctcgccttaccacgtggatcatcgccaccattgcgctggcgatcatcatcggtctgctgtctccgcagcaactacccgtcagtctttacaagctctcgttggtcaccatggctgccgtggtggcgtactggctcgaccgcgcgctgttcccctatgcccggccggccggctacctgtccagtgcggactggcgcaaagacgggcctatgtgcgatgacgccgaccacgccattgttaccggctacgagctggtgtttgccgccgccatgcttcgccgcgccgtcatcgtggccggtgcgatgctcgccatcggtctgggggcgtaaccatgcgccgccagtcgatcctcttgagcgtgttcgccgcgctccagctcgcggcggcgctgctgctcgcatcggcagatgcattcggtgctgatgccgccatcccgcacgtcgcgagacgctaccgtgcagacctgacccgcattgcacgcgaggcatggggacttgacgcacccgtggctgtgttcgccgcacagctccaccaggaaagcggctggaaccccgcagcggtctcgcgtgttggtgcgcaaggtatggcgcagttcatgcccgccacggcgaaatggtggtgtgtgctgaatggccttgccaacgatgcgtgccagcccaccaaccctgtgtgggctatgcgtgcattggtgggctatgaccgctggctgtatgagcgcgtgcgcggtgcatccgagtttgaccggctgtgggctgcactacgtgcgtacaacggcggactgggccactggcaacaggaagcggccacggtgcggcctgcgctgaaccgcgcaaccatcgatagcgcatgtggcacagcgcgccgccatcgcagcttctgccctgagaacctgggctatccgcatcgcatcttgaacgtcttgcagccgcgctatctggcgtgggggcgtggggtacggacatgacccgcacgctgattgcaacggcggtagccgtagcgtgcggtgcccttagcgcgtggagctacacacgcaatcactacgtggccgagattgctggcatgacggccgatcaagccacggcccgagagaaagccgagaagatcgcgcgtgagctgctcgaagccgagcaaaagcgcggcaacgcgctgtcggacacgttggccaagaaagaaaccgcgatcaccgaaaagacccaggagcttgccaatgcactatctcgcctcactactggccgcaagtgtctcgatgctcgcgttgtgcgcgtgctcaacgactccagcaccggcaccgctactgacaacgtgcgcgccaccaccggcacatctgatgcagcggatggacccgctgccaccgataccgacgtcgctagctggatcaaccacgccaaaggccaatacgaaatctgccgggctcgactcggcgcactgatcgactttgaaaaagggcgagttcaa

SEQ ID NO: 10 - DNA, 8078 bp, Ralstonia solanacearum
  misc_feature (5821)..(5835): n is a, c, g, or t
atgtctcgcttgggaatcgagtatcagaacggtctgatttatgagggcagggacaacccgagctccttagccgttccgacgccaatcatctcgcagtgcacactcattgagtccccctctgatcttgagaggctgcctcgtgggatagactcggatccgtttcgctggattttcagggaggactcgttcgaccccgtttccagggttcgacggggccgactatttcagcctttcagtaactcgaacaaggagttggtctccgttgacgcgcatccctttctgccttcggatttgggccggcgtggcgtcgatggtcaattgcgcaaggaaatgaccgtgttcattcactgcacgcaacttgtggtgcgccgggagcgcggggaaggtctgcagcttgcaatcggccacgaaggggcacactcgctctggcgcattctgcagactgagcaaacggttacccaggacgtgctggtcacccttcgagcggagtctgtattcggggtactgccacccctcgatctggcgcaaatcccagaggacggccgaacggcagtagctgcggcctacgaccgggtcatgcatgttgcatatcgagactccccgacatcggttgtggaccaatgcaggaacctctgcgcagttctcatggctcgctggttgcgccagcggacgagcaacgataggttgcttcacgacgatctcggcgcgtgtatatcggcagtcaagaagttcgccgaaggagagcaccaactcgtccgcgccgcacttgagacagtgaatcgtctccatccacgagggaaggagaacgagcgtgagcggtacagtttgcgggaggtgtcaaatgaggacgcagagcttgcattgcacgcaaccgggttcgtgattagggagcttggctgggggcgctgatgaaccccacctttcatttcttaggagttcctttttttgtggggcgcgtgaacgctttgtgagaattcaccccgcatcgccgctctagaatgccccgccgtattggagcaagcgagtgggtcggtagcgccacccgccgccggtcggcgtatagaacaggtacaggtggggtagttgccgtgaaggttttcaatgcccggcattttctgaggcatatcgctgccagcgtgctgcgcgagttcacgcaagcgcatgcgctggccacgcgtttggtggtggactggtccggaccagccgacatgttgtcgggactgctctgcgatgcggttgatgcattggagcagcaggttgctgccgccgaccttcccgaacgcgaccgcgaagcgctggagcacgacctgctgctgtggactgacgacctgcggcgtgcttgtctgatggccaacagccttgctgtagccgagttccggagcgcatgccaggacgatccagatgccctgggggcgttcgcctcccgtgacgagcgagagatcgcactgtggatgctggtcttccgcgacaagatcttccgcgacgtcgaactgcatctggccttccaatccaagacccacggcaagttttggaagaggcaccgcatccagcccggcctcgaactgacgcgcgagcgggtccggctcgaacagttctgccatgccgtggcgcagctttacaagaagtccggcggcggcgatggtgtgcacatcgaactctccgagcgacgcagcacggcaggcctggtggatgccatgtccagtttccagctgaccctctatgtcgagggaccagtgacggcgctcacgcacttcacgcagagccatttcactcgcgtcaccacccgcgtggcgctggagtccgcgttggtctaccacccagcgaccggcgaagtggaaacagtcgtcaagggtggcgccaagaaccatggtgcgatgctggagttgttcggcgagcatgtcgtccagcaggatctggcccccgagcggattgagccgcaacgctaccacctcaacgccttgcgcgatgacctgcagccctatgaggattggtcgatccacggcgtggatagggtccgtctgcgccgcgcacgcctagcgccggcggcgggatcaggtgtcagtttcactgtcgaagcctcgcccgacaaggaccaggacgacgccatccggatcgcccgcaaggcgctcaaggtcgagcatatgtttgaggcggaatatcacctcgaggctgcgaccgtcatcgtatacacgcaggccgccgacggcgggcgggcgggccatttcagcttcaatatccgcgcctcgggagcctcgaccatcaagaacctgtcgctgaggaaccaggtgctcgcgcgcaaggtcctgcaggcgctgatggtgatcgacgccgaggacgatctcatcgcgaccatgtccattcccagggaggctatcgcagcgtgagtcaggctcaggttgacgctaccgcgctgctctgccgattgctggagcgcgacaaacccgaaatcaacggccaggccctgttcgatggggacccgcaggcagcggctcacctgctgcgtgagcggttgctggtggtcggccgcccgctcgaatgggtcacatgcccggactgccgtgtggaaatcgcgcgggtcgtgcgtgatgtctcggccgacaggatcgccctgcgttgcccggaatgcgaggacatcgacgcctcgcgtcgtctgcgggaaacgtacacggcggtgccggctcgggccgtcgctgccttactgagcgggctcgggatgacagccggtggtatgaaggtcatcgaacccgaccgcgtctggcggctgggcacgacggagccgacgcgagggaagccgctgacttggtacttcgcacgccagcttgggcgaccggagatcggcgcccggctgcgcgagcagatccagctcgagcgcacggccggttcctgcgtggcgctgaccaccagcgagttgccgttgccgattggctcgccgctggtgggcttcgatgtgcgcatgctgtccagcgcggcgcggataagtcagagccgcttcgaattcttcgcggaccggcaggctgcgccgggggcgcagcaggtgggggaggcagagccgcggttgaccgcgcagaccaccctgcgctacgtgcgttcgctgggcaaagccttcatcgaaggaacggaatatcctctggagccgcgccagcaggccatgctgcttgctttgctcaacgatctcgatcacgagatgggtaaggccgcgctcaagatcgcctgcggctcgcaggcgcaacggttctcgccgagcaaggagttcgaccgcaacccggtcgtctacaagaccttcattcgctacctgcgcgacgacgaacgctacgcgcttatcgttcctgacgaggatcgggactggttggggtaggtgccgcaatacacccattcgccggcccagccggccaaatgcccgatactcatgtgctcgacgctattataagagcgggggcatgtcatcggcgcgaacgaggatttcgagcaaatcgaccctgcaggtttgacagccggtgcttgcgtttagaacgtggttagggctgcagaagcttccggagctcaggcaagggaccgtttctgtcaaaaatttgcgaagcaagaaaggcttcgtcaagggcgccaagtgagcccgaaggggaccgacgcaactgagcaaggacaagcttcaaagcggcaatggggaacgcgagcatcgcgtggtctgggtcaaaagtgtaaaggtgtattttgtcgagaacataggcttggaccaactgccgcactccgacctgttctacgaaagcaaaatgagcgggggctgtaggctcacagaacttcgggattttgtagttggctgcctctcgcttatccaccagccagtcgaacggtttatcggccccaatatcctgtgaggcgaggacgctgctcgtgtaatgatgggagaacgtgtctaacacgactttgtgcgtagtgcccgcacgctttgtcggagtagcgccggcggttgcccgccacgaatagggcgtagttcttgaatagaagatcccgacgccaccgagggcaagcgacgcccgcgtcaaatagaaggcgctgtaatagagcttcacggtcgcccacgtgtacaacttcctgtctagcccctgtattgcttcagcaatggacaaggttccggagtaaattgcatcgcgtgcgtcctcattcaaggaggcagcaagctggacagcctccctggcttggagctgcctggaaactatggcagccccggtaatcccagatagaatggagtccgcaacgcgttgcgcacgatgcataagattagaatttgaacttcgttgatgccttgactttggtggggctgaaggcctcttgcagtcgtgcgtatatcttcttctgcgccccgctaccaccctgccccttaacttccgcctggatgataagtgactcgttgaacttcagagctgcctgaattgttttcacctcaaatgaggcctgattcttacagaagggaatcaattgaagcctgaggaaatccattgcggcaagaaatcctgatgcgtagagaaagacgttctctttgtcttcccaacgatcgccatagatgccctggagcgcgatgaaccagttgagaatgatctgcttttgcaactctagggaaccgctgatcaatgaagtcggcgaatggaatcgcgagtcatgccatcttggtttttcggattgaggaagggatgcttcgattcagcacggtttttcgctgctttcgcacctatcttggcgcgctttgcgcgctcaggacgcaatgctcccaggactggccagcaactgcctcctggcgagcaccagattggccaaaccaaacaggctgaacagttgcgccgtgttcttggccaagcccttgtaacgaaccttgcgatgaccaaacagattcttgatgacatggaacggatgttcgacccgagcccgaatctgtgccttggttcgctccaccgcgatcagcaagtccttgattgggccttcgcgcatcgctttgatcttgccgcgcttgatcgcgacatgccacgtcgctcgcttgccttgcatcgcgtcgcgcttctcaacgccggtgtagcccgcatcgccaaacgcatccgtctcgtggccatgcagcagcgcgtgggcctgcgacacatcggacgcattggctgccgtgccaaccacgctgtgcaccaagcccgacgacgcatcgactccgatatgcgccttcattccgaagtgccactcgttgcctttcttggtctgatgcatctccgggtcgcggctcttgtcggcgttcttcgtcgacggcggcgcttcaatgatggtggcatccacgatcgtgccttccttcatcatcagcccccgctcgcacagcatgatgccgatctcgtcgaacagctttcgcgtcaagtcgtgttcgttgagcaggcgtcggaacttcaacagcgtggtcgcatccggcacgtcctcgaccgccagatcgatgccggcgaaggctcgcatcgccatactgtcgtgcagcgcgtcttccagaccttcgtccgacagcccgtaccactgctgcaggaagtagatccgcagcatcctctccagaccaatcggcgggcgacctcgctcgcctctcggatagtggggcccaatcgccgacagcaagcgctgccacggcacgaccttctccatctctgccaggaagcgctggcgtttggtcacgcgcttcttacctgcgctttccgcttctgcaaagctcatctgccgcttcatcactatggctccgttccgtgagctacctgctacaacggcctcagctgcgtcagcgatgacggctgagactgataaatcagcgtgtccnnnnnnnnnnnnnnntcagcgtttccctaggatgntngagatcacgnngaanncggggggtgttcatcacgaggnntnctcctngtttcctccgtttacttccgtgcgattcaagggcgaaaatttggctttatcaggacgcaatgctcccaggactggccagcaactgcctcctggcgagcaccagattggccaaaccaaacaggctgaacagttgcgccgtgttcttggccaagcccttgtaacgaaccttgcgatgaccaaacagattcttgatgacatggaacggatgttcgacccgagcccgaatctgtgccttggttcgctccaccgcgatcagcaagtccttgattgggccttcgcgcatcgctttgatcttgccgcgcttgatcgcgacatgccacgtcgctcgcttgccttgcatcgcgtcgcgcttctcaacgccggtgtagcccgcatcgccaaacgcatccgtctcgtggccatgcagcagcgcgtgggcctgcgacacatcggacgcattggctgccgtgccaaccacgctgtgcaccaagcccgacgacgcatcgactccgatatgcgccttcattccgaagtgccactcgttgcctttcttggtctgatgcatctccgggtcgcggctcttgtcggcgttcttcgtcgacggcggcgcttcaatgatggtggcatccacgatcgtgccttccttcatcatcagcccccgctcgcacagcatgatgccgatctcgtcgaacagctttcgcgtcaagtcgtgttcgttgagcaggcgtcggaacttcaacagcgtggtcgcatccggcacgtcctcgaccgccagatcgatgccggcgaaggctcgcatcgccatactgtcgtgcagcgcgtcttccagaccttcgtccgacagcccgtaccactgctgcaggaagtagatccgcagcatcctctccagaccaatcggcgggcgacctcgctcgcctctcggatagtggggcccaatcgccgacagcaagcgctgccacggcacgaccttctccatctctgccaggaagcgctggcgtttggtcacgcgcttcttacctgcgctttccgcttctgcaaagctcatctgccgcttcatcactatggctccgttccgtgagctacctgctacaacggcctcagctgcgtcagcgatgacggctgagactgataaatcagcgtgtccgcgccccgacaagctgcatgccgacaaggcttacgactgccgccgatgtcggcagtatcttaagcggcacggcataagggcccggatcgcccgcaagggcatcgagagcagggagcgcctgggccgttaccggtgggtggttgagcgcacgcacgcttggtttgccggctttggaaagatacgcgttcgtttcgaacgacggctcgatatccattgcgcgctcctctcgcttgctgccgccatcatctgcgctcgattcgtggatgacttgtgttagcgactctaagtacgtttgtaccggctctagtggaaagattgtgtggggcggtggcggattggaggggcgctcagagtttgggccgaataagtcggattttatggcggcgctttatttggatgagcaaggcaatttgattgtggaggatcgtattcaggttcacatgagcttgcgacttctgcatattccaacggggacggcgcagcattacacggtttacagatttaggcgattgaaataagaatctgttatggaaccacgcgaaagtagagggccgcgcagaatttatttgcgctcaatgtgaatcgtgagtgtttttattcaccccacaaggccagaacgcagagtcgagccaggcttttgctgggcagcgttgcttttcgggagcatttgggcttactcggaaggattgatcgttcatggcggacgactggctgccgccgatggggctgtcgggttgttgtacctctacggcgagcaaaccggtcatccatcagcggctggtggcgctctactgctgtttctcgccaagaacgtttattgcgccgtgcgaggcaggttttggctgcagtgcttgctgctcagtcagggctatcgcgtggtcagtcagtagaagcccgagcatcgggccactgatatgcaggcggcatgagcgacgtgccaccgctacaagtacacatcaagcagtttttggggaagtttctgtgcaatcaccacaccgcccggtccaactggccgtactcctcggcctatgcgccgtcttcatcccaccag

SEQ ID NO: 11 - DNA, 7663 bp, Ralstonia solanacearum
  misc_feature (2532)..(2681): n is a, c, g, or t
tttttattcttctccattattatttttattgtgagtttgtctccctgtattaattctattaaatcaattaatcattgcaataccaaaatcaacttacaaagatatccaatgttgtcaagcaaaaaatggataaataaaaaaagcacccttgttgggtgctttcttgacattcatggtgttatttgttaattaataggcaaataaggagaattcaatatgaatgtgagctaatggaaagggatgacctggtgtccaaaattcacagtcaaagatttaagatcatgcagcactacatgtttttgtacgccgatagattggagggtaagccgtgccgtttatgacagatacaacgagggcaagttgattgggtcaagagaaggtgatgcatgccacaattcattgcttgtgatacaccaggaggaattgaagctcggttcaatgttggatcgcttggaggatttatcgaaagagcttcaatctcattgaagaaaagtagaattagaaaaggcaccgaaaggtgcctttgttgtatgctctggtgcaaaaaagttccgccgccaatttttcttcatcttctttgctttggtccttcggacattcatactgaaggatgaaccctatcgggatcatgatttttgatgaagaagtccttcggaaacctctttgctttatcaacccataaatgggtatatatctgatatacacctacacataccatagtaggtaaaatcaactaagtacatcatcaagaatatgaattaatagtacatctgcaaagaagcgctatttgcgaaagcaaataaaattccctgagcgaaaaatcaggcacgttttttatacaacccaagctggctatccacttcattccaatactttttcacagaaggaatggagacatcagcacgaatggcaacagactgctgggtgactggtgttcccgctgctttcaatgcaatggcagcttcaaggatggcttccttggtgttctttgtcctagtcacagcactgaagccagctccagcagcttgcttggccttcatggacatctctggtggaagctccatgacaccacggttcttctggttgcctatggtgtccttgtgtctccatgtccatgtgcctacagatctcgcagtggaaagcacttccttggctggtagaacgccgtttaagcactctaggaaggtctggttgatagacagtgcttcagagtccgcagcctgctggaaatcagcataggagccgcacttcctgacctcgatgtaggcccagtgtctcagggtgtcaaagagggtggtgtttcggccctctagagagtccaagagcttgcgtttcttcttgtagcctaccaggtcaaggccccaatccttgaagtcttccaagtcatatgtggcatggtggcagatgacacgccagtgggggtgcagcgggttcttgatgaagtggcctacaaagccaaggtcagcccctagcatgcaggtcagagcattgtctgtagcctcaaagaacttctgtggtccgcggcgtgcatgctcggtgtagtagaccggggtcttcagttcataaaggtagtggcacttggcattctcaggattgaccatcacataggtgggtggtggcaacccaaggtggtaccaaagatcaaaagagcctgggatgtctatgtccagggctatgaacttcttgtagagctggggaagctctacatagagatgctgcaaagctttctctttcttcctgaacttgcaaccatattccaaatcatttgagcttcttaccttgtctggtagtgcctctataaaactgttgaacataaaaaattccttgtatttgtgcgaaataaaggaacttcttgttattagtcttggcaatttagattaaatacgttttaatatcaaatatcaggtcaagccgacacagcttcttctaaagttcctgaatttctaaatgcctctagcttccaactgggggcatttttcatttcttctccttcttttacgaaataaataattaaaagtcaaatcctatagatgctattgactttaaattgatttatattattcttgtactgttgcctggcttcttgttgttgattattgttgttgagtcttggcaatggtttgtatttatataaatcatgatcatccttttgcaaatcagaacaaggcgctcttcggagtgcttttttctttgtgtgcttgacttctgcactcaattttgtaatttagataaatgcaaaaggagtgctatgaagaagtatggagttggagttgtcttggctggtttaatgatctgtggtaatgcgcaagaacccaagtatgggttcgtgaaagtagccgagtctgaagaaatggtcttgttcgtgaatgttgattcatataggtatcgttacctggcagacattcaaagcttcggggcggtggttcagattttcaggaagatgggtggtggctcgtcattcagaaaagaatatgtactagagcctgactgtcgtagaggttatggaaggctatacagccacggccttgatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcgaagattggtcaatccggcggcaagaaacacagaatgttgattcatataggtatcgttacctggcagacattcaaagcttcggggcggtggttcagattttcaggaagatgggtggtggctcgtcattcagaaaagaatatgtactagagcctgactgtcgtagaggttatggaaggctatacagccacggccttgatgacagaaatcttcaatcatatgagtggctgaaggatagtggaaccgttgcaagtattcatgcagaggttctttgccatgtgtcttcttgggataagccgaagaaataaaaagcaccccgtagggtgcttttatctttgctggtctatcttcttcggcctgtcccgtagctgcctggtagcatgaacctagaatccatcaggtcatgacttacctttgcttcagatttcttcttagcaggtttaggctgagtgcttttgacggcaacaggtttgctcaaggcttgcttcgctgcctttgctacctgttctggtgtggccttcttgaactttcggggcttcgtatcagtgacaatgaagttgagcttgggacgtagagaggcaatgaacccgctggcatgagtgctgccgtccagcagagtcaggattttctccagatagccgaagtcatggtgtttggcagcatgttccgtggccatctgaaccgcaaggttgtaaatgcccgactggcacaacttcttcagatcaatgaaggcactgatcttcgggtcactgatagtgcccaaaaacttccgcagttctgcaatctgcttctcatccatccactgttccccctcaatcgttatcgttcttctatgctctgggcatcagcgtcataccgatagttgacgatgtctccgttgatcagcatgttggcagccgcctcaatgatgtgcagaggggcaatgaaccactcacggggtgaaaaccgtttgccctgctggtcatagacatccacgttgaggcatgccttgccaaagaaggtgtgcaggagcagttcaaatttctgtggattgaggttgtagcactcaaagacttctacgattttgaccttggccatcaggtaggtgggttcctcggccgcattcttgatgcgttcgggcactggcaccgtggagtagccaatcttgtagaggttcttggtgttggcaatctcaggcaccgtgctgagggactgaagcacatagaggtagcctgacttcttgtcctcggcttgaatcagttccagtgcctctagagctttctccgtggggtccaggatgcggcggcccgtttcgtccttgtatagctccgttgcaagggagcgaagcaacatgtcagactcggtgccgttctcaaagatacagcgcaaccgtgcgttgaccttgccgttcttgacttccttctcaccgacttcagccacataggcagtaatgccgtggaggatgaagaagtgaccaggctgaatttgctgctcaccagtgaagccacgtacctcacgaacgccggcggcaagctctgcatggcactgcttgaacaagggttcaaactgctcaaagtccttgcagcgtttgcgctgggcgatcttgtcgggcatgtccttcaccaaaggaacatgcttgagttggaagatgctggcagcttcgtcgtccagcaggcccagagagtcatccttgaagatgtcagctacagaggtgattggcttggctggctccacataggtgagcaacttgaactcatccacgtcctgcaaagcttcgcatttctccttgctggcacgaagccccttgagcctgttgaatagctggaactcaagaatgttgcccggattggactgcggctccttgccgtgctgacggtagaaatcgttgatctgctggaatgaagccagcagccgttcatccacactgatagcagcacgcttgggcttcacattgagcaagcccatttcatcatcttgcagaatctttagaaggtcgtccttctccacggtcagactccttgctgacggcgttgtgccttcagatagaccacagcatcagccatacgccgctccaacggatcgagcgaagcaatgttcggctccttgccgtgggtcttcacgaagtccttaattttgggccaaagcaggatggcttcctcgtcactcatctgcacacgagtagcttcaatggtgtcttgaatcaccttgaagattttcgtggtcagcgacttggacaggatttcaaacgccttctggaaggggttcacccggtcgattaggtcaatgtgcaggtcgtcaatgttgatgaacttgtcggccatgcgaacaaagcgtttgtcaccttcctgcttgatctcgccattcttgatgaccgaatccaccaccacgtgctgacgaacttcttcaacctcttcgtcactgagttcaggatacttgaccttgatgattttggggatcatgaccttgttaatcacttcgggatcaacattgccaggcagtgccttcacaatcttgtcgtcagacaggattgccgccttcaggtcattgaggtcagattcaatgatgtccttgactcgcttggaggtaggctccttcaagcctcgcaccttgatttcagtatcggtgctcttgtcgtcgtcactgaccttggtcttgaacttgaagttgggtgcaagcacctgctccatgagcaaggaacaggtgatggccttgagcatgttgttgaccgacagcttcaccaggtcgtcagtggcatcaggctgggcaatgagatttgtgaactgggcatggctcttgttgttgctatcgcgggtgcagcggccaatgatttggatgatctcagtcagcgatccgcggtagcctacggtcaaggcatgctcacagaatttccagtcaaagccttccttcgccatgcccaaggcaatgatgagatccatgtcttctggcttctcaatgcggcgtaggtattcaacgatacgttcgcgggctttgggctcgtcatgcacgagatcggcaaccttcaaatccttgccgtcaccatgccgcttgacgtgaatgacacaggtttcagggtcccggtgggtgacggtgccaatgatgtctagaatgcgatccacctcgtcatacttgtccttggtggattcaccagactgcacgcttggtatgtgcagaatgttcttctgatcggtgttcagaatttcatcgatggcactggtgtacttgccttggtagaagtggtagccaatgcccagagacttgaggaaggtatagccgtttaactgctcgtagtagttgtaggtgactttggtgaactgagcctcgtcttcaggcagcaggaccggcacgctgtcaccacggaagtaggagccagtcattgccacgatatgggcatccgacttcgccatgacggaacgtagtagctcaccaagtcggttctcaccatcagcagagacatggtggaactcgtcaatggcaagcagcacattatcaaagtccgattcctccaatgcctcaaaggcaaagcgaagagtggcgtgggtgcagatgagtatcttggctggacccttgatgaactccacgaaagcttgaaccttgctggagtcaccaccaggtgtgcagaggttgtttttgggctctacgatccagtcagaaaagaagccaaacttgctcagttccaccgtgtcaaaggaaccaccaatggaccgttcaggcacggcaatgattgccttggacagcccttggttgatgagcttgtccagtgcaatgaacatgagggcacgagacttaccagaagctggcggtgctttcagcagcaggtattgagcattgcgagcttcaaaggcacgctcctgcatctcacgcatgccatattggttagtgctggtgctgtgaccagtctggccgtaggtgacttcaacaatgttaggcatgctcggcttccatcattttttcgtatagtttgaagaggcattccaagcgttcggtatcgctgtagaacggcttaatctggtagcactgctcaaccaccacatccagttcgtggtgtgcctgcttcaagtcagcaggcatagtgtcagggtcatacaggtcctcgatgctcagttcaggatacgcttcacggatggcgacaatcgccatagccttctcagccactgcttgccgtttcttctcgctggcatcagggaaggggaaggtgttgtagcaaatctcagcagagtagcgaagtcgcgtttcaagctgcccccccaccgtgcgaacccagaggatgtgcagtttggaagcaatgatcccaaagagaacaggatcttgcttgtaaatgacatttgcagagtttgtgataactacattggcatcaaggtagccaataggcacatactctcttcgttccgaagaggttgcaggaacaatgatagagccgcccttgatttgaactcgctgggcaaacttatgagcaatcttggcatagccattggtggtcttggccttggacttcacccggaagtcatagcaggcatcaatgcgtgtcttgataggtgggattgagcgagcaagttcaagctgttcgtcctcgatccaaaggcactgacgtggattgccatcgatgaattcttgggtgccgtagagagttctgaatagtggcttggacttcggatactgctgaacgatggcatcgcgttcttcaggcttcagaatgaggttgccaccgtcacgagccatgcaaccgatcaccattgccgggaaccctgacagaacttcagaggtccgacgcacgaaaacatccttaccttcaatcaggtaaggattgatgttcttcacaatcttgtagctgtcggctccaaacagcagcttcggcttgtcagccgtgttcctcaccccaacaatcacacacgtaacaccagcattgttctttgcgaagttggaccacttgaatgaggtgtaggcaaagtcaatttcaagaccaggctggaagactctgcatgttagtagagcaggtggaaaagacg

SEQ ID NO: 12 - DNA, 1398 bp, Ralstonia solanacearum
agcgcgcaaagcgcgccaagataggtgcgaaagcagcgaaaaaccgtgctgaatcgaagcatcccttcctcaatccgaaaaaccaagatggcatgactcgcgattccattcgccgacttcattgatcagcggttccctagcttcatcatgagcatagatcggatcgcccacgattctgtctgtggcacttttacgctcgcccgactcacccttgataaggatgttcaacgacactgggcaaacctgcccgataggcagcttgacgtcaatcagaccctggcaggcgactgacattgttgccaaaaatgacgttgcgatgagcgcgtccggcgccttgaccttgttcatcacctcgtatatcacctcggcaatttctattggaaacgcggcgattggatatggaggtggcgcatgattgtaaccattgttcatctttcaatctcaaaaaagaggggcgccgaagcgccccatgatttattaaacctgttcgttgtgccttttcagcagttcgtcccatttcttccgaaatgttattggcgagtactttgcacctagtttttcatagtgcctacgaatcttcgccagactgacaccggcgttgaaacgctccagggctttcacgaagtgctcttccaggatgatctccttctgagccttgccctcagcatccaactcgtcccaaccatctgcagaagcgtcgagttggctcgtgttgctgccatgctcatcgcaagcattcgcctcttcgggcattctcccaatagctggagatatacccgacatcatctttttatcgccgtgcatgggcagtcctatttagagagaggccttcctcttcaggtagttggcggtcgcctcaccgagaacacgtgttgcgctaccaattctgaagggttttggaagatcgctacccagaacgtcaatccacgcagccccgagccaatactccagacacaaccaagaagttctatgggaaaacagcggacctgccgtgcagacccacacactgccccgcgccccgaatactgatcgccttgtcacacgatccgacgtaccggagccagtctatccttcaccccgatagtcggcacgcacagaacagccttacagggcacaggcctaacagcagaacggacccgtgactcatcccccgaggctgacgacctcatggccctcgcagagcacttcaggacctatcgccatcgaccttactggaatgcccacccagcgacgcgggagaagcgagcgacagaagaaaactacccatggctggatgatgggccgtttgatgggccagaaaaacaaaaaccccgtaagtccttgaacttacggggttttgtctttgcaactggcggagagggtccttttcgaactcctcccgtgcttagggcgacgggt

SEQ ID NO: 13 - DNA, 2525 bp, Ralstonia solanacearum
tggcggaactcctgtgatggtgaggaatggtgcaacgtcctgaacgttgcgcagtgatgccaagcggccggctcagccggtgcgatacgccgaccctgccggtaacgagcatctaccaagggttgcccgggtgcaaacagggcgcgtgccgggtcgttgccgcgtgtcggaaagtcggggaggaacgcacttcttgcgtagcgcttgaggggctccggccgcgcaggtttggggtggcgggcggtgcccttggttaaggcaggggccgcgaacttgcgagtgtttggggtctgggatgcttcggctgtcaggggcaatggcacccgagacgtctgtgcatttgttgggccgtttagccgtttaaacagcggcgttgagggttgagccagggatggttttcaaacctacgtatcctgcttgcaactcattgaattcattggaggcattggtgccggcaagcgttttggatcgggcgtctgcggcgaccattttatttaacataagataaacaggtcggccttctgaatggctcaacggcgcggctttggcggccgagggtgtcacaaaatttgtgacacctgcgaccaccaccaggaggagggctgccgtcgcactacctgccatcctcgtcgaaaaaacctcccacagagaccgttgtccgtgacgctcggcgcgatccatatcggctgccatgatcaccggcaaggggttttccagctcgagcagttgcgccaggcgcaggcacgcttcgttgtccatcttgcggtcgcccttgcggtattgcgtcaccgcgttgccggctacgcctagcagttcccctacttggttgtcagacgtcaagcccctttgagctcggagctggtcgaagtatttgccgagttgcattgcacctcctcttgcgtgctcacgctttgtgagtacactcacgatccgtgagcctcacggcttgtgagactgagcgaaatataaccggttaacccgggcgaggggtaaacgatgcagcgtcgagacatcaagcctgtggcgtcagctaccgcaggtacgcgtatgccggcctggctacgccaggtgctggagcagagggccgccgtgcgccgcgtgcgcgccgtcgagctggcataccgccatgcatcgggcggccgtgtggccgctccccgccgttccctttcccttgacgaggtgaatgcagcactttttgacgctgctgacgccgagtttttggcctcgcgcggcgaggctgctttttgtcaggcgggcgtggtagcaccgcctgacagtctcacggatgagactccgcctgattctggcgaggggttctgagcgcatggcctacgacaccgtccgtctgcgttccccgtaccttggcaacaccgttgttgagcgcatcaaacgtcagtgcctgctgcgctcgggcctggattgcgccagcggcgaaatcctctatgagttgttcacgggcgaactgcttggctcgtgggattcgcggatctcggtgatcccgaagaacgaggaatgggtggtcaacaagaacggccgccccgagctggtgccgtgcgagccgtacgtgctgattgaggcgtccgtgcacaagatcaagcacggccacaacgtgtacggcgggccgaccagtttccaccaggcgtgccgcgacttcgtgcatctggtcgaggatctgttggagaccgatcttccggccgccgactggtggaccgttcatcgcgtcgacgtggcgtcggtctatcggctctcgaaggctgcgtgcaaggagttcttcgacgggctgcagctcatgagcttcccgcgtcggaagaagggcgcggccaagtattcgatggcggtgtacttcgccggcaagaccacgacggtcaagttctatcacaagggcaccgagttccaggtgcacgaccgtggtcgcctgcggggcttcttctcccaggttttcaagcattcgcacggacacgacgacgagcggagtcgcgtgttggcggaacgcaagatcgcagcactccagcgcctggcggacggccgcttgcgcgccgaggtggaggtgcacagcgacaagctccagtacgactttggcaagcatccgcgagtggatgaggtgacggacgcctatttggagggcgtctttgacaccgagatagagaagctcctgcgcgaggggaaacaggcgatggcgacggtacgagagagcagggcggtgctgcacaggttgaagggcgtctatggggacgcgctcggcatgcgcttgtacggcttctggggctcgctcaccacgttgggcgacgaggtgacgcgtgagcatttcgcgaaggcgacgttctatcgctgccgtaagcagttggaggacgcgggcgtgtcgtggcgcggcacggacgtgcacgtcatcgcaaacgatgccgtgcttccacacgatttcagtccggtacggacggacccacggttgtgccatctcccggcgcgcaaccgcgaggaattccaggtgagccgggaaacgttgcggcttgccgcgtaaggagaagtga

SEQ ID NO: 14 - DNA, 1277 bp, Ralstonia solanacearum
catcaggatccttggtcgggttcagatctctttgctctgccgttgcagctcgccccgagcctgccgtcaaaacaagacaggccagcacgactgccatctcaggtcgcatggttcacctcccagtcgatgcgctcgggtaagacgaagctgactactggcgatgcagggcccgatatcggcggatcgccggccagcgtcgccggaattttccttgccgcccatgcagctcaggggaccgggacggctggtccttcaagaccagttgccccttgaccagccaagcgccggcaaaagacatcacggcaagcgtttccggccaaaaaatggacgcctcgccccccaggagccagagcatggcaatccagccgaacgcgaatcccatcaggcacccacagatcacgtatacccaattgcgccgccgcttttgtggcgtggggttcgccggcgtattgacgcgaaaaccgaaggtcacaagatagaacgccagcagaaagaacgcccccacgaagtagaagtgatacccgagcggcccatgcggcccgtgctgcagggcgtcaatgagcctggcctcggcatcgggtgttgcggcctgctgcggggtgatcgcgcttggtatgatgcccatcgggaacagcccgattccgatcgctgacacgcccgcgctgatcgtcacccagcgatcccgcggatggccggtccgaaagaacgcgagcaccaggccgccgacggccagagaaccgacgaacacatcgcgcaaactggtgtagtagtaagcgctgatcgaattcgtcgtccatatgtggtcgaaggccagcgcgaagacccgcacagtgaccggcatcagcagcgcgatccagcccacggtctgctgaagagcatgatccgaaatcagcaatggatgacgggtgctggcagtcatgatgccccctgctttcccgaacgctttcacattcatatggcttgtcgggccccgttcatcgccggcgcctctgccgttcgttgtcccgtgatgccaagtctcccaccatcgctcgtgttcttgcgcggtgcctgcgggcagtgcattcgcctggcactcggatgctctgcacattccccgccggcgtttcacgcattttcgatgggcggtctgcgctgagacaggtataggttcgaaacacgtagcgcgccaccggcatgaaggattaacgagaactgggtagcgcttgcgatgcagccgaaaaaaacaaagggccgcgatgaagcggccctctcggcagatcggaactgacggcgct

SEQ ID NO: 15 - DNA, 2308 bp, Ralstonia solanacearum
tcacttcctgattgctgctgctagtcataaacacggtcgtatgcctacccgtagtttaagtgggtgactgtgtccggagattcagggggccgctccaggaggtgagcattagacatccgccacgtaacgtttcgagacgacttgtaatggtattcgcgagcgagttcaccctcggcaactgcgaactctggaacgcccagatcatcgtaaacgacgcgttgctgctcgttacccaggcaccttggcgatagtccgagtgccatccagaccttttcagcgagggcatacgtgtagtgcgatccgtctccccaggacgcgacaatgtagtcataaccggacacgctgggcgaaaactccgcgctcaagcctttgttgccgagaatggggacagagtaggacaccacgccacttgcctctgcgtccaggataggaacgggactttcaaccgtctccgggacgactgccattagcatgtgcacgtattcgctgttgggcccgtgattgtcaaactcaaggtcattggtaacgtccacctgctccaggggatccagccctacaggactgagaagcgtgcgcaggcgtgccggtagaacagctccctgaatagggaacgtggcgagatggtcaatggtaggcatcggcttagcggctcaattgcatccgagtcttcgattccgctatggtagccgcgaccattctcgttgcgtgccttgccgtaagcggtccgctggaatgagctattgagcaaaactgcggattgagctcggctacttgaggccgtaatacttgagcaacgcttctttgacgacgtccgtggcaacagcaacgcccatctttcaagtgcctgacgcggccttcttgagattctcaactagtcagttacgcactttaggtcccatgggctccgcgttgctctccggcttttctgacgcagcgagatgcgccaactcctgggcaaacgggcagcctcatggctgctcaaaatgctgtgatactcgtcattactggcgtcatgctgatacacatagccagccttggggccgaaatgatcgtctggttggtcagggaaatcttcccggccaacgccgtccaaatacgtgtgccagtcggctaccgcgctgttgttgcctgtcgtgctcatggcttcccctgattctgtgggtgtccgcatttcccctgttgcaggctgcaatgcaagagccgtgcagccggaactacgactgcacggcggtactgccgcaatggaagcgcgacggcgatccgatgcgttgcttgttcatgggctgatcgaaggagtcgcctgctacgcggcttgtagcgcagtttcgctgttacgtaggcaggcacgagaatgagcgcgaggtacgctcccagataggtggctatcgagctccacatcggcgcaaacacgtaatagcgatccatccagtagaaataaagttggaagagaatggctggcagcgcactgaacagaacccaccggatcggcgcattgcagaacgcgatgcctagcgcaacaccctgcaggatagcggccaccgcttcagtaggcgaccagtacgtcctaatcaggtcgtctcggcctcgtattagcagcaggctgatcgtaccggaaaaaatcggccacaccagaagtaccttcgataataaggaaagttcaactggccttctccctccgagccatttcttgaacccgtcaatgtttccatagccttggttgcgaagtccggcgtaaggcaagcaaaaaacaatgatcactctcagtgccaaccctagccccagcccacaccacgcaacgagcaggaacagataccaacccccgagcgttatatcgtcagggtaggttttcgcatcgatctggcatgcgtcatatcaggcggcgactctcaggacgaccgatgcggctacgaacaaccagaaactcacctcaagctgcctgccccatctcaacagaagaggcgggatggcaaacggtccaagaaaaggcagacaaaacagaagaagacccaagacagccaacacgcaatcccccttttttggattcccattctttgccccgcgcatggggccttaacgcgagtggtacggcaacgaccaagacctgggactgaacacctagggagcaatgccaatgtcggcatcgccaccggacctgcgtcaggattattcgtgctggacgtagacccaggccatggtggagacgagagttttaaacatctctgccaggagcacggcgagctacgggacatcaattcattgacgtacacatgctcagcgaaacaaccttgacgatcaagaccgtgcactt

SEQ ID NO: 16 - DNA, 1693 bp, Ralstonia solanacearum
ctcgctcgcctctcggatagtggggcccaatcgccgacagcaagcgctgccacggcacgaccttctccatctctgccaggaagcgctggcgtttggtcacgcgcttcttacctgcgctttccgcttctgcaaagctcatctgccgcttcatcactatggctccgttccgtgagctacctgctacaacggcctcagctgcgtcagcgatgacggctgagactgataaatcagcgtttccctagcgtgacattctgcatgatggcctccgcaaagaaaaacaccctaccagcgttacccgctcgtaggatgggggtgaccatctcattagacccggccgaggccgagttttgtcagacgatgcccaccgcgtgggttggcgcctcgggacatctacgacgcgaggcctggattacgttgagcccattgttttgatgaagcagcaaacccatcggcatcaccaagagacgcctagtccgcctctttcattactctcgctctcacacgccttcacgcctaatctcgttcttgagaatttgaagtccttcatcagccagccctcgaacattggagggctttcgatcctccagaccacgcagatcctcaagcagctttcgctcatctacatgtgggaagttccgggccacaaaatcgcagaccgccaaacacaagtccgaatcttcgtacggggcatagtgccccctgaagattgctccgagttgctcaaagtggtcgggcctcaaccgatgtcgtccggcgctcagagcctgtttggaaatgcgcggatatcagcaagaacaggctccgcaacgacgggaaaggtccgttcaggcgccgaatttgcttacattttaggcaaatcaagcgccgatgcggtagctgtgcgtccccgtcgggcaatttccaaacgggctctcagcaccatcaggaaattccatacgccaggttgccgataaagcgttggttctgtgagcaaccgctcgatgaactcgaaaggaccttgtggtagcccctccaaatccatcaactcatcctcgaactcatcagcaatcttggtgcagatgctttcacttttcgatgtcgccgattggctaatcgccatcgcaaagttgttcagtttctcgttgctcaaggacatggcttgcaccccctgccgtaattgccttcgagctcgttcaatcgtccccactgccgattgataagcttttcgtgcccacggactgtgtcgcgcagtttctcacttggcccaatccggtaaggaatccgggaggcgtccacatgagcggctcctaatcatcagccaccgcactgcttgggcgccatgtcacgcggaaatccgtagcaaaaccagatgagcctcctatcatcaggcttagcgacaataataacggatacccgccgttgatagtttatagccataataatattttcaccagccaaaaaataatccgtatctcgtgtttttaacgttgatataaagtttttagtttccgagcaagaaattagagatgtgcgtttatccagccagcatcgctcagcggcatctctcgccggggacatcgcctgcagcatagtcataatcgcagctctcttctcaggaatggaatcatacccccgcagcgacaaccacccaatcaaggccagcccaaggaaaaatgcaatccaaattgacaatcttttcgacataacaaactcc

SEQ ID NO: 17 - DNA, 3733 bp, Ralstonia solanacearum
gtcgatgcccgaggcgttcccctggccatcaccgtaacaggggcgaatcgacatgactcgatcgcattcgaatccaccttggatgcgattcccgcgatacgtggcctggatggccggtcgcgtaagcgccccgacaagctgcatgccgacaaggcttacgactgccgccgatgccggcagtatttgaagcggcgcggcataagggcccggatcgcccgcaagggcatcgagagcagggagcgcctgggccgttaccggtgggtggttgagcgcacgcacgcttggtttgccggctttggaaagatacgcgttcgtttcgaacgacggctcgatatccattgcgcgctcctctcgcttgctgcctccatcatctgcgctcgattcgtggatgacttgtgttagcgactcttagtcctcggtactacgcctgactcgctcgaataactcctgccaagtctcgccgggtaccttggggcccatccaccagtccatctcgtcccactgacgatgaactcgaacgcgcgaggcggtcttgccgtggaccttcgctagtgcgagtttctcaacggaatcgtagccctcacggacgcagcttccaagcttctcgagttcgaccgaggcaagctcgtccatgtccaaggacagttcttccgccgcaaaatgccgacgcacaagttccggaagggagaagatgtaagcgagctccgggagaacccgcaggacgaaacggcgcgcttgctcgcagcgtttctgctgttgcaacgtgggcttcagcttctgaatctccgctaacgtctgaccgctaacccacgccctcagcgctgcaatcacaacgtcgacggcgaactctcctcgctccgcgtcgtcctcaagcgacttgaacggctcgcccatgaatccgtccatgctctcacgcctggtcatctgcgagatgaggatgggccgctcggtcagccacgtggccatccacgtgaaccagtccagcgtcgtgcgcccggcaggaggcgacgtcttgaggcgaaccttcagctcgcgcagcacgttcgacggcgcgccgtagttggctgccatcttgtcggcccaatcgggctcctcatccagttcttgaagcttcctgcgtgcgcgcagtgcatggttaatgcaatcgttcagttgctcttcccgcgcctgcttcctcagcttgaacgccgcgaacgacttcttcaatagcgtcctggccgggccatcgacgtcctcggcctgcgtcgagatgggaagccggcgcaaaaggtagtccgcatcgccaccggcacgctcgccaagttcttgaatacggtcgaggatagcttccagcggatcttcgatcgccaagcattggtcgctttgcgcaaagatgccttgaagatccttccaatgcttgccaatcctattattcgaatcgtcgatgtccacaacgttgctgggaatcaccagcaccagaccatacgaactctgcccggcccgcccagcgcgaccggcagcgttgagcagatcatgcgccttcatctgcgcgagctgcttgttctcctcatcgaacttactgtcgcccgcgatgatgaccacgtgactcggcaggttcattccctgcgccagcgtcgacgtggcaaccagcacggacacgccatcgggacgcttgaagagcgactcgtgcagttgccgctcggtcggcagaagaagcccatgatgacagaggctcggccaccgcgcgaacttcccttcgacggcatcgatgtagagcgccgactcgtcgcccagctcatcaagcgtcaggttgtacagatgctgctcatcttccgtgaaggcaatgtttgcctccccaagatgctcacctgcggtctcctgagcgctcatagcgtacgggacggtctgcgcgaagatgagcgatttgattcccttgttgcgggatgcgagcgacgcaagctcagctgccacgttgttcacattcggcgtcagcttccacgtggttgcattcaccgaaagcggcatctctctgtcagaaagcgggagtaacgcgtaatcatctcggcgcatcgagtcccaccggaacttcagtccaagcaggcccagcggccgtaccttgagttcacgcttcagcgcgaccggcggcccaggcttgcgcttgccggcggccaacaattcttggcgcttcgccgccagtcggatgcgcaggtcggcaatttggtcgctcggaaagaccacacagccgcgcacctgccgtgtcggcttccacgccatgtttagcggcaagcacttgcgaccggtaagctcagccaaccaagccgcgacctcctcagtgttgctcatcatggccgagagcagcaagtagtctgcctcaggtgcatgcgtggagaggttcagaatgcaaagcatcgcatcgacggcgcgacggctacgatcgttcacgcccgcatgcagcagatggcactcatcgaacacgagcaggccaacgtccttgaagagctccggctgaaaccccatcaacgccaagcagcgctccggcgtcatcaccgacatcggtggcagtcgagtgcccaggtcaaaatcgaacgggttgtcgctcgcccgctctcgactcagccgcgcctcaggaaaggccttctggagcgcgcgcgcggtctggtcgaccagcgccagagtgggcgccagaaagatggtgtccttcccttgacacaggttcgaaagaatcttgagctcagatagagtcgacttccccgccccagtgggaaagccaatggcagctgaaaccccaggggtcaggtagccctgctccactgcattccggtgatttgcccaaagatacggccgagtcttcgcaatgagcgccaggcccgaaatccaaaggggtgtgccgacactaggcggtgccggcaggcaagccaagctatcgatcggcagttcgcgacccacggccttcaacaaagacgccaagtggcgaggcccagggaagccgctgttgccaagtcggtcatgggacaagacgaccggctgtgcgcacatgtcttcgacacgctccagttcgggtaacgacggctgataacctgggcgtccaagcacctcagcaacgatgctttgaacgcaacgatgcagcatcaggtatagcgcgctcgctccgacttcggcgatcgtcaggtcgccatccaacgatggcggttccgggacggtcgagtcagcaagcagtgcatcaagatcgccggtcgccaaccgcttgatgtcctgcaagagcctgcgctccacctgtggcaagccatcagagagctgaatctgctgcgacatctctgcggcatccgggctagcgcctgcggcaaggaacaggagcgtcgcagagacctctggcgcaatcccgccggccgtgagcggagcaaggtcatcccggtcagccgggtccacaagcccccgcgcctgcagggtcacatagtgcgctgtcgccgcaaggaaggccgcgccgcgccgctgagggccttcagggttcacggaggcgatggcctcctgggtgaacgcaatcttcctaagcctctcaagttcgggccgcagggtgtcaagcagatcagtggcgtcggacgttccattcgccatccttcgaacgcgcagccgcactccaactatttcggcgtaggctttagtcagcgccttgggcagtgagttggggtcaagaccagacagttctggcgcagagcgaataagctctgaggtggagtcatcaaacacgcgtcgctct

SEQ ID NO: 18 - DNA, 1813 bp, Ralstonia solanacearum
cgggaaggcccaatcaaggacttggtgatcgcgggggagcgaaccaaggcacagattcgggctcgggtcgaacatccgttccaagtcatcaagaatctgttggttcatcgcaagtttcgttacaagggtttggccaagaacacggcgcaactgttcaccctgtttggtttggccaatctggtgctcgccaggaggcagttgctggccagtcctgggagcattgcgtcctgagcgcgcaaagcgcgccaagataggtgcgaaagcagcgaaaaaccgtgctgaatcgaagcatcccttcctcaatccgaaaaaccaagatggcatgactcgcgattccattcgccgacttcattgatcagcggttccctaaagtcatccgccagcctctcgatctccgtcagcaaccggcagccggagaccgacaggccagggcacgacgtcgaacggagcccgtcgttttcgcctcggtctcggatgatgtccccggatgcactgctgggcgaatattcgcagcgcagcgacagcagcctgagccagctgcgccgggcacccgactccatgcaggtgtcgtcggcccgatccagtgcattggattcgctcgggggaaacccgccaccgcccacggtccacacgccccatcccgggcaagacagccatggtgtcgacgttcccaaccggatgcagatcgccgagcagttgcgcgacatcttcagaccgcacgtcaacagtgccaacgatgcgcaattcgaagccctgatccagcagcgagccgagcggctcgacgagatgggagaaacgccggcgagcctcgccgaggtgctgtccaagggtgccaaccgggaccggctggcgcaggccacggtgggcttcgtgcgctcggtgccgttcggcgttgcctcgcgcctcttcgatgtgaagcagcaactcacggccttcgccaagacgccggcgcaggtgggcgcggtggttggcgccggatcgggcgtggcggacgccttcggcggcagcctggtgaccaaggccaccagcaacacgcaatggctggcggcctcggccgcgcacctcgaacccgtcatggcgcaagcgcaccaggccgtgcagcccagcctgcgccggctggcggtggaggtgagcggggccttccaggcctattcgctgcgcaacgtggtccgcaccggcgtcgcgccgctggcaacccatgtcctgggggcggcaacggcggccaacgtggattcgtggatcgccgcggtgggcgggccggtggccggcgcggcggcctacatggcgatgcagcacatgaacgagacccagcaccgcacgggcgcggaatacctgctgggccgaacggactgggaaacgcagttcacgctgctcaagcagtcgacgtggacggatccgctgaagggggccgcccagcgcgcggcaaaactgcccgtcgatctcctgaccgagacgctggccgccacgcgcagcctgttcaccgccaccaacatcatcaagaatgcgggcgcgctggccggcggctttgccggtgtgctgaccgcgcagaccgccgccggccggatggcgacgaacgccggtgccagcgaggaagccgtcgtggccatcaagcgggccgtcagcacggtgctgtcggcaccggtctatgccacgtggacgacggccgatgtcgcggccggccccatcgtcgatgcggcggccgggcgcatccagcaagcgttcgcacccaggccgcaagcggtgccccccgcgcataccgccatcgacatcgtgtgatgtgtgaagcgtgaggtttgacgccggcggggccggc

SEQ ID NO: 19 - DNA, 2459 bp, Ralstonia solanacearum
gccccgtccttgttgctgctcggatgttgggtatccagcgaccaaagtctcaccgggctttggttacggttaagcaggagaggtggaatgaaattcctgtcgcgtacaccggtcttgttggcggtgatgatcgggctgactcgcagcgcctatgccgacgttctgccccaacctgcaaccgcaaagggagagccgacaatcgacgtccggggcggcgactccattgtcgaaagcatcgacatgtctccacaggccactcgcttctggcgcaatctccgtgagacggccaacaacatcgacctgaacgaccatgagaaggtcgcatctcagctcgctctcaatggacacgatcgtcgggcgctgcaacgcccgatgcagttcagttatccaagtctgccgctgctccgcgaatccgtgcgggtgacgacctatcgtgtccggtcgggtgggtccgtaacacgatggaccttcttgcctgacctgaggctcctatgcctgtcacgtagagaggtcgagcgaaacttcggagccggcgagaacgattcacacattcccccacaggttgtcgacgcaggatatggcggaaccttcgagacggtaacgagccgacttggttacgtccattcgatgcggtacaaagcgaagggcggggagcgatacgttgaggtgtcgctctcccctggaggatgtgccgacttcatcgggttgattcaattcaacgccaatgattttgggttgcctgagccgccgaacgcaatctcgatagagtagcgttcgcgaaggtctggcgaatatcctcacagcgttcgtccgctcctgccaattcttggcggttcatattcacaggtggatgtagttgtaggcggtttggagcccattcgcgggcatcaatccacagtcgacaacctgtccgcccggacaatgacaggcgccacaaaaataaccttgcgtagcgatgattgtggcccgtacggctgcatgcggaaatgtccgcgccgccaatgcgccggcagttcctggcccggcgtcaagcgttcaccatgctccccgccatggacgccttcgagcgatgccggcccaacgatatagcgatcgtacagttggtccacctgcgccaacttctcctcacgcttgcgccggccaagccccggaaacgccttgggtgcggcgctgtacggctgaaggtggcgcagctcggcgccatccgagcgtaggtacagcaaggtcttcgtcacataatccaggcgttgtcgccagcgatcacgcgaagcctcgatttgatgcgattcccaggcgcttccagcctcactgcactgccgatccaattcctgatcgatgaccggttgcagcggtgcattctcgtcgtcgatggtcacacagaagccttcggctacgacgccccccttctccgacgccccgaaactcgacatcgccagcgcccgccccgtcgacgctgccgctgacgcactggggaactcgaacaggaaaatggcatgcgagccgccgcaatagtgacggtgttgccagggcgggacgatgcacagcgtgtgcagcggtggcgtcagcaactgtgcaggcaggtccgaggcgatatcacttccgctcagcagggtccccaacgacagcgtgggctcatacagcgcgccacctcgccaccaaaaggtcagcggcatcatgatcgcgctcatcttgccgcggaggtcctctgcggcaccttcgcggtccaccttcgaaaggacgatgctcggtgtgatcgcatcgatcagcgccatgaatgcttcgtgggaccctgtatcggggtcgctgacgattctgcgggccttcgccacccgcgcgtcacgttcgctcagaagatcccatgcacgcagcgctccgctggcccgaagccgcaccggcgccggcaaatgcttgaacgccggtgacaacgccgcaaccgacttggcgacggttgcattgaccaacggatcctgaaaaaaccaaggttcgcgacgaatggttattgtctgcatcgtgcaatcttcaccgtatcggatcaatagacaggcgccggccacgacgccggtgcactacatcgcccagtccgcttgaagcagacgtccgcgaatgcctggatcggcgcagccgctctgggccgctcctccggccaacgcacgatctatcgctgtgactccagattccagcacatgcgcgtgcgtgagcctgtcgccttcgtaggcgaacagcatcggcaggcccggcggcagatacgcgccgacgaattcggcgtggtcttcgaggtcgagccacagccacacctcgtgcggcccggcctcgcttgcccagcgatcaaacaccgggcgcaaggcacggcacttctcgcaccagctttcggcacccagggcaacgacgaggcgagctgagggatggccaagccgctcggcgatctcacgtgcatgggtccaagggtcgagttgggcagt

SEQ ID NO: 20 - DNA, 1114 bp, Ralstonia solanacearum
ataaccaccgctaaacgaacgccatgaagagcttcatcgcagtatcggcagcaaccgtatccatcgccgcgaacgcatataccgtcacgactacgacgacgaccacaacgcgctccgacgggactgtaaccacgcagacatcaaggaccgttccctacgatgccgcccgatgcgcgcgactaaccgtcgacctcagcatgctcaacgaccagctacgcagaacgggatggaaactggaacaggacaacgtgcgggcacagatcatgaccgttcagcaggaaatgagcgatcggggctgctaggccgcgagcagcacagccttagacgcactagcgaaatacgacgagccatcgagggcgacgaagcatcttatgtccgcgatatcgaactcggcgcacagcgtcgtgagcacgtccttcagtgcgtccgcacagtctgagcgactgttctcgatcgcatagaccacggcctcaggaacgttgatgccgcgactggtgtagtactttttcttgttgaaccgagtctcagcaatgtccttgccgatgtacttggcgacatagctggccagcgcatccaacttccattgcttgccgaagtgcgccccttgagggttgcgaatgtggcagtaccccttgccacgaccgcccacgaccttcagccatacccgccgcgcgaggttgagcgcgacccgacctgagacggcgacgtgcatatgccatgcgccgcgtttctgcagctcaggaaccgcgatgtagtggaattgactgtagcgagccatcgcacgccgaaaagcgtcccagagccccaggaaccagtccagatcggtaatgcactcgcgcgtagtcagcgtaatcatgtgcgtgacgcgagcagtcttgcagcgcaagcgaacgttctgtttagctcgcttcgcagcagatttgacgctagcggcccgtgcctctttatcgtcttcattttcaggcttggcgccgcgtgcacggcggggaagatcacgcagatcgttcaggcgctgcaccacggtacgacgataaccgctgaactcgacactgccatcttcgaacgcccgaaccttggcaaccatgtcatgacgcgccggcccctccgcccacaa

SEQ ID NO: 21 - DNA, 2428 bp, Ralstonia solanacearum
ccctgctcagatgtgcgatctatccggttcaccccagtccattgcaggccgatgtcgacccgcttcagcgacggtaaagctcgtcagtagtgcctcgccgccgatgccccaggtgccccaggtgccttgcagttcttcgcgctgacatcaggaattcgaaattctcacagaagctcacctactgtcgctccagccagatgctcgcttaaagtcacatatctcacaaaaataagcgactttggggttgggaatgaagcgatttgtcaaacggcagctttgggttgccgtattgggcctgagtgcagcatccggtgcatgggcgggttcgtccgactctggttttgacgggttcatgtggatcaacagtggcggcatcggtcaaactcttactccacctgagcgcagcccttcagagcagtgcatggtatcggtcgctgcccacgatgccatatatggaacccgctacaaatatgttggcctcgacacgacgaatagtaagaacatccaatgcatccttgatattggtagcggtaccccagcggggtatgcgactgtgcgccgcatcaatacatgcgtacctgggtatgggttgcaacctgatggttcatgtaagccactgtctccgcgtacccccgtgcaagtctgtacggccagcagctctgtcattcccggtacaggcaccaagaccttcatcgagggcgacgaggggggcagtccagacctgtctgtcacacttgcctaccagcagtcttacaacgactacggatcgaatgtcggcatcggtcaatggacaacaaactggcagcgcagcctggacactggttccgccagcgagtcgactccccaagtgtcggctcagcgtgaagatggttcagtagtcaccttcagcggaaacagcgcggcctggagtgccataggcagccaggacacgctgcggccaatcgcggatgcaaatggcaatacaaccgggtggcagtacaccgtggttgatacgggcgtggttgagacctacgatgtcaccggcaaactgcaatcggttcgggaacgcaacggccgcaccacgacactggcctataacgtggtagggcagttgaccgctgtcacgacgccaagcggccgcagtgtgacgttcgcgtacgacgcgtcaggccggacgactagtactatggctcccgacggcgccgtcactcgctacggctacaacagcgctggcatgctgtcgacgatcacgcggccggatggcgccgtacgccagtatgtctacgaagacagccgcttcccgaccgcactgacggggatcgtggatgaaaacggcagccgctacgccacctatgcgtatgacgaccagggccgggccatcagcagcacgttggcgggcggtgccaatacgtaccagttccagtatggcgacaactaccagaccactgtcaccgaccccaccggcaagaccagcgtctacagcttcctgaaacagaacggcgtgctgctgccgacttcgatcagtgctccatgtggcctgtgtggcagcacccgccagagcagcagctacgacgccaacaacaacctgacccaggaaaccgactacaacggcaccgtcacctcccacacctacgacagccagaaccgcgaaactcagcgcgtggacggctcgggtacgaccagcgcccgcaccacgacaacggtatggcactcgcaattctggaacctgccgactcaaatcgcctcgccgaccaagctggaaacgtacagctacgatagcaacggcaatctgacccgctacagcgaaacgccgaccgcggacagtgacggcagccagggcttcagcgcagcggcgaccggcccggctcggaccaccaactggacctacaccgccgacggcctggtggcgaccagcagcggcccgcgaaccgatgtggcgaccagcaccacctacgtttaccgcaccgccgacgacaccgccacgccgccgcaataccgcaagggcgacctgtaccagatcgttgacccgctgggccacgccaccacgatcaaccagtacgacgccaacggtcggccgctgcagatgacggatgccaacggcgcggtcaccgcgttcacctattcgaaccgtggctggctgaccagccagacgatcaccccggcgagcggggcggggcagaccaccaactacagctatgacgccgtggggcaattgaccaaggcgacgttgccggacggcagcacggtcagcttcacctatgacggtgcacatcgtctgacgggcgcagccgacagccagggcaacagcattgcttactcgctcgatgcgatgggcaaccgcacgcaggagcaggcgaaggatccgagcggcaacttggcccgccaagtcacgcgtgtcttcgattcgatgaaccggccgctcaaggtcacggtcggcactgcgtcttaa

SEQ ID NO: 22 - DNA, 1818 bp, Ralstonia solanacearum
agcgctcgcgcagcctggccagatatgtcgttggcctcgtttcaggagccgccattggtcatctcgaaatggatacctgtcaaccgtggtctggccttgtagccttggctcacctggaactgacgcggcatttcagccggccagaatagaacgacgcccaccgcgtgagtgagcgtcgttggcaatagcattgcgacttagcttgacgaagtgtcttctttcagcggaagcccgagcgcatcgctaattttgttgtcgatagacccgataccgctcactccatcaacaccgacgggctccgccgcttcagatagacgattcccatgctcatcaaagaaccacccgcgaaaatctcggaaacaatgctgcggcagcaaatcaaccgctgtgttggggccagacctcacaacctcaaagaatccccgtatccaagcgcgatcattggtgccgatagggggcaccagcaaatcggacggacggaggtcggggattttcatcttgtcgcccgaggtggccctgtacaagtaaatcaagatcacgcccccaccgtggacgttaccgatcttggtgtccgtgtccacgactcgcccaaagaagtaccgatccggaaactgctccaactggaacgcaaagatgtctccggcctcgggcttttgccgcgttttcttcagaacgactaaattggttggtggtgttttcatgtttagaagccgcgttcttcgagccagcagtcggcccaactcttcgcgtcttggtagcgccatcagatcccgtgtgtcaggtttgatttacccacgtgtgcttggtcgcaatccccaaatatattgttgtgcgcacgggttttgcagacctatgccccttcgttgctctttagcttagatgcgcaatacccaaccattaccgaaagaacgaacagcagcacagctaagagagatctggtgtagaacctcatcgaaagattgtgtatctgcgcgtcagttgcgaacgtagttagccaggcagcttgaattgcccatattgcaaggacaattgatgagagtaaaaaaataaccatcaatatccacagcaagaatcttttcatgtcgcctcacttaccaatttatctattatttaagatttactgcttttttggaccaccgcatttatcgataacatcctgcacaaacacgtgacagtcgttggcgaacgggacaaatggccccgtcttcctgcctggtttgagcaaatcattaacgcactgctcgttaagcgaacgcccagagtatggaacctggaatgtcccgggcttttccgattccccagaatgattgaccacttccgtcggagtaagtggcatatcaggtgcggcctgaccaggaacatctccaccagctggccccattcctccctctgctgtatcggttttgatccaccaatgatcaatgcctggaactttgttcaatacgtcgagtgggccatcaatgtgaatgtggcgaccatatattttcacttcgaggccacgagagtccgtattccgagtcggctgcccgccaacatagctgtaagtcgaccactgtccacccttcaggccaatgggatcagactggatgtagcgccctgtcaccggatcgtagtcccggttcgcgttgtagtgcttgcccgtctcctgatcgtagatttgaccggggaagcgcaggttgtagggaaccgctgatcaatgaagtcggcgaatggaatcgcgagtcatgccatcttggtttttcggattgaggaagggatgcttcgattcagcacggtttttcgctgctttcgcacctatcttggcgcgctttgcgcgct

SEQ ID NO: 23 - DNA, 382 bp, Ralstonia solanacearum
tatgccttttccaacggttgccagactcctcgacgtcgaagaagagcagcgaaagatcctggcggcggatgcggtgatcctgctgttcccactatggtggttcagcatgcccgcaatcatgaacgggtggatagatcgcgtgtgggccttcggtttggcttacggctacaaaggggctggcaatgcctatcgctacggtgagagtggctttgccgtaaagcgcgcactgctggcggtatcagttggtggacctgcggacgactattctttccgtggcatcaatgggccgttggagcagatgctctttcatatcacacacggcactttgttctttccgggcatgcaggtgatatccacattcgcggtatatgagactgtttga

SEQ ID NO: 24 - DNA, 1434 bp, Ralstonia solanacearum
tcaatccgaaaaaccaagatggcatgactcgcgattccattcgccgacttcattgatcagcggttccctaggattcagaagctgttgtcgacggaacaattcaattttctttcttaacgccaaaccggctggagtcagattttccttcgggatagatcgaacgatccatttgtggctcatctgcacacccttgttttctgtgtcccccaaccccttcttcagttccgattctccaccatcctgattgaccggcgccttggccgaactcacggcatcacgaccttcactgtgaaagatattgggctgaccgcttggcgtcggattctcgacctcgcccttgccaagcgcttgcagttgctcctggatgtccttgggcaacgcagccaggaaacggaacgcgccaaacaagctgctcttggtctcgccaatcacgccggggcggatcgggttgcctgtctgcatccagacatggtcgatcccctgtcgccccgccggatgccactcttcccagagtttcccgaaattgttggccttcttgaggttgcgcttgttgcgggcgcaccagtaatccgtgatgtgctcggcgggcacaccggtttgccatttctgcttcttcttggttgcccggcgcgtatcgctgcgcgctgcatccgtgggagcccgctccgtggccgtatggtcacgctcaactttctccgtcttgcgcgactccggtgccttggctttcggcgcttcgccggccgagggggcatgcggcgcatgttcggatggcgcgtgcggtgccgaaggggcatgcggcgtcgcgtgggcagcggtgctcttgggcacggcatcgctcatggacttcttccacagcgcctgcagttcttcgactgcctcatcgagcatcttgggggcgcgctgcctcaagtccttgatctgttcgatcagcagcgcgacttccttcttgccgatgaccgtctggacgatccagccatccaggccatggatgaatgcacccatgacgtcgtcgaagttccgtttgaggctgccggctatggacccccagtcgaccgtccggaaccagtgttcgaggttgcctttgagcgagggggagaccgcatcgagcacgcgtggcaatgcgtggcccgacttgatcatgcggaacgaggtcttcaacgcatcgcccagcgcagggacgcagccgaccagcgtaatggacatatcgagccagccggaatggctggtcgggctcttgctgatgacgatgacgccgacgatgatgtcgcgcacgtccatcgcctggcccaggccgggaatcacgctcaagacacccgagacaatgatttgcgatggcgagctgccttcgccggttaccagccctttcagccagccgctcagatcgtcatccgctgccttgaccgcgccgcgcagcgctatggcttccgcgttcaa

SEQ ID NO: 25 - DNA, 3126 bp, Ralstonia solanacearum
tcactgcagccgatacttcgattgcctgttcttgaatgcgggctcggtgccttcgagaatctggacaactcggcccttgatctccggttgctcaatcgcacccgcttcatactcaaatcgaacaccctcggtgttacgagacgcgtagacaatttgctcggcgtcacacaccaccgccaagttcggattgtgtgtaaccatgatgacctgccgcctctccttcgccttcttgatgcacttgactaggatcttgtagatggtctggttgtcgaggttctcctccggctggtcgatcacgatcggaatgtcgtccttgtcgaccagaaggtagaaaaccaagagcaataatccgcgctcgccaggcgagagttggccaatgtcttgccccccgaaggtcaacgagtagcgcggcgaaaggtattcaaggccaaagatgtagtcgagaacttcggtagcttcgactcccttgcgaagctgatcggccaaccgcgtctcgcggctgtcctgcccttcgcgctgatcggagcgcatggtttggtcaatccgctgtgcgaaggcaacggcgtcttcgggcgccgcgaagctagcctcttggagcatgcgcctaacgagctgattgctttcgtcgacgccagaaaaagttccccgcacttggcgattgagcttgccgagaaactgatcctggaatccagtctcctcaatgcggacgtgaaattcaagaggaagattcatgtccatctgcccagccgactgcacgaacgcctggacaggcaggtagagccgttggtattcgtcgaccgtgccgcgaatcaacccatggatctctcgtgatatcccggagcgttgctcaatgagcttctggagctcatcaggaagcgcggcaagcgccgcaatctcggattccaactgcgcgatagttcccggcttgtcggcaggccccatcacgtcggcctttgatttttcccacttgaccaactcctccttgtagagcacgaagagacgctgacgctcgccgagcttgctcttggcctcagcgagggaagcgaaaagctcctcgcgtcgcttcaagatgctgtcgggctctgcactttgaagctttgcgtcgattgaatcgatctccccttgcatcgctttggcgattccctcaacaggcccggcattgatccgggtctcgaacaatgagtcgatgggaatctcaaccccgagctctgctaagagctgtttcaactcgctttcgaaagcttcgctctgcttcttctggttagccaaagcctgggcgatgcggcgcgcgactgcttgcccctttgcagtagtggacttcgcctcccgaagccgtgcttcctccttgccgacatcctgcagttgctgctcaagaccgtcgatcttggttgcggccgctttggactcttccaatgcttcaggggaagcattcggatcctcaacctgtctaggcttggcttcttcgagggaggcgagctcagccttctttgccgcaagctgcttgaccaagctctccttgaattccgggaaaagcctgcgctccgtaccaagaatctcgacgttgagcgcagcaatctggccccgcagcccccccatggccttgttgatctcggtcaccttgaaattgagcagcgcgtccatcgatgactggccaagttggtcctccagcgggacgtgtgagtagatgatttttcgcaactcggcatcgaaagtcgccgatccgcccgcacccagctcattgcagagcgtctcaaggtaactctgcggcagatacttcacgcgctccacactactgggctcgggatcttggtccaaatcccgcgtagtcggcgagccgtcgcgccagaccaaggttcccacgaagttttgtgcaagccgtgttcgcggatcccggaagcgcaccgcgttcaaaaacgagaagctacgatagtttttggtgttccccgccaacgcgataatgtcagcaagagcgctctttccgctccccttgttcccgatgattgccaccaaatctgaattcagcggcagttccatgtcgaaccatgtgtcagcgagctttgagccttccttcttggcgaccctaactgagcggatgaacttcgttcgattgagctcaacgtactggcgcttggccgggttatcgccaacgcagacgcgatcatcgaactcttcgatagcctgctgcagacccctgaaggtgcagtccgccttgatccaggtaaagcagttaccgatgcggtccttgtcggtcgatgcgctcagccaatgtgcatcggaacagtcaagcaagttggagttaacaccggactccttcaacttggcgcgcgccttggcgtgatcctctggggattccgcagcggtaaagacaagatctgcactgttaatgagcgtctttttctccgcgatcgagtggtcatcacatttgaggttctcccactcggtcttgccgactgcaaggatgaacctccccttgaggacctcgttctccagggccttgcgcaagccatccgcgctgacgttgaggttgttgaaaccctcaatgagagggctcccataccccacccgcttgtctgctggggccgagtcaatgatggctgcaccaagagcttcaatgctttgccgggagatcactgagttccattttccttcgccagtcgacccaggaagaagacggtagctcggggcgattgcggaaataaactgctggcggatgaattcaggatccacctgatcgaagatgatgtggatgttgatcctgctccagcttgatggtgccttgccgctttttcccccttggacaacgccgccgaacttgtccaaccgcagttccacgaccggcagaatcagatcgatgttcttcagccttccctgctcgcgcttcgccttcagcaccttttcgtagccatcgacgagcacatagtcgttgataccgattactttgaactcctctggcagcttttcaacgtcagcgagaaacgcttcccaggcttcatcttggctccccgggtagttgtgatagaaggattcgggcgtgtggacgtgtaaatcccatttcttccaagtggagccggccagggcaaggggcatgttcttgtctcgctgaagttgttgttcacaagcgggcat

SEQ ID NO: 26 - DNA, 372 bp, Ralstonia solanacearum
ttgtgttggtcaagccaacgccggccttgttgcgggtccgctggcaattccggtggcgtgggcatcggtgcgttggacatcgcaacctgtgtggggatgaaatcctggcgaccgcccgggtgggtcgtctgggtcccactgggcaatatccggaccatcgttccgtcggccttgcgctgttggacatatggatggcccgcttcatcaaagccgcggctcaccgacccgctggcatcaatcgtcggtgcaggccccggcactacgggcttaggcggcacgacgacggccgtgccggcgaggaggctggtcttcgcgttcagcttgctgatggcccgcagcggcggtgctgcgccttcgaccaacacctcgtag

SEQ ID NO: 27 - DNA, 174 bp, Ralstonia solanacearum
gtgcggccaggatgcggcggcggcgctgggcgtcctcgaccatctggccgccggcatgcggctcggcactcgctccttcgccccgcccgacggcagcggccctgcgctgacacggcaggcgtccgacagcgacagcgtcgacgactttttcgacgccctcaacaaattccatga

SEQ ID NO: 28 - DNA, 546 bp, Ralstonia solanacearum
ttattgcttggcgggttcggcggccttcggagccgaggcggcatcggtcttggtgtccgacttcgcttcagccttctcggccttcttgtggtggtgatgcttcttcgcgtgcttggcttcggccttcggggtcgaagcggcttccgtcgtggtcgctgcagccggcgccgcagcgggggcggcagcggccggtgcggaggcggtgccagcggcgaacacgggggctgcgaaggcaccagcaacgatcagggcggccagggattgagcgactttcatgcgagactccttaacggatatgagcgggactccagatacatggatctcgtcccatgtgcgatccatgtctgcaaaaacgcctggcatcgcaagcgcgtggaccgccgtttgtaagggattcgcccacggtgtaagcaggcgtaacacgggcctgccggacccggcgacgggcgtgttgcctgcttgaggccggccgcgtgcaggccggttgaggcacacgttaaccggcccgcgccggcccgcaccggcgccggcacgggccgatcgcac

SEQ ID NO: 29 - DNA, 111 bp, Ralstonia solanacearum
tcatcgccgccagcaggctctggccgccgccgaggtacttgctgtccggatgcgtcttgagggcggtgacggcggtctgggcgtcggtggctttgtggtactcgaaggcat

SEQ ID NO: 30 - DNA, 807 bp, Ralstonia solanacearum
atgagcccccgcaagccttacccgacagacgtatccgatgaagaatggagcttcgcagcgccatacctgacgctgatgcgcgaggacgcgccgcaacgcacacatgacctacgcgagatgttcaacgcactgcgctggatggcgcgtgccggagctgcgtggcgcatgctgccgacgaactttcctccgtgggagctggtgtaccagcaaacacaacgctggctcaatgcgggttgctttgaggccatggtcaatgatctgcgttcggtgatacgggtcgcccaagagaggcaaggtcaacccagtgccgtcattctggatggtcgaaccttgcagtcgacctgcgagagtggcccgcgcgcgggctacgacggctacaaacgcaaacggggcagcaaggttcacatggcagtggatacgctgggccatctgctggcggtgcatgtcacgccggccaatgaacaagagcgtgcgcaggtagcggaattggctcgtcaggttcagcaggccacaggccaaaccgtgaaggtagcgtttgccgaccaggggtataccggtgaagcgcctgcacaagccgcgctggatgaaggaatcgaccttcaagtcatcaagctatcagaggccaagaaaggctttgtgctgctgcccaggcgctgggtcgttgagcgtagctttggctggctcaaccgcttcaggcgactcgcccgtgactatgaacgccttccagaaacccttgcgggtctgcactttgttgtcttcgccatgatcatgttggttcatgccgtgccgatcatgcaaagtgcctaa

SEQ ID NO: 31 - DNA, 234 bp, Ralstonia solanacearum
atgaaaaagtctctcctgatcgcttcgctgatggctgccgtggctctggctgcttgcggtaagaaggaagaagccgctgctccggctgccgccccggaagcttcggctccggccgccgcaccggctgctgctgccccggccgcttcggaagctgctgctgccgcttcggctccggccgctgctgctccggctgcttcggacgctgctgcttcggctccggctgccaagcaataa

SEQ ID NO: 32 - DNA, 102 bp, Ralstonia solanacearum
ttgccgccgggcgggctggcccctggccgcctccccggaagggttcaccctgctcggcgggggtgcccgttcgtcgcgccgcctgcggtgcgatcgcgctga

SEQ ID NO: 33 - DNA, 807 bp, Ralstonia solanacearum; identical to SEQ ID NO: 30

SEQ ID NO: 34 - DNA, 318 bp, Ralstonia solanacearum
atgcagaagctatttgcgctgtgcgcgctggccgccgccgcattgccgtctcaatccgccctggccggcgtcaacgtgggaatcggcatcggcatgcccgtgccggtctatgtcgcccccgccccggtctatgcgccgccgcccgtgatcgtcgcgccgcagccgatcatcgttccgcccgcgcccggctactacgaggacgactggcaagcccgcgcatggcgggagcagcaatggcgcgaacagcgctggcgcgagcacgaatggcggcggcaccaccgccatgaccgccacgatgatgacgacgatgacgagtga

SEQ ID NO: 35 - DNA, 180 bp, Ralstonia solanacearum
atgaccccggagcaagtaaaggcgcgctttgagcgcgagggcaagacctttgcaaagtgggcaaaggagaagggctttgaataccgcacggttatcgcggtcatcaacggcgtgaacaaggggcgttacggcgaggctcacaaagtcgccgtggcactgggtctcaagaaagctgcgtga

SEQ ID NO: 36 - DNA, 1683 bp, Ralstonia solanacearum
atggcacgggcacaactggttcagcgcgtggacgcaggcggcgcaagtggcgcggtagcggtcatgcccacagcaaaggtgctggcgcttcgtgctcgcgacccgtggcgcgaggcaaccgacagggcccggcaagtggcgacgtggcgtgagacggtggtggcgtacgtgcgcgccatgacggaagacggtgtgacgcagaacaacgcggtggcgctgctgctggagcgcggcgaggccggcagcctgcccagccacttcgccatggcgctggctggcgccgccaaggcaggccggaagacgccatcgcgctccgccatctgcgaatggtgcgctcagtatcgcgaaggcggcgcaactgcgctgctgcccgaccacaagggccgcgtggtggaggccgccggctggtgggggccggcgctggagtatttcaacgcgcccggcaaaccagatatggccgctgtacatcgccgcctggcggaagtggacggcttcgcggtcagctacgaccaagtacgcaactacctgacgggggttcctgccatgctcggccgcaacagccccgcacgcattggccgcaacctgtaccgcctgaccgagaaggcatacatccgccgctcgaccgaaaacgccttgcccggcgatgtgtatgtggccgacggctaccgcgcggacgtgtacctggcgcaccccgtcacgggcgatatctggcgcccggagctgacggtggcaatcgacatgcgcagccgcttccccgtgggatggcgggccgacgaacatgaaggcacgtatgccgtgcagaacatgtgggcagagtgcttcgcccgctggaaccacgtgccgccgatgctctacatcgacaacgggtccggctacaagaacaagctgatgagcgacgagatgacaggtttctacgcccgcgctggcgtgcagcaaatcatccacgccatcccaggtaacccgcacggcaagggctgggtggagcgcttcttccgcaccgtcaaagacgacttcctgaagctgtggcagccggctttctactgcggcgaggacatggcgccggaagtgctgaaccgcaccgtgcgcgaggtcaaggcgggccggcttgtgctgcccacattggcccagtttgccgacgccttcaacgcctggttggaccgctacgccaaccgcccgcacccagaagaccagaacacgacacgcgccgcgctgtggtcgcaactggcaccgcttccgccgcacgccaacgtgaccgaactgaagcgccaggcggtggtgctgacggtgaatcgcgcagccatcaagcacggcaagcgtgcctacacgcaccccgagctgcacgccttcaacggccacagggtggtcatggaatacgacctgatggatgaccgtgtcgccgtcatgcgcacgcaggaaggccggtggatttgcgacgcacacctcgtcacggccattgacgccatcgcgcccaaccgcctggaagaaaagcgccaggcgcgcgccgccgatgccatcaagcgcctgcaacaaaagatggacgagcagaaggcccgtgcgggcatggtgctcgatgtggacagcgtggccgacggcgtgctgcctgcaatcgaggtggaagcccgcctggtggacgacagcgacgacgcgccgctgctgctcgacctgacgatgaacgacgaataa

SEQ ID NO: 37 - DNA, 327 bp, Ralstonia solanacearum
atggcacagaaactgaacctgaaacgcccgctggcgttcggcaaagtcacggtcagccacctgacgttccgtgactacgccactgccgaagactatctgtcgttcgacaagcgcggcggcgtagcccaacgcattgcgctgatcgccagcctgacgggcaccgacgaagagctggtgaagaagctgcacggcattgactatcgccgggccgagaagatggccgacgctatcatcgaagccgacgaagacgaagccgaggaaccggcagccgaaagcggcgaagcgccgcccgaggatgaagccgccgccgcagcgcgaaaaaagtaa

SEQ ID NO: 38 - DNA, 807 bp, Ralstonia solanacearum; identical to SEQ ID NO: 30

SEQ ID NO: 39 - DNA, 807 bp, Ralstonia solanacearum; identical to SEQ ID NO: 30

SEQ ID NO: 40 - DNA, 210 bp, Ralstonia solanacearum
atgtcaacccttgctgagatcgaagctcagatcgctgagctggaagaggccaacgagaacttgcacgactcgttggatgccgctgaggcgatggacgacgctgaggctatcgagttcttcggcagcctggatgagcgtgatgaggcgattgaggcctgggaggcggcgatccagtcgaacctggagcagattgccgagctggaaggctga

SEQ ID NO: 41 - DNA, 369 bp, Ralstonia solanacearum
ctagtgaaaaatggtcgcgcggatctcgccggccatttttcgtctagcgtcaggagtgtccgcgcggatgtcgaagccgctagggggcccctttggggatgtgggagtgtggatgagggcaggacggttgtcgccggaggcgtctggcgggaccgagtagcgggtaggactggggcggtgaagacagggtgagggttgggaagacctccctcttgcaagccccctctgctgcgtttggcgggccgctgcgccgttttccggaggtggtaggtacgtggggaggggtgacggtcgcgcgccccgcccgcccgcagcgaagcgaggacggacggggcgcgcgcagcgcgcccctacttgtagtaataacac

SEQ ID NO: 42 - DNA, 1563 bp, Ralstonia solanacearum
atggaaattttcaagcgaattcaggaactggcacctgaaggaattttcccgctttccagaatcaagctctcccagttttacggcatgaaattggacgatttcgctcatgagatcgccatcctttctctgtggctggcagaacaccaaatgaaccaaaagttcaaagagacttttggacataccaatccagcccttcctttgaagagcagcggcaacgtcgtcagcgataacgcaatccatgtcgattgggacgagtttcttgctcccaaagcagagcatgagacataccttcttgggaatccaccctaccttggagcacgtaaccaggaaccagagcagaaagcagaccttgaactgatttttgaaggccacgaggaatacaaggactcagactatgtctgctgctggttcatcaaggccgcaaagttcattgctggcaaaaatgccaagtatgcgttcgtggcgaccaactcaatctgccaaggtgagcaggttgcctacctatggcctagagtcttccagcctggtcttgaaattgactttgcctacacctcattcaagtggtccaacttcgcaaagaacaatgctggtgttacgtgtgtgattgttggggtgaggaacacggctgacaagccgaagctgctgtttggagccgacagctacaagattgtgaagaacatcaatccttacctgattgaaggtaaggatgttttcgtgcgtcggacctctgaagttctgtcagggttcccggcaatggtgatcggttgcatggctcgtgacggtggcaacctcattctgaagcctgaagaacgcgatgccatcgttcagcagtatccgaagtccaagccactattcagaactctctacggcacccaagaattcatcgatggcaatccacgtcagtgcctttggatcgaggacgaacagcttgaacttgctcgctcaatcccacctatcaagacacgcattgatgcctgctatgacttccgggtgaagtccaaggccaagaccaccaatggctatgccaagattgctcataagtttgcccagcgagttcaaatcaagggcggctctatcattgttcctgcaacctcttcggaacgaagagagtatgtgcctattggctaccttgatgccaatgtagttatcacaaactctgcaaatgtcatttacaagcaagatcctgttctctttgggatcattgcttccaaactgcacatcctctgggttcgcacggtgggggggcagcttgaaacgcgacttcgctactctgctgagatttgctacaacaccttccccttccctgatgccagcgagaagaaacggcaagcagtggctgagaaggctatggcgattgtcgccatccgtgaagcgtatcctgaactgagcatcgaggacctgtatgaccctgacactatgcctgctgacttgaagcaggcacaccacgaactggatgtggtggttgagcagtgctaccagattaagccgttctacagcgataccgaacgcttggaatgcctcttcaaactatacgaaaaaatgatggaagccgagcatgcctaa

SEQ ID NO: 43 - DNA, 111 bp, Ralstonia solanacearum
ctactggtgatgttgacgatgcgaccccagccgcattcgatcatcgcatcgaccgtcgcctgaatcagttcgatgggcgtgagcatgttggcgttgagcgcggcgagccac

SEQ ID NO: 44 - DNA, 138 bp, Ralstonia solanacearum
ttgcgctgtacaacggcggcagctactacatcgacagcggcaagccgacgccgcaggcgttcgagcagacgctgcgcaacctgcacgacgtacagccgacggtgtacttcaacgtgcccaagggctgggagatgctga

SEQ ID NO: 45 - DNA, 312 bp, Ralstonia solanacearum
tcacggtgcgggagcaggaggcactgctgtcgccggcgttcgagcggatcgccgatgtgccgatcgccgagggcgaatcgattcccgtgtttgccgggcgcgagcgctggcaggtctggcggcggcgcgctgactgatccggtggcgtcggcgtcgcagcggcgcgttccggcgaccgttgtgttggtgcgaagcaacaagggcggcgaccggaaaaaaagcctgcggacgcccattacggtccatcgctgaaaaaagtggcagacggggccgcgttcgtggtcgcaccgatgccccgcgggggtgtcccat

SEQ ID NO: 46 - DNA, 174 bp, Ralstonia solanacearum
tcagcgcaccaactgattgatctcgatgatcggcatccgcactgccagcatgatcagcaacagcagcacgcccatcgtcaggatcagcgtaggctcgagccgcccggtcaggagcagcgtgcgccgctccagctcctgcgcctcgcgctccggcatggtggaaggatcaatcaa

SEQ ID NO: 47 - DNA, 375 bp, Ralstonia solanacearum
gtgctgctggtgctgctcgtgctggatgtggacgacgaactggaactgctgctggagctgctggcgctctgcagggcggagaacagatcgtgcgtcagcttctgcatgtcctggccgatggtcgaactgctgctgctgtcgtcgctgtcgccgtcgtcaccaccgtccgggggcggcatgccgccgggcggcggcgggggcggcgcggaggcgctcgtcgtgctgctggtgcccgtggtgctgctagtgctgctggtgctgtcggtcgacgacgtggacgatgccgacgacgtggatgacgtggacgaagtcgacagaccgagctggctcagtgcctgctcgatcgcctgcatgaagccgccgccaccgccatga

SEQ ID NO: 48 - DNA, 1026 bp, Ralstonia solanacearum
atgaccttgccggtcgaatcgcccatgcccccgccggccggcgtcggccaaacggcacccgcgccgctgtccgtacggcgcatgggctttgccatggccgtctctatgctcgaagcatcgaccgacccgcgcgcgccagcatgccgcgcatgcggccggcccatcgacaaagccgccacccaggcggcagtgagccaatccgcgcccagacggaaggctcgacggggaggtcaccatgcgtcaacgcttcatggacagcattccgtgggccacgctgctgctcggcgtgggagtcggcgcagcactcgccatcacgcccagcacggcgcacgccgatcgccggcagtcctgctatgccggcctcgtactcgtgttcgacggcacatggcacccgggcggcgtagtcggctgccgcaccgtcaacgtctccgacagcaacaacgtggtgggcgcggaggtcaacctgctgatcgcgcccggcacgatccggccgcgcgtgcaggccgtggccggcagcaccagcgtgcaggcgctggtcggcggcggctacgacttcgccagcggcaaggcgctggtcggcgtgggcgtcaccggcgatcactggctgggcggcgccgactacgagatcgccggccgctggaccgcatacgccggcgtcagcacgctcaaccgctatcacgccaacgcgcgtccggaccccatcgccccgacgccggtcccgtcgaacaatgtcacggcccccccgtcgacgaacaacacagcgccagcccccacaccggctccgacaccgggcgccacccctgcgcccacaccaggtcccggcccgacacccagtccggcaccgactcccacgccgagcccaagcccaagcccaagcccaagcccaagtccgagtccgagtccgagtccgagtccgagtccgacaccgacaccgacaccgacgccaagcccgacgcccgctccggcgccagcacccagcccaacgccgaccccggcaccctgcggctgccacggcggcgaccatga

SEQ ID NO: 49 - DNA, 384 bp, Ralstonia solanacearum
atgaatgcgaacaaattcaatacccttcaccaacaagcactggccgtcttcgcggccgacatggtcggggagtacgtcgcggaagtcccgggcgtcccttggtttgccgtgaccctcggggacggtgctctcatcgcggagcactacgaggccgaggacatgtacgcattgaagacgcatgaaagcgactacctcatccagcgcgaaggtgaggccggggcggatgaaatttgggcggccctacagtgtgtgggtcgggcgacaggctaccttgacatgcacgcggtgaccggaagcatgcaccgccttaccagttcggaggaatcgcggcttctgaagaagctggagcggcgtggcttgctccttacggagcagatcgcataa

SEQ ID NO: 50 - DNA, 639 bp, Ralstonia solanacearum
atgcttcactcgattcaaaatccgggtcaccccatgcgcattcttgcccttcttgctgctgttttcgcgagcattggattcgctgctcacgctgtcgccgacgagatgtctacacagcgcgttgacggtgtgtcactgtccgtgcctacggcctggcgcgtagacacggctgaattcgaaaaggatagagcacaggctgatgccgctcacaccaggcgaagccgcacgctgttggccgtctcgggaccaggtgacgataccgctgttgcgtctctgtcgtttgttgtccaagagggcttcaaaccagatacgttctccgcaatgccggaccaaacgaaaaaccgccttgctcgtcagttgcaagagtttgtcgagcgctcaacggggctgcccgcaaagcgcagcgtcatcatcgaatggacgccttacgagacagtcgatatctcggggcgacaaacgctgcacaacctcgcgactgtccatttgcgaaaccagccgggtgctcaagacctgttctatatcccaacgatgggcggcatgtacctgctcagcgtttcagtctttgaccggcctgtggaggctgggcaccagctggcaaaggaaatcgccagcagcatccgggttgatgggaagttctga

SEQ ID NO: 51 - DNA, 882 bp, Ralstonia solanacearum
atgacgggcgttactctgggctttgtgcctgagccacttgctgttcctctgacgtgcattctcccctctcgcaggactccctccggcatcacgggcaccagaaagttcaagcagatccgctcctcgatcgaagaggtgggcctgatcgagccactttccgtgaccgcggttgatcagcagtcgggaaaccacacgttgctggatgggcatatacggctgctggtgctgcacgaccttgggcatgaagaggcggcctgtcttgtcgctactgacgatgaggcatacacctacaacaaccgcatcaaccgcctgtcgagcgtccaggagcacttcatgatccggcgtgccattgagcgtggcgtttcaccggaacgccttgccaaggtgctgtccgtagacatctcccagatcgtgaaaaaattgaacctgttggaaggcatttgccctgaggccgtcgacctgttgagagaccggcagttttcgccagaggtagcccgcgcgattcgcaagatgcggccaacgcgacaggtcgagtgcgtggaattgatgatcgccgcgaacaatgtcaccgtcgcctatgcagaagcgttgcttgttgccacgcctgcctctcgtcttgttgatggaaagaagccggccaagctaacaggcgtgacgcacgagcagatggcgaagatggagcgggagatgagcaacctgcaggagcagtacaaaatcgtggaacagacctacggccaagacgtactcaatcttgtgctcgctaaagggtacctcgtgaggcttctcgagtgtgagccggtgcaagcctatctacaccaacaccagcctgacctcgtgcgcgagttcgaatcgattcgcgacgtcgtttcgctggagcagtag

SEQ ID NO: 52 - DNA, 870 bp, Ralstonia solanacearum
atgattccgatcgatcgtatcgacgtgatcaacccacgtgagcggaacagcaaggtctttgacgagatcgttggaaacatcaagaccattgggctaaagaagccaatcaccgtcacgccgagggccgcagcggatggcagtgagaagtttcttctcgtctgcggcgaaggacggctcaaggcattccgctcgctgggcgagaccacgataccggcaatagtggttcacgtcagcgacgaggatgcgttcatcatgagccttgccgagaacatcgcgcgccggcaatgccgtcctctggagctgttagccggcatccgccagttgcgtgatagcgggtactcgccgaagatcatcgcagaaaagacgggcttgaccgccgcctatgtgcagggtgttctgaccttgctccatcatggggaggagcgtctgctgattgccgtcgaaaagggcacgattccgctgacggcggcgctcgcgatcgttggggctggcgatgacgacaaggctatccaagccgccctccaggaggcatatgagtcggggaagctgcgcggcaagcagctgataagcgcgagacggctcatcgagcggcgacataccttagggagatctgcctcccgctatatgaccgggagtcgcgcagacgtgaccacgtcaagcttggtgcgtacgtatgagggagaggtggagcgccagaaggccatggtccgcaaggcggagttcactcagcaacggttgctgttcgtcgtcggagcgttgcggcagctatttgccgacgagaatttcgtcaaccttctgcgagcggaatctttggcgacacttcccaaatatctggctgagcgtgtttggtccactgggagttcgccatga

SEQ ID NO: 53 - DNA, 1566 bp, Ralstonia solanacearum
atggcaatcgatcagtacacctgcgctacgccgatcgcgccttctgcgcttcgggcggcggaatacgtacgcatgtcgactgagcaccagcagtactcgacccagaaccaacgagacaagatccgcgagtacgcagcgtcacgaggcatcgaagtcgtccgcacctatgccgatgagggcaagagtggcctgcggatcgacgggcgagaggcactgcaaagcctcattcgagatgtgaccaacggtcgcgcggacttccaggcgatcctggtctatgacgtcagccgctgggggcgcttccaggatgccgacgagagcgcgtactacgagtacatctgccgccgtgccggcatccaggtcatctactgtgccgagcagttcgacaacgatggctcgccggtgtcaacgatcgtcaaaggcgtcaagcgcgcaatggccggtgaatacagccgcgagctctcagcgaaggtcttcgcaggccagtgccggctgatcgaactggggttccggcaaggcggtcccgccggctacggcctgcgtcgcatgctggtcgacgagcatggcctcgtgaaagccgagctctgccgcggccagcgcaagagcctgcagaccgaccgcgttgtcctggtgccgggtcccgagagcgaggtgcgtattgtcaatctggtctacagctggtttatcgatgagtccctaaacgagttcgaaatcgccgcgagactgaacagcatgcatgcgcgcacggacttggaccgggactggactcgcgcaaccgtgagggaggtgctcaccaacgagaaatacatcggcaacaacatctataaccgtgtgtcgtacaagctcgcgcgggctcgcgtcgtgaacgcacctgaaatgtggatccgcaaggatggtgccttcgagcccatcgtagcgccggacatgttctacaccgctcagggcatcatccgggctcgcgcgcgtcggtacagcagtgaagagcttatcgagcggctacgcaacctctatcggaatcgcggctttctgtccggcctcatcatcgacgagaccgaggggatgccctcctctgcggcgtacgcctatcgcttcggcagcctgatacgcgcctaccaggcggtgggctttacgccggatcgggactatcgttacctggagatcaatcgattcctgcggcaactgcatccgaagatcgtgtcgcaagcggagcaagctattgccgatctcggcggtacggttcttcgagacccggccacggacatcctggacgttaatcaggagttcaccgtgtcgcttgtcttgtcgcgctgtcagccgcgaccgaacggcgggtatcactggaaggttcggttcgatacaagcctcttgccggacgtcactgtggccattcgtctgaacaaggcgaacgacgcgcctttggactactacctgctcccgcgtctcgacttcgcacgccctggcattcgcttggccgagcgcaatgccatcgagtatgagagctatcgcttcgacacgctggactacctgtatcacatggccgagcgtgcaaagctgagaagggcagcatga

SEQ ID NO: 54 - DNA, 1406 bp, Ralstonia solanacearum
tggtggtcaccggccaacgtgtggcgccgctctcccgcgtcaggaggtgccgttctgctccccggggcagcacacaggatcacgttagcaggtggccgcgcaccgtgcctcacatcatctctttgcaatcagccctggatggtgcgcgcaccggcggcagcattacgatctgcaccctccagacgtccatctttgcaaccccgtcatcggacgcgtgctggagcatgtaagtgcctgaaggcagaggcgttttcggagctgacgcgtacatctttgcaatcgtccttctccttaggttcctggcacgctgtagatctcggttgcgcgccgtataggccgggtgagcctcgcgataccgtcgccagtagtcgctatggcactgactccatgcagcgcgggaccgcgcctggttgtcgcgatagtcgtcatcagagcgcaagtgctgctgttgccaccgtcggcgacgttggcgctggcactcggcagcggagcaatagcgttggcctggagtttgtgggcggggaagaaagagggtgccgcatgctgcgcaccgccgccgaatttgattcatcgcgagctccgggcaaggtcactgcgcggggccagcaagtagatgtggcgttcggggcattcaacccgaaatggacagctcgcacatgagtcgagcactcttccaccgggcgcgagccgctacggattgcaaatgatgtatgcacatacatcaggaaaacagtgcccgacgcgctcgattttgccacggctcaagggttgctctaccatgggccaaaaagtaggagctcgacggccgctcgtcttgcaagtgattgatttttaaggtatcttttggccacttgaggtggcgtcaagattttgcacttttcttggcacgtgtgccgagtcttttcagtaattgcggcaagggacgcgcgactcagtgtctcaatttatgaaaaaacgcttttaaatcatagtgcgatttttcaggaattgtgacactcaacaggttgttgcagaccccagaaaagttgatgtaattcacaaataagtgaatcgatccaaggcggccgcattgccgtggcgcaccagatctacccattaacatttcgtgcagccccacaaaaagaccgggcctctcaagaggtccggtctttttttaggtgtcggtagccacctcgccagtaggaatcgctctgacagagaaaggtgatgtcggtcgagctgcgcgcaatcgctacaggggtatagctgggacttgccagtcaaccccgctcacgagaaagcgtcctccgaaattctagcaatgtttgcgggcccgacggtaacacgccagaaactagggaaccgctgatcaatgaagtcggcgaatggaatcgcgagtcatgccatcttggtttttcggattgagg

SEQ ID NO: 55 - DNA, 1906 bp, Ralstonia solanacearum
agggggctggtggagtcgctacatgcttgaagttactaaaattgtggattttttgacggcacaaccctagacccagcttggctctgactgacgatgacaaaaaaatctgagttatggatttttgggacaaatcaggactgagccggctggtccggtccatgggtaggggcgtactttgagcggtcagagcggccgtacgtggcaggcctgtttcccgcgtgcgttgcgtcgaggccgcgtgaatggcgtactggggcgcccgctaccgggccgaattgctctgccaccgttcccgatgacgggatgttgtggtgtgcgtcagtcagcatgattctcgttggtagaatatgagcaataatcacaagaaaccaaccgaagtcgtttgactgcctccgctgcgcggggccaatggggcgagatacgcataatgacggcagaacgtgccactccttcgcgaattgggactttccggttctgataatggcaatttcgctcgctttcgagaatgagcaaattcgccagacctgcgaaagtagccgttctgctaagcgccgccttggtgtaacggtagcggctcaactccaggtgcggctcgctgacctcaaggccttggatgccgcgagtgacgtaattgacatgggctttgctcggctcgacccgtccgaagatacccggcttgtcattccgctgggctgtgagtatgttctgagggtagccgcgcatcatcgtcccgaaccacgttcgtcaactggtaagttggactggaagaacgtgactcgcatcaaaattctggctatcgagtgcaccggtgaaagctgaccatttgttttctcccacttgggctgtgccgccagggcggaccgtagttgacttgctcgctgcgaggggttggtcgcgggatgagctggccaactccattgagctggtgcgagaagatctcgatgacctgattgacggaaccgcacagctatcggaaagcgttgcttcccggttggggggcgtctttggtacgagcgccgccttttggattcgtcgagaggagcagtaccgcgagcagctcgcgagtttgcatgtcaaagctgatgtggcggatgcgggctacaacgagtggctgaaggcactgcctcttgcggacatggctcgctttggctggttcgctccaggcgcgacctccgctgacaaactggcggcgtgtcttgatttctttgatgtcccgaacttggacgtgtggagtcgggactacgacgatgtgaagcgtgcggttgccttccgggcatcagccgcccacccagtgaatgtacctgcgaccaccgcgtggcttcgacaaggcgagcgtgaaagtgagcgtattgagtgctcagcgtgggacccggccaagttctcatcggcgctgaccaaagcgcgtgcgctaacccgtaccaaggacccgcaggtttttgtgccagctctacaagcgctatgcgctacgtcgggcgttgcagtggtaatcgtgcgagcaccgtccggttgcagagccagtggtgcgacgttcttctcggctccagataaggcggtcctgctactgtcgttcagatatctcacagacgaccagttttggttttccttttttcatgaggctggtcatctactgcttcatacggacagggatagtctcatcctggagacgtcggagcgaacgacgtccaaaatggaagctgaggcgaacgagttcgctgcgaaaactcttttgccagtcgagctcaaccgccggctacctgaggtggctggcgacgttcgagcgatacttcgtcttgctcgagaggccggcattgccccaggcatcgtcgtcggccaactgcagcatcgcggtttgatcaaacaccagcacctcaacaagctcaaggttcgctatagctggtagtagttc

SEQ ID NO: 56 - DNA, 909 bp, Ralstonia solanacearum
ttggcagctgacctcgaagcaaaactgcttgagtttaaggcggacaacaagccgctgccgggcatcgcgaacgacgacaatcgcattgcctatgttgagcagcttgtcgagagtatccgccgcgtcgacttcgtaacgactatcggacggcgcggcatctgcccacagagacgggatcctaagtcagacttgttcgaccccttgcgggctgcgactctctaccggcagcaggaaaatcttgatgaggcgttctggctcgcgttcctcttcgtccatttcgggaaaagcctgcgaaccagatggaaccttatccgcgcgatctatggcgcatggaatgaccagtttatttggacgtgggagcagttcaacagcgccccgaatattttcacaatctggttcaacgaccgccttccggagttgctggaaaatcgaaagcagctccaattcggcaaccaccgcaagtacgaaagtatcgagaaagtggatgtggtgcttgaaagctacgcggaatgggtaggccccgaacgttcgcatgcggcaatgctcgaacgtgccacggcagccgtcggcgacaacccaagggcgctgtttgggtatctatacaagagcatgaaagtcgtccaccggttcggcagaactgccaagttcgactacctcacgatggtcgcaaagctcggcctagcgagccttcgtgcaccttcggcttacatggttggtgctactggaccgctgtcgggtgcccgcttgctattcgccggaacgaccgaggcaatgcacatctcgtgcgcgcaagctgaccaactcctggttgtgcttgataaccatctgaacgttggaatgcaagtgctggaagactcgctatgcaactggcagaagagcccaaacaagttcgtggctttccgtggctag

SEQ ID NO: 57 - DNA, 852 bp, Ralstonia solanacearum
ttggccggcagcaacgagctcagcttcatccagtactacatcagccggtacgacgagttctcggacgacggaaacaccgttcacggggcatacggccctcgcttgttcaacatgcgtggtgtcaaccaagtcagcaacgtcatcgagaagttgagcggcaacaacgatacacgccgtgcggtcatccaactgttcaacgccgaagaccttggttgcgatgtggcggtaggcagaaaagatatcccctgcacgtgcaccatgcagttctcgctcagagcgcgtcagctccacatgttcgtgacgatgcggtcgaacgacgcctatctcggcctgcctcatgacatattcgcgttcacgatgctgcaggagatagttgcacgggccgtcggcgcggaactaggcacatacaagcacgccgtcggaagcctacatttgtactcgaagaacacggaccaggcgacaaagtatgtcgacgagggcttgcaggagcgcatcgccatgccatcgatgccggtcggtgaccctcagccggcccttaacgcgctggtagatgcggaacgggaaattcgcttggggcacgccgtgaacgtcaaccagttcgaaccctactgggcagacctcgtccgcttgttgcttattcagcggcatctgaaaaataagactcctaacagttcgcggtcaattcgtgcgttagccaagaagatgagcacggcgaactatgacgcgtgcatacagcaacgcgttcgcaagagccagcctgcgccggagctcgccgagcctattggtctgctgttcacgcctgctgaactggatgccggaatggcctcattggacacccccgccgacgaatag

SEQ ID NO: 58 - DNA, 1315 bp, Ralstonia solanacearum
atttgttcgcacaagagccatctgcaccactcgctgaagcccttcgccccaagaccttggacgaggtcattgggcagtcccatctgctgggtgacggcaagcctttgaggctggccttccagtcaggcaagccccactccatgatcttctgggggccgcccggtgttggtaaaaccaccttggcacggctcacagccacggccttcaagtgtgagttcatagccctctctgcggtgttttcaggtgtcaaagacatccgtgctgctatggagcaggcacagcagaatctatctatgggcaagagcaccatcttgttcgtagacgaaattcacaggttcaataagtcccaacaagatgctcttcttccttatgcagaaagtggcctcgtaacctttattggtgccaccactgagaacccttcttttgaggtgaactcggctctgctgtcacgtgctcaggtctatgtgctgaagtcactgacagaagaagaactgaagctgctcttgaagcgagttcaagaagaagatgctcttggtgacttggagtttgaagataaagcagtagataccatcattggctatgcagacggtgatgcaagaaggttcttgaacctgcttgagcaatgcaaaacggctgctggtgctgctggtgttcagcatgttaatagtgacttcattcagaatgccctaactctcaatagcaggcgttttgacaagggtggagacaacttctatgaccagatttcagccttgcacaagtctgttcgtggctccaaccctgatgctgccctttattggctgacacgcatgttggacggtggtgctgatccacgctatctatccagaagaatcgttcgcatggcttgggaagacattggcttggctgatccacgagccatgcagatagccaacgatgcagctttaacctatgagcggctgggtagtccagaaggtgagctggccctgggtcaagcggtcatctatctggctgtagctgccaagagcaatgccggttacaacgcctacaaccaagctagagccttcgtgaagcaggataagagccgtgaggttcctgttcatctgcgtaatgcaccaacaaagctcatgaaggaactcggctacgggcatgaataccgttatgctcatgacgaacccaatgcctatgctgctggtgaaacctaccttcctgaaggcatagaagagccgggttggtatcagcctgttcctagagggttggagatcaagattggtgagaagctgtccctgctgaagaagtgggatgaagaagccaaaaagaactgaataaggggatcaga

SEQ ID NO: 59 - DNA, 1950 bp, Ralstonia solanacearum
atggagcaaagaacacttctctacgatgagcggttccttgaaagctatgctggagccatcatcactgatcctgctacggcgattgtggagcttgtcgccaactgctgggacgcttatgccacagaagtgaagatcacatggcccgatgttcaacttgaaaagcagttcaagattgcggacaacggtcatggcatgacccgtgaagagtttcaatacatctggcggaccatagcctacaaccggttgtcgtcaggtggaaacacaacggctcccccagcagatgttcaaggcttgccacggcttgtttttggcaagaacggtaaaggccgttttgccagcttttgcttcgcttctgaatacttgattacttcaaggaagaacgggcaagagttcgtctgccgtgttcacagaacctcaactgatccgctcgttctagaagaaatatcgttcaaggaaaagggtgttgagggtcatggaacggagattgtgggtaacggcatcattccccggctcatgttctcggaagagaaagctcgtgaattgatcggtagccgtttccttgccaaccctgctttcaaggtgaagttgaacggtagtgaaatcacgttcaatgacatccctgacttgttgtccaagagcgaagtggatgttgaagggtacggcaaggcagttattcttcacattgacaccaagaaggctgacaagacaacgaagcaacatggactggcatggtgggttcaaggtcgtgctgtcggtgaatgcaagtggagtcgtagtgactacgaaagaattcttgacggccgaacttcagaggcgaagcgattcacattcattgttgaggctgactatctaaatgcccaagatgcagtgctgggtgattggagtggcttcaaggacgaaaaccaggcgtgggtgaagacccgcgaggccgttcaagatcgcatccgccaaatcatttttgattccagcaaggcagagcgtgaaagccgccgcagtgcagtcattgaaaagattggcacaacgatgaacaccctgtcccctgttagcaaggaccgggtggaaagcttcgtcaatgaggtggtagatacctgcccgaactttggtgaaaacgagatcgtccagttgtccagcatccttgcaaagctggagcagtccaagtctcgctatgggttgcttgatctgctgcacaagtgcgagccaaacgactacgacacccttcatgacatcctgaaggaatggaccatagggatggcaaagctggttttggacgaaattcagaaccgcttgaaattgattggggagcttcgcaccaaactgaaggaagttggcgttgatgaagtccatgagttgcagccgttgtttgagcgtggcctgtggatgttcggggctcaatttgaatccattgagttcacgtccaacaagggcatgaccaatgtaatcaagaccatcttcaaggatgaggatggtaagggcactcgtaaccgtcctgacttcgtagccctgcctgacagcagtgttggcttctatgctcgtgcttcatataacgaagattacgatgaagacggtgttgaacatctagttattattgacttgaaaaccactggtcttgctctcggtagcaaggagaaagatcaagtctggaagtatgtgaaggagctacgagaaaagggctacctgaagaagcatacccgtgttgatggatttgtcttgggagacaagattgaatcgggtgagaatgaccctacaacgcatggtgaagaagtcaaaatttcacccatgctgtatgacaccatcctcattagggctgagaagcgccttctgaacctgcacggtaaggtcaaggatgctccgttcctgatagagcagcaggaagtcctgaagaagtttttggagcctattgaagtcattcaggcggaattggtggaagctgacccagtggtggcgaaatga

SEQ ID NO: 60 - DNA, 765 bp, Ralstonia solanacearum
acctcgtccgtgaattggatcgcagcctacaactacctgtttgcttctttcaacagtgagaacaaggatctctatgttggcggttctgtattttgtcgcatggtgcagcaggtcgatcctggctcaccaagctaccaacagttgctgccgctgagacaaagccaaggaaagtccaattcgcgcaaggacttttattgggaccttatccaaggtcttccagaggcccagcgatttcaactctatcgtgtcttcatcaaccacattgaagttcacgacaagccagcggcagacaacatcaggaacattgtgttcggcggtggttatgcggtgcccactacggttgttcccgttgacctctggaactctgagaagctgaacaacagtctcaatgacattgaccatgccattgatgctcaccattacaaccgggcaacgacactgagctacacgtgtcttgaagggctctacaagacatacgttcgcaagcacgttcctggacaaatggcgctgacggatctcatgccattgtgcaaggtggtgaaggaggacatcagcaagaagcttcaggcacaaggcccgtttcctgtcgagattgtcaatgccatgccgaccttgaccaatgccattgcgaactcaagaaacggcttcagtgaatctcactttggtgacgactcccagcggtggcttgctttgtttgctcgtgatctgaccaactcaatcggtcgtctcatgctcaacttcatgtgaccaccggcgagaggaaga

SEQ ID NO: 61 - DNA, 1149 bp, Ralstonia solanacearum
atgactacttctagaacatgcgcactttgcccaaacatactgtcagccaaaaataaaacaaaagagcacatcatccccaatgcgattggcggcaggaagaagaccactgggtttatctgcaacgattgcaataacaagttgggtcagagctgggatgcggagcttgcaaggcaacttaactggttttccttggcagtcggcatcagtagagagcgaggtgagccacctaaacaggttgtgcaaacaatcgatggggacaggtattggcttctcagcgatggctctttcacgccagaaaaatcatcttattcagaaaaggatctgggtggggcaatcgagatcaacctgaccgccaagacggttgaagaggcaaagcagcggattcaaggtatcgccagaaaatacccgaagtttgacgttgaaaaagctctcaatgagttggagataaaaaccagctatttggattcgcctcttcatgtcagtctttctttgggtgggccagaggctggaaggtctgtagtcaaaactgcgttttcttttgcatctgaatgtggtgttccacatgctcaatgcgagaaggctcttgaatatcttctagatcctactgctgaaaatttcccctttggatttgcctacttgtctgacctggttgaaaaccgaccaaaggacaagatttttcattgtgtgtctttgcatggtgatccaaagaagaagcggctatggtcttacattgaatacttcggaatcttcaggcttgttgttctcctaagtgatgtgtatgtgggtccctacaagaatgaggtttatgccatcaatccagttgatggagaacggcttgccgtgaaggtgactacccagatcagcgaagaagaatttacttcgttgatgaggggcgaaggtttcgaccaagaaaagcatagagcagcggcggactatgccctgcctatcatcctggatagagggcgaagccgaaccttggagcgcacagtcaaggaaggctttgaacatgctgccaagcagcttggaatcagtgaaggcgaaatcattcccaaggaaaaggccactgagttcactgcctttatgatggagaaaatctctccatacatagagcatctagttcgtagtgggcacagaaatgaccaaccataa

SEQ ID NO: 62 - DNA, 693 bp, Ralstonia solanacearum
atgaccaaccataagccagcaaacggcaccaagaagctcctgcttattgaccttgagaacaagcacaaggtagacctgtctcccttggatgagagttacagagctatcatttttgtgggtgccaaccagaacccgccaaaagcttccaagaagccagcaacagcccataggttcaagagggtggacttcctgaagatttcaggtgctggcaagaatgccttggacttccacatagcctttgaactcggcaggaccttcgagactgcacctgatacccaatgcttcgtgctcagtggtgacaaaggctttgacccgctgctgaaccacttgaatagcaacggtatgagttgccgaagggtggaaagcattgatgagcttgtgccagctacggtggtgcctgtagcagttccatctgatgtggttgtctgccctcactgtaagaaggccagcaccattgagcatcacggcggccactggtgttctaactgtggaaggtttgctactccaccagatcccaagctgctgccgtccaatcaacccggctacaggcagtctcaaagagacaggtctgaagaagactatgccatctattcggtatgtggctggtgccatcaaagaactgatatgactggcggtatctatgacgatggggaatggatgtgtggaggctgcattgccaggtgcgccagatga

SEQ ID NO: 63 - DNA, 939 bp, Ralstonia solanacearum
atggacttgaaacctgcatatcaaccctcatgttgtggtggtttatcgttgccagagaggaaggccgtgccaagaaccattgaacagggtttcaacgattttcttgtaaatcttagagccactgctgctgagactgccgcaaccgtgtctcatcgggcatccatcaaggcatgcctggaaaataactttggcttgaaacgcttcacccgaatcggttcgtttggcaatggaacaaatgtgtcgggctacagcgatgttgactacttggcttgtctgccgactacctcattgaaacaatcatcaacctattctctcaggttggtgaaagaggcattagaaaaccgcttccccagtacgggtgttgtggttcgttgtccaacagtggtttgcccctttggtcaatacaaatctcagcatacagaagttgtcgttgcagactatgtgaaagaacaaaatggctacaaggtctatgaaatagctaatggcaatgatgggtggatgcagatttgccctgatgctcacaatgcttacgttgaacatgtggatggtaagcacggaggtaaagtcaggcagttgattagactgatcaaggcatggaagttctatcagaatgtacctatcaaatcgttctatctagagatgaaggtggcgaaatatgcgagcaatgagcaagccatcatccatcacctagatgtgaaatattttcttgaccgtctcaataatgaaggtctgtccgccatgcaagaccctatggggatcgctggctatatctatccttgcaacagtgacgttcagaagattgacgcactctcaaaactaaacacggcagctacacgggccaacaaggctgaagctgctagagcattggggaatataaaagaagcttttgattggtggcgactgctttataacgacaaattccccacctactacctatga

SEQ ID NO: 64 - DNA, 843 bp, Ralstonia solanacearum
atgagaagtataaccaaattagcattcggaatattattggcagcagccatattcacaccacaggcacatgctcaatatcagcaatcggacagatcattaaattgcgattcaacttgtgttttacagcagcagtcaatgctcgctttaatacctaaagcaaacacatttgttgcaccaacatatgtagccagccccacccctaaacctaaatgcggcccttctgctggatctatcttgccaggtgctcctaacagctttttgtgcgatgcaggaacacccagtgcagtttctactaccaccgcaaatggagaaacaacttactcttggacttgtagcaatagtttaggtagtcaattatgtacggcaaaccagagagaagttggtgcatgtggtgctgacaatggcaaaactctgtccagcaatcccactaatgtttgtgctactggtcagtcctacggcttcaacttcgccaacaatcaatacacatggggttgtgctggcaattatggaagcccagcaatgtgctctgctgcctatgctccaccaattccaccatctcagagcaaaaccatctatcgttactacgatagtatgttacccggtgaaggtggctcattcacttctacttatgttccattatctgcaagtgaaggctacatatcttctagagaaagccaaggaccttgggggagatgcagatatgagtggcaaacagcatcccaagaaagaatattacctagcaggacatacaacttgaatggtcttgcctactgggtcataagcttgagtcagtggaatagctatacatactggtataacgtgtgtgtagatggaggaagttaa

SEQ ID NO: 65 - DNA, 178 bp, Ralstonia solanacearum
aaaaaaaaaaaaaaaaaaggaaatcgtagcgcatttcacggtgcccctccgagagaaatgcctgagcctgaccggtttgcaccatgaggtgattgaggtcgccaagatttatgagaacaaccatatcttgaatttggcaaaatccagaaaacgcgaccagatgggcaagaggaaataa

SEQ ID NO: 66 - DNA, 1580 bp, Ralstonia solanacearum
  misc_feature (1202)..(1549): n is a, c, g, or t
agcgcgcaaagcgcgccaagataggtgcgaaagcagcgaaaaaccgtgctgaatcgaagcatcccttcctcaatccgaaaaaccaagatggcatgactcgcgattccattcgccgacttcattgagcagcgcttccctagcgtgatagaaatgaaggagtagaacgaatgaagaattgggtgggaatatttcttatgttgtggaatgtctgccacttcaaacatgaagaatggcatttaatgagacagcgattgaaattaaaaaagcacctctagggtgcttttgtttttttacatggcctgctttgttggaatgaatgcgtgtattcgtttcaatgaagtgggtgctccttagctgtgaaaaatatctagccacctctttctacgcttgatgctatctttatgtgccagcaggaaagggaaaaagtcctcatagtgtggaatcaccattgaggaaattccattttctgcttcatattcactcgccttgtgggccattcctgtaagtaagaagtggatgggtcgcagcatctcgatcagtcgccagagaaccagtggggcactcttctcaatgacatattgacggtgtaggcagtcaaacaccatcatggggtgtccatgctcatcaaggtggctggtgaagccgttgggtggcccttctctatccatgcggtacatgaggttcagtgctgacctctcacgcacctggtgcggtgtgtagtcttcataggtagctctggcaagatcgagccacttgtagagagggcttctgtccatcttggcttcgcagatttcattcagattgaagtagcgggcaccatcaccgtattcactaatgaagttcaatagaacttcgtcattggcggtcagagtcggtgctacagcattggggtggctgtagatgggcttcaacctgatgcactcttcatagagcacatcaatcttgtgtccaaagcctttaagctgcttggtggtcggtgtctggtagttgttcgtcagcatgtggtgtgtcaccacagccaacttcagaagcctctccataccaatggagagatgaaagaacgccgaataaaagtagccgtccttgtcctggaagaagttggccttaagcagaaggtcgaaacctgacagcaacgtgttctttgttaggtgagcttcttgtgcaagaaggctgaattttttatcaaaacctatctgcttcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatcaacgtgtccctagcgcgccggatgcaga

SEQ ID NO: 67 - DNA, 2445 bp, Ralstonia solanacearum
  misc_feature (1077)..(1966): n is a, c, g, or t
aggggtttgtagacatggagccatcgcaatcgagcaatgccgaagggctgagcggagacgatatggaggttgtctctgcagtatcccggcgatacaggcgttatcacaagatagccgcaaggcatctgccggaatgggcgccgtctggggttacgctaagcctcatacaagcgatgttgaacctccgaaacgttcgttgcgcaaagagaaacattcacgagaacgatgacggcacccagtcaacccatgcggagcatctgagatatgccgaggccgaaatagctcgtctcgtgcgtctgtgtgcctatagcaatgaaatgcagaaggcttggcggacaatatcaactatgccagctcgtggtggtggcgatccggcattctccatatgttcaaaaatctcctccacgctggggcaatttggtgacctccccaagaggcttccagcacaacgcaagaaatcgctttctcgtgtggcgaaggcagtggacgatctgcttgaggcaatcgaatcggagagcgaggcgcaggactctcttgagattgcactgattgaatacctgggaggtcagaacctcaagatgcgctctgacgacggagaggaaataggagtcgatgaatctctcgatcccttccgagcgcttagtccggccatcattccatgcgacgatacagaaggcttggaggactcccctcattgctccaagccctggcacgagtggtcatctttcgatagatacggttggcttcacgatgaagtatccatgacgggaatggccgagttgctgatcttttgtcgcgacagaataaatgccttggctcaggagccgcccaaaattctgcagcctggaagaccagatggtgggctacagccgtttctgattcggagtttgtccaactggatgaagcgcatatacggacagcccttggacgactctgtagcactgcttgtctctgccacgctagatctgccagagcctctgacaagagaagcgatccgcccgtatctgaaaggcaccgggaagatttcgagcaggaatcgatagatttcttcccgtttcaaaatatcagagtccacgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgggaaaaacgcgccaacgggtggccccggttggggtgggcgaacccttggcccgccaggaggcaatgctggccagtcctgggagcattgcgtcctgagcgcgcaaagcgcgccaagataggtgcgaaagcagcgaaaaaccgtgctgaatcgaagcatcccttcctcaatccgaaaaaccaagatggcatgactcgcgattccattcgccgacttcattgatcagcggttccctagatgaatcgtcgactgctacgccggcggtggggcttcgtgaatgacccccatgcgcaccatctcgtcgatttcggcacgagcaactgccacaatgtcttccagtgtgcggaaacgagggctttgagggcccatgacgtcttttgtgactggatggcccagcgcgtcgcggtaccgaatccggccagtgcaagtcgcttcgctaagctcttggactaggacgatctgcgcgcctttgtacgctt

SEQ ID NO: 68 - DNA, 306 bp, Ralstonia solanacearum
gtggcaaccaagaaggtcaccaagaagaagtacttgaagattctcaacaagcgcctgcgtgccgagccggatgcgccgtccaccgccctcttcgttttctacccgcctggcccaaaagcgaagcacgccaccggtgtggctgcaagcgagcccagtagcccgcgcgaactgcccatcatggctgagatccagcgtattgccgcaaccgagctcatcgttatcgacatgggcgacaggcttggcaacggccgacgcagaagctccaaatcaaaaccggaggccagccatggctcaactacccgttga

SEQ ID NO: 69 - DNA, 1308 bp, Ralstonia solanacearum
atatcgccgcctggatgataggaaaaaagatcaatggcgtttcgcattcgtcactatccacacaatgacttgctgaacctcgcgtcgcatcactgcgagacgatcaagatgaaggtgaaaaacaaccagctcgaaggtataacgctcgattgcatgagctgtttgatcgcacttgcctttgcggtggaggcgctagtcaattttgttgggtctcggaaagtgaccgactggaacgagatggccaaggctccgagcaaggtggcaaagatcgcgaaagcagtgggactcgacatccagccagagtttgagcccttttccgcaatctatacgctccggatcattcggaacggattggctcatgggaagccgcttcacaaaagcgccagcgccaaatcttctgacgagttggcgattgccatgcaggccccgtgggcaccatattgcacccctgaacaagttatagccctgtatgagcaggtacgcgaacttcgtagggtgttgttcaaaagcgcaaacatccgatgggttgacagcctgacctctgccattggctcgcacggataggaaaaccatggcaaacgttgtgaccttgctcgatcactggcagacgctcgtcggcgcgttcataggcggcctcatgggcgtagttggtgcgctaatcgttgcagtactgaccgtgcgccgccagcgctgggtgatggcctctgcgttgctccccgacatgcaacaactaagagccgccgatgatgcgctcgaacgagcgcttcactccattgatccgccgctcggagagtgggttcgatctcagtggtgtgcagagcgtcttgcacaaacgcaccccacgttgtcggcgcttcatgacggtgtagccgtgacccaggtgtctgaccttgatgcgcgactaagcgcacacttgatgctctgtcggatgaggcacaaggacttggacgcacttctgcagcaatttgtcgccgcgctccatggctcgcgggcgccgatgcctgcggccaatgttcccgcagcgatgaccgtggtgcggcgaagtgcaaaacgagtacagcaggcgtgggatctatgcgtcgaacacgggacgcttgcggaatacctgctggatcgattgattttcaataggtggccaaacatttggcaccggctgcgcatgcgttggtggccgaacgaccttgaccatcgctcgatgcatttgctgaattcggggtcgatcctgggcacacaggactcccatcaggacgacgtggccggataggaaaaatcaattctcgtactaatgacgagcttggagattcgaca

SEQ ID NO: 70 - DNA, 711 bp, Ralstonia solanacearum
atgcacgacatcgaaacagacggcttcttggcacccgaggccctggcgtttcgggatgcagtgcgagtccaatttgtcgagcaaatccgtgagtgcgaggatgtttcgcgccgcgccacgacgcaagtatttgagcaacacatcgcgaacgtgacggttgcaaatgcgacggcagcctcgctgtgggcgagatgtttgagtgcaagccaggccgccatcttgctggctgagcgcggcatgggtgtggaggcgctggcacttttgcgtacggcttatgagaacttgttcttctgcgctgcgctggtcaagcagccggccgtgatagtgcgcctcgccggtgaagatacgggtcagcgtctcaagcaggcgcgtgaaatgctcaaagacaaacagatcttcgcggcaatcacggatgcagatcgggcgctgcttgaggcgttcgtccgcgatacaccggctgccacgaaagccatcagtgcgtacgaagccgctgagatagctggcatgaccggcatctatcaaggtgcgtggcggacattctcattgattgcgacccatgccacgctgaccggggccgggcacgcatttggcaatgacctgtacgatctacagttcggcccctcgttcgcaaacgtcgactcggcattaggcttggcccgtgactgcattcggctcgggcttgatgcggttgccccgatttttactgcgtga

SEQ ID NO: 71 - DNA, 531 bp, Ralstonia solanacearum
atgcgctccgatcagcgcgaagggcaggacagccgcgagacgcggttggccgatcagcttcgcaagggagtcgaagctaccgaagttctcgactacatctttggccttgaatacctttcgccgcgctactcgttgaccttcggggggcaagacattggccaactctcgcctggcgagcgcggattattgctcttggttttctaccttctggtcgacaaggacgacattccgatcgtgatcgaccagccggaggagaacctcgacaaccagaccatctacaagatcctagtcaagtgcatcaagaaggcgaaggagaggcggcaggtcatcatggttacacacaatccgaacttggcggtggtgtgtgacgccgagcaaattgtctacgcgtctcgtaacaccgagggtgttcgatttgagtatgaagcgggtgcgattgagcaaccggagatcaagggccgagttgtccagattctcgaaggcaccgagcccgcattcaagaacaggcaatcgaagtatcggctgcagtga

SEQ ID NO: 72 - DNA, 2405 bp, Ralstonia solanacearum
tgccccttgccctggccggctccacttggaagaaatgggatttacacgtccacacgcccgaatccttctatcacaactacccggggagccaagatgaagcctgggaagcgtttctcgctgacgttgaaaagctgccagaggagttcaaagtaatcggtatcaacgactatgtgctcgtcgatggctacgaaaaggtgctgaaggcgaagcgcgagcagggaaggctgaagaacatcgatctgattctgccggtcgtggaactgcggttggacaagttcggcggcgttgtccaagggggaaaaagcggcaaggcaccatcaagctggagcaggatcaacatccacatcatcttcgatcaggtggatcctgaattcatccgccagcagtttatttccgcaatcgccccgagctaccgtcttcttcctgggtcgactggcgaaggaaaatggaactcagtgatctcccggcaaagcattgaagctcttggtgcagccatcattgactcggccccagcagacaagcgggtggggtatgggagccctctcattgagggtttcaacaacctcaacgtcagcgcggatggcttgcgcaaggccctggagaacgaggtcctcaaggggaggttcatccttgcagtcggcaagaccgagtgggagaacctcaaatgtgatgaccactcgatcgcggagaaaaagacgctcattaacagtgcagatcttgtctttaccgctgcggaatccccagaggatcacgccaaggcgcgcgccaagttgaaggagtccggtgttaactccaacttgcttgactgttccgatgcacattggctgagcgcatcgaccgacaaggaccgcatcggtaactgctttacctggatcaaggcggactgcaccttcaggggtctgcagcaggctatcgaagagttcgatgatcgcgtctgcgttggcgataacccggccaagcgccagtacgttgagctcaatcgaacgaagttcatccgctcagttagggtcgccaagaaggaaggctcaaagctcgctgacacatggttcgacatggaactgccgctgaattcagatttggtggcaatcatcgggaacaaggggagcggaaagagcgctcttgctgacattatcgcgttggcggggaacaccaaaaactatcgtagcttctcgtttttgaacgcggtgcgcttccgggatccgcgaacacggcttgcacaaaacttcgtgggaaccttggtctggcgcgacggctcgccgactacgcgggatttggaccaagatcccgagcccagtagtgtggagcgcgtgaagtatctgccgcagagttaccttgagacgctctgcaatgagctgggtgcgggcggatcggcgactttcgatgccgagttgcgaaaaatcatctactcacacgtcccgctggaggaccaacttggccagtcatcgatggacgcgctgctcaatttcaaggtgaccgagatcaacaaggccatgggggggctgcggggccagattgctgcgctcaacgtcgagattcttggtacggagcgcaggcttttcccggaattcaaggagagcttggtcaagcagcttgcggcaaagaaggctgagctcgcctccctcgaagaagccaagcctagacaggttgaggatccgaatgcttcccctgaagcattggaagagtccaaagcggccgcaaccaagatcgacggtcttgagcagcaactgcaggatgtcggcaaggaggaagcacggcttcgggaggcgaagtccactactgcaaaggggcaagcagtcgcgcgccgcatcgcccaggctttggctaaccagaagaagcagagcgaagctttcgaaagcgagttgaaacagctcttagcagagctcggggttgagattcccatcgactcattgttcgagacccggatcaatgccgggcctgttgagggaatcgccaaagcgatgcaaggggagatcgattcaatcgacgcaaagcttcaaagtgcagagcccgacagcatcttgaagcgacgcgaggagcttttcgcttccctcgctgaggccaagagcaagctcggcgagcgtcagcgtctcttcgtgctctacaaggaggagttggtcaagtgggaaaaatcaaaggccgacgtgatggggcctgccgacaagccgggaactatcgcgcagttggaatccgagattgcggcgcttgccgcgcttcctgatgagctccagaagctcattgagcaacgctccgggatatcacgagagatccatgggttgattcgcggcacggtcgacgaataccaacggctctacctgcctgtccaggcgttcgtgcagtcggctgggcagatggacatgaatcttcctcttgaatttcacgtccgcattgaggagacccgcgggtaa

SEQ ID NO: 73 - DNA, 809 bp, Ralstonia solanacearum
tgagtttgaacccgcattggcggtggacgggttctgctgctgcaactggcgctcacggttgattgacggtcgccagtgaaggtctcgtctacaagtgaagtactccccaggactcagcgggaaactgagctgttcgagagcgagcttgcgaatgtgatcgccttcgggttcgcgtgattgctctcgatacctcagcgtgatttcctgcttaacaggatcaatgacgaaccaccgggcccccctccgctcataaaatgccgcgcgcggcccttcatccgccccacctcctgcataaatattgtccccgaccacgacaacgtcgccactaccgctaacctgtgggcgaagtgacttgaacgttcgcccttggtcgaaggatacgtagactgcggcatcatcccgagcgaccacatagggcgaattgaccaccttgaacatccccgtcggattgcgcttgccattaccgccaaactgcgtccggatgcccaactgctcgtctacatagaacaattccgcctgacacggcaggttatatcccggccccggatcgttctcgatgtagcggtgatcgtcgaagcggtacacaacctgtttaggtggacgcgccatattactcagcgtcttctcgctgatcgtgtcgccgaaaccgaccgacgctgcagcccggtccgcctgggaaagaccgcagccagtcagcgatagcagcgtgacggttgcggcaaccagcaacatgcggcgcatggcgacgggatgtttctcctgtttcaacgagagctccttaagtagttctgaatggatgcgcacgctct

SEQ ID NO: 74 - DNA, 2658 bp, Ralstonia solanacearum
atggccgaattcgatcgaacggcacatttgcaagacctcttctttttccagcagaaccagcgcttgatgggcctgtcgttcccggacgacgatcccccttcggccgccgaccaccggggccgctcctttcccgtgcagatggttgtcgagcgcctggaagcccaggagggcctgggccaagacttccgattcgagttgacgctgttggcagaccatgcgggtctcgtgttggcggacatgttgggcaaactgctggcagtgtcgctggtcaagccggacggcacgccgcgctggttcaccggacatgtggcggaattctcgttggtgggcagtgaccccggcatagcgacctatcgggccgtactccggccgtggctgtggttcgcgcggcatcgggtgaacaaccgcgtattccgggaccagagcctgtaccagcagatcagcgaaatcctgcaagactacggggcttgggcgcaatggcgctgggacgtggcgggagaggatgcaccgttcaccatggccgtgcagggcggcggccagggcgaaagcgatcacaactacatctatcgacggttggaagcgcagggcaagacctgccgcgatgaacacgatgcgaccggccaccggctcgtcatcttcgacagcaatagccaatgcccgccggtggaggagagcgctccccggatacccttccaggccgagggcgggccgcaggaagaaaacgccatccagcgctggacaccgatacagacagcggtggccgcgttgttcgcggggagcgccttcgatttcaagcgcccgagagcgcagcaccatacggaatcgacgcagcgcaagcaggggcgcgttcccgagctggaggtgcacgaatacgcggggcactacggtttcccgaaagaggaccccaggggcgttgcgcagctcaacttgcgccgtgaagaaatcgaagcacacgcgcagcagtaccaagccgagggcaacaacgccagcgtggcgagcggcttatggttcgagttgtccgggcacttcagcaacacggagcgcgacgaggacagccaatatctgatcgtggaggccagccatagcgcgagcaacaactaccttgctgaacttggtcagcaggcggggcagaaggcgcagccgcttgccggccgcaagggcgaataccgcaaccgctttaccgcgatcaagcgcacggtgccctggcgccccggccggggacgaaacagcatcgaatggaaggtggccggacaccagacggcgctggtggtcggccgcgacggcctcgggtctctggatgtggacgaatacggccgcatcctgatccggttccactgggaccgggaaggaaaattcagtgcacgcgtgcgtgtggcgagcaattgggcgggcggcgaaaccggcatggtgtcgtggccgcgcgtgggcagcgaggtggtggtcgcactggatggcaatcccgatcatttcctcgtaatcagcgtcgtccataacgcccaccgcatgccgccgtggcagttgccgacgcagaaggcgctgatgggcatccgcagccgggaactgaccccggacggcggcaacgatcccggaggccgcagcaacgccttggtcttggacgacaccacgggcaagattcaggtgcaactcaagagcgatcatctggatagtgcattgagcctggggcacatcacgcggatcgacggcacgaccggccggcagaacccgcgcggccaaggtttcgcactggataccgagggccacggcgtgaaccgtgccgctgccgggatgcagctcaccaccgaagccctgcccggtgggcgggggcacatgatggacgcgggcgcgccggtcgcgcggctgacggtggcccacgaactgcacgaacgattgtcggaagcggcacgcacggcaggggtgcagcaagcggacgaccagggcgaggtcgccaagcaattgcaccagcagaatgccgacctgaaaggcaacggcggcgacccggcgcaggggcaattccccgagttccagcaaccgcacctgatgctggccagcccggccggcatccagagcagtacggcgggcagcacgcaccagcagagcaacgcgcaccatgcgatcaccagcggcgggcacgccagcattgccacggggcgcagcttcctcgccagcgccaaggacgcgattcgtttgtttgcctaccggctcggcctgaagctggtggccgccagcggggatatcgacatgcaggcgctcaagaacggcatcaacatcctggcgaatctggatatcaagatggagggcaagcgggtggatatcaccggggtggacggggtgcgcatcaacgggggcggaagctatatcgacgtgagccagtcggggattacgagtggcaccaacggccaatatcaagtgcatgccagcgggcatatcttcgcggggccggatagccggccggtggtgccgcacgcggtcacgtcagcactgaaacccgatgaacccctgcccgtggtggacaagctaggcaacaccatgcctgacagctacgccattgtgcggcccggtcagaacgaaacgcgtacggtcatcaggggtgggcaggttggcaccgacgtgtcgcttgaccaccatatgatccgccgctggcgcccccgtcgaggctga

SEQ ID NO: 75 - DNA, 3286 bp, Ralstonia solanacearum
tgctctcacttcttggaatgttggcgacaaacagaaacagccacggccaaaacgggcgcggctggatacgctgagctcagaggaagcgaagaaaggaacgggctgatattcagcgaatgaagatcagacacccaacaagccgaatatcggccctgactatgaagtgaacatcctttctttattatcggaaactctccttcccttgttatcggcgggcagattaccgatacaggaaaacccgcgcaagggcactttagtgccgattttaaggatcaaaaaccggcacttttgagttttgttaacaaatcataacaaatgtgccgccatgaggtgcaggtaggcgattcgtcgggcagggagggaagcagcgtgcgcgtagtgccaccgcacatcatgagcccggggctgcgggtacgccgggggcgtttcgaccctgtgcgcgcggttgggaccccgcctcacctgcgcgcttcattgagttgtttttatgcaggtgcataagccgctgttgaccgcctgtgacaaaggattgcccgcgccaaacacagtagcaaatgcatgcaaatctatgcaccaagcctatgcaacaccggtcagcgggctgctatgccgttgacaaggtatatgtccagcgaacgtccctgtccgggtgcgggcctgcatagagcaccaccacgtatgtcagcccttgatgcgtgaaaacttggtagtagagcctggccgctccctgcggggttgtcttcctcccctggctcaaatgcgtgttggacgtcccccggtggatagtgtttgtgccgtccagcacctccagggtgatggtgataccaccagtctgggcatgatcgaatcctcgagcgcggaagaatgcatcgatccgcccgccagctccgtccatcgcaaattcaaccagcgcatcaactgcactcctcaaaaagtcgtagcggtggtctgcgacggtatcgcaagtggcaagcactccagtggcaaaaataatctgaggattcgcttcagcgacaaggcggaagtactccgacggaggcggcgtcccttgctgttggctgacaggcagctcttgccgaaggcgcgcgtacgcgtcattgatgatcgtggtgtcaggcttctgcttctttcctaagagcggccatccgtcctgaccggcggcgacacgaataatgactcctcccatttccgcaaccgtccccagttcaacctctaggtcagccgggatgggcgcatacggaaggacaatcacatgggcgtcagtgtagtggtcaaggctgtcgatgtatgcacgaaacgcatacacagcatccttttcaatcggcagaagtgtgagcgtaccctccgctccgatgccgaccagaatgctccgcccttcaatgccattacgtccgcagtcggcatcgaattttcgctgaagcgacttaaacagaccgatgacgaacactgcaggcattctatgggcccatccagtatcacagacgtgatctgaccaagatggtgctcaacagggtcggcaaaacgaggcgggcaatatcctcgtcgctcagcaactccaagagcttcaacgcttgctgccgcgccggaatctgactcgctgcggcacgcggtgcagtagggatagatgggccggccaagtcaggcactggagacgtgggtacgaacgcggctggctctttggctgccgcgtgtagccggttcaatgcaatgccatttctttcagcccactcgctgacgacctcaacgacttttgctgtacggtgtcggcgccatccccaggcatagttcccaagtgcatccgtgaatgccttattcactgtgctcgagtatgcctcagcagccaacggcccgagcgcctccttttgcgccgctggaagtggcgcttcctgaagatactctcgcatccacgcaccctgctgatccctgtcgatcggcaaaatagcaatgaaggaatctggcgcggattcgacttcggcgcgctcccgactctgtcgttcgcgcagatagtgcaccaccttatcagtggccttcttgaagtagcgcaagcgatcttcgtctgggttggcaaatgcctgccagatatcgctccgaagcaccggcgtcgccacaagcctcggctgaggcaacgcaggctcgggggtagcgcgggtagcgccacgcaaggaaactaagatatcgcccttaccctgggggcgatcgacactcacgagatcgccgtggacgcgcagaaggtagtcagaaaacctcttgaaccctagagacttttcatcgaactcaccaaagccgctcacggcaaatgcccgtttgagccgttgcttaaggcgtgcgccaagttcgggggattgttcggaaagaagctgcgtaagcagttgtgtggacgcagcgtgcgaatcggacatagcggtctcagcagggtcagaggcaaaaggagatagcccgccacagagtgtggcgggtatttccaatactgaaatcatcaattgcttctgagttccctgacttgaagccacgtcgtctaaccggcagaactcggcacccggtagcacatcggcgctgctcaagctttcaagatggccctggatactttgatccggcgaatctcgaaatacttccaaggcggaaggttctggaattctactgccggtcgaccggttgtcaagcaggcctcaactcgtcacaacgaccagcgcacaccttgcacgccacaattatgactcatagagcactttatgtgatgtactacagcgtagctagacttcaccctgcgtagccgtgatttgcattggatgaccttgttcgaccttagttttgcagtttgtgagttgcacaaggtcttgatttatgaaaggtactgagtcatcccaaaaactccgaaagcaggtatcgccgcgaccgccctcgacgatagcgcagataggacaggaggcagtttggcgctcggtccagcatggtcgggttggagcaacctagactctggccgccccccctgatcgtggcaaatagccagccttccgttgctttggcacaacgtccgaatggccacgtggcactacttttgagaaggccgaaaaaccgggggcatcaatgggggcatcaaccccacccacccagaacaaatccgtttataaacaaccagctatagttcgtatgtgatacctgtcggcggaccaccggatacgacaaaaagccaggcaaccgcctagcttttccgtttccgcagcagcataacaacgaggcccgccatccatgcgcgggcctatggctgcggatgcgccggggcctcctt

SEQ ID NO: 76 - DNA, 6969 bp, Ralstonia solanacearum
  misc_feature (5055)..(5154): n is a, c, g, or t
tccgacaggctcttgagcacatacaccgccgcccgcgacagcaatgcgccgttcacctcgaacgacgggttctcggtggtcgcgccgatgaaggtgaacaggccgctttccacgtgcggcaggaaggcatcctgctggctcttgttgaagacctgtacgatgcaatgcacctgttgaaaatcaataggttggcgtgcatgcatccggatttttcgcacaaacaacccgttttcatgcacaattttttgcaccaacgattttgattgtgcaatggatcggccccttggcttgtgccggtccccaaggacctcactgaacaagcgcccccttggcctgcacactgccccaaagttaggtgcttgagactcaaaaaaacaggaaggagtcactgtgtcggtctacccaatcaagtacaaggttctgaacaaggagactggccgcaaggtcagcgtcaccaggtggcgtgttcagatccgcaaaggtggcgagagcatcagcaagctctttgatgatgaaagcaccgccaagtcttgggaagaacaagaacttcaccgcatcaacactggtgttcctcagaatctcatttttgatatgaaatatcaacttgagatgcccgacatgcgcaggcttctcaaggactactttgatcaatacatgagcaagcaggcagcttcttcactgaagacgaaccagaaccgttgcctgagtgccattccctcaatccctatctaccttaaagatttgggtccaaagattgacaactatcgttataaaagcacgattgttgatgccatgatgggcaggaccttcaacaaagaccacgttgaatttggtaacttcaaaattgatacggtagatttctggctcttgattgcctacatggattctcgtaggaaatctggcatcaaggacaacaccatccttcgtgaaatctccactatcagttctgcctttgagaaggtctacaagctctatccagaacaattccccaacagcatcatgaacccagtcaagatgcttcctaaaggtgaaaagcctaaagcctatcttggcaggaaacgggttctctctgatgatgaagctgttcagattgcagaatggatgaagcttaaagccaatcaggagccttactacctgttcgttacttgcttggagtctggagcacggaaaagtgaagtgctgggtgctcagtgggaaaacgtcaatcttaagctttggtctatccacattccgaaaaccaagaatggcaagccaagggacatcatgattcctgaagatgatgatttccgcgaatggttgaaggccaataggcttgctaaaggacctatctttaaactgacagcttggaacttccgtcaatactgggtggatgctttgaaggcacttggtctttatgatgatccagacacaagacttcacttccatgacacccgccgcactgcattgaccaagctcattcgtcagaagcgatctaacaactttcagattgccaaagagatgggagtgtcgcctcagactgttgagcagaccatccagaacatgcctgataggttgtcagaagtgtttgcgaagcttcgttctggtcaggcactcaatgaagaagaaatcatgctgctggctggtcacagttcttcatccatgaccaacgcctactttggcgacaggaactaacaagcagcaagggataaaaacggatagaaatgacagataaaataatcatttccaatgaagagaaaatggctgatgttcagcttgaaattctcttctctggcccattgagccttcttgatcctagacatccagcaatcagtgaagagcttcagaagcagattctgtatgacaggtaccacaacaaactgattgcagatggcctggaaacaggccattttttctacgattacaacaacgatttaaggtttcaaaccaagcaaccgctctatacctttgcagggttcatctttgaagcctttgccgtcaatgttttcaataacaacatgagaaccattggcaagaaggctttttcttggtgtacaaaccgagagcagtgcaaggacgattacattgatcaattcaaggtaattggaactggattcattacaacaaagtcactgtttccgaacttctacgggccacaaagcacttttgacctcatttttatcaagaaaaatatcaggcaagatgtgcatgagccagcaaccgttttgggaacaacaaaccaagctggaattcaaatcaaagccatcactggctcagaagaaactgaaataattgagccattaagaactggcagatactcccatgttctgacttttcttcgtcactcagatggagtgcatagctacacccattgcatggaagttttaaactccatgcatagaagaggtgttttggggctgccagaaaaatatgcccttgagcgacgtatcagccacccagaaatgtttgggatcgaccaacgtgaggttgatgattactaccaatacatacttcattggtacacaggtcatgccaatcccgatcaatttattactgaaggtgctggtttggaaatcaaaggctataaatattcaaacggcttgcttgtgccaaccgattaagttcaaccgttttacacccaacctaaaatgctagttctagcgactagctttttcatattgagaaacgggatcaacatagatacaactacccagttcttgaagtaaaaaatattcaagaggatagttgacgcaccttgttcttgcacatagagtacagtcatcaacaagcaagaagaactgtagaagcttgttgagtgtgttatccttcaagtatccatagttctatcttttttaacggagaatttttcatggccgaaaccaagaagctgacccttgctgagatccttgccaagcgcaaagagtttgatgacatggtggcggctcagttggaagcggaacgtcccgctgctctggcagccatgctggaacaaatcaagatgttcaacttcacggctgatgaacttggtttccatgctgctcctgatgcacgtgccgctgctggtggtgccaaggccggtgctacccgcaacatgagcaagccgctgaagtccaccaagaccgacgaaaccagtgtgtggctggctcacccgcccaagttcttggaagctgaaggtgctttcaccacctacaagagcggcaaacccgttgatgcatggctggtgactcccaccgacaagaaggccaagaccaacttcctgaagaagctggcaagccgtgaaggcaaggttcccaccaaggaacaactgggtgaaatcaccgaggcagaattcaaggcagcctaagctatagcagcttaagataaaagcacccttcggggtgctttttgccgttgacgattaagaatattgagtgaagtgacgtcttatgatcattgcccaagtagaagaaaaagttaaagcccttgtgggcaatattgataaggctgaatttctctatggccttttggaatgctatagcaagcctaaagcctccattactcgcctgaaaatggttggcaaaggtagctataacctctccaagaacgaaaatgaagttttatggaagaagcaggtctacttcaaagccactgacagcgatcagcttctatccgtcatcgatgaaatgaagaatgcagaggtggttacaaaacaccaacctcgcttcattattgccatcaatgatactgacctcttggcgattgataccaagacggcagacacgcttgatacatctcttgccgaactgaataagaagtttgactttttcctgccttgggcaaatatggaaaaagcccagtttcagcctgaaagtgctgctgatgttaaagcagccgaaaaaatggcgaagctgtttgaccttctccgagataacaacccggcaaccactgcacaagaaattcactggcagaacgtctttctgtccagaatcttgttctgctactttgcagaagacacaggcatctttgaccccaagctgtttaccaaccacatagcctcacacacggctgctgacggatcagacttggacgactacttgagtcgtgcttttgatgtcatgaaccaggaagaccgtaatggtgttcctgactatcttgcagacttcccttatgtgaatggtggtttgttcgctgacaagctgcctgttccgaaattcaatcggaagtctcgtgccatgctcattgagtgtggggccgatctggactggtctgaaatcaatcctgacatcttcggctccatgattcaggccgtagttgaccccagccaacggggcaacatgggcatgcactacacctccgtgcagaacatcatgaaggtggttgaacctctcttcctcaacgaattgaaggaagagtttgaaaagcactctgctagcaagaccaagctggaacaactgcttcttcgcttggaacacctgaagatttttgaccctgcttgcggatcaggcaacttcctcatcattgcatacaaagaacttcgccagcttgaaatggaaattttcaagcgaattcaggaactgacacctgaaggaattttcccgctttccagaatcaagctctcccagttctacggcattgaattggacgatttcgctcatgagatcgccatcctttctctgtggctggcagaacaccaaatgaaccaaaagttcaaagagacttttggacataccaatccagcccttcctttgaagagcagcggcaacgtcgtcagcgataacgcaatccatgtcgattgggacgagtttcttgctcccaaagcagagcatgagacataccttcttgggaatccaccctaccttggagcacgtaaccaggaaccagagcagaaagcagaccttgaactgatttttgaaggccacgaggaatacaaggactcagactatgtctgctgctggttcatcaaggccgcaaagttcattgctggcaaaaatgccaagtatgcgttcgtggcgaccaactcaatctgccaaggtgagcaggttgcctacctatggccggtggaagtttggtcaattcccgcgtcatgctatctacgaatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaactggtagtgcgcggcacttcgcacatgccgcgcaaaagagtgtcacggcaataaaaaaagcacccgaaggtgcttttttcttaatcggtagcgatcctacgtccgcctgtagtagccaacaatctcttccctcacggcctcagcaagctccagcttcatatgacctgtcagcttcatttgctgacccagtgcatcggcgcaaacataggcagtttggacatatccattggcagaaccactaactgattctgtaacaacagagaatttcttaatgtcagagcgagcaagttgcttcttgcccccaccaaactgaaccccatcaggaagaagacggatgcggtatttacgacctgcaaacacaaacttcaagagtagcaacggagcagccaatgcgaccagtgccgcctttcccatgtgattctcaatgaatccaatggaaatgtattcaccgagaaaaaccagaccgatccaagccgccacatagataaacagcagaatcacggtcagccctactggattagatgcggctccgaatgcgcctgcacggctacctttgaccacaacttcagccgagccatcagattgctgagtcacctggtattgcttcacaaacgagcttttcatatgtaggggatcaatcatgttcaaggttgttctggtaagtcgctggcatcaacaagctgtttttccagctactcatcctcaacccgacctttcgggttgatactcaactctaacgcatgttcatctttttgggttgacaggcaactatttgggttgtcaaagatcgtggagagaacatgccgccagagaacaaccaacacggagaactattcaagagcattgggcgcacgttggcccagcggcgggaggctaagggcctcacccaggatcaggtggctgaagcccttcacatcggcacagaggctgtatcccgcatggagcgaggcatcaccatgcctaccgtccaacgcctggcagagcttgccgaggtgtacgagtgcggcatcgatgagctactgatagcaagtagcaccaggaccggcgaccaggccgaactgatctctcagatcctccacacgcttcccgaagcagacagagccatgatcgttgaggtggtgcagcggattgccgcacgtttgaaggatcggctttaaatctaagaaggctatggatttatggacgatatgcgttgcctaacggcatgcttgccgtggacaactcttacgagttccccaccgcacataccttcgcccacaagctccacatcctgcgatcactttttttgattgatctttcttccatgaagccaaacaaagaagccgcagagcaacaagaagcttgtcgctcatacttacgatctttttcttgatttttatgatgtgatgcgcaaaagcttgatgttgtcagagatctgcttatagtgtacgtatcgattggagaaatcatgccccctcacctcgaaccaggagcatcacaaagcagcatcgcccatgactcagaacaacaagttctgtctcactggattgaaggtcaaccagctttcaaggaagtcctctccaagttgcccgacacggatcaatgtttctcccccaaccagattgatgccatccttgccattcggcgcaggatcacagaagcgaagcacgcccatgactgagcagtcacccttgccaaccgacgaggatatttgggaagaacttttgcactcggaagccagcaagcagttcttagataagctgatcaaaaaagtccaagagtttgtagagcaacaaacgcattgaagcaccttcgggcgcctttttattgcc

SEQ ID NO: 77 - DNA, 852 bp, Ralstonia solanacearum
atgacgagctcaagcaactggccgagcgcgcgcggcaggaactggggcggcctggaatgggtgcccgccgcgctcgacgccgtggtcgcctctgccgatggcgatggccgcaagctgctgaacaacctcgagatcgtcacccgcgccgcacgtgcgcaggccgaaggcgacaccgtgccgaccgtcgacgaggcgctgctggcctccgctctgtcggagaacctgcgccgcttcgacaagggcggcgatgccttctacgaccagatcagcgcgctgcacaagtcggtgcgcggctccgatccggatgcggcgctctactggttctgccgcatgctcgacggcggcgccgacccgcgctacttggcgcgccgcatcgtccgcatggcctgggaagacatcggcctggccgatccgcgcgccgcgcgcatcacgctggatgcggccgagacctacgagcgcctgggctcgccggagggcgaactggcgcaggcgcaggcgctgatctacctggccgtcgcgcccaagtccaatgccggctacaacgcctataacgcggcgcgcgcctttgtcggccaggacaagtcgcgccccgtgccggtccacctgcgcaacgcgccgacccggctgatgaaagagctcggctacggccacgcgtaccgctatgcccacgacgagcccgaggcctacgccgccggcgagacctactttccggacgacctgccggcccagggctggtacgcgccggtgccgcgcggcctggaaggcaagatcggcgacaagctgcggcacctgcgcgagctggacgcccagtggttggcgcagcagaagaacaaatcctcccgcgacgactga

SEQ ID NO: 78 - DNA, 579 bp, Ralstonia solanacearum
atgaaaaacggacgctaccaagaggacggcatagacgtttggtacttcaacgatcacctacatcgtgaagacggcccggcttgtgttgagccagatggaacccagtcctggtatatccacggaaaacttcaccgcaccgatggcccagcagttatctggggagacggaagtcaggagtggtggattgatggtgcgaggcaccgcgaagacggtcccgctgttctcaatgccgatggcagttgctcttggtatctcagaggcaagctgcaccgcacagatggtccggcagtagaagctgccagtggtgcaacggcgtggtatcagaaaggccagctacacagagaagacggccctgccgttgaaggctcagacggcactagagagtggcatcttcgtgccatcaagctctcagaggaagaattcaaccgccaacatccttctttcaacaagaagaaggtggtcgaggtgagaagtgtctacgacaaacctgtgaaagccgttgagaatcgaatattggcaatcaggaagaagttctttgaagaaggtagtacgtcaaagggagtcaagccaagatggtag

SEQ ID NO: 79 - DNA, 882 bp, Ralstonia solanacearum
atgaaagaacaaaaatattttgaaggtgttgccagcaactacttcagcaacgtggatggacaggctggccttgccgcagtcccttccaacttcgttgacttggtactgaccgacccaccctatggcattgcagacggtgccaaactcaccaagaagggcagcaagatcgtcaccactcagcaggcatggggtagtgactttaaggatagctgggcgaccattgatcagtattaccagtggctcaagcccttcattgctgaattcgttcgggtgatgaaggacaacggctccatgattctcttcttggatcgcaagtacaccgggttgattgcccactacttggaacgagactttgacttgaatttcaagaacaaggtctacttcaagaagaagaaccctgttcccagcattcgcaagaacaactaccgcagcaccattgaagaggctgtttggttcaccaagggaaaggagtacaccttcaacttcggtgcccagagcgacatgcttcaggtctatgagggtcccattgggaagaagaagacccagcaccctaccgaaaagtacacatggatggtggagcctttgatcaggaaccacagcaagcccggtgacgtggtgttggacgcctttgcaggctctgcaaccaccctcgtagttgcgaagcagcaggatcggcacgccattggctttgagaagagcaccgccctgtatgagatggccaaggcccgcatcgaggcagagcagcttgagtttgcattccatgcaccatgtgacgaagcagccatcgtgcgcgagctggatcagctcatggacgccgagcttgcagacatctccagcagccgcagcagtgccgcggatctggctgacctggtcacagcgtaa

SEQ ID NO: 80 - DNA, 895 bp, Ralstonia solanacearum
gtaaggagggcagcagccatgaaagaaatgaactgcctccaatactgcatcaacggcatgagtgaccgattgttcagcttcgctaagaccaaggaaggcaaggcactgcttgctgccttcaagaagctcatcgtcattcgtgagaaccagatcaaagagcttctgattgcttacaacagctacttcatggtggctgctgccatgcagctcaagggcatgcctcagcacccacgggccatgattgagttcatggcatcagaggaattcagtgccctgcatgctgaactggtgaagaccgttgaggacaactaccccttgctcatgtcctgcttggataggaagcagaagcgaaaacttgattccctctttgaataaacaaaagcaccctacgcggtgcttttttgttggcatcgactcaatccgcgagatttacaccaacaagaaagagaataggcacaagcctactctctgagcagaaaaaaacaccctgtggggtgatttttatttacgaacacaatctatcttgatgaccttcctgctcttcagaatttcatcaacttcaggtgtcagtgttttgtgaagctcaagcatggctgctgacaccatgcagccttcaaaccaatactccttcacttcacctttgacgactgctgggccatcttcacggtgcagcagcccgtgacgataccaagcagcatagtttctacccacgaccgcaggcccatcttcacggtgcagcacaccgttcttgacccattgcttcacttggttgtctacaacacattgcagcttggtcttcatttcagtcggcaatagataacgagatttttcattctagcgtgctttttgttgacttccatttatcatctgcaagaataaaaaaaaggagacacca

SEQ ID NO: 81 - DNA, 540 bp, Ralstonia solanacearum
atgggatacaagcaatggatcaatggcgccatcctccgtgaggaggacggcgcaatcattccgcaggatgccaggaatgtggactatcacgcgtacctggcatgggtggaaaacgggaactcggcggcgccattcccttctcaaaccctcgcgcaggtgatggcggcgaagctcgccgccatcaaagccgcgtgcgaggcatccatcgtggcaggcttcgcctcctcagcgctgggcgccgttcatacgtatccctcgtcggaaacagaccagcgcaacctcctgggctctgcgattgctgcgcagtggcgtgatcccgactgggccgtgccgctgtggtgcctgagtgacgaagggcaatggctttttctgtcgcatgacgctgcccaggtgcggcaggtcaacgtggattgggttgctttccgcgacacactgcggcagaagtatgctggcctgatcggtcagctcaactcagccgctagggaaacgctgatttatcagtctcagccgtcatcgctgacgcagctgaggccgttgtag

SEQ ID NO: 82 - DNA, 483 bp, Ralstonia solanacearum
atgccgactgcatcctgcacatttacgctcaacgggcaggcaaccagcacgctaacgtgcgacggccgcgtttatgtggctttctctggcaataaagggcatgagaacaagcccggcgataccggcatcgctggacaagggccattaccaaccgggcgttattacatcgtggaccgtgagagcggcgggcgcctggggccgatgatggactcgatccggtcagcggtcagcaactccaatcacgccgagtggttcgcgctctaccgcgatgatggcgcgattgacgactggacgtttgtgagtggcgtgcggcgcggtaatttccggctgcacccagttggcccgcgaggcattagcgagggctgcgtgacattggtcagcaaacagcaattcgcggctctgcgcacctacctcaagtctcgtcagaccggggttgtccccggcacgaacacgaagtattacggtgtgctggaggtcaaatag

SEQ ID NO: 83 - DNA, 300 bp, Ralstonia solanacearum
atgcggtttgccctcaagaccgtcgctgtcgtgttactggcgttcgcatatacgcgggccatttatctctggtcttggcttgacccattctttgccagttcggcatggcagcgaatctatggcttcctcgcgcacctgtttggcgtgagcggtgcggagcaaggcgaagacttgcagctcgccatgttccttgtggtggcactgctgctggcgatcttgtcagtttggctgttcgaccgctacgtgtgtcggccggcctgccacacattagcgcggcagagaaccgcgaaaaggccctaa

SEQ ID NO: 84 - DNA, 677 bp, Ralstonia solanacearum
ctaacgatgccgcctccgggcggcattcttgtatgtatggggcggaattcaggcgaggtctggtgggcgaccgtctaaaacgcgctcagagccgttttaggctgctgatggaccgtgggaggtccacgccccggaaaggcgtttttaaacggctttcgaacgcgaatggaggcatcccgcaaggcggcgctccccaaaggggcaccgtgcggggtcagaagaggtctagctgtcgatcgtcgtcctcacggcagatgttgacaatctgacgggacgaaaggtgatggcgccgagccaaagcgttgatgctggcgttgtgccgatctttgcgaatctgcgcgtcacgcacgcggataaacagcttgtcggccttgggcagccaacaacggccatccgaatccagatgcggagccagcgtcgtccgcaaacgggccagttcgtcggcctccaagcccagtgcgtgagtacggtactgcgggatgctgacattgacgccaccatggtgggtcagccagtcccgcgcccgaccgtaaccaagcgcgcggaccaccgcacgcagcacgggtggcaaggttctcagcaaatcttcatcgacagccgggtaggcgtctaccacgcgctcgatttccgacgcggcatgcgcagcgggatggtgatagtgcggaatgaccatggct

SEQ ID NO: 85 - DNA, 456 bp, Ralstonia solanacearum
atgtctacgttcctcatcaagcatcatcggcagcttgtcggcatcgccaaaggctgggccatggtcagcctgcctggctggtctgaagagacccaccgcgacctgcttacacgccatggcgcagccgtggttgacggccgcgtgtccgccagtaccctcaacctcgcgcagctcggcgctgtgctggacgactacgagcgtcgggggtggtcgcgctatcggcgcgtgttccgtcaggctggcgtgaccaaggaagtcccaccccgcatcgctcacctcgtgcgcatgtggttcaagctcgggcaggcgggcaaggtggctaaggcaacgcgcccggcgctgcttgagttctgtgcgcgtcaagtgggccgtcacgtgccggacttggacagcttggccgtagctgaatgccaaacgattgccgaagctctcaagagttggctggcgcgcgggtaa

SEQ ID NO: 86 - DNA, 702 bp, Ralstonia solanacearum
atggaccgcgaaaccgcaatcgacaagatcaagaagtgtttggcgcttgggcaatccagcaacccgcatgaggccgctgccgccatgcgtcaggcgcagaagatgatgaccgcctatggcgtcagcgaagacgacatgctggcggccggcgtggccgagctgtgggtgaagagcggtgcgacccggacgccggcccgctatgaggtgtggctggccagcatgatcgcatccgcctttggttgcgagctggtgttttccaaccgcctgtccggtcgaaccaccatccaaggcggctacgtatttatcggtatggccagcgcttcgacggtcgccggttacagctatcgggttctgttccgccagcttcgccgcgcgcgggccgactacatcaagaccgcgctcaagcgctgccgctccaagaacaaggtggcccgcgcagacatgttctgcgaaggatgggtgtttgccgtgcgcgagctggtgggtgccaccgtgccgcccgaagcccaccgcagcgcggtggatgcctacatgcgcgcgaactatgccgtgaccgagcaagttgagacgcggtgccgcgaacctgggcgccatgtttcccaggtgggcgactgcgtgcaaggcgttcgcgctgggcgcggcgccaccctccatcgtggggttggaacaggggtggatgcaccccaaatgctgggggcatga

SEQ ID NO: 87 - DNA, 276 bp, Ralstonia solanacearum
atgaccaagcaagaactcatcaagcatctggccgacaaggccgaagtaaccaagggcaaggctgaagacctgctcaacgccctgaccgaaaccattcttgacaccgtgcgcgccggtaacgagctgacgatcaccgacctgggcaagttcggctccgtggagcgtgccgccaagaccggccgcaacccgaagactggcgacgttattgccattgcggccaagcgcgcgccgaagttctcggctgccaaggccctgaaggatgcagcagcggcctag

SEQ ID NO: 88 - DNA, 633 bp, Ralstonia solanacearum
atgaccacagtaacgcaacaccccaccgagattccggctggctatcgcgccaacgcgcggggccacttggtgcccgaatcgaccatccaccccatcgacctccagcgcgacaagctggtgcgcgagctggtggccaaagccaaggcactgcaagcagccatggccgatttcaaacgccaagcctttaacgacaccgacgcatttgtatcgctctcggccgagcgctacgaagtgaagctgggcggtgcaaagggcaacgtcaccttgagcacgttcgacggtcgccagcaggtagtcgtgtcccaggccgagaacatcgctttcgatgaacgcctgcaagccgccaaggtgctcatcgacgagtgcatcaaggaatgggccaagggcagcgacccgaagattcaggtgctggtgcagcaagccttcgaaacggacaaggagggaaagatcaacaccggccgcgtgctcgcgctgcgccgcctggccatcagcgacgacaagtggacgtgcgccatgcaggccatcggcgagtcggtggatgtggtgggcaccacggcctacatccgcttctacgagcgcgccgagggtgccaagaagcccacagccgtcaccctggactttgccgctctgtaa

SEQ ID NO: 89 - DNA, 588 bp, Ralstonia solanacearum
atgccgctgcctgaagtcgtctgcccgaactgccgcgtgcggatgagcctggatgtggtgctagccgatgacggcgtgcgcgacatgctgctggcgctggtggacgtgcacccggcaggcgactcgttcatcaagccgctcctgcgctacatcggcctatttggaccgaagaagacgcagatggccaacggacgcatggcaagcctcatccgcgagctggagccggagatgcgagcggcccaggttaagcgcgacggtgccgtgtacgcggcgcccatggcgctgtgggcgtcggctttcagctacgcagtggaccaagcgaaccacggccgcctagaactccccctgaagtcgcacgggtggctgcgctctgtaatcgcggggcaggccgcccgcgcagcgagtcaagcggaagccgaccaagaggcgcagcggcgcggcattgctggggctggcacaccggaagcccgccgccaagcggccacgaccaccgtagggccggcagcggtaagcgagcaactgccgaaaaccgagatgcctcaagaagtgcgcgaagccctcaacaacctgaagaaaggacgccaatga

SEQ ID NO: 90 - DNA, 506 bp, Ralstonia solanacearum
ttgccacctccaaccagatcggggttcgtttcgcgcagacggcggtacagcacgcggcggcgcccggcgtacttcgcttgaaggcgcgtgaggttgtcccgagcacgggccatttcttcctgccgccactccacatcgcgcaccgcgtggatctcttcgcggtcgatggcgcgaagctggcgctgcacgcccacccgcacgcgcaaccggcgccactccgcgcagattcggcgcaagcccgccagcagttcggctgcaacaggtccctgggtgatgcctgctagctgtttcatgtcttcaccttccatttcacttcgtcggggatttggcatgtgccttcgatccaggcgccaaacgcggctgcctctgctgaaggtgcgccgttggcgatggcatccaagaagcgcacggccgctggaccggtggcatagatgcgcttccggcgccgtgtatgggtaatcgtgatgcccggtgggtcaaactgttccgccatggctgcctccgct

SEQ ID NO: 91 - DNA, 909 bp, Ralstonia solanacearum
atgtctcgcttgggaatcgagtatcagaacggtctgatttatgagggcagggacaacccgagctccttagccgttccgacgccaatcatctcgcagtgcacactcattgagtccccctctgatcttgagaggctgcctcgtgggatagactcggatccgtttcgctggattttcagggaggactcgttcgaccccgtttccagggttcgacggggccgactatttcagcctttcagtaactcgaacaaggagttggtctccgttgacgcgcatccctttctgccttcggatttgggccggcgtggcgtcgatggtcaattgcgcaaggaaatgaccgtgttcattcactgcacgcaacttgtggtgcgccgggagcgcggggaaggtctgcagcttgcaatcggccacgaaggggcacactcgctctggcgcattctgcagactgagcaaacggttacccaggacgtgctggtcacccttcgagcggagtctgtattcggggtactgccacccctcgatctggcgcaaatcccagaggacggccgaacggcagtagctgcggcctacgaccgggtcatgcatgttgcatatcgagactccccgacatcggttgtggaccaatgcaggaacctctgcgcagttctcatggctcgctggttgcgccagcggacgagcaacgataggttgcttcacgacgatctcggcgcgtgtatatcggcagtcaagaagttcgccgaaggagagcaccaactcgtccgcgccgcacttgagacagtgaatcgtctccatccacgagggaaggagaacgagcgtgagcggtacagtttgcgggaggtgtcaaatgaggacgcagagcttgcattgcacgcaaccgggttcgtgattagggagcttggctgggggcgctga

SEQ ID NO: 92 - DNA, 1296 bp, Ralstonia solanacearum
gtgaaggttttcaatgcccggcattttctgaggcatatcgctgccagcgtgctgcgcgagttcacgcaagcgcatgcgctggccacgcgtttggtggtggactggtccggaccagccgacatgttgtcgggactgctctgcgatgcggttgatgcattggagcagcaggttgctgccgccgaccttcccgaacgcgaccgcgaagcgctggagcacgacctgctgctgtggactgacgacctgcggcgtgcttgtctgatggccaacagccttgctgtagccgagttccggagcgcatgccaggacgatccagatgccctgggggcgttcgcctcccgtgacgagcgagagatcgcactgtggatgctggtcttccgcgacaagatcttccgcgacgtcgaactgcatctggccttccaatccaagacccacggcaagttttggaagaggcaccgcatccagcccggcctcgaactgacgcgcgagcgggtccggctcgaacagttctgccatgccgtggcgcagctttacaagaagtccggcggcggcgatggtgtgcacatcgaactctccgagcgacgcagcacggcaggcctggtggatgccatgtccagtttccagctgaccctctatgtcgagggaccagtgacggcgctcacgcacttcacgcagagccatttcactcgcgtcaccacccgcgtggcgctggagtccgcgttggtctaccacccagcgaccggcgaagtggaaacagtcgtcaagggtggcgccaagaaccatggtgcgatgctggagttgttcggcgagcatgtcgtccagcaggatctggcccccgagcggattgagccgcaacgctaccacctcaacgccttgcgcgatgacctgcagccctatgaggattggtcgatccacggcgtggatagggtccgtctgcgccgcgcacgcctagcgccggcggcgggatcaggtgtcagtttcactgtcgaagcctcgcccgacaaggaccaggacgacgccatccggatcgcccgcaaggcgctcaaggtcgagcatatgtttgaggcggaatatcacctcgaggctgcgaccgtcatcgtatacacgcaggccgccgacggcgggcgggcgggccatttcagcttcaatatccgcgcctcgggagcctcgaccatcaagaacctgtcgctgaggaaccaggtgctcgcgcgcaaggtcctgcaggcgctgatggtgatcgacgccgaggacgatctcatcgcgaccatgtccattcccagggaggctatcgcagcgtga

SEQ ID NO: 93 - DNA, 975 bp, Ralstonia solanacearum
gtgagtcaggctcaggttgacgctaccgcgctgctctgccgattgctggagcgcgacaaacccgaaatcaacggccaggccctgttcgatggggacccgcaggcagcggctcacctgctgcgtgagcggttgctggtggtcggccgcccgctcgaatgggtcacatgcccggactgccgtgtggaaatcgcgcgggtcgtgcgtgatgtctcggccgacaggatcgccctgcgttgcccggaatgcgaggacatcgacgcctcgcgtcgtctgcgggaaacgtacacggcggtgccggctcgggccgtcgctgccttactgagcgggctcgggatgacagccggtggtatgaaggtcatcgaacccgaccgcgtctggcggctgggcacgacggagccgacgcgagggaagccgctgacttggtacttcgcacgccagcttgggcgaccggagatcggcgcccggctgcgcgagcagatccagctcgagcgcacggccggttcctgcgtggcgctgaccaccagcgagttgccgttgccgattggctcgccgctggtgggcttcgatgtgcgcatgctgtccagcgcggcgcggataagtcagagccgcttcgaattcttcgcggaccggcaggctgcgccgggggcgcagcaggtgggggaggcagagccgcggttgaccgcgcagaccaccctgcgctacgtgcgttcgctgggcaaagccttcatcgaaggaacggaatatcctctggagccgcgccagcaggccatgctgcttgctttgctcaacgatctcgatcacgagatgggtaaggccgcgctcaagatcgcctgcggctcgcaggcgcaacggttctcgccgagcaaggagttcgaccgcaacccggtcgtctacaagaccttcattcgctacctgcgcgacgacgaacgctacgcgcttatcgttcctgacgaggatcgggactggttggggtag

SEQ ID NO: 94 - DNA, 2388 bp, Ralstonia solanacearum
  misc_feature (1457)..(2385): n is a, c, g, or t
gtaggtgccgcaatacacccattcgccggcccagccggccaaatgcccgatactcatgtgctcgacgctattataagagcgggggcatgtcatcggcgcgaacgaggatttcgagcaaatcgaccctgcaggtttgacagccggtgcttgcgtttagaacgtggttagggctgcagaagcttccggagctcaggcaagggaccgtttctgtcaaaaatttgcgaagcaagaaaggcttcgtcaagggcgccaagtgagcccgaaggggaccgacgcaactgagcaaggacaagcttcaaagcggcaatggggaacgcgagcatcgcgtggtctgggtcaaaagtgtaaaggtgtattttgtcgagaacataggcttggaccaactgccgcactccgacctgttctacgaaagcaaaatgagcgggggctgtaggctcacagaacttcgggattttgtagttggctgcctctcgcttatccaccagccagtcgaacggtttatcggccccaatatcctgtgaggcgaggacgctgctcgtgtaatgatgggagaacgtgtctaacacgactttgtgcgtagtgcccgcacgctttgtcggagtagcgccggcggttgcccgccacgaatagggcgtagttcttgaatagaagatcccgacgccaccgagggcaagcgacgcccgcgtcaaatagaaggcgctgtaatagagcttcacggtcgcccacgtgtacaacttcctgtctagcccctgtattgcttcagcaatggacaaggttccggagtaaattgcatcgcgtgcgtcctcattcaaggaggcagcaagctggacagcctccctggcttggagctgcctggaaactatggcagccccggtaatcccagatagaatggagtccgcaacgcgttgcgcacgatgcataagattagaatttgaacttcgttgatgccttgactttggtggggctgaaggcctcttgcagtcgtgcgtatatcttcttctgcgccccgctaccaccctgccccttaacttccgcctggatgataagtgactcgttgaacttcagagctgcctgaattgttttcacctcaaatgaggcctgattcttacagaagggaatcaattgaagcctgaggaaatccattgcggcaagaaatcctgatgcgtagagaaagacgttctctttgtcttcccaacgatcgccatagatgccctggagcgcgatgaaccagttgagaatgatctgcttttgcaactctagggaaccgctgatcaatgaagtcggcgaatggaatcgcgagtcatgccatcttggtttttcggattgaggaagggatgcttcgattcagcacggtttttcgctgctttcgcacctatcttggcgcgctttgcgcgctcaggacgcaatgctcccaagtagcattacgcaccgctccaccatcgcacatcttctcagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngga

SEQ ID NO: 95 - DNA, 259 bp, Ralstonia solanacearum
atggatatcgagccgtcgttcgaaacgaacgcgtatctttccaaagccggcaaaccaagcgtgcgtgcgctcaaccacccaccggtaacggcccaggcgctccctgctctcgatgcccttgcgggcgatccgggcccttatgccgtgccgcttaagatactgccgacatcggcggcagtcgtaagccttgtcggcatgcagcttgtcggggcgcttacgcggccggccatccaggccacgtatcgcgggaatcgcatcc

SEQ ID NO: 96 - DNA, 255 bp, Ralstonia solanacearum
atgacttgtgttagcgactctaagtacgtttgtaccggctctagtggaaagattgtgtggggcggtggcggattggaggggcgctcagagtttgggccgaataagtcggattttatggcggcgctttatttggatgagcaaggcaatttgattgtggaggatcgtattcaggttcacatgagcttgcgacttctgcatattccaacggggacggcgcagcattacacggtttacagatttaggcgattgaaataa

SEQ ID NO: 97 - DNA, 534 bp, Ralstonia solanacearum
agaatctgttatggaaccacgcgaaagtagagggccgcgcagaatttatttgcgctcaatgtgaatcgtgagtgtttttattcaccccacaaggccagaacgcagagtcgagccaggcttttgctgggcagcgttgcttttcgggagcatttgggcttactcggaaggattgatcgttcatggcggacgactggctgccgccgatggggctgtcgggttgttgtacctctacggcgagcaaaccggtcatccatcagcggctggtggcgctctactgctgtttctcgccaagaacgtttattgcgccgtgcgaggcaggttttggctgcagtgcttgctgctcagtcagggctatcgcgtggtcagtcagtagaagcccgagcatcgggccactgatatgcaggcggcatgagcgacgtgccaccgctacaagtacacatcaagcagtttttggggaagtttctgtgcaatcaccacaccgcccggtccaactggccgtactcctcggcctatgcgccgtcttcatcccaccag

SEQ ID NO: 98 - DNA, 993 bp, Ralstonia solanacearum
atgttcaacagttttatagaggcactaccagacaaggtaagaagctcaaatgatttggaatatggttgcaagttcaggaagaaagagaaagctttgcagcatctctatgtagagcttccccagctctacaagaagttcatagccctggacatagacatcccaggctcttttgatctttggtaccaccttgggttgccaccacccacctatgtgatggtcaatcctgagaatgccaagtgccactacctttatgaactgaagaccccggtctactacaccgagcatgcacgccgcggaccacagaagttctttgaggctacagacaatgctctgacctgcatgctaggggctgaccttggctttgtaggccacttcatcaagaacccgctgcacccccactggcgtgtcatctgccaccatgccacatatgacttggaagacttcaaggattggggccttgacctggtaggctacaagaagaaacgcaagctcttggactctctagagggccgaaacaccaccctctttgacaccctgagacactgggcctacatcgaggtcaggaagtgcggctcctatgctgatttccagcaggctgcggactctgaagcactgtctatcaaccagaccttcctagagtgcttaaacggcgttctaccagccaaggaagtgctttccactgcgagatctgtaggcacatggacatggagacacaaggacaccataggcaaccagaagaaccgtggtgtcatggagcttccaccagagatgtccatgaaggccaagcaagctgctggagctggcttcagtgctgtgactaggacaaagaacaccaaggaagccatccttgaagctgccattgcattgaaagcagcgggaacaccagtcacccagcagtctgttgccattcgtgctgatgtctccattccttctgtgaaaaagtattggaatgaagtggatagccagcttgggttgtataaaaaacgtgcctga

SEQ ID NO: 99 - DNA, 852 bp, Ralstonia solanacearum
  misc_feature (395)..(544): n is a, c, g, or t
atgatcatccttttgcaaatcagaacaaggcgctcttcggagtgcttttttctttgtgtgcttgacttctgcactcaattttgtaatttagataaatgcaaaaggagtgctatgaagaagtatggagttggagttgtcttggctggtttaatgatctgtggtaatgcgcaagaacccaagtatgggttcgtgaaagtagccgagtctgaagaaatggtcttgttcgtgaatgttgattcatataggtatcgttacctggcagacattcaaagcttcggggcggtggttcagattttcaggaagatgggtggtggctcgtcattcagaaaagaatatgtactagagcctgactgtcgtagaggttatggaaggctatacagccacggccttgatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcgaagattggtcaatccggcggcaagaaacacagaatgttgattcatataggtatcgttacctggcagacattcaaagcttcggggcggtggttcagattttcaggaagatgggtggtggctcgtcattcagaaaagaatatgtactagagcctgactgtcgtagaggttatggaaggctatacagccacggccttgatgacagaaatcttcaatcatatgagtggctgaaggatagtggaaccgttgcaagtattcatgcagaggttctttgccatgtgtcttcttgggataagccgaagaaataa

SEQ ID NO: 100 - DNA, 456 bp, Ralstonia solanacearum
atggatgagaagcagattgcagaactgcggaagtttttgggcactatcagtgacccgaagatcagtgccttcattgatctgaagaagttgtgccagtcgggcatttacaaccttgcggttcagatggccacggaacatgctgccaaacaccatgacttcggctatctggagaaaatcctgactctgctggacggcagcactcatgccagcgggttcattgcctctctacgtcccaagctcaacttcattgtcactgatacgaagccccgaaagttcaagaaggccacaccagaacaggtagcaaaggcagcgaagcaagccttgagcaaacctgttgccgtcaaaagcactcagcctaaacctgctaagaagaaatctgaagcaaaggtaagtcatgacctgatggattctaggttcatgctaccaggcagctacgggacaggccgaagaagatag

SEQ ID NO: 101 - DNA, 1110 bp, Ralstonia solanacearum
gtgaagcccaagcgtgctgctatcagtgtggatgaacggctgctggcttcattccagcagatcaacgatttctaccgtcagcacggcaaggagccgcagtccaatccgggcaacattcttgagttccagctattcaacaggctcaaggggcttcgtgccagcaaggagaaatgcgaagctttgcaggacgtggatgagttcaagttgctcacctatgtggagccagccaagccaatcacctctgtagctgacatcttcaaggatgactctctgggcctgctggacgacgaagctgccagcatcttccaactcaagcatgttcctttggtgaaggacatgcccgacaagatcgcccagcgcaaacgctgcaaggactttgagcagtttgaacccttgttcaagcagtgccatgcagagcttgccgccggcgttcgtgaggtacgtggcttcactggtgagcagcaaattcagcctggtcacttcttcatcctccacggcattactgcctatgtggctgaagtcggtgagaaggaagtcaagaacggcaaggtcaacgcacggttgcgctgtatctttgagaacggcaccgagtctgacatgttgcttcgctcccttgcaacggagctatacaaggacgaaacgggccgccgcatcctggaccccacggagaaagctctagaggcactggaactgattcaagccgaggacaagaagtcaggctacctctatgtgcttcagtccctcagcacggtgcctgagattgccaacaccaagaacctctacaagattggctactccacggtgccagtgcccgaacgcatcaagaatgcggccgaggaacccacctacctgatggccaaggtcaaaatcgtagaagtctttgagtgctacaacctcaatccacagaaatttgaactgctcctgcacaccttctttggcaaggcatgcctcaacgtggatgtctatgaccagcagggcaaacggttttcaccccgtgagtggttcattgcccctctgcacatcattgaggcggctgccaacatgctgatcaacggagacatcgtcaactatcggtatgacgctgatgcccagagcatagaagaacgataa

SEQ ID NO: 102 - DNA, 1371 bp, Ralstonia solanacearum
gtggcaatgactggctcctacttccgtggtgacagcgtgccggtcctgctgcctgaagacgaggctcagttcaccaaagtcacctacaactactacgagcagttaaacggctataccttcctcaagtctctgggcattggctaccacttctaccaaggcaagtacaccagtgccatcgatgaaattctgaacaccgatcagaagaacattctgcacataccaagcgtgcagtctggtgaatccaccaaggacaagtatgacgaggtggatcgcattctagacatcattggcaccgtcacccaccgggaccctgaaacctgtgtcattcacgtcaagcggcatggtgacggcaaggatttgaaggttgccgatctcgtgcatgacgagcccaaagcccgcgaacgtatcgttgaatacctacgccgcattgagaagccagaagacatggatctcatcattgccttgggcatggcgaaggaaggctttgactggaaattctgtgagcatgccttgaccgtaggctaccgcggatcgctgactgagatcatccaaatcattggccgctgcacccgcgatagcaacaacaagagccatgcccagttcacaaatctcattgcccagcctgatgccactgacgacctggtgaagctgtcggtcaacaacatgctcaaggccatcacctgttccttgctcatggagcaggtgcttgcacccaacttcaagttcaagaccaaggtcagtgacgacgacaagagcaccgatactgaaatcaaggtgcgaggcttgaaggagcctacctccaagcgagtcaaggacatcattgaatctgacctcaatgacctgaaggcggcaatcctgtctgacgacaagattgtgaaggcactgcctggcaatgttgatcccgaagtgattaacaaggtcatgatccccaaaatcatcaaggtcaagtatcctgaactcagtgacgaagaggttgaagaagttcgtcagcacgtggtggtggattcggtcatcaagaatggcgagatcaagcaggaaggtgacaaacgctttgttcgcatggccgacaagttcatcaacattgacgacctgcacattgacctaatcgaccgggtgaaccccttccagaaggcgtttgaaatcctgtccaagtcgctgaccacgaaaatcttcaaggtgattcaagacaccattgaagctactcgtgtgcagatgagtgacgaggaagccatcctgctttggcccaaaattaaggacttcgtgaagacccacggcaaggagccgaacattgcttcgctcgatccgttggagcggcgtatggctgatgctgtggtctatctgaaggcacaacgccgtcagcaaggagtctga

SEQ ID NO: 103 - DNA, 216 bp, Ralstonia solanacearum
atggcgattgtcgccatccgtgaagcgtatcctgaactgagcatcgaggacctgtatgaccctgacactatgcctgctgacttgaagcaggcacaccacgaactggatgtggtggttgagcagtgctaccagattaagccgttctacagcgataccgaacgcttggaatgcctcttcaaactatacgaaaaaatgatggaagccgagcatgcctaa

SEQ ID NO: 104 - DNA, 363 bp, Ralstonia solanacearum
atgaacaatggttacaatcatgcgccacctccatatccaatcgccgcgtttccaatagaaattgccgaggtgatatacgaggtgatgaacaaggtcaaggcgccggacgcgctcatcgcaacgtcatttttggcaacaatgtcagtcgcctgccagggtctgattgacgtcaagctgcctatcgggcaggtttgcccagtgtcgttgaacatccttatcaagggtgagtcgggcgagcgtaaaagtgccacagacagaatcgtgggcgatccgatctatgctcatgatgaagctagggaaccgctgatcaatgaagtcggcgaatggaatcgcgagtcatgccatcttggtttttcggattga

SEQ ID NO: 105 - DNA, 429 bp, Ralstonia solanacearum
gtgacgcaataccgcaagggcgaccgcaagatggacaacgaagcgtgcctgcgcctggcgcaactgctcgagctggaaaaccccttgccggtgatcatggcagccgatatggatcgcgccgagcgtcacggacaacggtctctgtgggaggttttttcgacgaggatggcaggtagtgcgacggcagccctcctcctggtggtggtcgcaggtgtcacaaattttgtgacaccctcggccgccaaagccgcgccgttgagccattcagaaggccgacctgtttatcttatgttaaataaaatggtcgccgcagacgcccgatccaaaacgcttgccggcaccaatgcctccaatgaattcaatgagttgcaagcaggatacgtaggtttgaaaaccatccctggctcaaccctcaacgccgctgtttaa

SEQ ID NO: 106 - DNA, 804 bp, Ralstonia solanacearum
atgaatgtgaaagcgttcgggaaagcagggggcatcatgactgccagcacccgtcatccattgctgatttcggatcatgctcttcagcagaccgtgggctggatcgcgctgctgatgccggtcactgtgcgggtcttcgcgctggccttcgaccacatatggacgacgaattcgatcagcgcttactactacaccagtttgcgcgatgtgttcgtcggttctctggccgtcggcggcctggtgctcgcgttctttcggaccggccatccgcgggatcgctgggtgacgatcagcgcgggcgtgtcagcgatcggaatcgggctgttcccgatgggcatcataccaagcgcgatcaccccgcagcaggccgcaacacccgatgccgaggccaggctcattgacgccctgcagcacgggccgcatgggccgctcgggtatcacttctacttcgtgggggcgttctttctgctggcgttctatcttgtgaccttcggttttcgcgtcaatacgccggcgaaccccacgccacaaaagcggcggcgcaattgggtatacgtgatctgtgggtgcctgatgggattcgcgttcggctggattgccatgctctggctcctggggggcgaggcgtccattttttggccggaaacgcttgccgtgatgtcttttgccggcgcttggctggtcaaggggcaactggtcttgaaggaccagccgtcccggtcccctgagctgcatgggcggcaaggaaaattccggcgacgctggccggcgatccgccgatatcgggccctgcatcgccagtag

SEQ ID NO: 107 - DNA, 2883 bp, Ralstonia solanacearum
atgtcgcagcagattcagctctctgatggcttgccacaggtggagcgcaggctcttgcaggacatcaagcggttggcgaccggcgatcttgatgcactgcttgctgactcgaccgtcccggaaccgccatcgttggatggcgacctgacgatcgccgaagtcggagcgagcgcgctatacctgatgctgcatcgttgcgttcaaagcatcgttgctgaggtgcttggacgcccaggttatcagccgtcgttacccgaactggagcgtgtcgaagacatgtgcgcacagccggtcgtcttgtcccatgaccgacttggcaacagcggcttccctgggcctcgccacttggcgtctttgttgaaggccgtgggtcgcgaactgccgatcgatagcttggcttgcctgccggcaccgcctagtgtcggcacacccctttggatttcgggcctggcgctcattgcgaagactcggccgtatctttgggcaaatcaccggaatgcagtggagcagggctacctgacccctggggtttcagctgccattggctttcccactggggcggggaagtcgactctatctgagctcaagattctttcgaacctgtgtcaagggaaggacaccatctttctggcgcccactctggcgctggtcgaccagaccgcgcgcgcgctccagaaggcctttcctgaggcgcggctgagtcgagagcgggcgagcgacaacccgttcgattttgacctgggcactcgactgccaccgatgtcggtgatgacgccggagcgctgcttggcgttgatggggtttcagccggagctcttcaaggacgttggcctgctcgtgttcgatgagtgccatctgctgcatgcgggcgtgaacgatcgtagccgtcgcgccgtcgatgcgatgctttgcattctgaacctctccacgcatgcacctgaggcagactacttgctgctctcggccatgatgagcaacactgaggaggtcgcggcttggttggctgagcttaccggtcgcaagtgcttgccgctaaacatggcgtggaagccgacacggcaggtgcgcggctgtgtggtctttccgagcgaccaaattgccgacctgcgcatccgactggcggcgaagcgccaagaattgttggccgccggcaagcgcaagcctgggccgccggtcgcgctgaagcgtgaactcaaggtacggccgctgggcctgcttggactgaagttccggtgggactcgatgcgccgagatgattacgcgttactcccgctttctgacagagagatgccgctttcggtgaatgcaaccacgtggaagctgacgccgaatgtgaacaacgtggcagctgagcttgcgtcgctcgcatcccgcaacaagggaatcaaatcgctcatcttcgcgcagaccgtcccgtacgctatgagcgctcaggagaccgcaggtgagcatcttggggaggcaaacattgccttcacggaagatgagcagcatctgtacaacctgacgcttgatgagctgggcgacgagtcggcgctctacatcgatgccgtcgaagggaagttcgcgcggtggccgagcctctgtcatcatgggcttcttctgccgaccgagcggcaactgcacgagtcgctcttcaagcgtcccgatggcgtgtccgtgctggttgccacgtcgacgctggcgcagggaatgaacctgccgagtcacgtggtcatcatcgcgggcgacagtaagttcgatgaggagaacaagcagctcgcgcagatgaaggcgcatgatctgctcaacgctgccggtcgcgctgggcgggccgggcagagttcgtatggtctggtgctggtgattcccagcaacgttgtggacatcgacgattcgaataataggattggcaagcattggaaggatcttcaaggcatctttgcgcaaagcgaccaatgcttggcgatcgaagatccgctggaagctatcctcgaccgtattcaagaacttggcgagcgtgccggtggcgatgcggactaccttttgcgccggcttcccatctcgacgcaggccgaggacgtcgatggcccggccaggacgctattgaagaagtcgttcgcggcgttcaagctgaggaagcaggcgcgggaagagcaactgaacgattgcattaaccatgcactgcgcgcacgcaggaagcttcaagaactggatgaggagcccgattgggccgacaagatggcagccaactacggcgcgccgtcgaacgtgctgcgcgagctgaaggttcgcctcaagacgtcgcctcctgccgggcgcacgacgctggactggttcacgtggatggccacgtggctgaccgagcggcccatcctcatctcgcagatgaccaggcgtgagagcatggacggattcatgggcgagccgttcaagtcgcttgaggacgacgcggagcgaggagagttcgccgtcgacgttgtgattgcagcgctgagggcgtgggttagcggtcagacgttagcggagattcagaagctgaagcccacgttgcaacagcagaaacgctgcgagcaagcgcgccgtttcgtcctgcgggttctcccggagctcgcttacatcttctcccttccggaacttgtgcgtcggcattttgcggcggaagaactgtccttggacatggacgagcttgcctcggtcgaactcgagaagcttggaagctgcgtccgtgagggctacgattccgttgagaaactcgcactagcgaaggtccacggcaagaccgcctcgcgcgttcgagttcatcgtcagtgggacgagatggactggtggatgggccccaaggtacccggcgagacttggcaggagttattcgagcgagtcaggcgtagtaccgaggactaa

SEQ ID NO: 108 - DNA, 1422 bp, Ralstonia solanacearum
ttgatcagcggttccctaaagtcatccgccagcctctcgatctccgtcagcaaccggcagccggagaccgacaggccagggcacgacgtcgaacggagcccgtcgttttcgcctcggtctcggatgatgtccccggatgcactgctgggcgaatattcgcagcgcagcgacagcagcctgagccagctgcgccgggcacccgactccatgcaggtgtcgtcggcccgatccagtgcattggattcgctcgggggaaacccgccaccgcccacggtccacacgccccatcccgggcaagacagccatggtgtcgacgttcccaaccggatgcagatcgccgagcagttgcgcgacatcttcagaccgcacgtcaacagtgccaacgatgcgcaattcgaagccctgatccagcagcgagccgagcggctcgacgagatgggagaaacgccggcgagcctcgccgaggtgctgtccaagggtgccaaccgggaccggctggcgcaggccacggtgggcttcgtgcgctcggtgccgttcggcgttgcctcgcgcctcttcgatgtgaagcagcaactcacggccttcgccaagacgccggcgcaggtgggcgcggtggttggcgccggatcgggcgtggcggacgccttcggcggcagcctggtgaccaaggccaccagcaacacgcaatggctggcggcctcggccgcgcacctcgaacccgtcatggcgcaagcgcaccaggccgtgcagcccagcctgcgccggctggcggtggaggtgagcggggccttccaggcctattcgctgcgcaacgtggtccgcaccggcgtcgcgccgctggcaacccatgtcctgggggcggcaacggcggccaacgtggattcgtggatcgccgcggtgggcgggccggtggccggcgcggcggcctacatggcgatgcagcacatgaacgagacccagcaccgcacgggcgcggaatacctgctgggccgaacggactgggaaacgcagttcacgctgctcaagcagtcgacgtggacggatccgctgaagggggccgcccagcgcgcggcaaaactgcccgtcgatctcctgaccgagacgctggccgccacgcgcagcctgttcaccgccaccaacatcatcaagaatgcgggcgcgctggccggcggctttgccggtgtgctgaccgcgcagaccgccgccggccggatggcgacgaacgccggtgccagcgaggaagccgtcgtggccatcaagcgggccgtcagcacggtgctgtcggcaccggtctatgccacgtggacgacggccgatgtcgcggccggccccatcgtcgatgcggcggccgggcgcatccagcaagcgttcgcacccaggccgcaagcggtgccccccgcgcataccgccatcgacatcgtg

SEQ ID NO: 109 - DNA, 816 bp, Ralstonia solanacearum
ttgtgggcggaggggccggcgcgtcatgacatggttgccaaggttcgggcgttcgaagatggcagtgtcgagttcagcggttatcgtcgtaccgtggtgcagcgcctgaacgatctgcgtgatcttccccgccgtgcacgcggcgccaagcctgaaaatgaagacgataaagaggcacgggccgctagcgtcaaatctgctgcgaagcgagctaaacagaacgttcgcttgcgctgcaagactgctcgcgtcacgcacatgattacgctgactacgcgcgagtgcattaccgatctggactggttcctggggctctgggacgcttttcggcgtgcgatggctcgctacagtcaattccactacatcgcggttcctgagctgcagaaacgcggcgcatggcatatgcacgtcgccgtctcaggtcgggtcgcgctcaacctcgcgcggcgggtatggctgaaggtcgtgggcggtcgtggcaaggggtactgccacattcgcaaccctcaaggggcgcacttcggcaagcaatggaagttggatgcgctggccagctatgtcgccaagtacatcggcaaggacattgctgagactcggttcaacaagaaaaagtactacaccagtcgcggcatcaacgttcctgaggccgtggtctatgcgatcgagaacagtcgctcagactgtgcggacgcactgaaggacgtgctcacgacgctgtgcgccgagttcgatatcgcggacataagatgcttcgtcgccctcgatggctcgtcgtatttcgctagtgcgtctaaggctgtgctgctcgcggcctag

SEQ ID NO: 110 - DNA, 2190 bp, Ralstonia solanacearum
ttggggttgggaatgaagcgatttgtcaaacggcagctttgggttgccgtattgggcctgagtgcagcatccggtgcatgggcgggttcgtccgactctggttttgacgggttcatgtggatcaacagtggcggcatcggtcaaactcttactccacctgagcgcagcccttcagagcagtgcatggtatcggtcgctgcccacgatgccatatatggaacccgctacaaatatgttggcctcgacacgacgaatagtaagaacatccaatgcatccttgatattggtagcggtaccccagcggggtatgcgactgtgcgccgcatcaatacatgcgtacctgggtatgggttgcaacctgatggttcatgtaagccactgtctccgcgtacccccgtgcaagtctgtacggccagcagctctgtcattcccggtacaggcaccaagaccttcatcgagggcgacgaggggggcagtccagacctgtctgtcacacttgcctaccagcagtcttacaacgactacggatcgaatgtcggcatcggtcaatggacaacaaactggcagcgcagcctggacactggttccgccagcgagtcgactccccaagtgtcggctcagcgtgaagatggttcagtagtcaccttcagcggaaacagcgcggcctggagtgccataggcagccaggacacgctgcggccaatcgcggatgcaaatggcaatacaaccgggtggcagtacaccgtggttgatacgggcgtggttgagacctacgatgtcaccggcaaactgcaatcggttcgggaacgcaacggccgcaccacgacactggcctataacgtggtagggcagttgaccgctgtcacgacgccaagcggccgcagtgtgacgttcgcgtacgacgcgtcaggccggacgactagtactatggctcccgacggcgccgtcactcgctacggctacaacagcgctggcatgctgtcgacgatcacgcggccggatggcgccgtacgccagtatgtctacgaagacagccgcttcccgaccgcactgacggggatcgtggatgaaaacggcagccgctacgccacctatgcgtatgacgaccagggccgggccatcagcagcacgttggcgggcggtgccaatacgtaccagttccagtatggcgacaactaccagaccactgtcaccgaccccaccggcaagaccagcgtctacagcttcctgaaacagaacggcgtgctgctgccgacttcgatcagtgctccatgtggcctgtgtggcagcacccgccagagcagcagctacgacgccaacaacaacctgacccaggaaaccgactacaacggcaccgtcacctcccacacctacgacagccagaaccgcgaaactcagcgcgtggacggctcgggtacgaccagcgcccgcaccacgacaacggtatggcactcgcaattctggaacctgccgactcaaatcgcctcgccgaccaagctggaaacgtacagctacgatagcaacggcaatctgacccgctacagcgaaacgccgaccgcggacagtgacggcagccagggcttcagcgcagcggcgaccggcccggctcggaccaccaactggacctacaccgccgacggcctggtggcgaccagcagcggcccgcgaaccgatgtggcgaccagcaccacctacgtttaccgcaccgccgacgacaccgccacgccgccgcaataccgcaagggcgacctgtaccagatcgttgacccgctgggccacgccaccacgatcaaccagtacgacgccaacggtcggccgctgcagatgacggatgccaacggcgcggtcaccgcgttcacctattcgaaccgtggctggctgaccagccagacgatcaccccggcgagcggggcggggcagaccaccaactacagctatgacgccgtggggcaattgaccaaggcgacgttgccggacggcagcacggtcagcttcacctatgacggtgcacatcgtctgacgggcgcagccgacagccagggcaacagcattgcttactcgctcgatgcgatgggcaaccgcacgcaggagcaggcgaaggatccgagcggcaacttggcccgccaagtcacgcgtgtcttcgattcgatgaaccggccgctcaaggtcacggtcggcactgcgtcttaa

SEQ ID NO: 111 - DNA, 1128 bp, Ralstonia solanacearum
ttgatcgaagccttccgttccaccttcatgccaggcttgaaggttgatgagaatttcgctgacttctgggtgatgcacagagacaaaggcaaccttgaacttcacttccttgttgccaataccgaattgaagaccggccagcaactgaacatccatccgcctggggagaagaacatagagttcttcaatgccttcagtgccgtgatgaatgacaacttcggttttgcacaggtggtgcccgatcccttgaagatcagcctcaagccatttgaagccaagtcccccaacggtaaacgcgagaaggaagccaagaatgactttgctgatgagcttcacactcgcatcgtcagtggctccatagccaaccgtaaccagctcattggctacatgaagaagcgaggcattggcattgccttggtaggtgacgagttcatcaccgttcgcttacctggtgctggaaggaacaccagattgaagggctcgctattcagcaaggacagtgactatgcggccttggtgaaggaacaccaccagtccaagatacccaagttcctcaccccggccgagtccgcggaccaacgttccaagctggtggacttcattgcagccagaacagccttcaacaccaaacgctacctcacccctagacctggtgcaaaacgtgcatatgcggccaagaaagggccttctggtggcaataggaaggatggcaagacacccaccaaagagaaggtgccaactactcttgaacgtgacaccaccgagatccgcagcaagcttgaaaagaagctcaaggagatcaaggaacatgccaaggaggttcagcttggccagcaaggcatcaaccactctgccccccacagagtggcccgcatgcagcacgagcgtgagaaccccaccaacctgcccaatgtgggttctgctgttggaggtttggaggctcagatcggtggtttgtccatgcagtatcacaacctgctgttgctgctggctggtgccaccggcacacgagccacgaagcttcaggctcagatcattgccattgagcaaaaacttgtcgcattgaaccttgagcttgagaagaggaagctcaaggaagccaaagacaatgaactcaaatataaataa

SEQ ID NO: 112 - DNA, 457 bp, Ralstonia solanacearum
atgaagcggcagatgagctttgcagaagcggaaagcgcaggtaagaagcgcgtgaccaaacgccagcgcttcctggcagagatggagaaggtcgtgccgtggcagcgcttgctgtcggcgattgggccccactatccgagaggcgagcgaggtcgcccgccgattggtctggagaggatgctgcggatctacttcctgcagcagtggtacgggctgtcggacgaaggtctggaagacgcgctgcacgacagtatggcgatgcgagccttcgccggcatcgatctggcggtcgaggacgtgccggatgcgaccacgctgttgaagttccgacgcctgctcaacgaacacgacttgacgcgaaagctgttcgacgagatcggcatcatgctgtgcgagcgggggctgatgatgaaggaaggcaccatcgtggatgcccccctcattgaagcgcccccgt

SEQ ID NO: 113 - DNA, 102 bp, Ralstonia solanacearum
ttggccaagaacacggcgcaactgttcagcctgtttggtttggccaatctggtgctcgccaggaggcagttgctggccagtcctgggagcattgcgtcctga

SEQ ID NO: 114 - DNA, 867 bp, Ralstonia solanacearum
gtggccttctacagcgacaagtccagcgtgttccgagtcaatgccaggggcgccaccgcgggtcgcgactatacgcagttcggccgagcgctttatgaactcaatatcgagagcatctgtgccaattccagccaggccaagggtcgtgtcgaacgtatgaatggcacgctgcaggatcgcctggtcaaggcgttgcgcctgcacaatatcagcacgctggaggccgccaatgcctttgcgccggtcttcctggcggacttcaacttgcgctttgccaagattccccgcagcgatttcaacgcgcatcgcccggtacaacctgaagaggatctcgaacgcatttttacctggcgggaatggcgcaaggtctcgaatcgactgaccctgcaatacgatcgtacgctgtacctcatcaccgatcagcccgagcatcgtccgctggtccaccgctatgtcgaggtggcggaatatcccgacggccacatcgaactgtgggccgacggcatctccctgccctatgtcctctatgaccggctttgcgagatcgatcagggggcgatcgtcgagaacaagcggctcggacacgtactgcagattgcccaacgcgtgcaagcgctgcgtgataatcggcgcaacctcgaccggccgtcacgcaccctcattggccagccaccacggcaccctgagttgcgtgcgccaggcaccaagtcaccgcgacagttgagttcagaagacttgcggcgcgccatcgaggagctggcaccaccgccccaccctgcggatgccgcaccgcctcaggccaactcgactgccctacggaaacatcccggaaaggcaccaagaaagaagagcaccacactgcactaa

SEQ ID NO: 115 - DNA, 660 bp, Ralstonia solanacearum
atgccaaagccatcagtcatcgaagagcgccaggttcgccacgtactgaaggttgcagccgtcactgggcagaacagcaagcgcgatgtcgccctgttattggttgcctatggcactggccttacgcccaacgaaatcgccaagctgcgcctgtccgactatctgcaggcgaatggggagacacgcatcgaatcgcacgtgagagcggagattgccttcaacggcagagtgcgaccgctgctatgggccagtgcaaaggtgcgcgctgcgatcgacgtctatctcgaacaccggctggcgtttcgacatggcatcaccacaagtccagccgcgtaccgtggattggattgtcggagtcccctctttctgactggcgagggtgagcctttcggctttacgcgtcgcgtgactgcggccggtgccactagctattcgtgcgagtccctggcagagatattcagacggctacaccagcaggctggcatcgccaacggcaatgccagttccgcgcgccggacattcgcggtgaggcttcatcggcaaggccgatcgaccaagctgatccaagagctgattggggtgtcgagcctctccgccgttaagagactggtagagagtgatcccgtgcgtttggcctccatcgtgagccgggttatttga

SEQ ID NO: 116 - DNA, 522 bp, Ralstonia solanacearum
atgcgaccggcctgcgaactcgtttctgcgaagctcggtgatatcgaaacggaccggcaggacgatcactggctgaacctggtggggaagggcagtcgcgctggtaaggttgcgctgccgccgctggctcgggcggcaatcgaccgctacctggttgaacgcgggctaccgattacgcgcgcgcgttgggatccgaagacgccgatgattggtgggttggggctcgatcccaatggcagcattacgggaacgcggttgtggagtgtggtgcggcgttttttctggtcggccgccgaaattatcgacagcgatcaccctttgctggctgagaagctgcgcaaggccagtccacattggatgcgccatacgcatgccacccacgcgctcgcacgcggtgcggaattgaccaccgtgcgcgacaacctgcgacacgcatcagtctcgaccacctcgatctacctgcacagcgatgaggccaagcgcgcgcggcagctcggggacgcctttggtaccagaaaatga

SEQ ID NO: 117 - DNA, 1167 bp, Ralstonia solanacearum
atgcaccggattgatggggccggaaacgtcaacggcacgtgggtggcggaagacgccgggaccaaccggccgccgactgagattacggcaccgttcatgaacgccatccaagaggaagtggcgtcgttcatcgagtggtgcggccttgtgctgaacaaggctgataacacccagtttcgccaagcgattgtggggctattgcaggccgctaccggctctgcgggctccacgtcgtaccgcaacaagctcatcaacagcggggcgcaggttgtcattcaggcgacatcgccgtcgctttctacgtcgcctcagtatgggccggttgagatgattgctggctgggcttctggtggcgcgattacggcagggacgctagtgcaggacacggcggcccctgtgggccgcagcggcaaggccgtccgctttcaggggtgcacgctcaccggggcggggcaactttcatggcgttaccgcatggaggctactgacgcagcaaacctcaagaatcagaccgttacgttccagattaaagtgcggcacaccgttggcgcggccatcaactacacggtggtactgagaacgcccacggcagccgacaatttcgcatcggtcaacgcaatcaacagcaagacggtggctgtggcgtccggaacgtcgcagttactgacgttcacagtggcgctcggtgactgctcgaacggcctggagatcggggtggacgctacgtgtggcgctgtcgtggccaaagatttctggttcaccgagtggagcctggaagaaggagctaccgcaacggccgtggagtttcggcccattccaattgagctggtggcgtgccagcgctactaccaggcggatgcattccactgcggcggcgcgccaatcgcaagtggcagtgggagtgcagcaatcgctgtcaactcgaccagtttgcaggggacaaagactcttccggttcaaatgcgcgcggggccatcgatgacgtttaaggatatggctggcaattctggggcgtacaccgtctacaacggcgcgacaatccacaaccaggggatgacgagcggcggcatcgggtgcagcgcatccacgatcgatatggatgcaacttggggcgtgtacggcggaacatggtgccgggttaactacatcctgaatgcgaggctgtaa

SEQ ID NO: 118 - DNA, 557 bp, Ralstonia solanacearum
tagttacctccaactcccgtaatgtcttcaaaccaaagaaacacgtgcgcttgctggaatgccgccagggcttgccagagcgcagccggatcgaccaccgagcggtagtagcgcacgcgcagcacgtagcgggatcgggtgccccataggcggttgccaatgtggctgcccacacgcgcaggccccaccaggtggtccacctgcaccagatcaatggggaacatcagcgttggcgcgtcatggctccacaaccggttgccgaccgtgctgccaatggcggctatccggcgcggcatcggctcgcccacgccggcaatggcctcggccgcaacgcggcggtactcgctgatgtgccagcttccgaacttgggccggtgcagctcaatggcggcgtccagaaccgcttgcgtgtcggtgcccaggcgctccagctcggccgcagtgcccgccagcatggcgtcgcccatgccgccgtccggccattcccacgcggcgccaggcggcaacagcgccttgagcgcgtcggtgtagtcgcgtgtggtgtgcgccgtgagctt

SEQ ID NO: 119 - DNA, 951 bp, Ralstonia solanacearum
atgtgggcacgtgccgcgcacggcatgcatggccatctcgaatggcttcatgcccagtgctcgccgctcacgtgcgagctggaacgcctctacgactgggcggcgctctacggtgttgaccgcctcatggccaccgctagcacgggcaacgcgctggccaccggtgtggcaggcatgccgatccttgctggcacgccgctgcgcggccccaacgggctggattacattgtggcggcggcagtcgatatcggcgctggccccactacccctgtttccattcgctgcaccaccaccggcctggccagcaacctggctgcgggtctggcactcacggtggtggacccgattcctggcgtcaatggcaccttgacggtaggcgacgcgggaatcacgggcggcgctgaagacgagagcgtggacgattggcgcgcacgggtggccgacgagtggaccaccatcaccacccgtggcgctcgttccggcaaggacgacgactacaggttttgggccagaagcgcccacccatccgttagcacggccctcgttcagcgccacgtgctgggcatgggaacaatcatcgtccggccgatctgcaacacgctggccgaccgcatgccgactgctgccgtactggccgcagtagctgcgcatctgcaagacattgcccccgccacggcggattggcgcgcggtggcaccactgcggcacgacgtgaacccgtctattcatctgctggccggcagtgacaccgccgccaaccgcaccgccgtgtcttcggcgctgatggccgccgtgctggccgaggtcagcgaaacatcggtgctggccatggccgaggtggatgctgccgttgccaccgtcaccacccaatacacccgcctcgcgccgctggccgatatcgccgtcgcagcgggcgaagtgctggtgatgaatcctgtggtgtgggcatga

SEQ ID NO: 120 - DNA, 579 bp, Ralstonia solanacearum
atgatcgggcaacaggtttgggcacggctccagttgctgttcgcgcagggcgttggccttgtagtcggcgcgagcaaggtgcaggtgcgcgtgctggacgaagagacgttggacaacatcaaccgtgtggagccctatggcttttcctaccggccgaaggccggctgccaaacgtacctggctttcccgtctggtgaccgctcctatggcgtggcgctcatcatcggggacaagcgctaccaaatggagctggtggagggcgaaatcgccattcacgacgacgaacagaactgggtgcacatcaagcgcggcggcatcatcgaggtcaaggcgagcacgaaggtgctggccgatacgcccatgttcgagacgacgaaagacgccaagatcggcggtaaccttacagtggcgggccaaaccagctcggtgggtggctacttcggcgacggcggcggcgctgcgcagatgaagggcggtgctgatgtgaccggagaactcagggtgaatggtaaggacgtgagcgacaagcacacccacacgagcacggcccctggcacgcccacttcggacgtgaattga

SEQ ID NO: 121 - DNA, 1056 bp, Ralstonia solanacearum
atggatgaggcattggcggaaatccgctttgacggcacgcgctacggctactggcagaaggtggaaattcgggagtcggtggacgatctgtgcgccgccgtacagctcgccatcgcgcggccgggcagcggcgcgaacggcggcagcggcatgggcatgacggcgaacaccgtcatcgacgtgctgatcggcgacgagctggtgacgaaggtgcgaccggatgtcaaccgccgccgcgtcgatcccaccagccacacaatcaactttcacgcccggtcgcttggtcgtgagcttgtggactgccagtattccaagacgctatccggcctgaaactgagcgagatcgtgaagcgcctttgcgccacgttcaaagtgtctgtgaagatcgacgctgaaacgcccgtggtgcccagtttttcgatgcagtgcgaggtgcctgccaacgcgctcatcaacgctgtgcgggcggcaaacctgctgctgtatccgttgccggacggtggcctggtgctcacgtcgcctaccagcgctgcgcccgtggccacgctgcaatacggcatgcacttcaaggtctacgaggtcatcgacgaatacaagctgcggttttcggactacgttgtgaagacgttcgactacagcagtgattccgcactcaagggtgccgtgaaggacaatggcatcacgtatttccggcccatgcacgttgtggcggaccgacacagcaacggcctgggcggctgcgagcggcgcgcactcctggagcggaaccggcgcctggcgcgtgcccaccgcatcgaactggaggtgccaggctggcgctatcaggacaccgacggcaagtggcacccgtgggcactcaacacccaggtgcgcgtcatcatcccggaagaagaaattgatggtgtgttcctgatcggtgaacggacattccgtatggatgacaagcacggccgcgtcacgctgctacaggtgatgaaccggggtgcgttcctgggcgaagaaaagaagaaatccaagcgcggcgcaggcgtgcgcggcaaggggcacaagaaatga

SEQ ID NO: 122 - DNA, 1161 bp, Ralstonia solanacearum
atgagctgggaagaccgtttggtcggcgcaagctttcgcggcgtcgattttctgaccgagagccacgaagcacgcggcggccgtcgcctcgctgtgcacgagctgccgggcgcggaagagcccgaggtggaagacctgggcgcgaaggcgtgggactggaagctgaacgcctatttcatcgggccggactacgatctggagcgcgatgcgttcttggaactgctcaaccagccgggcgccgattggctgacgcacccgtggctgggtgacaagtgggtccgtgcacatgattggtctgttcacgagagcaacgacaagggcggctattgctccatcacgattgacttcgtgccgggcggcggcacggtagccgctggggaaaccgatgcggtggacgtggcctatgaccgcaacagcagcttggccgacgccgccatggacgacttcacgctggagtcgatgagctttgacggcatgactgcattcgttgcggccgtgagtcagcggctcgaagcgctgcgccaggtcatttccctggccacgctgccgctcacctggtcgagccaggtcatgaacctgattgccggggccaagggcgatctggccacgctgatggccatacccagcgcgtattccaacgcattgcgcagcctcaccaacagcctgggcggtggtgccgatgggacggactacccggatacgtcgcgcgtgcgtgtggcggctcgcatcgcatccgtggccaccacggctgcgccgctggcgctgtctggtgttgcctcaacggatggtgccgtgcgtcgcaatctgctgcgcgaagaagcgttgcgcggccggctgctggtgacggccgccatcgaaatcgccatggcggactaccgcgccgaggcagacagagatgcagccctgggcgccgtggtgactgccctggatgcgctgctgcccagcatgcccgaccctgtgtttgacgcggccgtggccgcgcgggcggcggtcattgaagcgctgctggcccaagacctgaagccgaccgcgttgcgcgacgtgaccaacccgctgccggccatcgtgcttgcgcaccggctgggcgtggatgaatcggtgttcctggcgcgcaatgcggcgcgccatccgctgtttgtgaaggggcgtgtgtatggatga

SEQ ID NO: 123 - DNA, 2145 bp, Ralstonia solanacearum
atgtcgtcgcctgctgttgatgtcgccgtcaagatcaagattgttgatgatggcagcgcgaatgccgcgattcagaagactgccaaaacggcagagcagactgctgccaagacggccacggccaccgagaaggcggcgcagaaggctgccgatgcagccgagaaaagcgccgcgcgccaacgcagctcctatgaacggcttgcccaggcgcgcgaaaccttgggcgtgcgctctgagcgcgccattcagcgcgaaatccagcaaaccgaagcggcatacaaccggctggcgcgctccggcaccatgtcatggcgcgagcaagccgcagcggtcggccagatgcgccagaaggtcactgagctgaccaacgaaatggggcggctgactgctgcgcagaagaccgctgcgggcctgaagttcactggtgctgccatcgccggcatcggtgctgccgcctacacgctcagggggcctgcggaacgctccatgagctatgaccggcgtctggccaacatggccaacaccgcctattccgaacgcgacgccgcgggccgcaaggtcggcaccaagtcgctggaggatgctgtgaacaaggcgcgtcgcgaaggcggtggcacgcgcgagcaggcagcggaagcacttgacaccatgatcgcgtccggcaccgtgtctgatacggatgcaatgaagatgctgccgggcatcatgaaggccgccactgcatccggcaccgacgccaacgcactggccaccatcgccatccgcgcgaaacagtcattcaagatcagcgcggacgacatgccgcaaatcctgagcgctgccatggtggccggccaagcgggcggcttcgagctgcgagatatggcgaagtggctgccgcagcagatggcaatggccagcaacctgggcttatcaggcaaggaaggctttgcgaagctggccgcctggaatcaggcatcggtcattaccgcaggcacccgcgacgaggcaggcaacaacctgcgcgacctgctgaacgagctgaacacaccgcacttccgcaagtacatggcggagcagtacctggccaacggccagaagctgaaacgcggcgagaaggaaaagcgcctaaagggtgttgatgacgtgttcctggactaccagagccgaggcattgacaaggtgggcgccaccatcgacatgatgcagtcgattttctccaaggatgccaagtttcaagcgctgcaagccaagctgcgtgccaccgacaagaacgacaaggaaggccagcggcagattctggaagccatgagcgcccaggtgcagggtacggcggtcggcaaggtgttccacaaccagcagtcgctgatggcgttcctgggcgtgatgaacaatcaggaatacaccaaagacgtgttgggcaaggtgcggggccagtatggcttggcccccgagcgctcggaaatcgccacctccttcaagggcattgcagatacggccgacttcaagatggagcaggccaaggaagatgccgccgtggcgcaaaagtccgccatggacaaccttctacgtacaattggcaaggcagctgaagcctttggcgatctggcgaagaagcatccgcttctggtcggcactaccacgctcgccacggccgcactcggcgcactggcgggtgtggctggcctggcatcgattgcgatgggcgggaaaggtctgccaggcggacaggccatcaccaatgccgctgcttgggcgacaggtagcgcggtcggccggggcgcgatgaaaatgggcaaggtcggcggcatcgctggcgtgggcgctctggtgggtgactacgcccttgagaaagcttttggtgaagagtcggctatttcccgctacggctccagtgcgctcaatggcgctgctatgggcgccatggtgggtagcgtggtgcccgtcctaggcaccggtgttggtgccgctgtcggtggcggtctgggcttagcctgggaaggtctcaaagacctgctgaagccagccgagcagaagccggtggacgtgaacgcccggatgacagtgggccttgcccccggccttgtgctgcaaagccagtctgtgcaggccacgggcggcaacgtgcagatgaacaccggtaatgtttggaacggagcgccctga

SEQ ID NO: 124 - DNA, 354 bp, Ralstonia solanacearum
atggacaacatcgagaaggaaattcagtccatgggcctgactgcgccgcgtgttacgccggccgacatcgaagcgaacatcgcaagcgagcattacttcactgccgaagacggcatgatggccgggcgggacggccgcggccactttcgggatcgccacccgtccctgcacctgctgaccttctgtgtgctggtattgcgcaatggtttcaccgtgaccggtgaatcggcctgcgcaagccctgagaacttcaatgccgaaatcggccgcaagattgcgcgccagaacgctgcgcaaaaggtgtggccgctgatgggctacgccttgcgcgacgcactgcacgccgcaacttga

SEQ ID NO: 125 - DNA, 1473 bp, Ralstonia solanacearum
gtgaccattaccttcagtgaagtgccttcggcgctgcgctatccgggcgcttacatcgagattgatgggagccaggcgggcctgagcagtgatttgcctgtcgtactgctggtaggccagaaattggccagcggcacggcaccgaccggcgaactggtgcgtgtcgccagcgtagcggatgccaagacgctggcaggcgatggttcgatgctggcgcagatggccgcgcgctatcgcaatgtcgatcagacgctcgacctgtacatgctgccctacgcggacaacgcggccggcgtagcagcaaccggttccatcaacgtcaacagtgcggccaccggtgacggcacgctcgcgctgtacatcgcacaacgcgccatcaccgtgggcgtggctgcgggcaagacggctgcgcagattgcgacggccattgcgcaagccatcacggacgcaggtgcggacattccggtgactgccgttgccgctggtgctgcggtgacgctcagcgcgcgccataaaggcacctgcggcaacgccatcgacctgcggttgaacctgtacggcgaagacatgcccgcagggctcgcgctcgcgctgaccgccatggcaggcggtacgggcgacccgctgccgggcgacctggcaacgctgatcggtcagaagtggttccgctacgtcgtgctgggcatcaacgatgccgccacgctggcggcatggcatgcagagagccaacgccgctaccgggtgcccgtgcaggccggcttccgcgcattcgctgcctatcgtggggactacgcagcagcagccagctttggcgagacgaagaactacgagcacatcacggacctgtcgctgggcatcaacccgcccactacctgggaagccgccgcgacgctggccgccgccgctgcgccgaagctgtacaacaacccggttatctcgctggaaggtacatcgctgcctgggcttgtggccacgagctatcacgagtggaccagcgggaacagcctgctcttcaagggcatgagcctcatggaggtaggcacggacggttcctgctacatcaagcggctgatttccatgtacctgcatcgctccgatggcagcaccgacgacgcctatctggatatcaacgtggccgaggttacggagcgcatccgctacgaacagcgcacgggcgccatcaagaagttccggggcaccgttgccgccaagacggacgaaggctaccggccgggcctgcccatcaccacggaagatggcgtgaaagcgttcctgctgtcgctctacaagaacgtgctgatgtcgcgatacggctgggtgcaggcgtatgactactacaagagcactctggtggtggagcaagacccggacaaccccagtcgcttcaacttcctagatgacccggtggtcaactcgccgttctacatcctcgcgggccgctcgcgcttccggaaggctgtaccggtggtctga

SEQ ID NO: 126 - DNA, 480 bp, Ralstonia solanacearum
gtgatttcgctgacgccactgattgaacacgtgaagttgaagcctgccgagttcgcaggcatctggtttcggcaggtagctggtgctgccgagttcgcccaggttcgccctgaagcgttgccgctgccggccgcgtggatcgtccgggcagcagacaaggtgcagcacgcaggcgagcgggctgagaacgtgacgctggccttcgatgtggtgattgccattgaaaaccgccgcacgcacaagcaaggcgatacggacgatgagttgctgaagtaccgcattgccgtgaaaacgctgctgctgggctggcagcttcagcctggcgtgcgccccatcaagttcgtcggtggccaagtcctggagtacaccgacggcgatttgtactggcgcgatcgctacgagttcgacgccttgattaccaactacctaccggacccgcctgcatttgacaggctcatttacacgggagaaaagctgtga

SEQ ID NO: 127 - DNA, 480 bp, Ralstonia solanacearum; identical to SEQ ID NO: 126

SEQ ID NO: 128 - DNA, 903 bp, Ralstonia solanacearum
atgggtgcactcacgcaagcccagttggaccaactgaagaccacgctgattgcccgctggaatgctggcttggtgctgacgccggaagactggaagaagatcgccaagctagtcaccagtaccggcaaatccaacacctacgaatggctgagccagttcccggccttccgggaatgggtcggcgcacgcctgcacaagaagttcaaggaaaccgcctatcaggtggtcaaccgcaagttcgagtgcacggtggacgtgcagcgtacggacatcgaagacgacgaaatcggccagtacggcaccatcgccgagtcggccggccagtcggccaccgatctgaagaacgatctggtgtttcaagcgctgggcgctggcttcgcctccgtctgctacgacggccagtatttcttcgacaccgatcattcggtgtacgagaacgaagacggcaccggtgccgtcaccaacgtgagcaacatgcaggatggtgcgggcgcaccttgggtgctgctgtgcacgaagcgcgcagcgtctccgatctacctgcaacagcgcatgcccgcggagttcaacagcatcacctccacgcagaacaccaacgtcttcgacctggacgtgtacagcttcggcggccgttggcgcggcgaggccgcttacggcttctggcaatgcgcgttcggctccaaggctgcgctcaacgctgccaacttcaacgcggcctacgaggccatgatgaagttcaggggtgacggtcagcgcaagctgggcatcgtgccggacacgctgctctgcggcccggacaacatggccgatgccgaagcgctgctgaaggccgcgcagaacgccaacggctccagcaacaccaactacaacaaggtggaactggtcgttacgccctggctgtaa

SEQ ID NO: 129 - DNA, 984 bp, Ralstonia solanacearum
gtgacgcagaatccccccatcgcacaaaccgcaatagccgcgctagcgttcgagttggtgcccggcagcgatggtgccgttccgactgaagcccatttgctgccgctcggcccgttccgggcaacggatggtcgcccgttcgactgcgcggcttggcagctcgacgccgccatcgccgcgcgcgtgatcgcgctggccgagcagcagaagaacgacatcctggtcgactacgaccaccagagcctgaacaaggaacggaatggccagcgtgccgatgcggctggctggattccccgcacgctcgaatggcgcgaaggcaaagggctctacgccaccaacatcgcatgggtcggcgacgccgcccagctcatcgcgcagaagaagtaccgctacatcagcactgtcttcttctacagccccgtcacaggtgaagtgctcgaaatcctttcggtcgcactcaccaacacacccgccctggatgggctagaagccgtggccgctctggcccgcaagcaattcaattttcgagaaggggagttcgacatgtcgcagcaagaaatcgccgcgctcacgaccgagcgtgacggcctgaaatcgcagctcgcagcgctgaccactgagcgcgatggcctgaagaccaacgttgctgcccttacggcagagcgcgacacgctgaaggccaaggtggaaggcatcgagaaggaagaagccgaagccgcgctggccgcagagcgcaaacaacacggcgatctgctgaaagccgcgctgaccgatggccgcctgaccccggcgcagaagccgtgggctgaaaagcaatcgctggccgcgctgacagagtacctggacgccagcaagccgctggccatcctggacaagcaggccgatggcaagagcgaaggtgcccatggcctgacccacgaagagcttgcgatgtgcaagaagatgggggtcacccccgagcagtacgtcgccgccaaacgctga

SEQ ID NO: 130 - DNA, 948 bp, Ralstonia solanacearum
atggacaagcaaacgacctggcccgcccattacgccgccgcagacgttgcgcttatcggccgcattggcccctggatggaagagcgtggttacacccaagccgctctggcgcggctggcgcgcatcagcgcaagcagcttgaaccagattctcaaaggcagctatgccaccagtccgggcaagctgctggcgtcggtggactcggccatgcgccacgctgaagagacgaaggccgatgtggtggccccggtcgaaaccagcgtcttcaagctcgcgcacgccgcgtgcagcatggcccgccgctatcgcaacttcgcggtgttcaccggttacgtgggcacgggcaagacctttgccatcaagcaatacgtggcatcgcatccgaacacgcacctgattgaggccacgcccaccatgacgccgcagagccttgtgcgtctgctggcccgcgtggtggccggctatgacggcaagggcagcattgatgacaagttccgctccgtggtgacggcgcttcgcaacaccgacagtctgctcatcgtggatgaggccgaaacgctcacgccgcaccagctccacacgctgcgccgcctccgtgatctagccaacgtcggcatcgtgctctgcggcaccgaacacctgtcgggcctcatcaagccgctgcatggccagtttgaccaaatccgctcgcgcacgggcttctggcccgagacggtgcgcgccatcaacctggaagacgccgccgccctggtgcaagccggcttcggcacggaagacgtacctgaagaggtggtgcaacgcctttaccaatactgcaagggcagcgcgcggatgctggtggaggggttggtcgctggcatcaaggagttccgccgtgggcgtccgctggacgtgaagctggtggatgcggtggccaaacaggcgctgtgcctgcaatcgctggcctga

SEQ ID NO: 131 - DNA, 531 bp, Ralstonia solanacearum
gtggaccgctacgccaaccgcccgcacccagaagaccagaacacgacacgcgccgcgctgtggtcgcaactggcaccgcttccgccgcacgccaacgtgaccgaactgaagcgccaggcggtggtgctgacggtgaatcgcgcagccatcaagcacggcaagcgtgcctacacgcaccccgagctgcacgccttcaacggccacagggtggtcatggaatacgacctgatggatgaccgtgtcgccgtcatgcgcacgcaggaaggccggtggatttgcgacgcacacctcgtcacggccattgacgccatcgcgcccaaccgcctggaagaaaagcgccaggcgcgcgccgccgatgccatcaagcgcctgcaacaaaagatggacgagcagaaggcccgtgcgggcatggtgctcgatgtggacagcgtggccgacggcgtgctgcctgcaatcgaggtggaagcccgcctggtggacgacagcgacgacgcgccgctgctgctcgacctgacgatgaacgacgaataa

SEQ ID NO: 132 - DNA, 884 bp, Ralstonia solanacearum
atggcacgggcacaactggttcagcgcgtggacgcaggcggcgcaagtggcgcggtagcggtcatgcccacagcaaaggtgctggcgcttcgtgctcgcgacccgtggcgcgaggcaaccgacagggcccggcaagtggcgacgtggcgtgagacggtggtggcgtacgtgcgcgccatgacggaagacggtgtgacgcagaacaacgcggtggcgctgctgctggagcgcggcgaggccggcagcctgcccagccacttcgccatggcgctggctggcgccgccaaggcaggccggaagacgccatcgcgctccgccatctgcgaatggtgcgctcagtatcgcgaaggcggcgcaactgcgctgctgcccgaccacaagggccgcgtggtggaggccgccggctggtgggggccggcgctggagtatttcaacgcgcccggcaaaccagatatggccgctgtacatcgccgcctggcggaagtggacggcttcgcggtcagctacgaccaagtacgcaactacctgacgggggttcctgccatgctcggccgcaacagccccgcacgcattggccgcaacctgtaccgcctgaccgagaaggcatacatccgccgctcgaccgaaaacgccttgcccggcgatgtgtatgtggccgacggctaccgcgcggacgtgtacctggcgcaccccgtcacgggcgatatctggcgcccggagctgacggtggcaatcgacatgcgcagccgcttccccgtgggatggcgggccgacgaacatgaaggcacgtatgccgtgcagaacatgtgggcagagtgcttcgcccgctggaaccacgtgccgccgatgctctacatcgacaacgggtccggctacaagaacac

SEQ ID NO: 133 - DNA, 1125 bp, Ralstonia solanacearum
gtgaagccgaccatgacgaagaaaaccaaagccgtacaagcaaccgaagatatcgacctgggcatcacggaaggcgctctagttcacgccacgcagacggacgacgcgcagccagtggatattgcaaccgctgcggcggtgctgcccatcgcgctcgcaccagtggcgaatctcgccgcagtcgcgtcgcatttcaacgtgaccaccgatgacctcggcgagctggcccgcatcggcgctgactctatcggccgtgccatgttcgaaattacgcgtgctgggatggcatttctgcgcgcccaagagttgatttcgcttgggcatggtggggatcgcagatcgactcccgaacgttcgggagttgaaaaggacggctttctgacctggatcaacgcgcatggacttgcaaaccagcgcgtgtacgaagccatgcgcatcgcgaagtttgtggctcaactgccgcaagacgaactggaaggcgtgctggcgctcggcaaggtcaaggtgatgctcttggcctcgctgccgcaggaagtcatcgaccaagccgccgagtccggcaacgacatgatcggcaaagccgacctgatgacggtggccgaactcaaggaagagattcgcgcgctgaagcggcgtgaaaagaactacgaagctgagctggagcgtgcgcactcgcaggtcaaacgcctgtcgcaagtaaatcgcaccaccgaattcctcttgcgcacagaggaaatccgcgaagagtgcatggccctgcaacttggcgttgaactcaacacttccagcctgaaaaagctgttcgatgaggtcaacggcgacgagtcgcccgaatggcgcctccagatggaacagatttgggtgacggcacacgtcatggcttcgcgcggcctggatttgatcgaccacatggccagcctcgtgcgcgaagacgacatgcccgaacgcatccaaggcacccacatcctcagccctgacgaggcccgccaatggctgatcgactacccgatgatcgaaaaccgccacgccgccgaagcagcaaaccggcaagagaagcgtgatgcagccaaaccgcgcggcgccggccgtcccaagggttccaccaacaaggctaagggggagtaa

SEQ ID NO: 134 - DNA, 180 bp, Ralstonia solanacearum; identical to SEQ ID NO: 35

SEQ ID NO: 135 - DNA, 369 bp, Ralstonia solanacearum
atgaccatcggagagcgtttgaaggaagagcgcgagcgattgcgcctcacccagccggccattgcctcggccgccgacacgacgaagcaaacacagcacgcatacgaaaccgaccgcacgccgccgaaagccagctatctggccgcaatagcgatcctgggcgttgatgtggcttatgtgataacgggggagcgggcgcttaatacggcgcgcacgccgatggaagttgcactgctggagaactaccggcacagcccagcagaggtgcagcggggggttagcatgcttctcgcacaaacctccggagccgcagattcagtcgccggaaagggaaagaaaaatcgatcctcggaggaagtgaagaagtga

SEQ ID NO: 136 - DNA, 318 bp, Ralstonia solanacearum
gtggcaaacaacacgctgttgagcatcaacggcatgaacaagcgtttcgacggtgcttggctgatgctggacgcgagtgcattgccacatccttccgacttgacgcgggcgggcgggttgttgctccgcgttgcgaagggctaccactacggcaaccatctggcccttgctcaggaggcgattattggtgcgcaaagaagcaaggggaagcaatcctgcattgacgatcccgcatcaggtagccgcctctgcgtctcgaccgataacggcacgatctacaacatgatcttggagcaagccgagcccaagaagggctga

SEQ ID NO: 137 - DNA, 336 bp, Ralstonia solanacearum
atgaaactccctcgccttaccacgtggatcatcgccaccattgcgctggcgatcatcatcggtctgctgtctccgcagcaactacccgtcagtctttacaagctctcgttggtcaccatggctgccgtggtggcgtactggctcgaccgcgcgctgttcccctatgcccggccggccggctacctgtccagtgcggactggcgcaaagacgggcctatgtgcgatgacgccgaccacgccattgttaccggctacgagctggtgtttgccgccgccatgcttcgccgcgccgtcatcgtggccggtgcgatgctcgccatcggtctgggggcgtaa

SEQ ID NO: 138 - DNA, 633 bp, Ralstonia solanacearum
atgcgccgccagtcgatcctcttgagcgtgttcgccgcgctccagctcgcggcggcgctgctgctcgcatcggcagatgcattcggtgctgatgccgccatcccgcacgtcgcgagacgctaccgtgcagacctgacccgcattgcacgcgaggcatggggacttgacgcacccgtggctgtgttcgccgcacagctccaccaggaaagcggctggaaccccgcagcggtctcgcgtgttggtgcgcaaggtatggcgcagttcatgcccgccacggcgaaatggtggtgtgtgctgaatggccttgccaacgatgcgtgccagcccaccaaccctgtgtgggctatgcgtgcattggtgggctatgaccgctggctgtatgagcgcgtgcgcggtgcatccgagtttgaccggctgtgggctgcactacgtgcgtacaacggcggactgggccactggcaacaggaagcggccacggtgcggcctgcgctgaaccgcgcaaccatcgatagcgcatgtggcacagcgcgccgccatcgcagcttctgccctgagaacctgggctatccgcatcgcatcttgaacgtcttgcagccgcgctatctggcgtgggggcgtggggtacggacatga

SEQ ID NO: 139 - DNA, 495 bp, Ralstonia solanacearum
atgacccgcacgctgattgcaacggcggtagccgtagcgtgcggtgcccttagcgcgtggagctacacacgcaatcactacgtggccgagattgctggcatgacggccgatcaagccacggcccgagagaaagccgagaagatcgcgcgtgagctgctcgaagccgagcaaaagcgcggcaacgcgctgtcggacacgttggccaagaaagaaaccgcgatcaccgaaaagacccaggagcttgccaatgcactatctcgcctcactactggccgcaagtgtctcgatgctcgcgttgtgcgcgtgctcaacgactccagcaccggcaccgctactgacaacgtgcgcgccaccaccggcacatctgatgcagcggatggacccgctgccaccgataccgacgtcgctagctggatcaaccacgccaaaggccaatacgaaatctgccgggctcgactcggcgcactgatcgactttgaaaaagggcgagttcaa

SEQ ID NO: 140 - DNA, 153 bp, Ralstonia solanacearum
atgaagcggcagatgagctttgcagaagcggaaagcgcaggtaagaagcgcgtgaccaaacgccagcgcttcctggcagagatggagaaggtcgtgccgtggcagcgcttgctgtcggcgattgggccccactatccgagaggcgagcgaggt

SEQ ID NO: 141 - DNA, 23 bp, Artificial sequence
  Primer
ctgaattcgaaaaggatagagca

SEQ ID NO: 142 - DNA, 23 bp, Artificial sequence
  Primer
ctcgacaaactcttgcaactgac

SEQ ID NO: 143 - DNA, 22 bp, Artificial sequence
  Primer
ctatgcagaagcgttgcttgtt

SEQ ID NO: 144 - DNA, 23 bp, Artificial sequence
  Primer
ctttagcgagcacaagattgagt

SEQ ID NO: 145 - DNA, 23 bp, Artificial sequence
  Primer
gagatcgttggaaacatcaagac

SEQ ID NO: 146 - DNA, 22 bp, Artificial sequence
  Primer
gtgaaccactattgccggtatc

SEQ ID NO: 147 - DNA, 23 bp, Artificial sequence
  Primer
agaactgccaagttcgactacct

SEQ ID NO: 148 - DNA, 23 bp, Artificial sequence
  Primer
cattccaacgttcagatggttat

SEQ ID NO: 149 - DNA, 22 bp, Artificial sequence
  Primer
gcagaaaagatatcccctgcac

SEQ ID NO: 150 - DNA, 23 bp, Artificial sequence
  Primer
ttcgagtacaaatgtaggcttcc

SEQ ID NO: 151 - DNA, 23 bp, Artificial sequence
  Primer
gatcagatggagcaaagaacact

SEQ ID NO: 152 - DNA, 23 bp, Artificial sequence
  Primer
tattgaaactcttcacgggtcat

SEQ ID NO: 153 - DNA, 23 bp, Artificial sequence
  Primer
tttcgaccaagaaaagcatagag

SEQ ID NO: 154 - DNA, 23 bp, Artificial sequence
  Primer
atttctgtgcccactacgaacta

SEQ ID NO: 155 - DNA, 23 bp, Artificial sequence
  Primer
tggtgttctaactgtggaaggtt

SEQ ID NO: 156 - DNA, 23 bp, Artificial sequence
  Primer
taccgccagtcatatcagttctt

SEQ ID NO: 157 - DNA, 23 bp, Artificial sequence
  Primer
attagactgatcaaggcatggaa

SEQ ID NO: 158 - DNA, 23 bp, Artificial sequence
  Primer
ccttcattattgagacggtcaag

SEQ ID NO: 159 - DNA, 23 bp, Artificial sequence
  Primer
atgtttgtgctactggtcagtcc

SEQ ID NO: 160 - DNA, 23 bp, Artificial sequence
  Primer
ccttcacttgcagataatggaac

SEQ ID NO: 161 - DNA, 23 bp, Artificial sequence
  Primer
aaagaagctcaaggagatcaagg

SEQ ID NO: 162 - DNA, 23 bp, Artificial sequence
  Primer
aacagcaggttgtgatactgcat

SEQ ID NO: 163 - DNA, 21 bp, Artificial sequence
  Primer
cgagctcatcgttatcgacat

SEQ ID NO: 164 - DNA, 23 bp, Artificial sequence
  Primer
aagctcttggactaggacgatct

SEQ ID NO: 165 - DNA, 23 bp, Artificial sequence
  Primer
tgaaatgctcaaagacaaacaga

SEQ ID NO: 166 - DNA, 22 bp, Artificial sequence
  Primer
atcgtacaggtcattgccaaat

SEQ ID NO: 167 - DNA, 23 bp, Artificial sequence
  Primer
tacaacctgaagaggatctcgaa

SEQ ID NO: 168 - DNA, 23 bp, Artificial sequence
  Primer
aaagccggtcatagaggacatag

SEQ ID NO: 169 - DNA, 23 bp, Artificial sequence
  Primer
caaccagaccatctacaagatcc

SEQ ID NO: 170 - DNA, 23 bp, Artificial sequence
  Primer
gcttcatactcaaatcgaacacc

SEQ ID NO: 171 - DNA, 23 bp, Artificial sequence
  Primer
aactccaacttgcttgactgttc

SEQ ID NO: 172 - DNA, 22 bp, Artificial sequence
  Primer
ggatgaacttcgttcgattgag

SEQ ID NO: 173 - DNA, 21 bp, Artificial sequence
  Primer
gaagctcggtgatatcgaaac

SEQ ID NO: 174 - DNA, 21 bp, Artificial sequence
  Primer
ggtgatcgctgtcgataattt

SEQ ID NO: 175 - DNA, 23 bp, Artificial sequence
  Primer
gtatcagaaaggccagctacaca

SEQ ID NO: 176 - DNA, 23 bp, Artificial sequence
  Primer
ctgattgccaatattcgattctc

SEQ ID NO: 177 - DNA, 21 bp, Artificial sequence
  Primer
aagggcaatggctttttctgt

SEQ ID NO: 178 - DNA, 23 bp, Artificial sequence
  Primer
gagactgataaatcagcgtttcc

SEQ ID NO: 179 - DNA, 22 bp, Artificial sequence
  Primer
gctttctacgtcgcctcagtat

SEQ ID NO: 180 - DNA, 24 bp, Artificial sequence
  Primer
gtaacggtctgattcttgaggttt

SEQ ID NO: 181 - DNA, 21 bp, Artificial sequence
  Primer
ccactacccctgtttccattc

SEQ ID NO: 182 - DNA, 22 bp, Artificial sequence
  Primer
aaacctgtagtcgtcgtccttg

SEQ ID NO: 183 - DNA, 21 bp, Artificial sequence
  Primer
agacgttggacaacatcaacc

SEQ ID NO: 184 - DNA, 22 bp, Artificial sequence
  Primer
agttctgttcgtcgtcgtgaat

SEQ ID NO: 185 - DNA, 22 bp, Artificial sequence
  Primer
ccagtattccaagacgctatcc

SEQ ID NO: 186 - DNA, 21 bp, Artificial sequence
  Primer
aacggatacagcagcaggttt

SEQ ID NO: 187 - DNA, 20 bp, Artificial sequence
  Primer
atacccagcgcgtattccaa

SEQ ID NO: 188 - DNA, 20 bp, Artificial sequence
  Primer
gttgaggcaacaccagacag

SEQ ID NO: 189 - DNA, 23 bp, Artificial sequence
  Primer
cttgagaaagcttttggtgaaga

SEQ ID NO: 190 - DNA, 22 bp, Artificial sequence
  Primer
ctttgagaccttcccaggctaa

SEQ ID NO: 191 - DNA, 23 bp, Artificial sequence
  Primer
taagagcaggctatggacaacat

SEQ ID NO: 192 - DNA, 23 bp, Artificial sequence
  Primer
aataccagcacacagaaggtcag

SEQ ID NO: 193 - DNA, 22 bp, Artificial sequence
  Primer
ctgatttccatgtacctgcatc

SEQ ID NO: 194 - DNA, 23 bp, Artificial sequence
  Primer
acatcagcacgttcttgtagagc

SEQ ID NO: 195 - DNA, 22 bp, Artificial sequence
  Primer
gacgatgagttgctgaagtacc

SEQ ID NO: 196 - DNA, 23 bp, Artificial sequence
  Primer
tgaaggtaatggtcacagctttt

SEQ ID NO: 197 - DNA, 22 bp, Artificial sequence
  Primer
atccggttccttctgatgatct

SEQ ID NO: 198 - DNA, 23 bp, Artificial sequence
  Primer
cttcaacttcacgtgttcaatca

SEQ ID NO: 199 - DNA, 23 bp, Artificial sequence
  Primer
gctagtccacacgacaaaatcat

SEQ ID NO: 200 - DNA, 22 bp, Artificial sequence
  Primer
gtgactagcttggcgatcttct

SEQ ID NO: 201 - DNA, 21 bp, Artificial sequence
  Primer
aggtgaagtgctcgaaatcct

SEQ ID NO: 202 - DNA, 22 bp, Artificial sequence
  Primer
cttcttccttctcgatgccttc

SEQ ID NO: 203 - DNA, 23 bp, Artificial sequence
  Primer
ttatgtggctttctctggcaata

SEQ ID NO: 204 - DNA, 21 bp, Artificial sequence
  Primer
acaaacgtccagtcgtcaatc

SEQ ID NO: 205 - DNA, 23 bp, Artificial sequence
  Primer
catttatctctggtcttggcttg

SEQ ID NO: 206 - DNA, 21 bp, Artificial sequence
  Primer
acagccaaactgacaagatcg

SEQ ID NO: 207 - DNA, 23 bp, Artificial sequence
  Primer
gcatgaaaatgtctacgttcctc

SEQ ID NO: 208 - DNA, 21 bp, Artificial sequence
  Primer
atggtgatagtgcggaatgac

SEQ ID NO: 209 - DNA, 21 bp, Artificial sequence
  Primer
cagacatgttctgcgaaggat

SEQ ID NO: 210 - DNA, 23 bp, Artificial sequence
  Primer
gaggaacgtagacattttcatgc

SEQ ID NO: 211 - DNA, 23 bp, Artificial sequence
  Primer
tgtaatgaccaagcaagaactca

SEQ ID NO: 212 - DNA, 21 bp, Artificial sequence
  Primer
cacggtgtcaagaatggtttc

SEQ ID NO: 213 - DNA, 23 bp, Artificial sequence
  Primer
atttcaaacgccaagcctttaac

SEQ ID NO: 214 - DNA, 22 bp, Artificial sequence
  Primer
gttcatcgaaagcgatgttctc

SEQ ID NO: 215 - DNA, 22 bp, Artificial sequence
  Primer
gcctatttggaccgaagaagac

SEQ ID NO: 216 - DNA, 22 bp, Artificial sequence
  Primer
acttcttgaggcatctcggttt

SEQ ID NO: 217 - DNA, 21 bp, Artificial sequence
  Primer
gcagcattgatgacaagttcc

SEQ ID NO: 218 - DNA, 22 bp, Artificial sequence
  Primer
agtattggtaaaggcgttgcac

SEQ ID NO: 219 - DNA, 21 bp, Artificial sequence
  Primer
agcaaccgaagatatcgacct

SEQ ID NO: 220 - DNA, 21 bp, Artificial sequence
  Primer
gcccaagcgaaatcaactctt

SEQ ID NO: 221 - DNA, 23 bp, Artificial sequence
  Primer
ctttcccagtcaatacattccag

SEQ ID NO: 222 - DNA, 23 bp, Artificial sequence
  Primer
cgttatcacataagccacatcaa

SEQ ID NO: 223 - DNA, 21 bp, Artificial sequence
  Primer
aaacaacacgctgttgagcat

SEQ ID NO: 224 - DNA, 21 bp, Artificial sequence
  Primer
cttctttgcgcaccaataatc

SEQ ID NO: 225 - DNA, 23 bp, Artificial sequence
  Primer
cgtgtatatcggcagtcaagaag

SEQ ID NO: 226 - DNA, 23 bp, Artificial sequence
  Primer
ctaagaaatgaaaggtggggttc

SEQ ID NO: 227 - DNA, 23 bp, Artificial sequence
  Primer
ggatgacttgtgttagcgactct

SEQ ID NO: 228 - DNA, 23 bp, Artificial sequence
  Primer
gaatacgatcctccacaatcaaa

SEQ ID NO: 229 - DNA, 23 bp, Artificial sequence
  Primer
tgaagcactgtctatcaaccaga

SEQ ID NO: 230 - DNA, 23 bp, Artificial sequence
  Primer
tttgtcctagtcacagcactgaa

SEQ ID NO: 231 - DNA, 23 bp, Artificial sequence
  Primer
gacttcggctatctggagaaaat

SEQ ID NO: 232 - DNA, 23 bp, Artificial sequence
  Primer
tcttagcaggtttaggctgagtg

SEQ ID NO: 233 - DNA, 23 bp, Artificial sequence
  Primer
catcttcaaggatgactctctgg

SEQ ID NO: 234 - DNA, 23 bp, Artificial sequence
  Primer
gaagaagtgaccaggctgaattt

SEQ ID NO: 235 - DNA, 23 bp, Artificial sequence
  Primer
actcagtgacgaagaggttgaag

SEQ ID NO: 236 - DNA, 23 bp, Artificial sequence
  Primer
acgagtagcttcaatggtgtctt

SEQ ID NO: 237 - DNA, 22 bp, Artificial sequence
  Primer
acactatgcctgctgacttgaa

SEQ ID NO: 238 - DNA, 23 bp, Artificial sequence
  Primer
aggtgacttcaacaatgttaggc

SEQ ID NO: 239 - DNA, 23 bp, Artificial sequence
  Primer
caatagaaattgccgaggtgata

SEQ ID NO: 240 - DNA, 23 bp, Artificial sequence
  Primer
ccttgataaggatgttcaacgac

SEQ ID NO: 241 - DNA, 23 bp, Artificial sequence
  Primer
cttgacgtctgacaaccaagtag

SEQ ID NO: 242 - DNA, 23 bp, Artificial sequence
  Primer
ataagataaacaggtcggccttc

SEQ ID NO: 243 - DNA, 23 bp, Artificial sequence
  Primer
aattgggtatacgtgatctgtgg

SEQ ID NO: 244 - DNA, 22 bp, Artificial sequence
  Primer
tcgggtaagacgaagctgacta

SEQ ID NO: 245 - DNA, 21 bp, Artificial sequence
  Primer
agggaatcaaatcgctcatct

SEQ ID NO: 246 - DNA, 23 bp, Artificial sequence
  Primer
agaagaagcccatgatgacagag

SEQ ID NO: 247 - DNA, 70 bp, Artificial sequence
  Probe
ggctcgaacaacttggagagatttacggaggagacgtgtcattgctcatgccgaattcacgctacgccgc

SEQ ID NO: 248 - DNA, 70 bp, Artificial sequence
  Probe
ccggaggaaccgctgcgatggacgccatcactggaaagtctagtggccgaaccgcgaaagcgcgcgcatg

SEQ ID NO: 249 - DNA, 70 bp, Artificial sequence
  Probe
gtgacgataccgctgttgcgtctctgtcgtttgttgtccaagagggcttcaaaccagatacgttctccgc

SEQ ID NO: 250 - DNA, 70 bp, Artificial sequence
  Probe
gcgaacaatgtcaccgtcgcctatgcagaagcgttgcttgttgccacgcctgcctctcgtcttgttgatg

SEQ ID NO: 251 - DNA, 70 bp, Artificial sequence
  Probe
caaggtctttgacgagatcgttggaaacatcaagaccattgggctaaagaagccaatcaccgtcacgccg

SEQ ID NO: 252 - DNA, 70 bp, Artificial sequence
  Probe
gacgagaatttcgtcaaccttctgcgagcggaatctttggcgacacttcccaaatatctggctgagcgtg

SEQ ID NO: 253 - DNA, 70 bp, Artificial sequence
  Probe
cgcaaccgtgagggaggtgctcaccaacgagaaatacatcggcaacaacatctataaccgtgtgtcgtac

SEQ ID NO: 254 - DNA, 70 bp, Artificial sequence
  Probe
gcgcctggttgtcgcgatagtcgtcatcagagcgcaagtgctgctgttgccaccgtcggcgacgttggcg

SEQ ID NO: 255 - DNA, 70 bp, Artificial sequence
  Probe
ggtgcgacgttcttctcggctccagataaggcggtcctgctactgtcgttcagatatctcacagacgacc

SEQ ID NO: 256 - DNA, 70 bp, Artificial sequence
  Probe
ggctgtgagtatgttctgagggtagccgcgcatcatcgtcccgaaccacgttcgtcaactggtaagttgg

SEQ ID NO: 257 - DNA, 70 bp, Artificial sequence
  Probe
cctggttgtgcttgataaccatctgaacgttggaatgcaagtgctggaagactcgctatgcaactggcag

SEQ ID NO: 258 - DNA, 70 bp, Artificial sequence
  Probe
cggaactaggcacatacaagcacgccgtcggaagcctacatttgtactcgaagaacacggaccaggcgac

SEQ ID NO: 259 - DNA, 70 bp, Artificial sequence
  Probe
gcaggataagagccgtgaggttcctgttcatctgcgtaatgcaccaacaaagctcatgaaggaactcggc

SEQ ID NO: 260 - DNA, 70 bp, Artificial sequence
  Probe
gagcggttccttgaaagctatgctggagccatcatcactgatcctgctacggcgattgtggagcttgtcg

SEQ ID NO: 261 - DNA, 70 bp, Artificial sequence
  Probe
gtcttcatcaaccacattgaagttcacgacaagccagcggcagacaacatcaggaacattgtgttcggcg

SEQ ID NO: 262 - DNA, 70 bp, Artificial sequence
  Probe
ctgcctatcatcctggatagagggcgaagccgaaccttggagcgcacagtcaaggaaggctttgaacatg

SEQ ID NO: 263 - DNA, 70 bp, Artificial sequence
  Probe
cggctacaggcagtctcaaagagacaggtctgaagaagactatgccatctattcggtatgtggctggtgc

SEQ ID NO: 264 - DNA, 70 bp, Artificial sequence
  Probe
cgttgaacatgtggatggtaagcacggaggtaaagtcaggcagttgattagactgatcaaggcatggaag

SEQ ID NO: 265 - DNA, 70 bp, Artificial sequence
  Probe
cgctggtgctgtggtgggctcccaagtgagtaagagcagtgaagtgaagaatgtcactgacttgcaagtg

SEQ ID NO: 266 - DNA, 70 bp, Artificial sequence
  Probe
ccagcaatcccactaatgtttgtgctactggtcagtcctacggcttcaacttcgccaacaatcaatacac

SEQ ID NO: 267 - DNA, 70 bp, Artificial sequence
  Probe
ggaggtttggaggctcagatcggtggtttgtccatgcagtatcacaacctgctgttgctgctggctggtg

SEQ ID NO: 268 - DNA, 70 bp, Artificial sequence
  Probe
gctgtagcaccgactctgaccgccaatgacgaagttctattgaacttcattagtgaatacggtgatggtg

SEQ ID NO: 269 - DNA, 69 bp, Artificial sequence
  Probe
cgtcttgtgcctatagcaatgaaatgcagaaggcttggcggacaatatcaactatgccagctcgtggtg

SEQ ID NO: 270 - DNA, 70 bp, Artificial sequence
  Probe
atcatggctgagatccagcgtattgccgcaaccgagctcatcgttatcgacatgggcgacaggcttggca

SEQ ID NO: 271 - DNA, 70 bp, Artificial sequence
  Probe
gaggtcaagctggatcaagctggagtcgaagtggaggaagcgaaagtccacaagttcagcaagaacgatc

SEQ ID NO: 272 - DNA, 70 bp, Artificial sequence
  Probe
ccacgatcaactactaccgtgtatcgaccaaggatcaatccatcgaatcccagcgtcagaagctaggccc

SEQ ID NO: 273 - DNA, 70 bp, Artificial sequence
  Probe
catcaacgacccttactgtccgagtaactgccgatgggccaccaacaacgatcagcagaacaacaagcgg

SEQ ID NO: 274 - DNA, 70 bp, Artificial sequence
  Probe
gaccgactggaacgagatggccaaggctccgagcaaggtggcaaagatcgcgaaagcagtgggactcgac

SEQ ID NO: 275 - DNA, 70 bp, Artificial sequence
  Probe
cttcatgacggtgtagccgtgacccaggtgtctgaccttgatgcgcgactaagcgcacacttgatgctct

SEQ ID NO: 276 - DNA, 70 bp, Artificial sequence
  Probe
gccggtgaagatacgggtcagcgtctcaagcaggcgcgtgaaatgctcaaagacaaacagatcttcgcgg

SEQ ID NO: 277 - DNA, 70 bp, Artificial sequence
  Probe
gccacatcgaactgtgggccgacggcatctccctgccctatgtcctctatgaccggctttgcgagatcga

SEQ ID NO: 278 - DNA, 70 bp, Artificial sequence
  Probe
ggagaacctcgacaaccagaccatctacaagatcctagtcaagtgcatcaagaaggcgaaggagaggcgg

SEQ ID NO: 279 - DNA, 52 bp, Artificial sequence
  Probe
ggcagatggacatgaatcttcctcttgaatttcacgtccgcattgaggagac

SEQ ID NO: 280 - DNA, 70 bp, Artificial sequence
  Probe
gtgccactagctattcgtgcgagtccctggcagagatattcagacggctacaccagcaggctggcatcgc

SEQ ID NO: 281 - DNA, 70 bp, Artificial sequence
  Probe
gcgctgccgccgctggctcgggcggcaatcgaccgctacctggttgaacgcgggctaccgattacgcgcg

SEQ ID NO: 282 - DNA, 70 bp, Artificial sequence
  Probe
gagaaacatcccgtcgccatgcgccgcatgttgctggttgccgcaaccgtcacgctgctatcgctgactg

SEQ ID NO: 283 - DNA, 70 bp, Artificial sequence
  Probe
cgtttcgtgcatacatcgacagccttgaccactacactgacgcccatgtgattgtccttccgtatgcgcc

SEQ ID NO: 284 - DNA, 70 bp, Artificial sequence
  Probe
gatatctggcaggcatttgccaacccagacgaagatcgcttgcgctacttcaagaaggccactgataagg

SEQ ID NO: 285 - DNA, 70 bp, Artificial sequence
  Probe
gacgtcaagctgcctatcgggcaggtttgcccagtgtcgttgaacatccttatcaagggtgagtcgggcg

SEQ ID NO: 286 - DNA, 70 bp, Artificial sequence
  Probe
cttgaccgatacgtcaagggcagccgactgccgacatatgcagcggcactcatcctggcgaaagaagcgg

SEQ ID NO: 287 - DNA, 70 bp, Artificial sequence
  Probe
cgcaatgcatcctcaccgaaggtgttgctgacgcaactggcggcgtctccgaacagatcaaggttggccg

SEQ ID NO: 288 - DNA, 70 bp, Artificial sequence
  Probe
cgttcaggaggtcgtcatggtcgatccgaacgacaagtacacgatgcagttgccgctgatgcgttatcgc

SEQ ID NO: 289 - DNA, 70 bp, Artificial sequence
  Probe
gggctcgtgcgtcaatgctttggttatggcgctgttcgttctgctgctgacgctgttggtcccggcgtgg

SEQ ID NO: 290 - DNA, 70 bp, Artificial sequence
  Probe
gaggcgatggacgacgctgaggctatcgagttcttcggcagcctggatgagcgtgatgaggcgattgagg

SEQ ID NO: 291 - DNA, 70 bp, Artificial sequence
  Probe
ccacagagctgcagcggctatctgctgttgcagccgagcgaatactcgaacgtgatggctctgtccggcg

SEQ ID NO: 292 - DNA, 70 bp, Artificial sequence
  Probe
gggtgctgcgatggcgcaaaccacgaccagcggtatcgatttctcgtccatgacgggtgccgtgagcgcg

SEQ ID NO: 293 - DNA, 70 bp, Artificial sequence
  Probe
cgacagtgtcttcgtcgctatcttcacggcgctttggcagatctcagaggatctgttcatcgactcgctg

SEQ ID NO: 294 - DNA, 70 bp, Artificial sequence
  Probe
cgagattcctgaagctgaggtgctgcgttggaatgagctggtcgagaacgacagcctggtcatcttggac

SEQ ID NO: 295 - DNA, 70 bp, Artificial sequence
  Probe
cgccgcactgcattgaccaagctcattcgtcagaagcgatctaacaactttcagattgccaaagagatgg

SEQ ID NO: 296 - DNA, 70 bp, Artificial sequence
  Probe
ggaacaaatcaagatgttcaacttcacggctgatgaacttggtttccatgctgctcctgatgcacgtgcc

SEQ ID NO: 297 - DNA, 70 bp, Artificial sequence
  Probe
ctgtttaccaaccacatagcctcacacacggctgctgacggatcagacttggacgactacttgagtcgtg

SEQ ID NO: 298 - DNA, 70 bp, Artificial sequence
  Probe
ggtggtcgaggtgagaagtgtctacgacaaacctgtgaaagccgttgagaatcgaatattggcaatcagg

SEQ ID NO: 299 - DNA, 70 bp, Artificial sequence
  Probe
cgtcattcgtgagaaccagatcaaagagcttctgattgcttacaacagctacttcatggtggctgctgcc

SEQ ID NO: 300 - DNA, 70 bp, Artificial sequence
  Probe
gaagtatgctggcctgatcggtcagctcaactcagccgctagggaaacgctgatttatcagtctcagccg

SEQ ID NO: 301 - DNA, 70 bp, Artificial sequence
  Probe
gcgttaccgcatggaggctactgacgcagcaaacctcaagaatcagaccgttacgttccagattaaagtg

SEQ ID NO: 302 - DNA, 70 bp, Artificial sequence
  Probe
ccggatagccgccattggcagcacggtcggcaaccggttgtggagccatgacgcgccaacgctgatgttc

SEQ ID NO: 303 - DNA, 70 bp, Artificial sequence
  Probe
gtcaatggcaccttgacggtaggcgacgcgggaatcacgggcggcgctgaagacgagagcgtggacgatt

SEQ ID NO: 304 - DNA, 70 bp, Artificial sequence
  Probe
gccttgtagtcggcgcgagcaaggtgcaggtgcgcgtgctggacgaagagacgttggacaacatcaaccg

SEQ ID NO: 305 - DNA, 70 bp, Artificial sequence
  Probe
gcttggtcgtgagcttgtggactgccagtattccaagacgctatccggcctgaaactgagcgagatcgtg

SEQ ID NO: 306 - DNA, 70 bp, Artificial sequence
  Probe
gcgctgtctggtgttgcctcaacggatggtgccgtgcgtcgcaatctgctgcgcgaagaagcgttgcgcg

SEQ ID NO: 307 - DNA, 70 bp, Artificial sequence
  Probe
cggtgttggtgccgctgtcggtggcggtctgggcttagcctgggaaggtctcaaagacctgctgaagcca

SEQ ID NO: 308 - DNA, 70 bp, Artificial sequence
  Probe
cgccggccgacatcgaagcgaacatcgcaagcgagcattacttcactgccgaagacggcatgatggccgg

SEQ ID NO: 309 - DNA, 70 bp, Artificial sequence
  Probe
ttcctgctacatcaagcggctgatttccatgtacctgcatcgctccgatggcagcaccgacgacgcctat

SEQ ID NO: 310 - DNA, 70 bp, Artificial sequence
  Probe
cgtcggtggccaagtcctggagtacaccgacggcgatttgtactggcgcgatcgctacgagttcgacgcc

SEQ ID NO: 311 - DNA, 70 bp, Artificial sequence
  Probe
ccgcatccgatgatccggttccttctgatgatctggccatcatcgaaagcgcaccgcgccgctttggccg

SEQ ID NO: 312 - DNA, 70 bp, Artificial sequence
  Probe
cggaagactggaagaagatcgccaagctagtcaccagtaccggcaaatccaacacctacgaatggctgag

SEQ ID NO: 313 - DNA, 70 bp, Artificial sequence
  Probe
ggtgaagtgctcgaaatcctttcggtcgcactcaccaacacacccgccctggatgggctagaagccgtgg

SEQ ID NO: 314 - DNA, 70 bp, Artificial sequence
  Probe
cgccgagtggttcgcgctctaccgcgatgatggcgcgattgacgactggacgtttgtgagtggcgtgcgg

SEQ ID NO: 315 - DNA, 70 bp, Artificial sequence
  Probe
catgttccttgtggtggcactgctgctggcgatcttgtcagtttggctgttcgaccgctacgtgtgtcgg

SEQ ID NO: 316 - DNA, 70 bp, Artificial sequence
  Probe
cgtggtagacgcctacccggctgtcgatgaagatttgctgagaaccttgccacccgtgctgcgtgcggtg

SEQ ID NO: 317 - DNA, 70 bp, Artificial sequence
  Probe
gcgcgtcaagtgggccgtcacgtgccggacttggacagcttggccgtagctgaatgccaaacgattgccg

SEQ ID NO: 318 - DNA, 70 bp, Artificial sequence
  Probe
gcagacatgttctgcgaaggatgggtgtttgccgtgcgcgagctggtgggtgccaccgtgccgcccgaag

SEQ ID NO: 319 - DNA, 70 bp, Artificial sequence
  Probe
gaccgaaaccattcttgacaccgtgcgcgccggtaacgagctgacgatcaccgacctgggcaagttcggc

SEQ ID NO: 320 - DNA, 70 bp, Artificial sequence
  Probe
tcaccttgagcacgttcgacggtcgccagcaggtagtcgtgtcccaggccgagaacatcgctttcgatga

SEQ ID NO: 321 - DNA, 70 bp, Artificial sequence
  Probe
ccgctgcctgaagtcgtctgcccgaactgccgcgtgcggatgagcctggatgtggtgctagccgatgacg

SEQ ID NO: 322 - DNA, 70 bp, Artificial sequence
  Probe
gaaacagctagcaggcatcacccagggacctgttgcagccgaactgctggcgggcttgcgccgaatctgc

SEQ ID NO: 323 - DNA, 70 bp, Artificial sequence
  Probe
cgcttcttggatgccatcgccaacggcgcaccttcagcagaggcagccgcgtttggcgcctggatcgaag

SEQ ID NO: 324 - DNA, 70 bp, Artificial sequence
  Probe
cttcgcaacaccgacagtctgctcatcgtggatgaggccgaaacgctcacgccgcaccagctccacacgc

SEQ ID NO: 325 - DNA, 70 bp, Artificial sequence
  Probe
cgctgtacatcgccgcctggcggaagtggacggcttcgcggtcagctacgaccaagtacgcaactacctg

SEQ ID NO: 326 - DNA, 70 bp, Artificial sequence
  Probe
caagcaaccgaagatatcgacctgggcatcacggaaggcgctctagttcacgccacgcagacggacgacg

SEQ ID NO: 327 - DNA, 70 bp, Artificial sequence
  Probe
gccgacacgacgaagcaaacacagcacgcatacgaaaccgaccgcacgccgccgaaagccagctatctgg

SEQ ID NO: 328 - DNA, 70 bp, Artificial sequence
  Probe
ggcgatcttggaatgtcagaccgtggcaaacaacacgctgttgagcatcaacggcatgaacaagcgtttc

SEQ ID NO: 329 - DNA, 70 bp, Artificial sequence
  Probe
ggactgggccactggcaacaggaagcggccacggtgcggcctgcgctgaaccgcgcaaccatcgatagcg

SEQ ID NO: 330 - DNA, 70 bp, Artificial sequence
  Probe
gtcgctagctggatcaaccacgccaaaggccaatacgaaatctgccgggctcgactcggcgcactgatcg

SEQ ID NO: 331 - DNA, 70 bp, Artificial sequence
  Probe
ggaaggtctatcacgatcatctcagtgaccctaacaagaggccaccagcgaacctggaacttgttgctgc

SEQ ID NO: 332 - DNA, 70 bp, Artificial sequence
  Probe
cgcacttacctcgacgccagcattgagtcattggatcgccacattaagctgctgactagcctaatcgaag

SEQ ID NO: 333 - DNA, 70 bp, Artificial sequence
  Probe
caaattaaacactacaaagggctggtagtttatgcgcctctaatcgcgctatctgtggtctttgtggccg

SEQ ID NO: 334 - DNA, 70 bp, Artificial sequence
  Probe
gcggtgcttgccttgtctggcgccaaacgcttccagcacttcatcaaggtcgttgcagattggcaggagg

SEQ ID NO: 335 - DNA, 70 bp, Artificial sequence
  Probe
acgcgaataaggcatcgctaccaggtgacgtaagccgggtatatagaaatgctttagacatgatctggag

SEQ ID NO: 336 - DNA, 70 bp, Artificial sequence
  Probe
ctatcggttccgctgcgcacatgggcatgaaacgtcccgcaccggcgactacgccctccgtagtctgatc

SEQ ID NO: 337 - DNA, 70 bp, Artificial sequence
  Probe
cttcgctaaataataatgctgaggctcatgatcttctatactcgctgcttgcccatgacagtgaggcggc

SEQ ID NO: 338 - DNA, 70 bp, Artificial sequence
  Probe
gacattgcagcatccgtcttgcgtgacattgaagtcggcaaacacggcttaatgtcggcttcggcgagcg

SEQ ID NO: 339 - DNA, 70 bp, Artificial sequence
  Probe
gtgcgcaaacaaggcacgcggcgaagcgtctctgcgcaaagacggattggacgaactgcgccgcatcgcg

SEQ ID NO: 340 - DNA, 70 bp, Artificial sequence
  Probe
ggccggagacatctttgcgttccagttggagcagtttccggatcggtacttctttgggcgagtcgtggac

SEQ ID NO: 341 - DNA, 70 bp, Artificial sequence
  Probe
cagcggacgagcaacgataggttgcttcacgacgatctcggcgcgtgtatatcggcagtcaagaagttcg

SEQ ID NO: 342 - DNA, 70 bp, Artificial sequence
  Probe
cgttgggaagacaaagagaacgtctttctctacgcatcaggatttcttgccgcaatggatttcctcaggc

SEQ ID NO: 343 - DNA, 70 bp, Artificial sequence
  Probe
cgcgggcgtcgcttgccctcggtggcgtcgggatcttctattcaagaactacgccctattcgtggcgggc

SEQ ID NO: 344 - DNA, 70 bp, Artificial sequence
  Probe
caaggcaatttgattgtggaggatcgtattcaggttcacatgagcttgcgacttctgcatattccaacgg

SEQ ID NO: 345 - DNA, 70 bp, Artificial sequence
  Probe
gtcgggttgttgtacctctacggcgagcaaaccggtcatccatcagcggctggtggcgctctactgctgt

SEQ ID NO: 346 - DNA, 70 bp, Artificial sequence
  Probe
cgagtatcaccttgttgaggctcgatcagttactgaatgccgcctatgccaacctgttgaccaagatgcg

SEQ ID NO: 347 - DNA, 70 bp, Artificial sequence
  Probe
gatggcatggcgaaccatcacctgattcctgaggagttgatgaaacatcctcgatacgcggcgatgttcc

SEQ ID NO: 348 - DNA, 70 bp, Artificial sequence
  Probe
ggcttgaaatggtggagattcaactagacaagcatggccgcgctgcctttcgtctgaacattggtgtcgc

SEQ ID NO: 349 - DNA, 70 bp, Artificial sequence
  Probe
gccacacagcttctcggcggctcaatgcggtcgtgtgtctcttgagatcgctgacagaacgctcagggcg

SEQ ID NO: 350 - DNA, 70 bp, Artificial sequence
  Probe
gtgcatacgagggactgtatatgctctcgtgcatggaaacactcacaaaggaacgcgccgaccacttggc

SEQ ID NO: 351 - DNA, 70 bp, Artificial sequence
  Probe
caatagccttgccaagcactgcaacatgaaggcggtcacggatttcaacgacctgcatcagaagcaactg

SEQ ID NO: 352 - DNA, 70 bp, Artificial sequence
  Probe
gcggaagtgtgggcagaaggaagctgttgagtatatcgctgatttgaatcggtatacgacgcttcgaccg

SEQ ID NO: 353 - DNA, 70 bp, Artificial sequence
  Probe
cgatcacttcatcgagctatcaaatattggtgaagaagatcgttcagtggaggcaatggcggttgggatg

SEQ ID NO: 354 - DNA, 70 bp, Artificial sequence
  Probe
gttggtgagcgaagcgcagatgcgtgccgctggtgaagcgatctatggtgaacgtaaagtcgttacgtgc

SEQ ID NO: 355 - DNA, 70 bp, Artificial sequence
  Probe
cgtcgtgaacacctcggttcgcaagtacgacaagaagtactacaagttctatcgcagccacaccaagagc

SEQ ID NO: 356 - DNA, 70 bp, Artificial sequence
  Probe
cggcgtgatcggcgttgcgatcatcggtatctgcctcggtttcaaggcgatcgacctgggcaagcgcggc

SEQ ID NO: 357 - DNA, 70 bp, Artificial sequence
  Probe
gcgctggtggctctcttctacgcgttggtggccatgatcggtgctatcagcgcgctcgtcttcatttggg

SEQ ID NO: 358 - DNA, 70 bp, Artificial sequence
  Probe
gccgcaatggaggtgtcggccattggtattgcggatatggctgctgttgtggcgcgtgcctctacgccgg

SEQ ID NO: 359 - DNA, 70 bp, Artificial sequence
  Probe
ggcaattacggtgttcgcagcatcgacgtgccgtgctcgttgttccagaccatcggtctgatcattctgg

SEQ ID NO: 360 - DNA, 70 bp, Artificial sequence
  Probe
gctgctatctcggcgttgatcgccaaggttgtcgggctggctacttggtttgggcagctcgcgattgccg

SEQ ID NO: 361 - DNA, 70 bp, Artificial sequence
  Probe
gatctgattgagcggttgacggaattggcgttcggctacgccgctgagatgcattaccaccaggagaagg

SEQ ID NO: 362 - DNA, 70 bp, Artificial sequence
  Probe
ctgctgcactgctgagcagtttgccgtgatcgggatcgatggcccgaccatctttcagtggtggagctgg

SEQ ID NO: 363 - DNA, 70 bp, Artificial sequence
  Probe
ctgcttgtgatggtcgtaatggcgagtcgggtggcgtactgggagcggcgtttctggcagtcgctggtcg

SEQ ID NO: 364 - DNA, 70 bp, Artificial sequence
  Probe
ggtggttatcagcgcaaggcgtaccagttcaagaacaaggacaccggcgaggtcgagacggtcacgccgg

SEQ ID NO: 365 - DNA, 70 bp, Artificial sequence
  Probe
acagcgacaagctccagtacgactttggcaagcatccgcgagtggatgaggtgacggacgcctatttgga

SEQ ID NO: 366 - DNA, 70 bp, Artificial sequence
  Probe
gacgcaataccgcaagggcgaccgcaagatggacaacgaagcgtgcctgcgcctggcgcaactgctcgag

SEQ ID NO: 367 - DNA, 70 bp, Artificial sequence
  Probe
cggcgttctaccagccaaggaagtgctttccactgcgagatctgtaggcacatggacatggagacacaag

SEQ ID NO: 368 - DNA, 70 bp, Artificial sequence
  Probe
cggcagcactcatgccagcgggttcattgcctctctacgtcccaagctcaacttcattgtcactgatacg

SEQ ID NO: 369 - DNA, 70 bp, Artificial sequence
  Probe
cggcgttcgtgaggtacgtggcttcactggtgagcagcaaattcagcctggtcacttcttcatcctccac

SEQ ID NO: 370 - DNA, 70 bp, Artificial sequence
  Probe
gtggtggattcggtcatcaagaatggcgagatcaagcaggaaggtgacaaacgctttgttcgcatggccg

SEQ ID NO: 371 - DNA, 70 bp, Artificial sequence
  Probe
ccacgaactggatgtggtggttgagcagtgctaccagattaagccgttctacagcgataccgaacgcttg

SEQ ID NO: 372 - DNA, 70 bp, Artificial sequence
  Probe
aggctatacggacagcacagccgcaaattatgcctgccgactgggaggtgaaagaggttggtggcacgtt

SEQ ID NO: 373 - DNA, 70 bp, Artificial sequence
  Probe
acatgttgtttatcggtggatatggctgggatggctcgaatctgtatattgaaattaatacaggaaaggt

SEQ ID NO: 374 - DNA, 70 bp, Artificial sequence
  Probe
caattgggtatacgtgatctgtgggtgcctgatgggattcgcgttcggctggattgccatgctctggctc

SEQ ID NO: 375 - DNA, 70 bp, Artificial sequence
  Probe
gcggtagccgactggcacacgtatttggacggcgttggccgggaagatttccctgaccaaccagacgatc

SEQ ID NO: 376 - DNA, 70 bp, Artificial sequence
  Probe
cagccattctcttccaactttatttctactggatggatcgctattacgtgtttgcgccgatgtggagctc

SEQ ID NO: 377 - DNA, 70 bp, Artificial sequence
  Probe
cttgaaccaagcatagagcgcgtcgcatatcgccaggccgtgttcaagcgtgacctgatcatcggtaagc

SEQ ID NO: 378 - DNA, 70 bp, Artificial sequence
  Probe
gcctatcgtcaacatggtttcctatgttgctcagaagtcagcgtggaatatgctcaatttgaacgtgccg

SEQ ID NO: 379 - DNA, 48 bp, Artificial sequence
  Probe
cgcgtgaccaaacgccagcgcttcctggcagagatggagaaggtcttg

SEQ ID NO: 380 - DNA, 70 bp, Artificial sequence
  Probe
caaacattgccttcacggaagatgagcagcatctgtacaacctgacgcttgatgagctgggcgacgagtc

SEQ ID NO: 381 - DNA, 70 bp, Artificial sequence; identical to SEQ ID NO: 351
  Probe

SEQ ID NO: 382 - DNA, 70 bp, Artificial sequence
  Probe
gccggttgctgacggagatcgagaggctggcggatgactttagggaaccgctgatcaatgaagtcggcga

SEQ ID NO: 383 - DNA, 70 bp, Artificial sequence
  Probe
cacaaccctgcgcatttccgcgccgaactcttgatggaatacatggagaatgcatccccctcaaaaaacg

SEQ ID NO: 384 - DNA, 70 bp, Artificial sequence
  Probe
gatatcgcggacataagatgcttcgtcgccctcgatggctcgtcgtatttcgctagtgcgtctaaggctg

SEQ ID NO: 385 - DNA, 70 bp, Artificial sequence
  Probe
gtccagacctgtctgtcacacttgcctaccagcagtcttacaacgactacggatcgaatgtcggcatcgg

SEQ ID NO: 386 - DNA, 70 bp, Artificial sequence
  Probe
ggtcgccacattcacattgatggcccactcgacgtattgaacaaagttccaggcattgatcattggtgga

SEQ ID NO: 387 - DNA, 23 bp, Artificial sequence
  Primer
acttggagagatttacggaggag

SEQ ID NO: 388 - DNA, 23 bp, Artificial sequence
  Primer
aagcaaacgagataagggagaac

SEQ ID NO: 389 - DNA, 23 bp, Artificial sequence
  Primer
aaacacaattcaccttcctgatg

SEQ ID NO: 390 - DNA, 22 bp, Artificial sequence
  Primer
ggccactagactttccagtgat

SEQ ID NO: 391 - DNA, 23 bp, Artificial sequence
  Primer
agcttacctgctgttcgacatct

SEQ ID NO: 392 - DNA, 22 bp, Artificial sequence
  Primer
gttcttcgtgatagcggagact

SEQ ID NO: 393 - DNA, 23 bp, Artificial sequence
  Probe
tatgaatgggttgatagcgttct

SEQ ID NO: 394 - DNA, 23 bp, Artificial sequence
  Primer
ccatatccaccgataaacaacat

SEQ ID NO: 395 - DNA, 23 bp, Artificial sequence
  Primer
cacgatatgaccacgatcaacta

SEQ ID NO: 396 - DNA, 23 bp, Artificial sequence
  Primer
gtagacacgaatcacgtctccat

SEQ ID NO: 397 - DNA, 22 bp, Artificial sequence
  Primer
acatcaacgacccttactgtcc

SEQ ID NO: 398 - DNA, 23 bp, Artificial sequence
  Primer
gaccatagtcatcgctgcttaac

SEQ ID NO: 399 - DNA, 23 bp, Artificial sequence
  Primer
ctatcccgcagaaggtattcaac

SEQ ID NO: 400 - DNA, 22 bp, Artificial sequence
  Primer
agtcataggcgtctcggtactt

SEQ ID NO: 401 - DNA, 22 bp, Artificial sequence
  Primer
acactctgttcaccaagtacgg

SEQ ID NO: 402 - DNA, 23 bp, Artificial sequence
  Primer
ctttgaaactggaggaacagctt

SEQ ID NO: 403 - DNA, 23 bp, Artificial sequence
  Primer
aacactcaaaagctgaccatcat

SEQ ID NO: 404 - DNA, 22 bp, Artificial sequence
  Primer
caaccttgatctgttcggagac

SEQ ID NO: 405 - DNA, 21 bp, Artificial sequence
  Primer
gttgcaatgctggtttccaag

SEQ ID NO: 406 - DNA, 22 bp, Artificial sequence
  Primer
gtcatggacgagaaatcgatac

SEQ ID NO: 407 - DNA, 23 bp, Artificial sequence
  Primer
ctcagaggatctgttcatcgact

SEQ ID NO: 408 - DNA, 21 bp, Artificial sequence
  Primer
gttgaagacgccgaagaaaaa

SEQ ID NO: 409 - DNA, 22 bp, Artificial sequence
  Primer
acgagattcctgaagctgaggt

SEQ ID NO: 410 - DNA, 21 bp, Artificial sequence
  Primer
cttgcagaacctgacacatga

SEQ ID NO: 411 - DNA, 23 bp, Artificial sequence
  Primer
aaggctaagggggagtaagtcat

SEQ ID NO: 412 - DNA, 23 bp, Artificial sequence
  Primer
aggtagttgcgtacttggtcgta

SEQ ID NO: 413 - DNA, 23 bp, Artificial sequence
  Primer
gcgcaaattaaacactacaaagg

SEQ ID NO: 414 - DNA, 23 bp, Artificial sequence
  Primer
atccccaatctcctttatcactc

SEQ ID NO: 415 - DNA, 23 bp, Artificial sequence
  Primer
gaaggtaaatccaaaggaaatgg

SEQ ID NO: 416 - DNA, 23 bp, Artificial sequence
  Primer
aattgacttcggctcgtattctt

SEQ ID NO: 417 - DNA, 24 bp, Artificial sequence
  Primer
aattccatgagtggagtgattttt

SEQ ID NO: 418 - DNA, 24 bp, Artificial sequence
  Primer
agatcatgagcctcagcattatta

SEQ ID NO: 419 - DNA, 22 bp, Artificial sequence
  Primer
gaacggagccatagtgatgaag

SEQ ID NO: 420 - DNA, 23 bp, Artificial sequence
  Primer
agagtcgctaacacaagtcatcc

SEQ ID NO: 421 - DNA, 23 bp, Artificial sequence
  Primer
cacgatcatatatgggtccagtt

SEQ ID NO: 422 - DNA, 23 bp, Artificial sequence
  Primer
ggttctttttgatcgtagccttt

SEQ ID NO: 423 - DNA, 23 bp, Artificial sequence
  Primer
tctgttccgagtatcaccttgtt

SEQ ID NO: 424 - DNA, 23 bp, Artificial sequence
  Primer
catagtattcgcagtccagatcc

SEQ ID NO: 425 - DNA, 23 bp, Artificial sequence
  Primer
ggatgtggggaggtttattagtc

SEQ ID NO: 426 - DNA, 23 bp, Artificial sequence
  Primer
ttcatcaactcctcaggaatcag

SEQ ID NO: 427 - DNA, 23 bp, Artificial sequence
  Primer
aagatatgtgccagctaccactg

SEQ ID NO: 428 - DNA, 23 bp, Artificial sequence
  Primer
agcatatacagtccctcgtatgc

SEQ ID NO: 429 - DNA, 21 bp, Artificial sequence
  Primer
atttcaacgacctgcatcaga

SEQ ID NO: 430 - DNA, 22 bp, Artificial sequence
  Primer
ccacaccaggttcttcttgttc

SEQ ID NO: 431 - DNA, 23 bp, Artificial sequence
  Primer
gtaaggtctgcaaggacatcatc

SEQ ID NO: 432 - DNA, 23 bp, Artificial sequence
  Primer
gtggctgcgatagaacttgtagt

SEQ ID NO: 433 - DNA, 20 bp, Artificial sequence
  Primer
gaccatctttcagtggtgga

SEQ ID NO: 434 - DNA, 23 bp, Artificial sequence
  Primer
taatgccctaaactttcctgatg

SEQ ID NO: 435 - DNA, 23 bp, Artificial sequence
  Primer
tgtttgatctgagcaagtttgtg

SEQ ID NO: 436 - DNA, 20 bp, Artificial sequence
  Primer
agaaactcatccgcaaggtc

SEQ ID NO: 437 - DNA, 23 bp, Artificial sequence
  Primer
gtaaggagaagtgatgtcggaac

SEQ ID NO: 438 - DNA, 23 bp, Artificial sequence
  Primer
gtccttgttcttgaactggtacg

SEQ ID NO: 439 - DNA, 21 bp, Artificial sequence
  Primer
catgccaaggaacacatcaag

SEQ ID NO: 440 - DNA, 23 bp, Artificial sequence
  Primer
aagtatttgttgccgtggtactc

SEQ ID NO: 441 - DNA, 23 bp, Artificial sequence
  Primer
aggtactcggctatcacaatcac

SEQ ID NO: 442 - DNA, 23 bp, Artificial sequence
  Primer
atgcattctccatgtattccatc

SEQ ID NO: 443 - DNA, 23 bp, Artificial sequence
  Primer
gagactcggttcaacaagaaaaa

SEQ ID NO: 444 - DNA, 23 bp, Artificial sequence
  Primer
cttagacgcactagcgaaatacg

SEQ ID NO: 445 - DNA, 5306 bp, Ralstonia solanacearum
  misc_feature (3202)..(3728): n is a, c, g, or t
agggggctggtggagtcgctacatgcttgaagttactaaaattgtggattttttgacggcacaaccctagacccagcttggctctgactgacgatgacaaaaaaatctgagttatggatttttgggacaaatcaggactgagccggctggtccggtccatgggtaggggcgtactttgagcggtcagagcggccgtacgtggcaggcctgtttcccgcgtgcgttgcgtcgaggccgcgtgaatggcgtactggggcgcccgctaccgggccgaattgctctgccaccgttcccgatgacgggatgttgtggtgtgcgtcagtcagcatgattctcgttggtagaatatgagcaataatcacaagaaaccaaccgaagtcgtttgactgcctccgctgcgcggggccaatggggcgagatacgcataatgacggcagaacgtgccactccttcgcgaattgggactttccggttctgataatggcaatttcgctcgctttcgagaatgagcaaattcgccagacctgcgaaagtagccgttctgctaagcgccgccttggtgtaacggtagcggctcaactccaggtgcggctcgctgacctcaaggccttggatgccgcgagtgacgtaattgacatgggctttgctcggctcgacccgtccgaagatacccggcttgtcattccgctgggctgtgagtatgttctgagggtagccgcgcatcatcgtcccgaaccacgttcgtcaactggtaagttggactggaagaacgtgactcgcatcaaaattctggctatcgagtgcaccggtgaaagctgaccatttgttttctcccacttgggctgtgccgccagggcggaccgtagttgacttgctcgctgcgaggggttggtcgcgggatgagctggccaactccattgagctggtgcgagaagatctcgatgacctgattgacggaaccgcacagctatcggaaagcgttgcttcccggttggggggcgtctttggtacgagcgccgccttttggattcgtcgagaggagcagtaccgcgagcagctcgcgagtttgcatgtcaaagctgatgtggcggatgcgggctacaacgagtggctgaaggcactgcctcttgcggacatggctcgctttggctggttcgctccaggcgcgacctccgctgacaaactggcggcgtgtcttgatttctttgatgtcccgaacttggacgtgtggagtcgggactacgacgatgtgaagcgtgcggttgccttccgggcatcagccgcccacccagtgaatgtacctgcgaccaccgcgtggcttcgacaaggcgagcgtgaaagtgagcgtattgagtgctcagcgtgggacccggccaagttctcatcggcgctgaccaaagcgcgtgcgctaacccgtaccaaggacccgcaggtttttgtgccagctctacaagcgctatgcgctacgtcgggcgttgcagtggtaatcgtgcgagcaccgtccggttgcagagccagtggtgcgacgttcttctcggctccagataaggcggtcctgctactgtcgttcagatatctcacagacgaccagttttggttttccttttttcatgaggctggtcatctactgcttcatacggacagggatagtctcatcctggagacgtcggagcgaacgacgtccaaaatggaagctgaggcgaacgagttcgctgcgaaaactcttttgccagtcgagctcaaccgccggctacctgaggtggctggcgacgttcgagcgatacttcgtcttgctcgagaggccggcattgccccaggcatcgtcgtcggccaactgcagcatcgcggtttgatcaaacaccagcacctcaacaagctcaaggttcgctatagctggtagtagttctagccacggaaagccacgaacttgtttgggctcttctgccagttgcatagcgagtcttccagcacttgcattccaacgttcagatggttatcaagcacaaccaggagttggtcagcttgcgcgcacgagatgtgcattgcctcggtcgttccggcgaatagcaagcgggcacccgacagcggtccagtagcaccaaccatgtaagccgaaggtgcacgaaggctcgctaggccgagctttgcgaccatcgtgaggtagtcgaacttggcagttctgccgaaccggtggacgactttcatgctcttgtatagatacccaaacagcgcccttgggttgtcgccgacggctgccgtggcacgttcgagcattgccgcatgcgaacgttcggggcctacccattccgcgtagctttcaagcaccacatccactttctcgatactttcgtacttgcggtggttgccgaattggagctgctttcgattttccagcaactccggaaggcggtcgttgaaccagattgtgaaaatattcggggcgctgttgaactgctcccacgtccaaataaactggtcattccatgcgccatagatcgcgcggataaggttccatctggttcgcaggcttttcccgaaatggacgaagaggaacgcgagccagaacgcctcatcaagattttcctgctgccggtagagagtcgcagcccgcaaggggtcgaacaagtctgacttaggatcccgtctctgtgggcagatgccgcgccgtccgatagtcgttacgaagtcgacgcggcggatactctcgacaagctgctcaacataggcaatgcgattgtcgtcgttcgcgatgcccggcagcggcttgttgtccgccttaaactcaagcagttttgcttcgaggtcagctgccaaggcttgattcctaagagtcgctaacacaagtcatccacgaatcgagcgcagatgatggcggcagcaagcgagaggagcgcgcaatggatatcgagccgtcgttcgaaacgaacgcgtatctttccaaagccggcaaaccaagcgtgcgtgcgctcaaccacccaccggtaacggcccaggcgctccctgctctcgatgcccttgcgggcgatccgggcccttatgccgtgccgcttaagatactgccgacatcggcggcagtcgtaagccttgtcggcatgcagcttgtcggggcgcttacgcggccggccatccaggccacgtatcgcgggaatcgcatccaaggtggattcgaatgcgatcgagtcatgtcgattcgcccctgttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaccgctaagtctcatatcttcatccttctgattggctaaaccaacaggggcgtggctattcgtcggcgggggtgtccaatgaggccattccggcatccagttcagcaggcgtgaacagcagaccaataggctcggcgagctccggcgcaggctggctcttgcgaacgcgttgctgtatgcacgcgtcatagttcgccgtgctcatcttcttggctaacgcacgaattgaccgcgaactgttaggagtcttatttttcagatgccgctgaataagcaacaagcggacgaggtctgcccagtagggttcgaactggttgacgttcacggcgtgccccaagcgaatttcccgttccgcatctaccagcgcgttaagggccggctgagggtcaccgaccggcatcgatggcatggcgatgcgctcctgcaagccctcgtcgacatactttgtcgcctggtccgtgttcttcgagtacaaatgtaggcttccgacggcgtgcttgtatgtgcctagttccgcgccgacggcccgtgcaactatctcctgcagcatcgtgaacgcgaatatgtcatgaggcaggccgagataggcgtcgttcgaccgcatcgtcacgaacatgtggagctgacgcgctctgagcgagaactgcatggtgcacgtgcaggggatatcttttctgcctaccgccacatcgcaaccaaggtcttcggcgttgaacagttggatgaccgcacggcgtgtatcgttgttgccgctcaacttctcgatgacgttgctgacttggttgacaccacgcatgttgaacaagcgagggccgtatgccccgtgaacggtgtttccgtcgtccgagaactcgtcgtaccggctgatgtagtactggatgaagctgagctcgttgctgccggccaagtaccacaggaactcaccaaggcagctgaacaatgtactgcgctgttcagtgcggctcacacgggcacgtgggttcctgatttggagcaagaccccgtgcaggtcactgcattcgccttttgatggcgtgatggacgatttcccgcgcaaaagacgtctgtagaccttgagcaataggtcatcaagcgtttctgcagtcatgaacatgctgggcaccccaattcatgtcacgcgctccctctgtggctcagcgttcgctggcctccaaagcgaggtcaatcaaagagagccagttgcgctgtctcgcgggcttgctcgctgagtagctcttgcttcagtcgccggccgctcgttatcaatgtgcgtcggtctaccaactttgctcccttgctcagtgcaatttcccgagtttcgagggtgatgtcgtagtgggggagggacgcattggcttggaaccatccgcggtgcaatccgattgagcgagcgaatgagtggagctcgtcgaggttgtcagctactagatggcaccacttgcgcccgcgccagggaattcgggcgttgtcggtatacacagccatcgtcagccccaaaataattctgattttactgattctgcattcttgcgagtgctgaccaagcataggccagcacaatgcattctagagcgtgt

SEQ ID NO: 446 - DNA, 18073 bp, Ralstonia solanacearum
  misc_feature (9530)..(9679): n is a, c, g, or t
atttgttcgcacaagagccatctgcaccactcgctgaagcccttcgccccaagaccttggacgaggtcattgggcagtcccatctgctgggtgacggcaagcctttgaggctggccttccagtcaggcaagccccactccatgatcttctgggggccgcccggtgttggtaaaaccaccttggcacggctcacagccacggccttcaagtgtgagttcatagccctctctgcggtgttttcaggtgtcaaagacatccgtgctgctatggagcaggcacagcagaatctatctatgggcaagagcaccatcttgttcgtagacgaaattcacaggttcaataagtcccaacaagatgctcttcttccttatgcagaaagtggcctcgtaacctttattggtgccaccactgagaacccttcttttgaggtgaactcggctctgctgtcacgtgctcaggtctatgtgctgaagtcactgacagaagaagaactgaagctgctcttgaagcgagttcaagaagaagatgctcttggtgacttggagtttgaagataaagcagtagataccatcattggctatgcagacggtgatgcaagaaggttcttgaacctgcttgagcaatgcaaaacggctgctggtgctgctggtgttcagcatgttaatagtgacttcattcagaatgccctaactctcaatagcaggcgttttgacaagggtggagacaacttctatgaccagatttcagccttgcacaagtctgttcgtggctccaaccctgatgctgccctttattggctgacacgcatgttggacggtggtgctgatccacgctatctatccagaagaatcgttcgcatggcttgggaagacattggcttggctgatccacgagccatgcagatagccaacgatgcagctttaacctatgagcggctgggtagtccagaaggtgagctggccctgggtcaagcggtcatctatctggctgtagctgccaagagcaatgccggttacaacgcctacaaccaagctagagccttcgtgaagcaggataagagccgtgaggttcctgttcatctgcgtaatgcaccaacaaagctcatgaaggaactcggctacgggcatgaataccgttatgctcatgacgaacccaatgcctatgctgctggtgaaacctaccttcctgaaggcatagaagagccgggttggtatcagcctgttcctagagggttggagatcaagattggtgagaagctgtccctgctgaagaagtgggatgaagaagccaaaaagaactgaataaggggatcagatggagcaaagaacacttctctacgatgagcggttccttgaaagctatgctggagccatcatcactgatcctgctacggcgattgtggagcttgtcgccaactgctgggacgcttatgccacagaagtgaagatcacatggcccgatgttcaacttgaaaagcagttcaagattgcggacaacggtcatggcatgacccgtgaagagtttcaatacatctggcggaccatagcctacaaccggttgtcgtcaggtggaaacacaacggctcccccagcagatgttcaaggcttgccacggcttgtttttggcaagaacggtaaaggccgttttgccagcttttgcttcgcttctgaatacttgattacttcaaggaagaacgggcaagagttcgtctgccgtgttcacagaacctcaactgatccgctcgttctagaagaaatatcgttcaaggaaaagggtgttgagggtcatggaacggagattgtgggtaacggcatcattccccggctcatgttctcggaagagaaagctcgtgaattgatcggtagccgtttccttgccaaccctgctttcaaggtgaagttgaacggtagtgaaatcacgttcaatgacatccctgacttgttgtccaagagcgaagtggatgttgaagggtacggcaaggcagttattcttcacattgacaccaagaaggctgacaagacaacgaagcaacatggactggcatggtgggttcaaggtcgtgctgtcggtgaatgcaagtggagtcgtagtgactacgaaagaattcttgacggccgaacttcagaggcgaagcgattcacattcattgttgaggctgactatctaaatgcccaagatgcagtgctgggtgattggagtggcttcaaggacgaaaaccaggcgtgggtgaagacccgcgaggccgttcaagatcgcatccgccaaatcatttttgattccagcaaggcagagcgtgaaagccgccgcagtgcagtcattgaaaagattggcacaacgatgaacaccctgtcccctgttagcaaggaccgggtggaaagcttcgtcaatgaggtggtagatacctgcccgaactttggtgaaaacgagatcgtccagttgtccagcatccttgcaaagctggagcagtccaagtctcgctatgggttgcttgatctgctgcacaagtgcgagccaaacgactacgacacccttcatgacatcctgaaggaatggaccatagggatggcaaagctggttttggacgaaattcagaaccgcttgaaattgattggggagcttcgcaccaaactgaaggaagttggcgttgatgaagtccatgagttgcagccgttgtttgagcgtggcctgtggatgttcggggctcaatttgaatccattgagttcacgtccaacaagggcatgaccaatgtaatcaagaccatcttcaaggatgaggatggtaagggcactcgtaaccgtcctgacttcgtagccctgcctgacagcagtgttggcttctatgctcgtgcttcatataacgaagattacgatgaagacggtgttgaacatctagttattattgacttgaaaaccactggtcttgctctcggtagcaaggagaaagatcaagtctggaagtatgtgaaggagctacgagaaaagggctacctgaagaagcatacccgtgttgatggatttgtcttgggagacaagattgaatcgggtgagaatgaccctacaacgcatggtgaagaagtcaaaatttcacccatgctgtatgacaccatcctcattagggctgagaagcgccttctgaacctgcacggtaaggtcaaggatgctccgttcctgatagagcagcaggaagtcctgaagaagtttttggagcctattgaagtcattcaggcggaattggtggaagctgacccagtggtggcgaaatgacctcgtccgtgaattggatcgcagcctacaactacctgtttgcttctttcaacagtgagaacaaggatctctatgttggcggttctgtattttgtcgcatggtgcagcaggtcgatcctggctcaccaagctaccaacagttgctgccgctgagacaaagccaaggaaagtccaattcgcgcaaggacttttattgggaccttatccaaggtcttccagaggcccagcgatttcaactctatcgtgtcttcatcaaccacattgaagttcacgacaagccagcggcagacaacatcaggaacattgtgttcggcggtggttatgcggtgcccactacggttgttcccgttgacctctggaactctgagaagctgaacaacagtctcaatgacattgaccatgccattgatgctcaccattacaaccgggcaacgacactgagctacacgtgtcttgaagggctctacaagacatacgttcgcaagcacgttcctggacaaatggcgctgacggatctcatgccattgtgcaaggtggtgaaggaggacatcagcaagaagcttcaggcacaaggcccgtttcctgtcgagattgtcaatgccatgccgaccttgaccaatgccattgcgaactcaagaaacggcttcagtgaatctcactttggtgacgactcccagcggtggcttgctttgtttgctcgtgatctgaccaactcaatcggtcgtctcatgctcaacttcatgtgaccaccggcgagaggaagatgactacttctagaacatgcgcactttgcccaaacatactgtcagccaaaaataaaacaaaagagcacatcatccccaatgcgattggcggcaggaagaagaccactgggtttatctgcaacgattgcaataacaagttgggtcagagctgggatgcggagcttgcaaggcaacttaactggttttccttggcagtcggcatcagtagagagcgaggtgagccacctaaacaggttgtgcaaacaatcgatggggacaggtattggcttctcagcgatggctctttcacgccagaaaaatcatcttattcagaaaaggatctgggtggggcaatcgagatcaacctgaccgccaagacggttgaagaggcaaagcagcggattcaaggtatcgccagaaaatacccgaagtttgacgttgaaaaagctctcaatgagttggagataaaaaccagctatttggattcgcctcttcatgtcagtctttctttgggtgggccagaggctggaaggtctgtagtcaaaactgcgttttcttttgcatctgaatgtggtgttccacatgctcaatgcgagaaggctcttgaatatcttctagatcctactgctgaaaatttcccctttggatttgcctacttgtctgacctggttgaaaaccgaccaaaggacaagatttttcattgtgtgtctttgcatggtgatccaaagaagaagcggctatggtcttacattgaatacttcggaatcttcaggcttgttgttctcctaagtgatgtgtatgtgggtccctacaagaatgaggtttatgccatcaatccagttgatggagaacggcttgccgtgaaggtgactacccagatcagcgaagaagaatttacttcgttgatgaggggcgaaggtttcgaccaagaaaagcatagagcagcggcggactatgccctgcctatcatcctggatagagggcgaagccgaaccttggagcgcacagtcaaggaaggctttgaacatgctgccaagcagcttggaatcagtgaaggcgaaatcattcccaaggaaaaggccactgagttcactgcctttatgatggagaaaatctctccatacatagagcatctagttcgtagtgggcacagaaatgaccaaccataagccagcaaacggcaccaagaagctcctgcttattgaccttgagaacaagcacaaggtagacctgtctcccttggatgagagttacagagctatcatttttgtgggtgccaaccagaacccgccaaaagcttccaagaagccagcaacagcccataggttcaagagggtggacttcctgaagatttcaggtgctggcaagaatgccttggacttccacatagcctttgaactcggcaggaccttcgagactgcacctgatacccaatgcttcgtgctcagtggtgacaaaggctttgacccgctgctgaaccacttgaatagcaacggtatgagttgccgaagggtggaaagcattgatgagcttgtgccagctacggtggtgcctgtagcagttccatctgatgtggttgtctgccctcactgtaagaaggccagcaccattgagcatcacggcggccactggtgttctaactgtggaaggtttgctactccaccagatcccaagctgctgccgtccaatcaacccggctacaggcagtctcaaagagacaggtctgaagaagactatgccatctattcggtatgtggctggtgccatcaaagaactgatatgactggcggtatctatgacgatggggaatggatgtgtggaggctgcattgccaggtgcgccagatgaaagcaccttcgggtgctatttttttgcctcctgaagcgtcattgctttcttgatcgccttggctgtcgccggtccccggacctcacaaacaagaagaacgtcggagcgttccagcctcacggcatcctgcaagggagcatggacttgaaacctgcatatcaaccctcatgttgtggtggtttatcgttgccagagaggaaggccgtgccaagaaccattgaacagggtttcaacgattttcttgtaaatcttagagccactgctgctgagactgccgcaaccgtgtctcatcgggcatccatcaaggcatgcctggaaaataactttggcttgaaacgcttcacccgaatcggttcgtttggcaatggaacaaatgtgtcgggctacagcgatgttgactacttggcttgtctgccgactacctcattgaaacaatcatcaacctattctctcaggttggtgaaagaggcattagaaaaccgcttccccagtacgggtgttgtggttcgttgtccaacagtggtttgcccctttggtcaatacaaatctcagcatacagaagttgtcgttgcagactatgtgaaagaacaaaatggctacaaggtctatgaaatagctaatggcaatgatgggtggatgcagatttgccctgatgctcacaatgcttacgttgaacatgtggatggtaagcacggaggtaaagtcaggcagttgattagactgatcaaggcatggaagttctatcagaatgtacctatcaaatcgttctatctagagatgaaggtggcgaaatatgcgagcaatgagcaagccatcatccatcacctagatgtgaaatattttcttgaccgtctcaataatgaaggtctgtccgccatgcaagaccctatggggatcgctggctatatctatccttgcaacagtgacgttcagaagattgacgcactctcaaaactaaacacggcagctacacgggccaacaaggctgaagctgctagagcattggggaatataaaagaagcttttgattggtggcgactgctttataacgacaaattccccacctactacctatgagtatcattcaaagacaggacctgccagagaacattgagcttctggctgctcaaaggaacctctattcaagggccaagaacatcattggacttcaaatggtgctcagtggcccggttgccatttgtgctgcgatcacaacgattgtgcgaccagatttaaaggggtatgtggcactctggggcatcttggctgtggtgtttgatctctttgtcttcacgccttgggtcaagaagctaagggatagtgcagctagggttcaagagctttttgacaccagggttttggggttagactggaatgaaatttccgtgggcaaaaaacctgagcctgaactgattcatgaggaagccaaaaaatacggagtgggtgaagaaaaaagggccagcctaaagaattggtatccactcatcatagacaaagttcctgaaatttttggggtaatcattagccaaagatccaatgtgtggtgggattgcaagatgcgaaggaaatactcctttttcgtaagagtagtccttgtctccatcgcactaggattgatcggttatggcctctatgaaaagaaggatatgtttgaatttttggcctacatcgtggcacctcttgcctctacctatgtctttggctaccggcaaatgatggagcatagtgatgccgcagataggctagataagctgagggacttgtctgagaaagtgtggtctgacgcggtttctggaaaagatataccgacgctgaagattaagtgcaggacattgcaggaccagatttttgatcatagaaagagaaatccaccagtctttgatttcttgtttaagtggttcagggatggaaatgaagcactgatgaacaaaggggctgaagctcttgtggcagaaatcaagaagtaaagaagtgcctatggaatattttttattggctgaattagttagttaaattcatatgaatttaactaaccgtcaaccaatcaagaaaacagtacgttagagtcatactttctatgaagttgggagagagtatgaggaaaactttgttgtcgttggccgtggtgttgggtggttgtgcctcggcacccaaagagcctccattggtgctttatgggcaggtgctggctgtacagcctcatcaagtggtagagcagaaggtcaacatgactggtgcggtcgtgggtggtgtagctggtggggtcgtcggcaaccagtttggcaaaggaaatggcagaaaggccatgactgcccttggtgtggtcgctggtgctgtggtgggctcccaagtgagtaagagcagtgaagtgaagaatgtcactgacttgcaagtgaggatgcctgatagcaccgtcattgatgtgactaccaatgatgttgggttcagggtgggtcagaaggtgaagatcacccagcaaggtaagcaggccaccattcaagccatgaactgaaggcaccatgctcaaaaagaaagcaccctgaatgggtgcttttttgtttatctatttaacttcctccatctacacacacgttataccagtatgtatagctattccactgactcaagcttatgacccagtaggcaagaccattcaagttgtatgtcctgctaggtaatattctttcttgggatgctgtttgccactcatatctgcatctcccccaaggtccttggctttctctagaagatatgtagccttcacttgcagataatggaacataagtagaagtgaatgagccaccttcaccgggtaacatactatcgtagtaacgatagatggttttgctctgagatggtggaattggtggagcataggcagcagagcacattgctgggcttccataattgccagcacaaccccatgtgtattgattgttggcgaagttgaagccgtaggactgaccagtagcacaaacattagtgggattgctggacagagttttgccattgtcagcaccacatgcaccaacttctctctggtttgccgtacataattgactacctaaactattgctacaagtccaagagtaagttgtttctccatttgcggtggtagtagaaactgcactgggtgttcctgcatcgcacaaaaagctgttaggagcacctggcaagatagatccagcagaagggccgcatttaggtttaggggtggggctggctacatatgttggtgcaacaaatgtgtttgctttaggtattaaagcgagcattgactgctgctgtaaaacacaagttgaatcgcaatttaatgatctgtccgattgctgatattgagcatgtgcctgtggtgtgaatatggctgctgccaataatattccgaatgctaatttggttatacttctcatgaatttcccttcctttaaataatcataagaaatatattcaattagtcaaatttcacgcataaaaaacatccatgtagggtgcttttttatttcctcttgcccatctggtcgcgttttctggattttgccaaattcaagatatggttgttctcataaatcttggcgacctcaatcacctcatggtgcaaaccggtcaggctcaggcatttctctcggaggggcaccgtgaaatgcgctacgatttccttttttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctctgagatggtggaataggtggagcataggcagcagagcacatgctggggcttcataaattgccagcacaaccccattgtgtatgatgtggcgaaaagttgaagccgtaggactgaccagtagcacaaacattagtgggattgctggacagagttttgccattgtcagcaccacatgcaccaacttctctctggtttgccgtacataattgactacctaaactattgctacaagtccaagagtaagttgtttctccatttgcggtggtagtagaaactgcactgggtgttcctgcatcgcacaaaaagctgttaggagcacctggcaagatagatccagcagaagggccgcatttaggtttaggggtggggctggctacatatgttggtgcaacaaatgtgtttgctttaggtattaaagcgagcattgactgctgctgtaaaacacaagttgaatcgcaatttaatgatctgtccgattgctgatattgagcatgtgcctgtggtgtgaatatggctgctgccaataatattccgaatgctaatttggttatacttctcatgaatttcccttcctttaaataatcataagaaatatattcaattagtcaaatttcacgcataaaaaacatccatgtagggtgcttttttatttcctcttgcccatctggtcgcgttttctggattttgccaaattcaagatatggttgttctcataaatcttggcgacctcaatcacctcatccaatgaggctccttgcttgacctggtggagcacaatcacttcttggttgtgtttgacagcagccttgataaaacctctgtctgtacaattgacaccaaggttctcaagctctatgactcggccaaggttgccgtcaacacaggcaaaggccatcttgtcaaaggtgttgccatgctcatccacaagatccatgaccttgccaatgttgccttcctttgaatgcttttccatttcatcttctatttcttttagggttctttctttcatattgcctccggggattgtttttttcgaaatttacttataaagtcaaatcttgttgatcttaggtttggctgctacctgttgttctttgagctgaagcttctccacaagcctttggttagtaataagcttgctgatttcgtgccccatgtccggttcattgagtagatttgctacatacaactcaatcatgttgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaaaacatgatttaatgatttttgtggtacatgaataaacccttttgtttatcacgacattgttttaaggcacttacttcggtaggtgcctttttatttatatttgagttcattgtctttggcttccttgagcttcctcttctcaagctcaaggttcaatgcgacaagtttttgctcaatggcaatgatctgagcctgaagcttcgtggctcgtgtgccggtggcaccagccagcagcaacagcaggttgtgatactgcatggacaaaccaccgatctgagcctccaaacctccaacagcagaacccacattgggcaggttggtggggttctcacgctcgtgctgcatgcgggccactctgtggggggcagagtggttgatgccttgctggccaagctgaacctccttggcatgttccttgatctccttgagcttcttttcaagcttgctgcggatctcggtggtgtcacgttcaagagtagttggcaccttctctttggtgggtgtcttgccatccttcctattgccaccagaaggccctttcttggccgcatatgcacgttttgcaccaggtctaggggtgaggtagcgtttggtgttgaaggctgttctggctgcaatgaagtccaccagcttggaacgttggtccgcggactcggccggggtgaggaacttgggtatcttggactggtggtgttccttcaccaaggccgcatagtcactgtccttgctgaatagcgagcccttcaatctggtgttccttccagcaccaggtaagcgaacggtgatgaactcgtcacctaccaaggcaatgccaatgcctcgcttcttcatgtagccaatgagctggttacggttggctatggagccactgacgatgcgagtgtgaagctcatcagcaaagtcattcttggcttccttctcgcgtttaccgttgggggacttggcttcaaatggcttgaggctgatcttcaagggatcgggcaccacctgtgcaaaaccgaagttgtcattcatcacggcactgaaggcattgaagaactctatgttcttctccccaggcggatggatgttcagttgctggccggtcttcaattcggtattggcaacaaggaagtgaagttcaaggttgcctttgtctctgtgcatcacccagaagtcagcgaaattctcatcaaccttcaagcctggcatgaaggtggaacggaaggcttcgatcaatgtggcgatctgctctggtgtgacagactgggattcaagtccatcacgaaaagcgatgacaccagaggtgtacttgtgctgccttttcgtggcattggcaatctctgtgaaggtggttggatcgccaaaaagaatttcaggcatgatagatcgcttcttgccttcatgatcggtgtcactgagcaggtatctaactgctgattcggaacctcctttgccagtggggaaaatggtgacgttcatatcttccctcgttttgaagatcctttctcaccaaggttggactcaagtttttctttgagtgctttcttctctaaaaagtggccatactcttcttctgaatattgatgtccattgacccaccattggtgtggcttgccatcaagaaggacagcaggcccgtcttctctatgaagttcgccattgaaaaagtgcttgatgtagacggcagatacggtttctactccggtggcaaaatcagttgaaaccatcttgaattctgctaccccagaaaaatcatctggtaacgcttttgttatgctcatttcttccccttgaataattctgcgatagtgctcactccttcctgaatcttggtgagttcctggaacatccctttggtggtgtcgttccacatcttcatgcgattcaaagaacgagcaatctgatttatgttgctgccgatctttccaagagaagcaagaagctgaacacgcaacttgtgttctgccttggtttcctggttggcttgctggtgtgatggaagcaggattgaacgggcataaatcgctgtggaagatgcacctgcttctacggctcgctgttggagagccatccattcttcaggtgtgaacctgatctcaaaacgcttggttcgctttttattgttggcttctgtgccagcttgacctgcccttattttgagagtcgggtctttttcttttttgtggtatgccattggggcctcctttgttcttgtttttatcgagcctcgcgcagcagcgggtgtgggtgaaacccacgatattcaaaccaagaatagcccgaaagggatatacgtaggattgattgaccttgcttaaactacttttcccccannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcatagggctgtcggctcaccaaagggtcttgtttggtttggccaatctggtgctcgccaggaggcagttgctggccagtcctgggagcattgcgtcctgagcgcgcaaagcgcgccaagataggtgcgaaagcagcgaaaaaccgtgctgaatcgaagcatcccttcctcaatccgaaaaaccaagatggcatgactcgcgattccattcgccgacttcattgatcagcggttccctagcgcattcaaaatctaagtcaacgatcatcagcagtatgagcagatgttttatgactcaaaatttttatgaataaaaaacggactgtgtcatcgtcaataaaatcaatagaagatgatgtcgccatgagacatgggacagattcatcatatatgtgttcatgcaacataagacaaattcatcatatacgtagccatgcgacataagtgcaatttgtcattgtggttgacttcgataaaaaatacgtttcaatgagtcatgagcgatttggagaaaattttgtcatgactaagaagagcataaagaatgaaagaacaacagtaaggattgggcctcgaatgtccagaggcctttatcagcttgcgaagtatgtttatggcgtgaaaggcgtcaactttgaggatcaacttgaagagttggtggaaaaagatctggactatcagatgaggcaaaagtggtttctcaattccacttctggaaaagagggtcagtattttgagaaattaaaaaaagcaattggtgactcgaaagatgatctttcatctgaggatgtcagtgactttgattcaaacaacccaaaaaaggatggaggcgagaatgagtgatgaatacaaagattgcttccagccgaaagaagtgaagatcagtgccaagatgccttttgaactccatttggctgcgaagcaactttactcaatgcaaggcacaagcattggggaaagggttcaaaaattaatcaggactgacctgaaaaaaatgatgagtcgtgattggttcaaggagttgccagaggaagaagtagccatctataagatgttgcttgaacttagcgagcgagaaagagtgaaatgaaatttcaatacgataaaaccataatcaccactcaatctgatttgcaagtgtcagtggcagacttgagggatctggttcaagctttcactattccaaatgaagcccaaagggttcaagaaattcaattgattcttgaatcagttgtgaggaagaatggtctgcaaccaggtgctttaggtgtggagtaatcaacctgctgcgcacacgccttgtggcggccccggcctcacgtggttaaggaacgacgaagcgttctcactagggaaacgctgatttatcagtctcagccgtcatcgctgacgcagctgaggccgttgtagcaggtagctcacggaacggagccatagtgatgaagcggcagatgagctttgcagaagcggaaagcgcaggtaagaagcgcgtgaccaaacgccagcgcttcctggcagagatggagaaggtcgtgccgtggcagcgcttgctgtcggcgattgggccccactatccgagaggcgagcgaggtcgcccgccgattggtctggagaggatgctgcggatctacttcctgcagcagtggtacgggctgtcggacgaaggtctggaagacgcgctgcacgacagtatggcgatgcgagccttcgccggcatcgatctggcggtcgaggacgtgccggatgcgaccacgctgttgaagttccgacgcctgctcaacgaacacgacttgacgcgaaagctgttcgacgagatcggcatcatgctgtgcgagcgggggctgatgatgaaggaaggcaccatcgtggatgcccccctcattgaagcgcccccgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaacgcggagggacagtcctcagggatcaaagggcttggccaagaacacggcgcaactgttcagcctgtttggtttggccaatctggtgctcgccaggaggcagttgctggccagtcctgggagcattgcgtcctgagcgcgcaaagcgcgccaagataggtgcgaaagcagcgaaaaaccgtgctgaatcgaagcatcccttcctcaatccgaaaaaccaagatggcatgactcgcgattccattcgccgacttcattgagcagcgcttccctagcgtgatagaaatgaaggagtagaacgaatgaagaattgggtgggaatatttcttatgttgtggaatgtctgccacttcaaacatgaagaatggcatttaatgagacagcgattgaaattaaaaaagcacctctagggtgcttttgtttttttacatggcctgctttgttggaatgaatgcgtgtattcgtttcaatgaagtgggtgctccttagctgtgaaaaatatctagccacctctttctacgcttgatgctatctttatgtgccagcaggaaagggaaaaagtcctcatagtgtggaatcaccattgaggaaattccattttctgcttcatattcactcgccttgtgggccattcctgtaagtaagaagtggatgggtcgcagcatctcgatcagtcgccagagaaccagtggggcactcttctcaatgacatattgacggtgtaggcagtcaaacaccatcatggggtgtccatgctcatcaaggtggctggtgaagccgttgggtggcccttctctatccatgcggtacatgaggttcagtgctgacctctcacgcacctggtgcggtgtgtagtcttcataggtagctctggcaagatcgagccacttgtagagagggcttctgtccatcttggcttcgcagatttcattcagattgaagtagcgggcaccatcaccgtattcactaatgaagttcaatagaacttcgtcattggcggtcagagtcggtgctacagcattggggtggctgtagatgggcttcaacctgatgcactcttcatagagcacatcaatcttgtgtccaaagcctttaagctgcttggtggtcggtgtctggtagttgttcgtcagcatgtggtgtgtcaccacagccaacttcagaagcctctccataccaatggagagatgaaagaacgccgaataaaagtagccgtccttgtcctggaagaagttggccttaagcagaaggtcgaaacctgacagcaacgtgttctttgttaggtgagcttcttgtgcaagaaggctgaattttttatcaaaacctatctgcttcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatcaacgtgtccctagcgcgccggatgcaga

SEQ ID NO: 447 - DNA, 4775 bp, Ralstonia solanacearum
  misc_feature (1077)..(1966): n is a, c, g, or t
aggggtttgtagacatggagccatcgcaatcgagcaatgccgaagggctgagcggagacgatatggaggttgtctctgcagtatcccggcgatacaggcgttatcacaagatagccgcaaggcatctgccggaatgggcgccgtctggggttacgctaagcctcatacaagcgatgttgaacctccgaaacgttcgttgcgcaaagagaaacattcacgagaacgatgacggcacccagtcaacccatgcggagcatctgagatatgccgaggccgaaatagctcgtctcgtgcgtctgtgtgcctatagcaatgaaatgcagaaggcttggcggacaatatcaactatgccagctcgtggtggtggcgatccggcattctccatatgttcaaaaatctcctccacgctggggcaatttggtgacctccccaagaggcttccagcacaacgcaagaaatcgctttctcgtgtggcgaaggcagtggacgatctgcttgaggcaatcgaatcggagagcgaggcgcaggactctcttgagattgcactgattgaatacctgggaggtcagaacctcaagatgcgctctgacgacggagaggaaataggagtcgatgaatctctcgatcccttccgagcgcttagtccggccatcattccatgcgacgatacagaaggcttggaggactcccctcattgctccaagccctggcacgagtggtcatctttcgatagatacggttggcttcacgatgaagtatccatgacgggaatggccgagttgctgatcttttgtcgcgacagaataaatgccttggctcaggagccgcccaaaattctgcagcctggaagaccagatggtgggctacagccgtttctgattcggagtttgtccaactggatgaagcgcatatacggacagcccttggacgactctgtagcactgcttgtctctgccacgctagatctgccagagcctctgacaagagaagcgatccgcccgtatctgaaaggcaccgggaagatttcgagcaggaatcgatagatttcttcccgtttcaaaatatcagagtccacgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgggaaaaacgcgccaacgggtggccccggttggggtgggcgaacccttggcccgccaggaggcaatgctggccagtcctgggagcattgcgtcctgagcgcgcaaagcgcgccaagataggtgcgaaagcagcgaaaaaccgtgctgaatcgaagcatcccttcctcaatccgaaaaaccaagatggcatgactcgcgattccattcgccgacttcattgatcagcggttccctagatgaatcgtcgactgctacgccggcggtggggcttcgtgaatgacccccatgcgcaccatctcgtcgatttcggcacgagcaactgccacaatgtcttccagtgtgcggaaacgagggctttgagggcccatgacgtcttttgtgactggatggcccagcgcgtcgcggtaccgaatccggccagtgcaagtcgcttcgctaagctcttggactaggacgatctgcgcgcctttgtacgcttcaacgggtagttgagccatggctggcctccggttttgatttggagcttctgcgtcggccgttgccaagcctgtcgcccatgtcgataacgatgagctcggttgcggcaatacgctggatctcagccatgatgggcagttcgcgcgggctactgggctcgcttgcagccacaccggtggcgtgcttcgcttttgggccaggcgggtagaaaacgaagagggcggtggacggcgcatccggctcggcacgcaggcgcttgttgagaatcttcaagtacttcttcttggtgaccttcttggttgccacagagcctcctcagagggtttcaggtacctcatgcagctgacagtacgtgttgaccagttccttcacctcggccgcttcattgggcgttccgaggcgctcaacgaacgcattgagcgcgaccagaagcgagcagatggccagcaccacggtgtcggcgtactgcgtgtagtcggggccgatctgtaagccctcaattttccctgatgcgttcaggatcagatgatgttgcaacgacgtggccgaagggtgtgcatcttgagacagttgccggtagatcaccgtgtagagatctcccaggttgagatcttcggccaatccacgaaggtctatgtcctggtgcttgcccggcgcgccaatttcatcgagcaatgcttcggccgctgcactctgttcggtcgttaccccaggcatctcgcgcaggtcggtggccacttgtactcggaaccgttggtggccgccctcgaaggcatcgaagaaactggtggtcggttcgtcgcccttcttgggtttgaggcgagccccttgcgaaagcagtaggtcgcgcgaggccgcggcgcagtacagcgctgtctcgaatgcggatcgcgccatcgatcgcgattgcgcccgcaagccccgtgccgccagaatcaccgccgcctcgaagtcctgcaccgcacggctcagcgaggtcacggccaaaagatcggcgtcgcgttggaagatgtcgcggggttgaattagcagtgcaacagcaaagcggttgactcgctccatcagtgttgtatggcatggaaaaccactcggtacttggtttgcgagggtatcaacgtcaggcgacaggaacccatctctgctcagcatacgggagtgccctccgcactacacgctagcagttgcgtggctgccagcggcctttcgagccttcgacggctagaaattgtgtgatccgagcttgaccacatctagcgtcgaattaacccgtcgcatttcgaatgcggcccggcggatgcgctcagagtgagcctcgaagaccgcaagatcgttgtgcggctttccctgcgctagcgtggcaagagctgttcgtgagatcgacgcgggaatctgcttctcgccgtccgtgatcgtaaaggccagatagtcgccagttatgactgccttcggggctgaaatctgaaagctctcgtccatatcgcctcccactctcagtctcggggcatcctagcatgacccatctgacggaagggaccgtgccggcgaggccggtgccactaaacccgccatgaagatggggcaaatccactgtccgtcgctcgggaaatcaatcagcgacaccgatgcctgtggtgtgagccacccgaggtggtggagttcaggagagccggaaggtggggcggctgctcttcaaagccagccggacacgccaccgtggcgtgagcacgtcccgcggtgcgactcgctgaagctgtacgacccgtcccggcacttggcggtcgcgccgttcggcacgctcccatccttgctgtgcgctggcgagtggacgtcatgcccctgccggttggtgtagtgtccatgctcaacgagcgcggattcgttcggagttggccggtagttggggtgacgtggcgctctggcgaatgagggctgcgcgaccatcgcggcgacggccataacgacggccgccatcgtgggtatggatttcatgtgtctccccctgatggtgtttgtgcccaccatcctgatccatccccggggaaagaaagcccccacagaagtggggctcggctgtcggtggccacgggttccacggattcaaccatctgtcggttgatgcaaccgcggtgcatgagtggccggctactccagagaattcctgccgccgaagcgcgatcagagccggcgtatcgggatgtaggcccgggcgagaggcaggctgcattgagctcgagccagctgcgtgcgggctctttaaatttctgagattcgcagcttgtaggcggtgccgcttgactcgccactaatagctagggaaccgctgatcaatga

SEQ ID NO: 448 - DNA, 7305 bp, Ralstonia solanacearum
  misc_feature (1325)..(1594): n is a, c, g, or t
gtcttggcccaggaggcagttgctggccatttcttgggagcatatggttcttgggccgccaaagcgcgccaatgataggtgcgaaagcagcgaaaaaccgtgctgaatcgaagcatcccttcctcaatccgaaaaaccaagatggcatgactcgcgattccattcgccgacttcattgatcagcggttccatagcaatgccctccgaatcctgcagtagaccgcatcgatctttaaaccatcctgtgcagcggctacggtggtcgaaaactgtcgctcaatcatcacaagctccatcaaagtttttttctgtgacgcagtcagctatggttcttgcagacaggacaagtccactggccgaagtgtgcgtgtatgccgacggagagaggcaagcgaatctttcttcccgaagtctcgtagcaacttgcgcaataagagccgctaacataactggccctacggcaagaagcgggttatcgtacgaggcatgtcgagaagaaaagtcagcaaagagttgtggttggcgctggagccgctgatcccggagtttgtcgcctcgcccaaaggtggccggcggcgctcggtcgatgatcgtgcagcgttgagcggcatcctgtatgtgttgcatacgggtattccgtgggaagaccttccccaggaacttggctttggcagcggcatgacctgtcacctgaaacgcgttcactgcagccgatacttcgattgcctgttcttgaatgcgggctcggtgccttcgagaatctggacaactcggcccttgatctccggttgctcaatcgcacccgcttcatactcaaatcgaacaccctcggtgttacgagacgcgtagacaatttgctcggcgtcacacaccaccgccaagttcggattgtgtgtaaccatgatgacctgccgcctctccttcgccttcttgatgcacttgactaggatcttgtagatggtctggttgtcgaggttctcctccggctggtcgatcacgatcggaatgtcgtccttgtcgaccagaaggtagaaaaccaagagcaataatccgcgctcgccaggcgagagttggccaatgtcttgccccccgaaggtcaacgagtagcgcggcgaaaggtattcaaggccaaagatgtagtcgagaacttcggtagcttcgactcccttgcgaagctgatcggccaaccgcgtctcgcggctgtcctgcccttcgcgctgatcggagcgcatggtttggtcaatccgctgtgcgaaggcaacggcgtcttcgggcgccgcgaagctagcctcttggagcatgcgcctaacgagctgattgctttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatacagttggcatcaccttcagtagtgcgccgactattacccgcgggtctcctcaatgcggacgtgaaattcaagaggaagattcatgtccatctgcccagccgactgcacgaacgcctggacaggcaggtagagccgttggtattcgtcgaccgtgccgcgaatcaacccatggatctctcgtgatatcccggagcgttgctcaatgagcttctggagctcatcaggaagcgcggcaagcgccgcaatctcggattccaactgcgcgatagttcccggcttgtcggcaggccccatcacgtcggcctttgatttttcccacttgaccaactcctccttgtagagcacgaagagacgctgacgctcgccgagcttgctcttggcctcagcgagggaagcgaaaagctcctcgcgtcgcttcaagatgctgtcgggctctgcactttgaagctttgcgtcgattgaatcgatctccccttgcatcgctttggcgattccctcaacaggcccggcattgatccgggtctcgaacaatgagtcgatgggaatctcaaccccgagctctgctaagagctgtttcaactcgctttcgaaagcttcgctctgcttcttctggttagccaaagcctgggcgatgcggcgcgcgactgcttgcccctttgcagtagtggacttcgcctcccgaagccgtgcttcctccttgccgacatcctgcagttgctgctcaagaccgtcgatcttggttgcggccgctttggactcttccaatgcttcaggggaagcattcggatcctcaacctgtctaggcttggcttcttcgagggaggcgagctcagccttctttgccgcaagctgcttgaccaagctctccttgaattccgggaaaagcctgcgctccgtaccaagaatctcgacgttgagcgcagcaatctggccccgcagcccccccatggccttgttgatctcggtcaccttgaaattgagcagcgcgtccatcgatgactggccaagttggtcctccagcgggacgtgtgagtagatgatttttcgcaactcggcatcgaaagtcgccgatccgcccgcacccagctcattgcagagcgtctcaaggtaactctgcggcagatacttcacgcgctccacactactgggctcgggatcttggtccaaatcccgcgtagtcggcgagccgtcgcgccagaccaaggttcccacgaagttttgtgcaagccgtgttcgcggatcccggaagcgcaccgcgttcaaaaacgagaagctacgatagtttttggtgttccccgccaacgcgataatgtcagcaagagcgctctttccgctccccttgttcccgatgattgccaccaaatctgaattcagcggcagttccatgtcgaaccatgtgtcagcgagctttgagccttccttcttggcgaccctaactgagcggatgaacttcgttcgattgagctcaacgtactggcgcttggccgggttatcgccaacgcagacgcgatcatcgaactcttcgatagcctgctgcagacccctgaaggtgcagtccgccttgatccaggtaaagcagttaccgatgcggtccttgtcggtcgatgcgctcagccaatgtgcatcggaacagtcaagcaagttggagttaacaccggactccttcaacttggcgcgcgccttggcgtgatcctctggggattccgcagcggtaaagacaagatctgcactgttaatgagcgtctttttctccgcgatcgagtggtcatcacatttgaggttctcccactcggtcttgccgactgcaaggatgaacctccccttgaggacctcgttctccagggccttgcgcaagccatccgcgctgacgttgaggttgttgaaaccctcaatgagagggctcccataccccacccgcttgtctgctggggccgagtcaatgatggctgcaccaagagcttcaatgctttgccgggagatcactgagttccattttccttcgccagtcgacccaggaagaagacggtagctcggggcgattgcggaaataaactgctggcggatgaattcaggatccacctgatcgaagatgatgtggatgttgatcctgctccagcttgatggtgccttgccgctttttcccccttggacaacgccgccgaacttgtccaaccgcagttccacgaccggcagaatcagatcgatgttcttcagccttccctgctcgcgcttcgccttcagcaccttttcgtagccatcgacgagcacatagtcgttgataccgattactttgaactcctctggcagcttttcaacgtcagcgagaaacgcttcccaggcttcatcttggctccccgggtagttgtgatagaaggattcgggcgtgtggacgtgtaaatcccatttcttccaagtggagccggccagggcaaggggcatgttcttgtctcgctgaagttgttgttcacaagcgggcattgaatcaaataacccggctcacgatggaggccaaacgcacgggatcactctctaccagtctcttaacggcggagaggctcgacaccccaatcagctcttggatcagcttggtcgatcggccttgccgatgaagcctcaccgcgaatgtccggcgcgcggaactggcattgccgttggcgatgccagcctgctggtgtagccgtctgaatatctctgccagggactcgcacgaatagctagtggcaccggccgcagtcacgcgacgcgtaaagccgaaaggctcaccctcgccagtcagaaagaggggactccgacaatccaatccacggtacgcggctggacttgtggtgatgccatgtcgaaacgccagccggtgttcgagatagacgtcgatcgcagcgcgcacctttgcactggcccatagcagcggtcgcactctgccgttgaaggcaatctccgctctcacgtgcgattcgatgcgtgtctccccattcgcctgcagatagtcggacaggcgcagcttggcgatttcgttgggcgtaaggccagtgccataggcaaccaataacagggcgacatcgcgcttgctgttctgcccagtgacggctgcaaccttcagtacgtggcgaacctggcgctcttcgatgactgatggctttggcatcccgtaatgctcccgcttgtggttgcgttacgagacagtatggaaaaatttacatccgagtgcaaaacgaggccgccccatccggtcaaccctcggaaaattgtgcgcagtggcagcgcgccgatctccggcaaaatcgggcgatgagtcccctactcagcctcgggtcgccgggcagccgttcattttctggtaccaaaggcgtccccgagctgccgcgcgcgcttggcctcatcgctgtgcaggtagatcgaggtggtcgagactgatgcgtgtcgcaggttgtcgcgcacggtggtcaattccgcaccgcgtgcgagcgcgtgggtggcatgcgtatggcgcatccaatgtggactggccttgcgcagcttctcagccagcaaagggtgatcgctgtcgataatttcggcggccgaccagaaaaaacgccgcaccacactccacaaccgcgttcccgtaatgctgccattgggatcgagccccaacccaccaatcatcggcgtcttcggatcccaacgcgcgcgcgtaatcggtagcccgcgttcaaccaggtagcggtcgattgccgcccgagccagcggcggcagcgcaaccttaccagcgcgactgcccttccccaccaggttcagccagtgatcgtcctgccggtccgtttcgatatcaccgagcttcgcagaaacgagttcgcaggccggtcgcatagccgaagtccagcacgaagcgcagtcgctgcgcggcggacacctcccagccgtacgaccactcaaggccatcggcgatggtgcggactagcgcccattccccctcgctgaaagcatgcgaggcatcgagcgcagtcgttcgcccacctccgcgcaccttgattccagcaaaggggttagccagcacgtagcgctgctggatcagccagcggaacatggcccccaatacggacaacgcataggcgatcgagcgcgctgacaatccgtctgcaaacggccgccattcgacggacgtgcgcggccgcgccgggccgacccaccgctcgcgtggcgtcggtcggcgcaggaagctccggtaggcaatcgcgtcctcggtggtgagcgacgacagtggccgaccgcgttcgacgatcgcccacaagatcaggcgttcggcctctttgcggtacgcccgctgggtggcgggcgattcgtgcagcgatagccacgcttggaccgcctcatagtcgttgtcggcagtgaggacgcaggtctcgcgcggcgcacggaattgcccccgcgagccgtcgacctcgttcggcaggtgcaattgctcccacggcaccaccacgccgcggacctgttcagcaatcagggcccgggcgcgctcggtcaatcgaggatgttcggcgaagaacggccggcgcggcgcgcggacggtcagatccgccagggtggtgatgccggcggcccgcagcgcggcgactgtacgcgcatccaaccacagcgccacgtcatcactgatgaccggctctggcagtcgcaacgttcggagcaactcgatcgcctggtcggctgccctgccctgtttggcgcgttcggccgctggtatctcgaacagcaccgcgaggtcttcgcgctgacgctgccgtgccagtaccgcgagctggcgccggatgcgaccaagcacgccgcgtgaggactgccccggcgtaaggcgctcgcccagatagcgctcaacggcatcgtgggtcgacatgcctgcatgccaggcgcgcaatgccgccagttcggcggcatcggggaactccggcgaagtgctgacagcgggattgacgggaagcagacgcgttttcattgaagccagcttagcgcttcaacgttgctatttcgataagtattcttatcgcaatagatcaattagacagcgaaacttccttccgtaaaaacgcatcagacatggcgacaccagcttcgcttgatgctccggacaacaacggccgaccagcgctccacaaggcgccttttcgcgtcagccaggatcggcaaggttttctgccgtgagagcgatggcgcatcacgtcacgtcgttcgcgaagtacgccgctgccctttttagaaccgggctcaccggcttcgcacccgcatcggtcaaccgcctctcccggtcagttttgaccccgttgcatgttgtcgggtccgctacgcccggcgtggcccctgctgccgccatgccccgcccggcttgaccgcttgccgcttgccgcttgccacttgccacttgccacttgccacttgccgcttgccacttgccacttgccgcttgaatccgagcatgtgccgtgccgctttgccttgcttgtctttgttcttctcatcgctcgggctgatcggctctttggtcagcaccctggtttcaataaattatttcaatcaatccaattaaacattcctatgcgcatcccggtccggtgttttgtagcatgcaattgattcatgttgatgaccatcaaaccgtccgattttggatttaggtagagtcatcggaatttcattgcagtcattgcttttcaacttctccatcttgaaaccgggatccgtta

SEQ ID NO: 449 - DNA, 27090 bp, Ralstonia solanacearum
  misc_feature (11955)..(12104): n is a, c, g, or t
cgcacagcatgatgccgatctcgtcgaacagctttcgcgtcaagtcgtgttcgttgagcaggcgtcggaacttcaacagcgtggtcgcatccggcacgtcctcgaccgccagatcgatgccggcgaaggctcgcatcgccatactgtcgtgcagcgcgtcttccagaccttcgtccgacagcccgtaccactgctgcaggaagtagatccgcagcatcctctccagaccaatcggcgggcgacctcgctcgcctctcggatagtggggcccaatcgccgacagcaagcgctgccacggcacgaccttctccatctctgccaggaagcgctggcgtttggtcacgcgcttcttacctgcgctttccgcttctgcaaagctcatctgccgcttcatcactatggctccgttccgtgagctacctgctacaacggcctcagctgcgtcagcgatgacggctgagactgataaatcagcgtttccctagcggctgagttgagctgaccgatcaggccagcatacttctgccgcagtgtgtcgcggaaagcaacccaatccacgttgacctgccgcacctgggcagcgtcatgcgacagaaaaagccattgcccttcgtcactcaggcaccacagcggcacggcccagtcgggatcacgccactgcgcagcaatcgcagagcccaggaggttgcgctggtctgtttccgacgagggatacgtatgaacggcgcccagcgctgaggaggcgaagcctgccacgatggatgcctcgcacgcggctttgatggcggcgagcttcgccgccatcacctgcgcgagggtttgagaagggaatggcgccgccgagttcccgttttccacccatgccaggtacgcgtgatagtccacattcctggcatcctgcggaatgattgcgccgtcctcctcacggaggatggcgccattgatccattgcttgtatcccatgacttacagcctcgcattcaggatgtagttaacccggcaccatgttccgccgtacacgccccaagttgcatccatatcgatcgtggatgcgctgcacccgatgccgccgctcgtcatcccctggttgtggattgtcgcgccgttgtagacggtgtacgccccagaattgccagccatatccttaaacgtcatcgatggccccgcgcgcatttgaaccggaagagtctttgtcccctgcaaactggtcgagttgacagcgattgctgcactcccactgccacttgcgattggcgcgccgccgcagtggaatgcatccgcctggtagtagcgctggcacgccaccagctcaattggaatgggccgaaactccacggccgttgcggtagctccttcttccaggctccactcggtgaaccagaaatctttggccacgacagcgccacacgtagcgtccaccccgatctccaggccgttcgagcagtcaccgagcgccactgtgaacgtcagtaactgcgacgttccggacgccacagccaccgtcttgctgttgattgcgttgaccgatgcgaaattgtcggctgccgtgggcgttctcagtaccaccgtgtagttgatggccgcgccaacggtgtgccgcactttaatctggaacgtaacggtctgattcttgaggtttgctgcgtcagtagcctccatgcggtaacgccatgaaagttgccccgccccggtgagcgtgcacccctgaaagcggacggccttgccgctgcggcccacaggggccgccgtgtcctgcactagcgtccctgccgtaatcgcgccaccagaagcccagccagcaatcatctcaaccggcccatactgaggcgacgtagaaagcgacggcgatgtcgcctgaatgacaacctgcgccccgctgttgatgagcttgttgcggtacgacgtggagcccgcagagccggtagcggcctgcaatagccccacaatcgcttggcgaaactgggtgttatcagccttgttcagcacaaggccgcaccactcgatgaacgacgccacttcctcttggatggcgttcatgaacggtgccgtaatctcagtcggcggccggttggtcccggcgtcttccgccacccacgtgccgttgacgtttccggccccatcaatccggtgcatagttacctccaactcccgtaatgtcttcaaaccaaagaaacacgtgcgcttgctggaatgccgccagggcttgccagagcgcagccggatcgaccaccgagcggtagtagcgcacgcgcagcacgtagcgggatcgggtgccccataggcggttgccaatgtggctgcccacacgcgcaggccccaccaggtggtccacctgcaccagatcaatggggaacatcagcgttggcgcgtcatggctccacaaccggttgccgaccgtgctgccaatggcggctatccggcgcggcatcggctcgcccacgccggcaatggcctcggccgcaacgcggcggtactcgctgatgtgccagcttccgaacttgggccggtgcagctcaatggcggcgtccagaaccgcttgcgtgtcggtgcccaggcgctccagctcggccgcagtgcccgccagcatggcgtcgcccatgccgccgtccggccattcccacgcggcgccaggcggcaacagcgccttgagcgcgtcggtgtagtcgcgtgtggtgtgcgccgtgagcttcatgcccacaccacaggattcatcaccagcacttcgcccgctgcgacggcgatatcggccagcggcgcgaggcgggtgtattgggtggtgacggtggcaacggcagcatccacctcggccatggccagcaccgatgtttcgctgacctcggccagcacggcggccatcagcgccgaagacacggcggtgcggttggcggcggtgtcactgccggccagcagatgaatagacgggttcacgtcgtgccgcagtggtgccaccgcgcgccaatccgccgtggcgggggcaatgtcttgcagatgcgcagctactgcggccagtacggcagcagtcggcatgcggtcggccagcgtgttgcagatcggccggacgatgattgttcccatgcccagcacgtggcgctgaacgagggccgtgctaacggatgggtgggcgcttctggcccaaaacctgtagtcgtcgtccttgccggaacgagcgccacgggtggtgatggtggtccactcgtcggccacccgtgcgcgccaatcgtccacgctctcgtcttcagcgccgcccgtgattcccgcgtcgcctaccgtcaaggtgccattgacgccaggaatcgggtccaccaccgtgagtgccagacccgcagccaggttgctggccaggccggtggtggtgcagcgaatggaaacaggggtagtggggccagcgccgatatcgactgccgccgccacaatgtaatccagcccgttggggccgcgcagcggcgtgccagcaaggatcggcatgcctgccacaccggtggccagcgcgttgcccgtgctagcggtggccatgaggcggtcaacaccgtagagcgccgcccagtcgtagaggcgttccagctcgcacgtgagcggcgagcactgggcatgaagccattcgagatggccatgcatgccgtgcgcggcacgtgcccacatggcagccaggggaccacgcaggaccgcaggcattgcagccagatcggcggcaatgcgtgctttcaggtcggtataaagcggtcgaacgtaggcggtcacaaaggggctcgcacaatgaactttcgtccattgtgaaagccgctgacctctagaactacgctggaaacgtttcctgccgcctcggtggtgacctcttccacggtcacgtctgtgagggcgggcgcctgggctgtaaatgcccgacgcaccatgttgatagcttcgcggctggcgacggacgtaccaaaggccggttccggcctcggtatcttcataccagccgcgccggtcgtaggtgtctgccacgcggccggcgggcgcctctacgtccgtgaacaacacggcgtagaccaacgtggccgccgcggcatccgcgtcgcccagcgccgggtcatcaagcgccacgtcgaacttcccccagtcgctttgaatgagcttcagcatgtcaattcacgtccgaagtgggcgtgccaggggccgtgctcgtgtgggtgtgcttgtcgctcacgtccttaccattcaccctgagttctccggtcacatcagcaccgcccttcatctgcgcagcgccgccgccgtcgccgaagtagccacccaccgagctggtttggcccgccactgtaaggttaccgccgatcttggcgtctttcgtcgtctcgaacatgggcgtatcggccagcaccttcgtgctcgccttgacctcgatgatgccgccgcgcttgatgtgcacccagttctgttcgtcgtcgtgaatggcgatttcgccctccaccagctccatttggtagcgcttgtccccgatgatgagcgccacgccataggagcggtcaccagacgggaaagccaggtacgtttggcagccggccttcggccggtaggaaaagccatagggctccacacggttgatgttgtccaacgtctcttcgtccagcacgcgcacctgcaccttgctcgcgccgactacaaggccaacgccctgcgcgaacagcaactggagccgtgcccaaacctgttgcccgatcatttcttgtgccccttgccgcgcacgcctgcgccgcgcttggatttcttcttttcttcgcccaggaacgcaccccggttcatcacctgtagcagcgtgacgcggccgtgcttgtcatccatacggaatgtccgttcaccgatcaggaacacaccatcaatttcttcttccgggatgatgacgcgcacctgggtgttgagtgcccacgggtgccacttgccgtcggtgtcctgatagcgccagcctggcacctccagttcgatgcggtgggcacgcgccaggcgccggttccgctccaggagtgcgcgccgctcgcagccgcccaggccgttgctgtgtcggtccgccacaacgtgcatgggccggaaatacgtgatgccattgtccttcacggcacccttgagtgcggaatcactgctgtagtcgaacgtcttcacaacgtagtccgaaaaccgcagcttgtattcgtcgatgacctcgtagaccttgaagtgcatgccgtattgcagcgtggccacgggcgcagcgctggtaggcgacgtgagcaccaggccaccgtccggcaacggatacagcagcaggtttgccgcccgcacagcgttgatgagcgcgttggcaggcacctcgcactgcatcgaaaaactgggcaccacgggcgtttcagcgtcgatcttcacagacactttgaacgtggcgcaaaggcgcttcacgatctcgctcagtttcaggccggatagcgtcttggaatactggcagtccacaagctcacgaccaagcgaccgggcgtgaaagttgattgtgtggctggtgggatcgacgcggcggcggttgacatccggtcgcaccttcgtcaccagctcgtcgccgatcagcacgtcgatgacggtgttcgccgtcatgcccatgccgctgccgccgttcgcgccgctgcccggccgcgcgatggcgagctgtacggcggcgcacagatcgtccaccgactcccgaatttccaccttctgccagtagccgtagcgcgtgccgtcaaagcggatttccgccaatgcctcatccatacacacgccccttcacaaacagcggatggcgcgccgcattgcgcgccaggaacaccgattcatccacgcccagccggtgcgcaagcacgatggccggcagcgggttggtcacgtcgcgcaacgcggtcggcttcaggtcttgggccagcagcgcttcaatgaccgccgcccgcgcggccacggccgcgtcaaacacagggtcgggcatgctgggcagcagcgcatccagggcagtcaccacggcgcccagggctgcatctctgtctgcctcggcgcggtagtccgccatggcgatttcgatggcggccgtcaccagcagccggccgcgcaacgcttcttcgcgcagcagattgcgacgcacggcaccatccgttgaggcaacaccagacagcgccagcggcgcagccgtggtggccacggatgcgatgcgagccgccacacgcacgcgcgacgtatccgggtagtccgtcccatcggcaccaccgcccaggctgttggtgaggctgcgcaatgcgttggaatacgcgctgggtatggccatcagcgtggccagatcgcccttggccccggcaatcaggttcatgacctggctcgaccaggtgagcggcagcgtggccagggaaatgacctggcgcagcgcttcgagccgctgactcacggccgcaacgaatgcagtcatgccgtcaaagctcatcgactccagcgtgaagtcgtccatggcggcgtcggccaagctgctgttgcggtcataggccacgtccaccgcatcggtttccccagcggctaccgtgccgccgcccggcacgaagtcaatcgtgatggagcaatagccgcccttgtcgttgctctcgtgaacagaccaatcatgtgcacggacccacttgtcacccagccacgggtgcgtcagccaatcggcgcccggctggttgagcagttccaagaacgcatcgcgctccagatcgtagtccggcccgatgaaataggcgttcagcttccagtcccacgccttcgcgcccaggtcttccacctcgggctcttccgcgcccggcagctcgtgcacagcgaggcgacggccgccgcgtgcttcgtggctctcggtcagaaaatcgacgccgcgaaagcttgcgccgaccaaacggtcttcccagctcatcagggcgctccgttccaaacattaccggtgttcatctgcacgttgccgcccgtggcctgcacagactggctttgcagcacaaggccgggggcaaggcccactgtcatccgggcgttcacgtccaccggcttctgctcggctggcttcagcaggtctttgagaccttcccaggctaagcccagaccgccaccgacagcggcaccaacaccggtgcctaggacgggcaccacgctacccaccatggcgcccatagcagcgccattgagcgcactggagccgtagcgggaaatagccgactcttcaccaaaagctttctcaagggcgtagtcacccaccagagcgcccacgccagcgatgccgccgaccttgcccattttcatcgcgccccggccgaccgcgctacctgtcgcccaagcagcggcattggtgatggcctgtccgcctggcagacctttcccgcccatcgcaatcgatgccaggccagccacacccgccagtgcgccgagtgcggccgtggcgagcgtggtagtgccgaccagaagcggatgcttcttcgccagatcgccaaaggcttcagctgccttgccaattgtacgtagaaggttgtccatggcggacttttgcgccacggcggcatcttccttggcctgctccatcttgaagtcggccgtatctgcaatgcccttgaaggaggtggcgatttccgagcgctcgggggccaagccatactggccccgcaccttgcccaacacgtctttggtgtattcctgattgttcatcacgcccaggaacgccatcagcgactgctggttgtggaacaccttgccgaccgccgtaccctgcacctgggcgctcatggcttccagaatctgccgctggccttccttgtcgttcttgtcggtggcacgcagcttggcttgcagcgcttgaaacttggcatccttggagaaaatcgactgcatcatgtcgatggtggcgcccaccttgtcaatgcctcggctctggtagtccaggaacacgtcatcaacaccctttaggcgcttttccttctcgccgcgtttcagcttctggccgttggccaggtactgctccgccatgtacttgcggaagtgcggtgtgttcagctcgttcagcaggtcgcgcaggttgttgcctgcctcgtcgcgggtgcctgcggtaatgaccgatgcctgattccaggcggccagcttcgcaaagccttccttgcctgataagcccaggttgctggccattgccatctgctgcggcagccacttcgccatatctcgcagctcgaagccgcccgcttggccggccaccatggcagcgctcaggatttgcggcatgtcgtccgcgctgatcttgaatgactgtttcgcgcggatggcgatggtggccagtgcgttggcgtcggtgccggatgcagtggcggccttcatgatgcccggcagcatcttcattgcatccgtatcagacacggtgccggacgcgatcatggtgtcaagtgcttccgctgcctgctcgcgcgtgccaccgccttcgcgacgcgccttgttcacagcatcctccagcgacttggtgccgaccttgcggcccgcggcgtcgcgttcggaataggcggtgttggccatgttggccagacgccggtcatagctcatggagcgttccgcaggccccctgagcgtgtaggcggcagcaccgatgccggcgatggcagcaccagtgaacttcaggcccgcagcggtcttctgcgcagcagtcagccgccccatttcgttggtcagctcagtgaccttctggcgcatctggccgaccgctgcggcttgctcgcgccatgacatggtgccggagcgcgccagccggttgtatgccgcttcggtttgctggatttcgcgctgaatggcgcgctcagagcgcacgcccaaggtttcgcgcgcctgggcaagccgttcataggagctgcgttggcgcgcggcgcttttctcggctgcatcggcagccttctgcgccgccttctcggtggccgtggccgtcttggcagcagtctgctctgccgttttggcagtcttctgaatcgcggcattcgcgctgccatcatcaacaatcttgatcttgacggcgacatcaacagcaggcgacgacatgaaaaaagctccagtaccggaaggtatctggagcttatcgggggtgcttctttcctggcaggcaggaaatgtttcctaccagtctgtgcgcgctactcgaatgtcttgccgtccatctgtgctgcaatctttgcccaggtgaacagctcgggcaacggcatggctttaacgacgggcagcggctggtgcaaaccgcgcgccaccaatgccgcagcgagcagcacgcgcgttacttttttcgcgctgcggcggcggcttcatcctcgggcggcgcttcgccgctttcggctgccggttcctcggcttcgtcttcgtcggcttcgatgatagcgtcggccatcttctcggcccggcgatagtcaatgccgtgcagcttcttcaccagctcttcgtcggtgcccgtcaggctggcgatcagcgcaatgcgttgggctacgccgccgcgcttgtcgaacgacagatagtcttcggcagtggcgtagtcacggaacgtcaggtggctgaccgtgactttgccgaacgccagcgggcgtttcaggttcagtttctgtgccatgttgttggtctcttcagtggcgggtaaaggtcaagttgcggcgtgcagtgcgtcgcgcaaggcgtagcccatcagcggccacaccttttgcgcagcgttctggcgcgcaatcttgcggccgatttcggcattgaagttctcagggcttgcgcaggccgattcaccggtcacggtgaaaccattgcgcaataccagcacacagaaggtcagcaggtgcagggacgggtggcgatcccgaaagtggccgcggccgtcccgcccggccatcatgccgtcttcggcagtgaagtaatgctcgcttgcgatgttcgcttcgatgtcggccggcgtaacacgcggcgcagtcaggcccatggactgaatttccttctcgatgttgtccatagcctgctcttaggaaatccgctcggacgtgttggcgatgatggtcaccttgccatcaccatcgcccacgggcacgggttccgacgtgaatgcctggctcatgaggtacacagtgccatcagccaggcgcacagtcacatcttcgtccttgatgtcgttgatcgcgtcaacgtccagccctggcgtcagattcaggttcagctccagcttggccggcgcgccggactcggtgtagccaccgtcttgcggcaggcggcctggcttgtgctcgcgcttcttgccactcggcgtgaacgtaccgggcttgtcggccagcggcagcttgccgatggacggcaccgacacggtacggatgttgttgagttgggacacgttgaactcccttcaaattgcgttggaaaccaagacttcgtgcgcgtcgatcagaccaccggtacagccttccggaagcgcgagcggcccgcgaggatgtagaacggcgagttgaccaccgggtcatctaggaagttgaagcgactggggttgtccgggtcttgctccaccaccagagtgctcttgtagtagtcatacgcctgcacccagccgtatcgcgacatcagcacgttcttgtagagcgacagcaggaacgctttcacgccatcttccgtggtgatgggcaggcccggccggtagccttcgtccgtcttggcggcaacggtgccccggaacttcttgatggcgcccgtgcgctgttcgtagcggatgcgctccgtaacctcggccacgttgatatccagataggcgtcgtcggtgctgccatcggagcgatgcaggtacatggaaatcagccgcttgatgtagcaggaaccgtccgtgcctacctccatgaggctcatgcccttgaagagcaggctgttcccgctggtccactcgtgatagctcgtggccacaagcccaggcagcgatgtaccttccagcgagataaccgggttgttgtacagcttcggcgcagcggcggcggccagcgtcgcggcggcttcccaggtagtgggcgggttgatgcccagcgacaggtccgtgatgtgctcgtagttcttcgtctcgccaaagctggctgctgctgcgtagtccccacgataggcagcgaatgcgcggaagccggcctgcacgggcacccggtagcggcgttggctctctgcatgccatgccgccagcgtggcggcatcgttgatgcccagcacgacgtagcggaaccacttctgaccgatcagcgttgccaggtcgcccggcagcgggtcgcccgtaccgcctgccatggcggtcagcgcgagcgcgagccctgcgggcatgtcttcgccgtacaggttcaaccgcaggtcgatggcgttgccgcaggtgcctttatggcgcgcgctgagcgtcaccgcagcaccagcggcaacggcagtcaccggaatgtccgcacctgcgtccgtgatggcttgcgcaatggccgtcgcaatctgcgcagccgtcttgcccgcagccacgcccacggtgatggcgcgttgtgcgatgtacagcgcgagcgtgccgtcaccggtggccgcactgttgacgttgatggaaccggttgctgctacgccggccgcgttgtccgcgtagggcagcatgtacaggtcgagcgtctgatcgacattgcgatagcgcgcggccatctgcgccagcatcgaaccatcgcctgccagcgtcttggcatccgctacgctggcgacacgcaccagttcgccggtcggtgccgtgccgctggccaatttctggcctaccagcagtacgacaggcaaatcactgctcaggcccgcctggctcccatcaatctcgatgtaagcgcccggatagcgcagcgccgaaggcacttcactgaaggtaatggtcacagcttttctcccgtcaacagctcccggtctcttgtggtagggcccttgtcatcgtcagcctgaaatctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaggggggggggggtttacccgcaagcccccccagggggaaaagggcagtttcccccggaaatgttccggcaccggggttcccgggaggggttgggcaaaagggcggttcccattttggcccagcgtttttgcccggcacccacgccccccgggggagggggcgttgtgcgatgtccagcgcgagcgggccgtccccggtgggccccactgtggacgttgatgaaaccggttgctgttacgcccggccgcgttgtccgcgtagggcagcatgtacaggtcgagggtctgatcgacattgcgatagcgcgcggccatttgcgccagcatcgaaccatcgcctgccagcgtcttggcatccgctacgctggcgacacgcaccagttcgccggtcggtgccgtgccgctggccaatttctggcctaccagcagtacgacaggcaaatcactgctcaggcccgcctggctcccatcaatctcgatgtaagcgcccggatagcgcagcgccgaaggcacttcactgaaggtaatggtcacagcttttctcccgtgtaaatgagcctgtcaaatgcaggcgggtccggtaggtagttggtaatcaaggcgtcgaactcgtagcgatcgcgccagtacaaatcgccgtcggtgtactccaggacttggccaccgacgaacttgatggggcgcacgccaggctgaagctgccagcccagcagcagcgttttcacggcaatgcggtacttcagcaactcatcgtccgtatcgccttgcttgtgcgtgcggcggttttcaatggcaatcaccacatcgaaggccagcgtcacgttctcagcccgctcgcctgcgtgctgcaccttgtctgctgcccggacgatccacgcggccggcagcggcaacgcttcagggcgaacctgggcgaactcggcagcaccagctacctgccgaaaccagatgcctgcgaactcggcaggcttcaacttcacgtgttcaatcagtggcgtcagcgaaatcaccgcccgacctcatccacataggtgcggccaaagcggcgcggtgcgctttcgatgatggccagatcatcagaaggaaccggatcatcggatgcgggcgggacgaggctgatatcgccacgggcgtgcgccttgagcgtgtccagcacaccttcataggcgcggcgcacgtcgtcggtcatggcctccgcaccttgcaggtagtacagcgccacggtggatgccagccgcgccagcagcgtggtccgcacccgctcgggaatgctgtacgacagcagcagcgcatcggcgtctgccaatgcgcggtcgatagcatccagggcaagcgccaccgcctcttgctcgcgcggcgtgaggtcgcccaggtcgcctccctcaatggccgtgcgcagcgcgtcttccggcaccatgtccatgtcggcgggaaccgccaactgggctagccgcctagcgttgctgcgcgcaagcagatcagagcgggaggcgaagcccattacttgcccgccttcttggcggccgccttggtctcgggcgagtcgtcttgcgattgcgtggtcgagtcttcagcctcttcgggcatatctgccgggcgcgtttccaacacctccagcatctgctcttcgtccaggcgcttggcggttgctttgtccacttccactttttgccatgccttgctgaacttgaagccgcagcggtggaacgtctcgctgctcttcttcggttgcacgcgcacaaagagtgtcgtcacgttgattctcctaaatcggttcctgtacccaacccggcggaacaattcccgccggggacgcacctgctctcgctacggggtatgtgttgttacagccagggcgtaacgaccagttccaccttgttgtagttggtgttgctggagccgttggcgttctgcgcggccttcagcagcgcttcggcatcggccatgttgtccgggccgcagagcagcgtgtccggcacgatgcccagcttgcgctgaccgtcacccctgaacttcatcatggcctcgtaggccgcgttgaagttggcagcgttgagcgcagccttggagccgaacgcgcattgccagaagccgtaagcggcctcgccgcgccaacggccgccgaagctgtacacgtccaggtcgaagacgttggtgttctgcgtggaggtgatgctgttgaactccgcgggcatgcgctgttgcaggtagatcggagacgctgcgcgcttcgtgcacagcagcacccaaggtgcgcccgcaccatcctgcatgttgctcacgttggtgacggcaccggtgccgtcttcgttctcgtacaccgaatgatcggtgtcgaagaaatactggccgtcgtagcagacggaggcgaagccagcgcccagcgcttgaaacaccagatcgttcttcagatcggtggccgactggccggccgactcggcgatggtgccgtactggccgatttcgtcgtcttcgatgtccgtacgctgcacgtccaccgtgcactcgaacttgcggttgaccacctgataggcggtttccttgaacttcttgtgcaggcgtgcgccgacccattcccggaaggccgggaactggctcagccattcgtaggtgttggatttgccggtactggtgactagcttggcgatcttcttccagtcttccggcgtcagcaccaagccagcattccagcgggcaatcagcgtggtcttcagttggtccaactgggcttgcgtgagtgcacccatcgttttctcctgttgggttttgggatgattttgtcgtgtggactagcggcgcgagctaggccgcttttcagcgtttggcggcgacgtactgctcgggggtgacccccatcttcttgcacatcgcaagctcttcgtgggtcaggccatgggcaccttcgctcttgccatcggcctgcttgtccaggatggccagcggcttgctggcgtccaggtactctgtcagcgcggccagcgattgcttttcagcccacggcttctgcgccggggtcaggcggccatcggtcagcgcggctttcagcagatcgccgtgttgtttgcgctctgcggccagcgcggcttcggcttcttccttctcgatgccttccaccttggccttcagcgtgtcgcgctctgccgtaagggcagcaacgttggtcttcaggccatcgcgctcagtggtcagcgctgcgagctgcgatttcaggccgtcacgctcggtcgtgagcgcggcgatttcttgctgcgacatgtcgaactccccttctcgaaaattgaattgcttgcgggccagagcggccacggcttctagcccatccagggcgggtgtgttggtgagtgcgaccgaaaggatttcgagcacttcacctgtgacggggctgtagaagaagacagtgctgatgtagcggtacttcttctgcgcgatgagctgggcggcgtcgccgacccatgcgatgttggtggcgtagagccctttgccttcgcgccattcgagcgtgcggggaatccagccagccgcatcggcacgctggccattccgttccttgttcaggctctggtggtcgtagtcgaccaggatgtcgttcttctgctgctcggccagcgcgatcacgcgcgcggcgatggcggcgtcgagctgccaagccgcgcagtcgaacgggcgaccatccgttgcccggaacgggccgagcggcagcaaatgggcttcagtcggaacggcaccatcgctgccgggcaccaactcgaacgctagcgcggctattgcggtttgtgcgatggggggattctgcgtcacagcgatgctcctaatcgatgaggagcattcttgacttcagcgagactttggtcaggtagtgaaatgtttcatgcgcgcggtaatctgggttaccgtgtttgttatgctttcgcgaaacccttgataatccaggagatttgatagatatgccgactgcatcctgcacatttacgctcaacgggcaggcaaccagcacgctaacgtgcgacggccgcgtttatgtggctttctctggcaataaagggcatgagaacaagcccggcgataccggcatcgctggacaagggccattaccaaccgggcgttattacatcgtggaccgtgagagcggcgggcgcctggggccgatgatggactcgatccggtcagcggtcagcaactccaatcacgccgagtggttcgcgctctaccgcgatgatggcgcgattgacgactggacgtttgtgagtggcgtgcggcgcggtaatttccggctgcacccagttggcccgcgaggcattagcgagggctgcgtgacattggtcagcaaacagcaattcgcggctctgcgcacctacctcaagtctcgtcagaccggggttgtccccggcacgaacacgaagtattacggtgtgctggaggtcaaatagccatgcggtttgccctcaagaccgtcgctgtcgtgttactggcgttcgcatatacgcgggccatttatctctggtcttggcttgacccattctttgccagttcggcatggcagcgaatctatggcttcctcgcgcacctgtttggcgtgagcggtgcggagcaaggcgaagacttgcagctcgccatgttccttgtggtggcactgctgctggcgatcttgtcagtttggctgttcgaccgctacgtgtgtcggccggcctgccacacattagcgcggcagagaaccgcgaaaaggccctaacgatgccgcctccgggcggcattcttgtatgtatggggcggaattcaggcgaggtctggtgggcgaccgtctaaaacgcgctcagagccgttttaggctgctgatggaccgtgggaggtccacgccccggaaaggcgtttttaaacggctttcgaacgcgaatggaggcatcccgcaaggcggcgctccccaaaggggcaccgtgcggggtcagaagaggtctagctgtcgatcgtcgtcctcacggcagatgttgacaatctgacgggacgaaaggtgatggcgccgagccaaagcgttgatgctggcgttgtgccgatctttgcgaatctgcgcgtcacgcacgcggataaacagcttgtcggccttgggcagccaacaacggccatccgaatccagatgcggagccagcgtcgtccgcaaacgggccagttcgtcggcctccaagcccagtgcgtgagtacggtactgcgggatgctgacattgacgccaccatggtgggtcagccagtcccgcgcccgaccgtaaccaagcgcgcggaccaccgcacgcagcacgggtggcaaggttctcagcaaatcttcatcgacagccgggtaggcgtctaccacgcgctcgatttccgacgcggcatgcgcagcgggatggtgatagtgcggaatgaccatggcttacccgcgcgccagccaactcttgagagcttcggcaatcgtttggcattcagctacggccaagctgtccaagtccggcacgtgacggcccacttgacgcgcacagaactcaagcagcgccgggcgcgttgccttagccaccttgcccgcctgcccgagcttgaaccacatgcgcacgaggtgagcgatgcggggtgggacttccttggtcacgccagcctgacggaacacgcgccgatagcgcgaccacccccgacgctcgtagtcgtccagcacagcgccgagctgcgcgaggttgagggtactggcggacacgcggccgtcaaccacggctgcgccatggcgtgtaagcaggtcgcggtgggtctcttcagaccagccaggcaggctgaccatggcccagcctttggcgatgccgacaagctgccgatgatgcttgatgaggaacgtagacattttcatgcccccagcatttggggtgcatccacccctgttccaaccccacgatggagggtggcgccgcgcccagcgcgaacgccttgcacgcagtcgcccacctgggaaacatggcgcccaggttcgcggcaccgcgtctcaacttgctcggtcacggcatagttcgcgcgcatgtaggcatccaccgcgctgcggtgggcttcgggcggcacggtggcacccaccagctcgcgcacggcaaacacccatccttcgcagaacatgtctgcgcgggccaccttgttcttggagcggcagcgcttgagcgcggtcttgatgtagtcggcccgcgcgcggcgaagctggcggaacagaacccgatagctgtaaccggcgaccgtcgaagcgctggccataccgataaatacgtagccgccttggatggtggttcgaccggacaggcggttggaaaacaccagctcgcaaccaaaggcggatgcgatcatgctggccagccacacctcatagcgggccggcgtccgggtcgcaccgctcttcacccacagctcggccacgccggccgccagcatgtcgtcttcgctgacgccataggcggtcatcatcttctgcgcctgacgcatggcggcagcggcctcatgcgggttgctggattgcccaagcgccaaacacttcttgatcttgtcgattgcggtttcgcggtccataactcccctctggctgctcatcagtgtccggccaccacgccgggcagacacccgcttgcgcgggcgtttcgcgggtgggccggctaggccgctgctgcatccttcagggccttggcagccgagaacttcggcgcgcgcttggccgcaatggcaataacgtcgccagtcttcgggttgcggccggtcttggcggcacgctccacggagccgaacttgcccaggtcggtgatcgtcagctcgttaccggcgcgcacggtgtcaagaatggtttcggtcagggcgttgagcaggtcttcagccttgcccttggttacttcggccttgtcggccagatgcttgatgagttcttgcttggtcattacaaaagttctcctatggtttgccgcgatacgctcgcggcgggcgttactgaaatcgaaccgccagggcgttacagagcggcaaagtccagggtgacggctgtgggcttcttggcaccctcggcgcgctcgtagaagcggatgtaggccgtggtgcccaccacatccaccgactcgccgatggcctgcatggcgcacgtccacttgtcgtcgctgatggccaggcggcgcagcgcgagcacgcggccggtgttgatctttccctccttgtccgtttcgaaggcttgctgcaccagcacctgaatcttcgggtcgctgcccttggcccattccttgatgcactcgtcgatgagcaccttggcggcttgcaggcgttcatcgaaagcgatgttctcggcctgggacacgactacctgctggcgaccgtcgaacgtgctcaaggtgacgttgccctttgcaccgcccagcttcacttcgtagcgctcggccgagagcgatacaaatgcgtcggtgtcgttaaaggcttggcgtttgaaatcggccatggctgcttgcagtgccttggctttggccaccagctcgcgcaccagcttgtcgcgctggaggtcgatggggtggatggtcgattcgggcaccaagtggccccgcgcgttggcgcgatagccagccggaatctcggtggggtgttgcgttactgtggtcattggcgtcctttcttcaggttgttgagggcttcgcgcacttcttgaggcatctcggttttcggcagttgctcgcttaccgctgccggccctacggtggtcgtggccgcttggcggcgggcttccggtgtgccagccccagcaatgccgcgccgctgcgcctcttggtcggcttccgcttgactcgctgcgcgggcggcctgccccgcgattacagagcgcagccacccgtgcgacttcagggggagttctaggcggccgtggttcgcttggtccactgcgtagctgaaagccgacgcccacagcgccatgggcgccgcgtacacggcaccgtcgcgcttaacctgggccgctcgcatctccggctccagctcgcggatgaggcttgccatgcgtccgttggccatctgcgtcttcttcggtccaaataggccgatgtagcgcaggagcggcttgatgaacgagtcgcctgccgggtgcacgtccaccagcgccagcagcatgtcgcgcacgccgtcatcggctagcaccacatccaggctcatccgcacgcggcagttcgggcagacgacttcaggcagcggcattgccacctccaaccagatcggggttcgtttcgcgcagacggcggtacagcacgcggcggcgcccggcgtacttcgcttgaaggcgcgtgaggttgtcccgagcacgggccatttcttcctgccgccactccacatcgcgcaccgcgtggatctcttcgcggtcgatggcgcgaagctggcgctgcacgcccacccgcacgcgcaaccggcgccactccgcgcagattcggcgcaagcccgccagcagttcggctgcaacaggtccctgggtgatgcctgctagctgtttcatgtcttcaccttccatttcacttcgtcggggatttggcatgtgccttcgatccaggcgccaaacgcggctgcctctgctgaaggtgcgccgttggcgatggcatccaagaagcgcacggccgctggaccggtggcatagatgcgcttccggcgccgtgtatgggtaatcgtgatgcccggtgggtcaaactgttccgccatggctgcctccgctcaggccagcgattgcaggcacagcgcctgtttggccaccgcatccaccagcttcacgtccagcggacgcccacggcggaactccttgatgccagcgaccaacccctccaccagcatccgcgcgctgcccttgcagtattggtaaaggcgttgcaccacctcttcaggtacgtcttccgtgccgaagccggcttgcaccagggcggcggcgtcttccaggttgatggcgcgcaccgtctcgggccagaagcccgtgcgcgagcggatttggtcaaactggccatgcagcggcttgatgaggcccgacaggtgttcggtgccgcagagcacgatgccgacgttggctagatcacggaggcggcgcagcgtgtggagctggtgcggcgtgagcgtttcggcctcatccacgatgagcagactgtcggtgttgcgaagcgccgtcaccacggagcggaacttgtcatcaatgctgcccttgccgtcatagccggccaccacgcgggccagcagacgcacaaggctctgcggcgtcatggtgggcgtggcctcaatcaggtgcgtgttcggatgcgatgccacgtattgcttgatggcaaaggtcttgcccgtgcccacgtaaccggtgaacaccgcgaagttgcgatagcggcgggccatgctgcacgcggcgtgcgcgagcttgaagacgctggtttcgaccggggccaccacatcggccttcgtctcttcagcgtggcgcatggccgagtccaccgacgccagcagcttgcccggactggtggcatagctgcctttgagaatctggttcaagctgcttgcgctgatgcgcgccagccgcgccagagcggcttgggtgtaaccacgctcttccatccaggggccaatgcggccgataagcgcaacgtctgcggcggcgtaatgggcgggccaggtcgtttgcttgtccatggggttctcctgttattcgtcgttcatcgtcaggtcgagcagcagcggcgcgtcgtcgctgtcgtccaccaggcgggcttccacctcgattgcaggcagcacgccgtcggccacgctgtccacatcgagcaccatgcccgcacgggccttctgctcgtccatcttttgttgcaggcgcttgatggcatcggcggcgcgcgcctggcgcttttcttccaggcggttgggcgcgatggcgtcaatggccgtgacgaggtgtgcgtcgcaaatccaccggccttcctgcgtgcgcatgacggcgacacggtcatccatcaggtcgtattccatgaccaccctgtggccgttgaaggcgtgcagctcggggtgcgtgtaggcacgcttgccgtgcttgatggctgcgcgattcaccgtcagcaccaccgcctggcgcttcagttcggtcacgttggcgtgcggcggaagcggtgccagttgcgaccacagcgcggcgcgtgtcgtgttctggtcttctgggtgcgggcggttggcgtagcggtccacccaggcgtgaaggcgtcggcaaactggggcaatgtgggcagcannnnnnnnnnnnnnnnngtgttcttgtagccggacccgttgtcgatgtagagcatcggcggcacgtggttccagcgggcgaagcactctgcccacatgttctgcacggcatacgtgccttcatgttcgtcggcccgccatcccacggggaagcggctgcgcatgtcgattgccaccgtcagctccgggcgccagatatcgcccgtgacggggtgcgccaggtacacgtccgcgcggtagccgtcggccacatacacatcgccgggcaaggcgttttcggtcgagcggcggatgtatgccttctcggtcaggcggtacaggttgcggccaatgcgtgcggggctgttgcggccgagcatggcaggaacccccgtcaggtagttgcgtacttggtcgtagctgaccgcgaagccgtccacttccgccaggcggcgatgtacagcggccatatctggtttgccgggcgcgttgaaatactccagcgccggcccccaccagccggcggcctccaccacgcggcccttgtggtcgggcagcagcgcagttgcgccgccttcgcgatactgagcgcaccattcgcagatggcggagcgcgatggcgtcttccggcctgccttggcggcgccagccagcgccatggcgaagtggctgggcaggctgccggcctcgccgcgctccagcagcagcgccaccgcgttgttctgcgtcacaccgtcttccgtcatggcgcgcacgtacgccaccaccgtctcacgccacgtcgccacttgccgggccctgtcggttgcctcgcgccacgggtcgcgagcacgaagcgccagcacctttgctgtgggcatgaccgctaccgcgccacttgcgccgcctgcgtccacgcgctgaaccagttgtgcccgtgccatgacttactcccccttagccttgttggtggaacccttgggacggccggcgccgcgcggtttggctgcatcacgcttctcttgccggtttgctgcttcggcggcgtggcggttttcgatcatcgggtagtcgatcagccattggcgggcctcgtcagggctgaggatgtgggtgccttggatgcgttcgggcatgtcgtcttcgcgcacgaggctggccatgtggtcgatcaaatccaggccgcgcgaagccatgacgtgtgccgtcacccaaatctgttccatctggaggcgccattcgggcgactcgtcgccgttgacctcatcgaacagctttttcaggctggaagtgttgagttcaacgccaagttgcagggccatgcactcttcgcggatttcctctgtgcgcaagaggaattcggtggtgcgatttacttgcgacaggcgtttgacctgcgagtgcgcacgctccagctcagcttcgtagttcttttcacgccgcttcagcgcgcgaatctcttccttgagttcggccaccgtcatcaggtcggctttgccgatcatgtcgttgccggactcggcggcttggtcgatgacttcctgcggcagcgaggccaagagcatcaccttgaccttgccgagcgccagcacgccttccagttcgtcttgcggcagttgagccacaaacttcgcgatgcgcatggcttcgtacacgcgctggtttgcaagtccatgcgcgttgatccaggtcagaaagccgtccttttcaactcccgaacgttcgggagtcgatctgcgatccccaccatgcccaagcgaaatcaactcttgggcgcgcagaaatgccatcccagcacgcgtaatttcgaacatggcacggccgatagagtcagcgccgatgcgggccagctcgccgaggtcatcggtggtcacgttgaaatgcgacgcgactgcggcgagattcgccactggtgcgagcgcgatgggcagcaccgccgcagcggttgcaatatccactggctgcgcgtcgtccgtctgcgtggcgtgaactagagcgccttccgtgatgcccaggtcgatatcttcggttgcttgtacggctttggttttcttcgtcatggtcggcttcacgcagctttcttgagacccagtgccacggcgactttgtgagcctcgccgtaacgccccttgttcacgccgttgatgaccgcgataaccgtgcggtattcaaagcccttctcctttgcccactttgcaaaggtcttgccctcgcgctcaaagcgcgcctttacttgctccggggtcattgctacgctccatccgttacgattaagaatgactcattatgggaaagaaaactttcccagtcaatacattccagggaaattttttatgaccatcggagagcgtttgaaggaagagcgcgagcgattgcgcctcacccagccggccattgcctcggccgccgacacgacgaagcaaacacagcacgcatacgaaaccgaccgcacgccgccgaaagccagctatctggccgcaatagcgatcctgggcgttgatgtggcttatgtgataacgggggagcgggcgcttaatacggcgcgcacgccgatggaagttgcactgctggagaactaccggcacagcccagcagaggtgcagcggggggttagcatgcttctcgcacaaacctccggagccgcagattcagtcgccggaaagggaaagaaaaatcgatcctcggaggaagtgaagaagtgaagcgcacaatgctcattcttgcgatagcaactattgctagcgcaccagccatggctaaaggaccgatttcgccagaggtcttcagagacagctataacgcgcatgcgaaagacctacttcccgcgttgcagacaacacagtgcaggaccatcagcgctgcgaaggagccgggcaaggcgatcttggaatgtcagaccgtggcaaacaacacgctgttgagcatcaacggcatgaacaagcgtttcgacggtgcttggctgatgctggacgcgagtgcattgccacatccttccgacttgacgcgggcgggcgggttgttgctccgcgttgcgaagggctaccactacggcaaccatctggcccttgctcaggaggcgattattggtgcgcaaagaagcaaggggaagcaatcctgcattgacgatcccgcatcaggtagccgcctctgcgtctcgaccgataacggcacgatctacaacatgatcttggagcaagccgagcccaagaagggctgatacatgcactaagtggatacccgcccccaggaaacgtttcacgcctaatcgcgctccgcgcgcgtgcgtaacctctggtcattccaatgacccggaggttacgcatgaaactccctcgccttaccacgtggatcatcgccaccattgcgctggcgatcatcatcggtctgctgtctccgcagcaactacccgtcagtctttacaagctctcgttggtcaccatggctgccgtggtggcgtactggctcgaccgcgcgctgttcccctatgcccggccggccggctacctgtccagtgcggactggcgcaaagacgggcctatgtgcgatgacgccgaccacgccattgttaccggctacgagctggtgtttgccgccgccatgcttcgccgcgccgtcatcgtggccggtgcgatgctcgccatcggtctgggggcgtaaccatgcgccgccagtcgatcctcttgagcgtgttcgccgcgctccagctcgcggcggcgctgctgctcgcatcggcagatgcattcggtgctgatgccgccatcccgcacgtcgcgagacgctaccgtgcagacctgacccgcattgcacgcgaggcatggggacttgacgcacccgtggctgtgttcgccgcacagctccaccaggaaagcggctggaaccccgcagcggtctcgcgtgttggtgcgcaaggtatggcgcagttcatgcccgccacggcgaaatggtggtgtgtgctgaatggccttgccaacgatgcgtgccagcccaccaaccctgtgtgggctatgcgtgcattggtgggctatgaccgctggctgtatgagcgcgtgcgcggtgcatccgagtttgaccggctgtgggctgcactacgtgcgtacaacggcggactgggccactggcaacaggaagcggccacggtgcggcctgcgctgaaccgcgcaaccatcgatagcgcatgtggcacagcgcgccgccatcgcagcttctgccctgagaacctgggctatccgcatcgcatcttgaacgtcttgcagccgcgctatctggcgtgggggcgtggggtacggacatgacccgcacgctgattgcaacggcggtagccgtagcgtgcggtgcccttagcgcgtggagctacacacgcaatcactacgtggccgagattgctggcatgacggccgatcaagccacggcccgagagaaagccgagaagatcgcgcgtgagctgctcgaagccgagcaaaagcgcggcaacgcgctgtcggacacgttggccaagaaagaaaccgcgatcaccgaaaagacccaggagcttgccaatgcactatctcgcctcactactggccgcaagtgtctcgatgctcgcgttgtgcgcgtgctcaacgactccagcaccggcaccgctactgacaacgtgcgcgccaccaccggcacatctgatgcagcggatggacccgctgccaccgataccgacgtcgctagctggatcaaccacgccaaaggccaatacgaaatctgccgggctcgactcggcgcactgatcgactttgaaaaagggcgagttcaa

SEQ ID NO: 450 - DNA, 10384 bp, Ralstonia solanacearum
  misc_feature (8360)..(9288): n is a, c, g, or t
tgtctagaccgcgctcatcatgagtgataactcattttactatgctcatagttgagcaatagcatatggacgtgcgtagcaattaacaaagaggaaccacaacttcgtggtgacacacatgacgacatcgagcacttccatgccgctggcacctacgccgcgaccttcaagcgagccgccagcgctcagcccgatgctgagcgtcaagctgatcacggtcgccttgttctgggggggaaccttcattgctggcagagttctggcccaggctctgcctctgatggtggccgcattcggcaggttcctcgtggcggcaatactgctggtctgggccgcattccacatcgaaggaaggccgcctcgtttgaaccggtcgcagttgctcacgactgctgcgatgggactgaccggcatctgtctctacaacgtctgcttcttcggtgcgttggcgcgccttccagccggccggactgccttatttgtcagcctgacgccgatcgtcaccgcattgatggcgagcttggtattccgggagcggctcgaatcgaagcgctgggtgggcatcggtattgccttgatgggcgccttgattgtgatcacccgaggtgatcttgccggggcaatgcaagacattggcgcatcgatcggacttggggaaatgctgatgtcgctggccgtcttgaactgggccgcgtacacgctgattgccagaaaagccaccgagtcgatgtcctccattgccgccaccacctacgccgctttatgggggctcgcctttctttccgtgggagccgtgggcgaacttggagcggttcaatggttgacccttggctggcaagtctgggcgtcggttctttacttgggggccattggcaccgtcgtcgctttcatctggtactcccaggggattcgcgcggttggcccctctcggacggctgtgttcactaacctggtacccgcgttcggtgttgtgctctccgctggcctgctcggcgaggcggttctggcctccatgctggtcggaggtgccctgtctgctatcggtgtttccttggcgaatcggaaataggcgccgctgaagggtatggatccgggcgttctcgggccagccccatcgcgtcgtggccatggcgaatggtcagttcgagtacaacggcaagccttacaacagcctcaccgcgactgcctgcgaaatcgccggcacccgctggtcgggtccggcgttctttgggctgcgcgatggagccaagaagcagcggaagggggcgggcccatgagcgtggattccagcaagaaacgccgccgttgcgcggtctacacgcgcaaatccatgggcgaggggctcaatcaggaattcaacggacgactatgacgacccgcgtattccgacggcaacatggagcggccgggcctgaagcgtttgctggcggacatcgagcgcggcctgatcgagatcgttgtggtctacaagatcgagcctggtggacttctccaggatggtggtggactgcgggagcgaatacagaaagactcttgcaaaggttgcggggttttgcgtgccaatgcctctcgctgcagaaagcgttagaaccggctgatttgtcgtacaacgcccatcaccagccgcctaattgtctaaggacctcctgcctaggtggtccttcgagcgccgaccgactcgcgtgacaggcctctaaatcacgctgagtacggaggcaattcacatttgtctgataccggtgtatcggtaactgagaaatatcaggggcctgatgaatatgagcggagtgagtaatcgtgtggccgttccggatttatcaagaagcaggctgagcgggtctggcttgattgagtatatctaatacatgttcaacgcgccccagtcgttccttgctctcctcggatcgcagtgccgcgattaaaaatctaatcggagatatctccaattcatacgagccaattttatcaatcaaccctcgaagttcagtagctaggtcggcaaggtttttatccgataaaccagccaattcgtaggcaatgatgcgcagggcgagatgatttacttcgacgaaggagccgggatgaggggtgatactatccaagaacgattccagatcattaattttttcttctaaacatgcgcacatggcaaaattgaaagtgctgtgcacctttccaagtccaagtttttcggcttctaaatagccggacttggcttcggcgatattccccaaggccagactgtgatggccctgcgcggcgtactgtgtggccacggtggatgaactccttccaaataggcgtccctcctcaatccgttgccggaccgctgctatctgtgagttgtccggagatatttcttcggccgcgtccagatctcgaagcgctccggatcgatctcctagcagaaatcttgcctttgctcgcccgacgtaatgtccatactgacctggctcggtatcgatgagatcattgtatcgttccaaagctcgcactatgcggtcatcagaggatccgctcttctccgatggagtcatgacctcgctggcacttactgtactggtcgacgaaacaaatccgctaatctccagctgcgacaatgcttgcgttagctcgatacgaagctcggctctcaggcgctcatcttcttgctggatgctctggaattcacggtcggctatttcaagtaacccggccagtgcaacaaaacgcctcttaagatcgtttggaatttgctgatccgatttgtattggatgtcgtgttccatttcggcccaggcatgctgaagaatggttcggacttgtatttcggcaacaagcggagtaaatctagagtattccgtcgcctggatacgtggatcttttaggcgaacaagaaagtgaatgcttttgtagccgatctggccggagcccagtagggccgcgtccttgtcgagcctctcctgcacgttaaactcgcggcgaattacatcctcaacgtctctgacagtttgcggtacgtatgtgattactcggacgccggcaaggtctgttatatcttgaagcggttgagggtattttagttgccctgtatctgggtcgatcttcgatactttttctgaaaaactgtcaaaatcctttgctcgccactgaactgtgtgtattgagttgctaatcagggctgtatccagaatgtttttaattgcttctgcgaaagaggcatacaaatcgcgtacggggagatatgccagaagacttttgttggcgtgcgactcgactgcttgcggatcaatgctcataataattttagtatcagcggtgcgtttcgtggcgagtcttccgtgcaatgtatccccggtcgtctgcagcggagaagcaggcggctagatggcctccaacgcagtcatcaggtgtaaagtgaccatcgggaatggccgtgtctcgccaccaaatttggccttgctcatcaactacttggttgtgcatttgacccctccgaccgtgtgctttctttaatatatagtacacgcagtcgaacggtatagtgacgcattcgttcatcgcataaatgccatgtctcgcttgggaatcgagtatcagaacggtctgatttatgagggcagggacaacccgagctccttagccgttccgacgccaatcatctcgcagtgcacactcattgagtccccctctgatcttgagaggctgcctcgtgggatagactcggatccgtttcgctggattttcagggaggactcgttcgaccccgtttccagggttcgacggggccgactatttcagcctttcagtaactcgaacaaggagttggtctccgttgacgcgcatccctttctgccttcggatttgggccggcgtggcgtcgatggtcaattgcgcaaggaaatgaccgtgttcattcactgcacgcaacttgtggtgcgccgggagcgcggggaaggtctgcagcttgcaatcggccacgaaggggcacactcgctctggcgcattctgcagactgagcaaacggttacccaggacgtgctggtcacccttcgagcggagtctgtattcggggtactgccacccctcgatctggcgcaaatcccagaggacggccgaacggcagtagctgcggcctacgaccgggtcatgcatgttgcatatcgagactccccgacatcggttgtggaccaatgcaggaacctctgcgcagttctcatggctcgctggttgcgccagcggacgagcaacgataggttgcttcacgacgatctcggcgcgtgtatatcggcagtcaagaagttcgccgaaggagagcaccaactcgtccgcgccgcacttgagacagtgaatcgtctccatccacgagggaaggagaacgagcgtgagcggtacagtttgcgggaggtgtcaaatgaggacgcagagcttgcattgcacgcaaccgggttcgtgattagggagcttggctgggggcgctgatgaaccccacctttcatttcttaggagttcctttttttgtggggcgcgtgaacgctttgtgagaattcaccccgcatcgccgctctagaatgccccgccgtattggagcaagcgagtgggtcggtagcgccacccgccgccggtcggcgtatagaacaggtacaggtggggtagttgccgtgaaggttttcaatgcccggcattttctgaggcatatcgctgccagcgtgctgcgcgagttcacgcaagcgcatgcgctggccacgcgtttggtggtggactggtccggaccagccgacatgttgtcgggactgctctgcgatgcggttgatgcattggagcagcaggttgctgccgccgaccttcccgaacgcgaccgcgaagcgctggagcacgacctgctgctgtggactgacgacctgcggcgtgcttgtctgatggccaacagccttgctgtagccgagttccggagcgcatgccaggacgatccagatgccctgggggcgttcgcctcccgtgacgagcgagagatcgcactgtggatgctggtcttccgcgacaagatcttccgcgacgtcgaactgcatctggccttccaatccaagacccacggcaagttttggaagaggcaccgcatccagcccggcctcgaactgacgcgcgagcgggtccggctcgaacagttctgccatgccgtggcgcagctttacaagaagtccggcggcggcgatggtgtgcacatcgaactctccgagcgacgcagcacggcaggcctggtggatgccatgtccagtttccagctgaccctctatgtcgagggaccagtgacggcgctcacgcacttcacgcagagccatttcactcgcgtcaccacccgcgtggcgctggagtccgcgttggtctaccacccagcgaccggcgaagtggaaacagtcgtcaagggtggcgccaagaaccatggtgcgatgctggagttgttcggcgagcatgtcgtccagcaggatctggcccccgagcggattgagccgcaacgctaccacctcaacgccttgcgcgatgacctgcagccctatgaggattggtcgatccacggcgtggatagggtccgtctgcgccgcgcacgcctagcgccggcggcgggatcaggtgtcagtttcactgtcgaagcctcgcccgacaaggaccaggacgacgccatccggatcgcccgcaaggcgctcaaggtcgagcatatgtttgaggcggaatatcacctcgaggctgcgaccgtcatcgtatacacgcaggccgccgacggcgggcgggcgggccatttcagcttcaatatccgcgcctcgggagcctcgaccatcaagaacctgtcgctgaggaaccaggtgctcgcgcgcaaggtcctgcaggcgctgatggtgatcgacgccgaggacgatctcatcgcgaccatgtccattcccagggaggctatcgcagcgtgagtcaggctcaggttgacgctaccgcgctgctctgccgattgctggagcgcgacaaacccgaaatcaacggccaggccctgttcgatggggacccgcaggcagcggctcacctgctgcgtgagcggttgctggtggtcggccgcccgctcgaatgggtcacatgcccggactgccgtgtggaaatcgcgcgggtcgtgcgtgatgtctcggccgacaggatcgccctgcgttgcccggaatgcgaggacatcgacgcctcgcgtcgtctgcgggaaacgtacacggcggtgccggctcgggccgtcgctgccttactgagcgggctcgggatgacagccggtggtatgaaggtcatcgaacccgaccgcgtctggcggctgggcacgacggagccgacgcgagggaagccgctgacttggtacttcgcacgccagcttgggcgaccggagatcggcgcccggctgcgcgagcagatccagctcgagcgcacggccggttcctgcgtggcgctgaccaccagcgagttgccgttgccgattggctcgccgctggtgggcttcgatgtgcgcatgctgtccagcgcggcgcggataagtcagagccgcttcgaattcttcgcggaccggcaggctgcgccgggggcgcagcaggtgggggaggcagagccgcggttgaccgcgcagaccaccctgcgctacgtgcgttcgctgggcaaagccttcatcgaaggaacggaatatcctctggagccgcgccagcaggccatgctgcttgctttgctcaacgatctcgatcacgagatgggtaaggccgcgctcaagatcgcctgcggctcgcaggcgcaacggttctcgccgagcaaggagttcgaccgcaacccggtcgtctacaagaccttcattcgctacctgcgcgacgacgaacgctacgcgcttatcgttcctgacgaggatcgggactggttggggtaggtgccgcaatacacccattcgccggcccagccggccaaatgcccgatactcatgtgctcgacgctattataagagcgggggcatgtcatcggcgcgaacgaggatttcgagcaaatcgaccctgcaggtttgacagccggtgcttgcgtttagaacgtggttagggctgcagaagcttccggagctcaggcaagggaccgtttctgtcaaaaatttgcgaagcaagaaaggcttcgtcaagggcgccaagtgagcccgaaggggaccgacgcaactgagcaaggacaagcttcaaagcggcaatggggaacgcgagcatcgcgtggtctgggtcaaaagtgtaaaggtgtattttgtcgagaacataggcttggaccaactgccgcactccgacctgttctacgaaagcaaaatgagcgggggctgtaggctcacagaacttcgggattttgtagttggctgcctctcgcttatccaccagccagtcgaacggtttatcggccccaatatcctgtgaggcgaggacgctgctcgtgtaatgatgggagaacgtgtctaacacgactttgtgcgtagtgcccgcacgctttgtcggagtagcgccggcggttgcccgccacgaatagggcgtagttcttgaatagaagatcccgacgccaccgagggcaagcgacgcccgcgtcaaatagaaggcgctgtaatagagcttcacggtcgcccacgtgtacaacttcctgtctagcccctgtattgcttcagcaatggacaaggttccggagtaaattgcatcgcgtgcgtcctcattcaaggaggcagcaagctggacagcctccctggcttggagctgcctggaaactatggcagccccggtaatcccagatagaatggagtccgcaacgcgttgcgcacgatgcataagattagaatttgaacttcgttgatgccttgactttggtggggctgaaggcctcttgcagtcgtgcgtatatcttcttctgcgccccgctaccaccctgccccttaacttccgcctggatgataagtgactcgttgaacttcagagctgcctgaattgttttcacctcaaatgaggcctgattcttacagaagggaatcaattgaagcctgaggaaatccattgcggcaagaaatcctgatgcgtagagaaagacgttctctttgtcttcccaacgatcgccatagatgccctggagcgcgatgaaccagttgagaatgatctgcttttgcaactctagggaaccgctgatcaatgaagtcggcgaatggaatcgcgagtcatgccatcttggtttttcggattgaggaagggatgcttcgattcagcacggtttttcgctgctttcgcacctatcttggcgcgctttgcgcgctcaggacgcaatgctcccaagtagcattacgcaccgctccaccatcgcacatcttctcagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggatgcgattcccgcgatacgtggcctggatggccggccgcgtaagcgccccgacaagctgcatgccgacaaggcttacgactgccgccgatgtcggcagtatcttaagcggcacggcataagggcccggatcgcccgcaagggcatcgagagcagggagcgcctgggccgttaccggtgggtggttgagcgcacgcacgcttggtttgccggctttggaaagatacgcgttcgtttcgaacgacggctcgatatccattgcgcgctcctctcgcttgctgccgccatcatctgcgctcgattcgtggatgacttgtgttagcgactctaagtacgtttgtaccggctctagtggaaagattgtgtggggcggtggcggattggaggggcgctcagagtttgggccgaataagtcggattttatggcggcgctttatttggatgagcaaggcaatttgattgtggaggatcgtattcaggttcacatgagcttgcgacttctgcatattccaacggggacggcgcagcattacacggtttacagatttaggcgattgaaataagaatctgttatggaaccacgcgaaagtagagggccgcgcagaatttatttgcgctcaatgtgaatcgtgagtgtttttattcaccccacaaggccagaacgcagagtcgagccaggcttttgctgggcagcgttgcttttcgggagcatttgggcttactcggaaggattgatcgttcatggcggacgactggctgccgccgatggggctgtcgggttgttgtacctctacggcgagcaaaccggtcatccatcagcggctggtggcgctctactgctgtttctcgccaagaacgtttattgcgccgtgcgaggcaggttttggctgcagtgcttgctgctcagtcagggctatcgcgtggtcagtcagtagaagcccgagcatcgggccactgatatgcaggcggcatgagcgacgtgccaccgctacaagtacacatcaagcagtttttggggaagtttctgtgcaatcaccacaccgcccggtccaactggccgtactcctcggcctatgcgccgtcttcatcccaccag

(19) United States
(12) Patent Application Publication
(10) Pub. No.: US 2010/0285976 A1
(43) Pub. Date: Nov. 11, 2010

(54) METHOD FOR DETECTING RALSTONIA SOLANACEARUM RACE 3 BIOVAR 2

(76) Inventors: Christian Boucher, Auzielle (FR); Mounira Elbaz, Tunis (TN); Stéphane Genin, Donneville (FR); Alice Guidot, Caluire (FR); Philippe Prior, Ravine Des Cabris (FR)

Correspondence Address: STITES & HARBISON PLLC, 1199 NORTH FAIRFAX STREET, SUITE 900, ALEXANDRIA, VA, 22314, US

(21) Appl. No.: 12/681,136

(22) PCT Filed: Oct. 3, 2008
(86) PCT No.: PCT/EP08/63298

§ 371 Date: Jun. 18, 2010

Related U.S. Application Data
(60) Provisional application No. 60/998,050, filed on Oct. 5, 2007.

(30) Foreign Application Priority Data
Oct. 5, 2007 (EP) 07291213.2

(51) Int. Cl. C40B 30/00; C12Q 1/68; C07H 21/00; C40B 40/06

(52) U.S. Cl. 506/7; 435/6; 536/22.1; 506/16

(57) ABSTRACT

The invention concerns a method for the detection of Ralstonia solanacearum race 3 biovar 2 strains in a medium, comprising the determination of the presence or the absence in a sample of the medium, of: (i) at least one first nucleic acid target having a sequence selected from the group constituted of SEQ ID NO: 1-49, complementary sequences thereof, and homologous sequences thereof, or (ii) at least one fragment of said first target nucleic acid, wherein said fragment is not constituted of or comprised in a sequence selected from the group constituted of SEQ ID NO: 111-140; whereby, if said first nucleic acid target or fragment thereof is present in the sample, it is determined that Ralstonia solanacearum race 3 biovar 2 strain is present in the medium.

The present application claims the benefit of U.S. Provisional Patent Application No. 60/998,050, filed Oct. 5, 2007.

### FIELD OF THE INVENTION

The present invention relates to a method for detecting race 3 biovar 2 Ralstonia solanacearum strains.

### BACKGROUND OF THE INVENTION

Ralstonia solanacearum is a Gram-negative soil-borne plant pathogen with thousands of distinct strains in a heterogeneous species complex. Ralstonia solanacearum causes bacterial wilt, which is globally distributed and economically destructive. It is thus considered the single most destructive bacterial plant disease because of its unusually broad host range. The bacterium attacks plants in over 200 different families, including dicots and monocots, and annual plants as well as trees. Economically important crop hosts include: tomato, potato, pepper, tobacco, peanut, ornamentals, banana, plantain, and eucalyptus. Losses due to bacterial wilt are known to be enormous but cannot be accurately estimated because of its large but undocumented impact on subsistence agriculture and because planting of wilt-susceptible crops have been abandoned altogether in many parts of the world. Specifically, economic losses to the potato industry in the world have been estimated 950 million US $ and the potato brown rot strain of Ralstonia solanacearum was listed in the USA as a Bioterrorism Select Agent (BSA).

The pathogen usually enters host roots from the soil, multiplies in the root cortex, and colonizes the xylem vessels, so the bacteria spread rapidly throughout the host via the plant's own vascular system. Symptoms vary according to host, but rapid wilting and death are the common elements.

To unravel the genetic diversity within this species complex, a new classification scheme was proposed that distinguishes four phylotypes:
phylotype I corresponds to the “Asiaticum” division 1 of Cook et al (1989) and contains strains belonging to biovars 3 (GMI1000), 4 and 5;
phylotype II corresponds to the “Americanum” division 2 of Cook and contains strains belonging to race 1 biovar 1, race 2 biovar 1 (e.g. Moko disease-causing strains such as Molk2), race 3 biovar 2 (e.g. IPO1609 and UW551) and race 3 biovar 2T strains;
phylotype III contains strains from Africa and the Indian Ocean, which belong to biovars 1, 2 and 2T;
phylotype IV is reported highly heterogeneous; it contains strains from Indonesia, some strains from Japan, and a single strain from Australia, belonging to biovars 1, 2 and 2T; phylotype IV also contains the closely related species Ralstonia syzigii and the blood disease bacterium (BDB).

Each phylotype can be further subdivided into sequevars based on differences in partial sequence of the endoglucanase gene (egl). The phylotyping scheme is broadly consistent with the former phenotypic and molecular typing schemes (Fegan and Prior, 2005: Prior and Fegan 2005), and adds valuable information about the geographical origin and in some cases the pathogenicity of strains. It is believed that, after the race/biovar classification, the phylotype classification scheme is to become the core organizing principle for assigning a particular strain a phylogenetic position with a predictive value on potential host range (Fegan and Prior, 2006; Wicker et al., 2007). Whole genome sequencing was decisive in unravelling the broad genetic diversity encompassed within this organism with unusually broad host range. Thus a metagenomic microarray from sequence data of a broad host range tomato phylotype I strain (GMI1000) has been developed which has enabled comparative genomic hybridizations demonstrating that a third of the Ralstonia solanacearum genome is constituted of variable genes probably acquired by horizontal gene transfers. The distribution of variable genes between strains is related to the phylotype classification (Guidot et al, 2007).

Recent phylogenetic evidences indicated that strains that fit with the definition of the potato brown rot agent were placed into the phylotype IIB sequevar 1 and 2, i.e. the biovar 2 Andean strains of Ralstonia solanacearum historically known as race 3 biovar 2. These strains are highly pathogenic to potato and adapted to low temperatures. Some strains in that group were also reported to carry an enlarged host range including tomato, and Geranium rosa (Carmeille at al., 2006).

Given, the important economic impact of potato brown rot, it is highly desirable to develop methods for specifically detecting race 3 biovar 2 Ralstonia solanacearum strains.

Thus, WO 2004/042016 relates to real-time PCR primers and probes useful for the detection of such strains. The primers and probes enable the detection of a nucleic acid sequence which is specific for race 3 biovar 2 Ralstonia solanacearum strains.

However, it appears that the nucleic acid sequence detected by the primers and probes of WO 2004/042016 is that of a mobile genetic element, since this sequence encodes part of a protein homologous to the Mu-like phage of the ORF35 of the B3 bacteriophage from Pseudomonas aeruginosa (Accession Q7AX27).

In general, mobile genetic elements should be avoided in the frame of the specific detection of a pathogenic microorganism, since particular strains of the pathogenic microorganism could lack the element or, conversely, other unrelated microorganisms could harbour the element, thereby yielding respectively false negative and false positive results.

Accordingly, it is an object of the present invention to develop a method for detecting race 3 biovar 2 Ralstonia solanacearum strains which involves the detection of specific nucleic acid sequences which do not belong to mobile genetic elements.

### DESCRIPTION OF THE INVENTION

The present invention arises from the identification, by the inventors, of genomic portions of Ralstonia solanacearum which are specific of the race 3 biovar 2 strains of Ralstonia solanacearum.

Thus the present invention relates to a method for the detection of Ralstonia solanacearum race 3 biovar 2 in a medium, comprising the determination of the presence or the absence in a sample of the medium, of:

(i) at least one first nucleic acid target having a sequence selected from the group constituted of SEQ ID NO: 1-49, complementary sequences thereof, and homologous sequences thereof, or
(ii) at least one fragment of said first nucleic acid target, wherein said fragment is preferably not constituted of or comprised in a sequence selected from the group constituted of SEQ ID NO: 111-140 and more preferably not constituted of or comprised in a sequence selected from the group constituted of SEQ ID NO: 111-140, SEQ ID NO: 50-53, SEQ ID NO: 61-65, SEQ ID NO: 71-72, SEQ ID NO: 81-90, SEQ ID NO: 98-105, SEQ ID NO: 107 and SEQ ID NO: 109; whereby, if said first nucleic acid target or fragment thereof is present in the sample, it is determined that Ralstonia solanacearum race 3 biovar 2 is present in the medium.

The present invention also relates to the use of:

(i) at least one first nucleic acid target having a sequence selected from the group constituted of SEQ ID NO: 1-49, complementary sequences thereof, and homologous sequences thereof, or
(ii) at least one fragment of said first target nucleic acid, wherein said fragment is preferably not constituted of or comprised in a sequence selected from the group constituted of SEQ ID NO: 111-140 and more preferably not constituted of or comprised in a sequence selected from the group constituted of SEQ ID NO: 111-140, SEQ ID NO: 50-53, SEQ ID NO: 61-65, SEQ ID NO: 71-72, SEQ ID NO: 81-90, SEQ ID NO: 98-105, SEQ ID NO: 107 and SEQ ID NO: 109; for detecting Ralstonia solanacearum race 3 biovar 2.

As intended herein the expression “SEQ ID NO: X-Y” (where Y═X+n) represents all the sequences represented by SEQ ID NO: X to SEQ ID NO: Y, that is SEQ ID NO: X, SEQ ID NO: X+1, SEQ ID NO: X+2 . . . SEQ ID NO: X+n−1, and SEQ ID NO: Y.

As intended herein the “medium” can be of natural or synthetic origin. Where the medium is of synthetic origin, it can in particular be a solid or liquid bacterial culture medium. Where the medium is of natural origin, it can notably be water, soil, or a biological tissue, in particular a plant tissue. Preferably, the medium is selected from the group constituted of a potato tissue, a tomato tissue, and a geranium tissue. These plant tissues are particularly prone to infection by Ralstonia solanacearum race 3 biovar 2. However, it is particularly preferred that the medium is a potato tissue, since Ralstonia solanacearum race 3 biovar 2 is the causative agent of potato brown rot. Preferably, the potato tissue should be the tuber, which constitutes the potato tissue most likely to contain Ralstonia solanacearum race 3 biovar 2.

As intended herein a “sample” relates to a portion of the medium liable to contain nucleic acids. In particular, it is preferred that the sample is obtained from the medium by a nucleic acid extraction. Numerous methods exist, which are well known to one of skill in the art, for extracting nucleic acids from a medium, in particular from a plant tissue.

As intended herein “nucleic acid” preferably relates to RNA or DNA.

As intended herein “determination” of the presence or the absence of a nucleic acid in a sample relates to the detection of the full length nucleic acid in itself, but also to fragments of the nucleic acid. The present inventors have shown that genetic material transfers which have occurred in the course of evolution and which have structured the genome of Ralstonia solanacearum race 3 biovar 2 concerned genomic patches often involving several genes. These genomic patches correspond to SEQ ID NO: 1-49. Accordingly, the detection of fragments of these genomic patches is generally indicative of the presence of the nucleic acid as a whole. Besides, even if portions of SEQ ID NO: 1-49 should be lacking in a particular race 3 biovar 2 strain of Ralstonia solanacearum, it will be clear to one of skill in the art that the detection of fragments of conserved portions of SEQ ID NO: 1-49 is sufficient to unambiguously determine the presence of Ralstonia solanacearum race 3 biovar 2.

Thus, as intended herein a “fragment” should be of a length such that it can be considered by one of skill in the art that it presents essentially no identity with nucleic acids of the same length which can be found in Ralstonia solanacearum strains which are not of race 3 biovar 2. Preferably, fragments according to the invention comprise at least 9 nucleotides, more preferably at least 15 nucleotides and most preferably at least 18 nucleotides.

Exemplary fragments of the above-mentioned genomic patches notably encompass gene and inter-gene sequences SEQ ID NO: 50-110 and SEQ ID NO: 111-140.

Some short portions of SEQ ID NO: 1-49 are of a known length but of undetermined sequence. It will be apparent to one of skill in the art that the above-defined fragments of SEQ ID NO: 1-49 should preferably not be constituted of these short portions. Besides, it should be noted that SEQ ID NO: 2, 3, 4, 6, 9 and 10 are respectively identical to SEQ ID NO: 445, 446, 447, 448, 449 and 450, except for some undetermined portions of SEQ ID NO: 445-450 which are determined in SEQ ID NO: 2, 3, 4, 6, 9 and 10. Accordingly, where appropriate, SEQ ID NO: 2, 3, 4, 6, 9 and 10 are respectively interchangeable with SEQ ID NO: 445, 446, 447, 448, 449 and 450.

The inventors have also found that the genomic patches they identified comprised sequence derived from mobile genetic elements. These mobile genetic elements are represented by SEQ ID NO: 111-140. Other putative mobile genetic elements are represented by SEQ ID NO: 50-53, SEQ ID NO: 61-65, SEQ ID NO: 71-72, SEQ ID NO: 81-90, SEQ ID NO: 98-105, SEQ ID NO: 107 and SEQ ID NO: 109. Accordingly, the method and use according to the invention should preferably not determine the presence or absence of these mobile genetic elements or fragments thereof as first target nucleic acids.

As intended herein “homologous sequences” relate to natural variants of sequences SEQ ID NO: 1-49, which can be found in strains of Ralstonia solanacearum race 3 biovar 2. Thus the homologous sequences notably encompass:
sequences which are derived from sequences SEQ ID NO: 1-49 by insertion, deletion or substitution of at least one nucleotide;
sequences which are liable to hybridize under stringent conditions to the complementary sequences of SEQ ID NO: 1-49;
sequences which present at least 95% identity to sequences SEQ ID NO: 1-49;
   provided the homologous sequences are specific to Ralstonia solanacearum race 3 biovar 2.

In a particular embodiment of the above-defined method and use, the determination comprises at least one step of hybridization of the nucleic acid target or fragment thereof with a probe or a primer. Preferably, the probe or primer is a fragment of nucleic acid having a sequence selected from the group constituted of SEQ ID NO: 1-49, homologous sequences thereof, and complementary sequences thereof. More preferably, the probe or primer is selected from the group constituted of SEQ ID NO: 141-386.

In another particular embodiment of the above-defined method and use, the determination comprises at least one step of nucleic acid amplification.

Preferably, where the determination comprises a step of nucleic acid amplification, the determination is implemented by a method selected from PCR and NASBA. NASBA is notably described in Compton (1991) Nature 350:91-92.

Also preferably, the determination is implemented by a method selected from Southern blotting, Northern blotting, dot blots, and nucleic acid micro or macro-array hybridization,

In a particular embodiment of the invention the above-defined method and use also comprise the determination of the presence or the absence in a sample of the medium, of:

(i) at least one second nucleic acid target having a sequence selected from the group constituted of SEQ ID NO: 111-140 and/or SEQ ID NO: 50-53, SEQ ID NO: 61-65, SEQ ID NO: 71-72, SEQ ID NO: 81-90, SEQ ID NO: 98-105, SEQ ID NO: 107 and SEQ ID NO: 109, complementary sequences thereof, and homologous sequences thereof, or
(ii) at least one fragment of said second nucleic acid target.

Indeed, since the at least one first target sequence is preferably not a mobile genetic elements, the determination of the presence or absence of nucleic acids corresponding to mobile genetic elements can be effected in a further step.

The present invention also relates to a nucleic acid having a sequence selected from the group constituted of SEQ ID NO: 1-22, SEQ ID NO: 50-110, SEQ ID NO: 111-140, SEQ ID NO: 141-246, and SEQ ID NO: 247-386 and their complementary sequences.

The present invention also relates to a nucleic acid micro or macro-array comprising a plurality of nucleic acid probes arranged onto a solid support, wherein the nucleic acid probes are fragments of nucleic acids having sequences selected from the group constituted of SEQ ID NO: 1-49, complementary sequences thereof, and homologous sequences thereof, provided that preferably at least one of the nucleic acid probes is not a fragment of a nucleic acid having a sequence selected from the group consisting of SEQ ID NO: 111-140 and more preferably at least one of the nucleic acid probes is not a fragment of a nucleic acid having a sequence selected from the group consisting of SEQ ID NO: 111-140, SEQ ID NO: 50-53, SEQ ID NO: 61-65, SEQ ID NO: 71-72, SEQ ID NO: 81-90, SEQ ID NO: 98-105, SEQ ID NO: 107 and SEQ ID NO: 109.

Preferably, in the above-defined nucleic acid micro-array, the nucleic acid probes comprise SEQ ID NO: 247-386.

The present invention also relates to a kit intended for the detection of Ralstonia solanacearum race 3 biovar 2 in a medium, comprising at least:
two primers suitable to amplify a portion of a nucleic acid having a sequence selected from the group constituted of SEQ ID NO: 1-49 and complementary sequences thereof, provided that said portion is preferably not comprised in a nucleic acid having a sequence selected from the group consisting of SEQ ID NO: 111-140 and complementary sequences thereof, and more preferably not comprised in a nucleic acid having a sequence selected from the group consisting of SEQ ID NO: 111-140, SEQ ID NO: 50-53, SEQ ID NO: 61-65, SEQ ID NO: 71-72, SEQ ID NO: 81-90, SEQ ID NO: 98-105, SEQ ID NO: 107 and SEQ ID NO: 109, and complementary sequences thereof;
optionally one detectable nucleic acid probe suitable to hybridize to said amplified portion.

### EXAMPLE

### Materials and Methods:

Bacterial Strains Used in this Study

Table 1 provides the list of the 14 race 3 biovar 2 strains and 45 non-race 3 biovar 2 strains used herein together with their geographical origin, host of origin. The 14 race 3 biovar 2 strains corresponded to phylotype IIB, sequevar 1 based on the classification scheme proposed by Fegan & Prior (2005).

Microarray:

A 12× draft of the genome sequence from strain IPO1609 (a race 3 biovar 2 strain accessible from the Collection Française de Bactéries Phytopathogènes under accession number CFBP 6926) has been established in collaboration with Genoscope and annotated by the inventors. The deduced amino acid sequence of the genes thus identified was compared with the amino acid sequence of the genes previously identified in strain GMI1000 (Salanoubat et al., 2002). All the genes from IPO1609 that did not have a homolog (less than 40% identity over at least 80% of both the query and subject sequences) will be further referred to as “potential IPO1609-specific genes”. For each of these genes a specific representative 70 mer-oligonucleotide was designed using ROSO algorithm (Reymond, 2004). These oligonucleotides were chosen as having no significant homology with any part of the genome of strain GMI1000 and were used to generate a microarray as previously reported for the construction of the GMI1000 microarray (Occhialini et al., 2005). The “IPO1609 partial microarray” thus generated also includes additional oligonucleotides representative of the IPO1609 allelic variants for a limited number of genes conserved but significantly divergent between strains GMI1000 and IPO1609 (mostly including type III-secretion dependant pathogenicity effectors) therefore permitting the distinction of the two allelic forms of a given gene. Finally, a limited number of oligonucleotides representative of particular intergenic regions were also included on the microarray. The sequence of each individual oligonucleotide spotted on the microarray has been deposited at Agence de Protection des Programmes (APP) under accession number IDDN.FR.001.300024.000.R.P.2006.000.10300.

Genomic DNA Extraction, DNA Labelling, Microarray Hybridization, Hybridization Signal Measurement and Analysis.

These have been performed as previously described (Guidot et al., 2007) except that standard control DNA used for all genome hybridizations experiments which were performed consisted of an equimolar combination of the genomic DNA from three sequenced strains GMI1000, IPO1609 and Molk2 (IPO1609 and Molk2 genomes being unpublished) (Molk2 is accessible from the Collection Française de Bactéries Phytopathogènes under accession number CFBP 6925 and from the ATCC under accession number BAA-1115). Analysis was conducted as previously described using ImaGene and GeneShight (BioDiscovery) softwares. A gene was considered as being absent in the tested strain when the base 2 logarithm of the ratio of the normalized hybridization signal of the tested strain over the normalized hybridization signal with the control DNA was lower to −1.

PCR Validation

The list of candidate race 3 biovar 2-specific genes deduced from comparative genomic hybridizations (CGH) experiments was checked by PCR amplification. The PCR primers used are given in Table 2. The primers were designed to amplify one genomic fragment from each gene. When possible, one of the two primers for each gene was designed inside the oligonucleotide spotted on the microarray. PCR were conducted in 25 μl reaction mixture containing 10 ng DNA from each tested strain, 25 pmol of each primer (L/R), 1.5 mM MgCl₂, 200 μM of each four dNTP, 0.5 U of Red Gold Star Taq DNA polymerase (Eurogentec) and the buffer supplied by the manufacturer. PCR amplifications were performed as follows: an initial denaturation step at 96° C. for 5 min followed by 30 cycles of 94° C. for 15 s., 59° C. for 30 s., and 72° C. for 30 s., with a final ex tension step of 72° C. for 10 min. Negative (PCR reaction mixture without DNA) and positive (IPO1609 DNA) controls were included in each experiment. The multiplex PCR described by Fegan & Prior (2005) for the Ralstonia solanacearum phylotype identification was conducted on each tested DNA as an amplification positive control.

In Silico Comparison of Strains UW551 with IPO1609, GMI1000 and Molk2 Strains

The deduced amino acid sequence for each predicted gene from strain UW551 was BlastX compared to the genomic sequence of strain IPO1609 in order to identify the predicted proteins of UW551 that have no counterpart covering at least 80% of their length. These proteins designated as “potential UW551-specific proteins” were then compared with BlastX to the genomic sequence of strain GMI1000 and Molk2. All the proteins that did not have a counterpart covering at least 80% of the length of the query sequence with at least 40% identity where then individually compared using BlastP with the predicted proteins from strains GMI1000 and Molk2. This was performed in order to eliminate false candidates that could remain in the list due to frameshift in the nucleotide sequence.

### Results:

Analysis of the Distribution of “Potential IPO1609-Specific Genes” Among a Collection of Strains Representative of the Diversity of Ralstonia Solanacearum.

Comparative genomic hybridizations (CGH) on the microarray have been performed to compare the lists of genes between 11 race 3 biovar 2 strains and 20 non-race 3 biovar 2 strains (Table 1). This analysis identifies a set of 137 oligonucleotides which are present in at least 10 of the 11 race 3 biovar 2 strains and absent from at least 19 of the 20 non-race 3 biovar 2 strains. These oligonucleotides were representative of 79 genes and 38 intergenic regions from the IPO1609 genome. These genes and intergenes therefore were considered as “candidate race 3 biovar 2 specific genomic regions”. A large proportion of these regions forms clusters in the IPO1609 genome and sometime a few genes mapping within these clusters were not found in the list of thus defined “candidate race 3 biovar 2 specific regions”. Based on
i) the known mosaic structure of Ralstonia solanacearum genome that suggests that sets of genes could have been acquired through horizontal gene transfers or lost through deletions (Salanoubat et al., 2002; Guidot et al., 2007),
ii) on the consideration that some genes might be missing from this list due to the fact that hybridization data are missing in a limited number of cases, and
iii) taking into account the possibility that certain genes that score next to the cutoff value could have been miss-categorized,
   the inventors included into the list of the “race 3 biovar 2 specific regions” all the sets of 1 or 2 contiguous genes that were not originally detected as being race 3 biovar 2-specific but that are located within a race 3 biovar 2-specific gene cluster. Based on these criteria 34 additional genes were included into the list of race 3 biovar 2-specific genes. These additional genes are identified with a star (*) in Table 3.

When compared using BlastP with the sequence of the predicted proteins from strains GMI1000 and Molk2, 5 of these genes were found to have a counterpart in at least one of these strains and were therefore eliminated from the final list of race 3 biovar 2-specific genes presented in Table 3. This final list includes a total of 151 genes or intergenic regions organized in 18 clusters and 15 additional individual genes or intergenic regions.

Validation of Candidate Race 3 Biovar 2-Specific Genomic Regions.

The list of candidate race 3 biovar 2-specific genomic regions given in Table 3 has been established based on the analysis of a limited number of strains. Therefore the inventors validated this list on a larger collection of strains representative of the diversity found in Ralstonia solanacearum.

A total of eight race 3 biovar 2-strains (among-which six were also used for CGH experiments) and 32 non-race 3 biovar 2 strains (seven of which were also used for CGH experiments) were used for this validation (Table 1). This validation was conducted by PCR amplification of one genomic fragment from each candidate race 3 biovar 2-specific region. All tested DNA could be amplified using the multiplex PCR for the Ralstonia solanacearum phylotype identification as described by Fegan and Prior (2005) therefore confirming that the strains tested actually correspond to Ralstonia solanacearum isolates.

Each genomic region that gave a positive amplification from non-race 3 biovar 2 strains or a negative amplification from race 3 biovar 2 strains was excluded from the list of “race 3 biovar 2 specific genomic regions”. The results validated the specificity of eleven gene clusters and eleven individual genes or intergenic regions (Table 4). Among these genes, 27 were predicted to be parts of mobile genetic elements (bacteriophage or insertion sequences).

Identification of Additional Candidate Race 3 Biovar 2-Specific Genes in Strain UW551

A genomic draft for the race 3 biovar 2 virulent isolate UW551 is publicly available (Gabriel et al. 2006). Because strain IPO1609 probably harbours a genome deletion that significantly impairs its virulence compared to UW551, the inventors decided to compare the genome sequences for these two strains. In doing this 328 predicted genes from UW551 that had no counterpart in IPO1609 were identified. Based on the same criteria, in silico comparison of these 328 genes with the genome sequence of strain Molk2 and GMI1000 reduced this set to 94 UW551 specific genes. Comparison of these 94 predicted proteins using BlastP with the proteins of GMI1000 and Molk2 eliminated 67 proteins that were not previously detected as having a counterpart in GMI1000 or Molk2 due to the presence of a potential frameshift mutation in the corresponding genes, therefore leading to a final list of the 27 UW551 specific genes shown in Table 5.

### List of Tables

Table 1: List of Ralstonia solanacearum strains used in this study (ND not determined, NO no object)

Table 2: List of the oligonucleotides used for PCR amplification of candidate race 3 biovar 2 specific genes. (a) Sequences in bold correspond to part of the oligonucleotide spotted on the microarray

Table 3: List of candidate race 3 biovar 2-specific genomic regions.*Added genes correspond to the genes that have been included into the list based on their location within clusters of race 3 biovar 2 specific gene clusters.

Table 4: Validated race 3 biovar 2-specific genomic regions together with sequences of the PCR primers used for gene amplification. Black boxes highlight genes from mobile genetic elements (bacteriophage or insertion sequences). Underlined boxes highlight genes from putative mobile elements.

Table 5: List of additional candidate race 3 biovar 2-specific genes identified from strain UW551.

TABLE 1

| | | | | | CGH on | PCR |
|---|---|---|---|---|---|---|
| Strain ID | Host | Origin | Race | Biovar | microarray | verification |
| IPO1609 | Potato | Netherlands | 3 | 2 | Yes | control+ |
| JT516 | Potato | Reunion Is. | 3 | 2 | Yes | Yes |
| CMR34 | Tomato | Cameroon | 3 | 2 | Yes | Yes |
| RE | Potato | Uruguay | 3 | 2 | Yes | Yes |
| AP31H | Potato | Uruguay | 3 | 2 | Yes | |
| AP42H | Potato | Uruguay | 3 | 2 | Yes | Yes |
| TB1H | Potato | Uruguay | 3 | 2 | Yes | |
| TB2H | Potato | Uruguay | 3 | 2 | Yes | |
| TC1H | Potato | Uruguay | 3 | 2 | Yes | Yes |
| TB10 | Potato | Uruguay | 3 | 2 | Yes | |
| ETAC | Potato | Uruguay | 3 | 2 | Yes | |
| RM | Potato | Uruguay | 3 | 2 | Yes | Yes |
| PSS525 | Potato | Taiwan | 3 | 2 | | Yes |
| CMR24 | Potato | Cameroon | 3 | 2 | | Yes |
| CIP10 | Potato | Peru | 3 | 2T | | Yes |
| NCPPB3987 | Potato | Brazil | 3 | 2T | | Yes |
| Molk2 | Banana | Philippines | 2 | 1 | Yes | |
| CIP418 | Peanut | Indonesia | 2 | 1 | Yes | |
| UW9 | Heliconia | Costa Rica | 2 | 1 | | Yes |
| CFBP1183 | Heliconia | Costa Rica | 2 | 1 | | Yes |
| UW163 | Plantain | Peru | 2 | 1 | Yes | |
| Ant75 | Heliconia | Martinique | NO | 1 | Yes | |
| Ant80 | Anthurium | Martinique | NO | 1 | Yes | |
| Ant307 | Anthurium | Martinique | NO | 1 | Yes | |
| JY200 | Anthurium | Martinique | NO | 1 | Yes | |
| JY201 | Anthurium | Martinique | NO | 1 | Yes | |
| Ant1121 | Anthurium | Martinique | NO | 1 | Yes | Yes |
| CFBP6797 | Solanum | Martinique | NO | 1 | | Yes |
| CFBP7014 | Anthurium | Trinidad | NO | 1 | | Yes |
| TOM = T1 | Tomato | Uruguay | 2 | 1 | Yes | Yes |
| B34 | Banana | Brazil | 2 | 1 | Yes | Yes |
| A3909 | Heliconia | Hawai | 2 | 1 | Yes | Yes |
| CIP239 | Potato | Brazil | 1 | 1 | | Yes |
| CIP301 | Potato | Peru | 1 | 1 | | Yes |
| CFBP2957 | Tomato | Martinique | 1 | 1 | | Yes |
| CMR39 | Tomato | Cameroon | 1 | 1 | | Yes |
| ICMP7963 | Potato | Kenya | 1 | 1 | | Yes |
| CFBP6942 | Solanum | Cameroon | NO | 2T | Yes | Yes |
| CFBP6941 | Tomato | Cameroon | NO | 2T | Yes | |
| CMR43 | Potato | Cameroon | NO | 2T | | Yes |
| CIP358 | Potato | Cameroon | NO | 2T | | Yes |
| CFBP3059 | Eggplant | Burkina | NO | 1 | Yes | Yes |
| | | Faso | | | | |
| CMR66 | Solanum | Cameroon | NO | 2T | | Yes |
| JT525 | Pelargonium | Reunion Is. | NO | 1 | | Yes |
| JT528 | Potato | Reunion Is. | NO | 1 | | Yes |
| J25 | Potato | Kenya | NO | 2T | | Yes |
| NCPPB332 | Potato | Zimbabwe | NO | 1 | | Yes |
| GMI1000 | Tomato | Guyana | 1 | 3 | Yes | |
| CMR134 | Solanum | Cameroon | 1 | 3 | Yes | |
| CIP365 | Potato | Philippines | 1 | 3 | | Yes |
| R288 | Morus alba | China | 1 | 5 | | Yes |
| PSS358 | Tomato | Taiwan | 1 | 3 | Yes | |
| PSS190 | Tomato | Taiwan | 1 | 3 | Yes | |
| PSS219 | Tomato | Taiwan | 1 | 3 | | Yes |
| ACH732 | Tomato | Australia | NO | 2 | | Yes |
| Psi07 | Tomato | Indonesia | NO | 2T | | Yes |
| Psi36 | Tomato | Indonesia | NO | 2T | | Yes |
| MAFF301558 | Potato | Japan | NO | 2T | | Yes |
| R. syzygii | Clove | Indonesia | NO | NO | Yes | Yes |
| R28 | | | | | | |

TABLE 2

| | | | Product | SEQ ID |
|---|---|---|---|---|
| Gene name | Primer name | Primer sequence (a) | size (bp) | NO: |
| IPO_00030 | IPO_00030_L | ACTTGGAGAGATTTACGGAGGAG | 200 | 387 |
| | IPO_00030_R | AAGCAAACGAGATAAGGGAGAAC | | 388 |
| IPO_00031 | IPO_00031_L | AAACACAATTCACCTTCCTGATG | 185 | 389 |
| | IPO_00031_R | GGCCACTAGACTTTCCAGTGAT | | 390 |
| IPO_00034 | IPO_00034_L | AGCTTACCTGCTGTTCGACATCT | 818 | 391 |
| | IPO_00034_R | GTTCTTCGTGATAGCGGAGACT | | 392 |
| IPO_00043 | IPO_00043_L | CTGAATTCGAAAAGGATAGAGCA | 206 | 141 |
| | IPO_00043_R | CTCGACAAACTCTTGCAACTGAC | | 142 |
| IPO_00044 | IPO_00044_L | CTATGCAGAAGCGTTGCTTGTT | 191 | 143 |
| | IPO_00044_R | CTTTAGCGAGCACAAGATTGAGT | | 144 |
| IPO_00045 | IPO_00045_L | GAGATCGTTGGAAACATCAAGAC | 165 | 145 |
| | IPO_00045_R | GTGAACCACTATTGCCGGTATC | | 146 |
| IPO_00233 | IPO_00233_L | AGAACTGCCAAGTTCGACTACCT | 207 | 147 |
| | IPO_00233_R | CATTCCAACGTTCAGATGGTTAT | | 148 |
| IPO_00234 | IPO_00234_L | GCAGAAAAGATATCCCCTGCAC | 214 | 149 |
| | IPO_00234_R | TTCGAGTACAAATGTAGGCTTCC | | 150 |
| IPO_00875 | IPO_00875_L | GATCAGATGGAGCAAAGAACACT | 221 | 151 |
| | IPO_00875_R | TATTGAAACTCTTCACGGGTCAT | | 152 |
| IPO_00876 | IPO_00876_L | TTTCGACCAAGAAAAGCATAGAG | 238 | 153 |
| | IPO_00876_R | ATTTCTGTGCCCACTACGAACTA | | 154 |
| IPO_00877 | IPO_00877_L | TGGTGTTCTAACTGTGGAAGGTT | 163 | 155 |
| | IPO_00877_R | TACCGCCAGTCATATCAGTTCTT | | 156 |
| IPO_00878 | IPO_00878_L | ATTAGACTGATCAAGGCATGGAA | 152 | 157 |
| | IPO_00878_R | CCTTCATTATTGAGACGGTCAAG | | 158 |
| IPO_00879 | IPO_00879_L | ATGTTTGTGCTACTGGTCAGTCC | 223 | 159 |
| | IPO_00879_R | CCTTCACTTGCAGATAATGGAAC | | 160 |
| IPO_00881 | IPO_00881_L | AAAGAAGCTCAAGGAGATCAAGG | 201 | 161 |
| | IPO_00881_R | AACAGCAGGTTGTGATACTGCAT | | 162 |
| IPO_01030 | IPO_01030_L | TATGAATGGGTTGATAGCGTTCT | 165 | 393 |
| | IPO_01030_R | CCATATCCACCGATAAACAACAT | | 394 |
| IPO_01058 | IPO_01058_L | CGAGCTCATCGTTATCGACAT | 140 | 163 |
| | IPO_01058_R | AAGCTCTTGGACTAGGACGATCT | | 164 |
| IPO_01131 | IPO_01131_L | CACGATATGACCACGATCAACTA | 195 | 395 |
| | IPO_01131_R | GTAGACACGAATCACGTCTCCAT | | 396 |
| IPO_01132 | IPO_01132_L | ACATCAACGACCCTTACTGTCC | 147 | 397 |
| | IPO_01132_R | GACCATAGTCATCGCTGCTTAAC | | 398 |
| IPO_01137 | IPO_01137-1_L | CTATCCCGCAGAAGGTATTCAAC | 186 | 399 |
| | IPO_01137-1_R | AGTCATAGGCGTCTCGGTACTT | | 400 |
| | IPO_01137-2_L | ACACTCTGTTCACCAAGTACGG | 217 | 401 |
| | IPO_01137-2_R | CTTTGAAACTGGAGGAACAGCTT | | 402 |
| IPO_01259 | IPO_01259_L | TGAAATGCTCAAAGACAAACAGA | 235 | 165 |
| | IPO_01259_R | ATCGTACAGGTCATTGCCAAAT | | 166 |
| IPO_01260 | IPO_01260_L | TACAACCTGAAGAGGATCTCGAA | 225 | 167 |
| | IPO_01260_R | AAAGCCGGTCATAGAGGACATAG | | 168 |
| IPO_01311 | IPO_01311_L | CAACCAGACCATCTACAAGATCC | 165 | 169 |
| | IPO_01311_R | GCTTCATACTCAAATCGAACACC | | 170 |
| IPO_01312 | IPO_01312_L | AACTCCAACTTGCTTGACTGTTC | 208 | 171 |
| | IPO_01312_R | GGATGAACTTCGTTCGATTGAG | | 172 |
| IPO_01314 | IPO_01314_L | GAAGCTCGGTGATATCGAAAC | 287 | 173 |
| | IPO_01314_R | GGTGATCGCTGTCGATAATTT | | 174 |
| IPO_01362 | IPO_01362_L | AATTGGGTATACGTGATCTGTGG | 280 | 243 |
| | IPO_01362_R | TCGGGTAAGACGAAGCTGACTA | | 244 |
| IPO_02090 | IPO_02090_L | CAATAGAAATTGCCGAGGTGATA | 171 | 239 |
| | IPO_02090_R | CCTTGATAAGGATGTTCAACGAC | | 240 |
| IPO_02092 | IPO_02092_L | AACACTCAAAAGCTGACCATCAT | 151 | 403 |
| | IPO_02092_R | CAACCTTGATCTGTTCGGAGAC | | 404 |
| IPO_02095 | IPO_02095_L | GTTGCAATGCTGGTTTCCAAG | 157 | 405 |
| | IPO_02095_R | GTCATGGACGAGAAATCGATAC | | 406 |
| IPO_02097 | IPO_02097_L | CTCAGAGGATCTGTTCATCGACT | 160 | 407 |
| | IPO_02097_R | GTTGAAGACGCCGAAGAAAAA | | 408 |
| IPO_02098 | IPO_02098_L | ACGAGATTCCTGAAGCTGAGGT | 192 | 409 |
| | IPO_02098_R | CTTGCAGAACCTGACACATGA | | 410 |
| IPO_02102 | IPO_02102_L | GTATCAGAAAGGCCAGCTACACA | 207 | 175 |
| | IPO_02102_R | CTGATTGCCAATATTCGATTCTC | | 176 |
| IPO_02140 | IPO_02140_L | AAGGGCAATGGCTTTTTCTGT | 153 | 177 |
| | IPO_02140_R | GAGACTGATAAATCAGCGTTTCC | | 178 |
| IPO_02141 | IPO_02141_L | GCTTTCTACGTCGCCTCAGTAT | 225 | 179 |
| | IPO_02141_R | GTAACGGTCTGATTCTTGAGGTTT | | 180 |
| IPO_02142 | IPO_02142_L | CCACTACCCCTGTTTCCATTC | 245 | 181 |
| | IPO_02142_R | AAACCTGTAGTCGTCGTCCTTG | | 182 |
| IPO_02143 | IPO_02143_L | AGACGTTGGACAACATCAACC | 189 | 183 |
| | IPO_02143_R | AGTTCTGTTCGTCGTCGTGAAT | | 184 |
| IPO_02144 | IPO_02144_L | CCAGTATTCCAAGACGCTATCC | 189 | 185 |
| | IPO_02144_R | AACGGATACAGCAGCAGGTTT | | 186 |
| IPO_02145 | IPO_02145_L | ATACCCAGCGCGTATTCCAA | 164 | 187 |
| | IPO_02145_R | GTTGAGGCAACACCAGACAG | | 188 |
| IPO_02146 | IPO_02146_L | CTTGAGAAAGCTTTTGGTGAAGA | 166 | 189 |
| | IPO_02146_R | CTTTGAGACCTTCCCAGGCTAA | | 190 |
| IPO_02147 | IPO_02147_L | TAAGAGCAGGCTATGGACAACAT | 209 | 191 |
| | IPO_02147_R | AATACCAGCACACAGAAGGTCAG | | 192 |
| IPO_02148 | IPO_02148_L | CTGATTTCCATGTACCTGCATC | 235 | 193 |
| | IPO_02148_R | ACATCAGCACGTTCTTGTAGAGC | | 194 |
| IPO_02149 | IPO_02149_L | GACGATGAGTTGCTGAAGTACC | 237 | 195 |
| | IPO_02149_R | TGAAGGTAATGGTCACAGCTTTT | | 196 |
| IPO_02150 | IPO_02150_L | ATCCGGTTCCTTCTGATGATCT | 124 | 197 |
| | IPO_02150_R | CTTCAACTTCACGTGTTCAATCA | | 198 |
| IPO_02151 | IPO_02151_L | GCTAGTCCACACGACAAAATCAT | 163 | 199 |
| | IPO_02151_R | GTGACTAGCTTGGCGATCTTCT | | 200 |
| IPO_02152 | IPO_02152_L | AGGTGAAGTGCTCGAAATCCT | 284 | 201 |
| | IPO_02152_R | CTTCTTCCTTCTCGATGCCTTC | | 202 |
| IPO_02153 | IPO_02153_L | TTATGTGGCTTTCTCTGGCAATA | 228 | 203 |
| | IPO_02153_R | ACAAACGTCCAGTCGTCAATC | | 204 |
| IPO_02154 | IPO_02154_L | CATTTATCTCTGGTCTTGGCTTG | 173 | 205 |
| | IPO_02154_R | ACAGCCAAACTGACAAGATCG | | 206 |
| IPO_02155 | IPO_02155_L | GCATGAAAATGTCTACGTTCCTC | 491 | 207 |
| | IPO_02155_R | ATGGTGATAGTGCGGAATGAC | | 208 |
| IPO_02156 | IPO_02156_L | CAGACATGTTCTGCGAAGGAT | 283 | 209 |
| | IPO_02156_R | GAGGAACGTAGACATTTTCATGC | | 210 |
| IPO_02157 | IPO_02157_L | TGTAATGACCAAGCAAGAACTCA | 112 | 211 |
| | IPO_02157_R | CACGGTGTCAAGAATGGTTTC | | 212 |
| IPO_02158 | IPO_02158_L | ATTTCAAACGCCAAGCCTTTAAC | 165 | 213 |
| | IPO_02158_R | GTTCATCGAAAGCGATGTTCTC | | 214 |
| IPO_02159 | IPO_02159_L | GCCTATTTGGACCGAAGAAGAC | 394 | 215 |
| | IPO_02159_R | ACTTCTTGAGGCATCTCGGTTT | | 216 |
| IPO_02160 | IPO_02160_L | GCAGCATTGATGACAAGTTCC | 339 | 217 |
| | IPO_02160_R | AGTATTGGTAAAGGCGTTGCAC | | 218 |
| IPO_02162 | IPO_02162_L | AAGGCTAAGGGGGAGTAAGTCAT | 548 | 411 |
| | IPO_02162_R | AGGTAGTTGCGTACTTGGTCGTA | | 412 |
| IPO_02163 | IPO_02163_L | AGCAACCGAAGATATCGACCT | 284 | 219 |
| | IPO_02163_R | GCCCAAGCGAAATCAACTCTT | | 220 |
| IPO_02165 | IPO_02165_L | CTTTCCCAGTCAATACATTCCAG | 220 | 221 |
| | IPO_02165_R | CGTTATCACATAAGCCACATCAA | | 222 |
| IPO_02166 | IPO_02166_L | AAACAACACGCTGTTGAGCAT | 195 | 223 |
| | IPO_02166_R | CTTCTTTGCGCACCAATAATC | | 224 |
| IPO_02923 | IPO_02923_L | GCGCAAATTAAACACTACAAAGG | 225 | 413 |
| | IPO_02923_R | ATCCCCAATCTCCTTTATCACTC | | 414 |
| IPO_02924 | IPO_02924_L | GAAGGTAAATCCAAAGGAAATGG | 232 | 415 |
| | IPO_02924_R | AATTGACTTCGGCTCGTATTCTT | | 416 |
| IPO_02927 | IPO_02927_L | AATTCCATGAGTGGAGTGATTTTT | 245 | 417 |
| | IPO_02927_R | AGATCATGAGCCTCAGCATTATTA | | 418 |
| IPO_03123 | IPO_03123_L | GAACGGAGCCATAGTGATGAAG | 946 | 419 |
| | IPO_03123_R | AGAGTCGCTAACACAAGTCATCC | | 420 |
| IPO_03132 | IPO_03132_L | AGGGAATCAAATCGCTCATCT | 226 | 245 |
| | IPO_03132_R | AGAAGAAGCCCATGATGACAGAG | | 246 |
| IPO_03302 | IPO_03302_L | CGTGTATATCGGCAGTCAAGAAG | 227 | 225 |
| | IPO_03302_R | CTAAGAAATGAAAGGTGGGGTTC | | 226 |
| IPO_03306 | IPO_03306_L | GGATGACTTGTGTTAGCGACTCT | 171 | 227 |
| | IPO_03306_R | GAATACGATCCTCCACAATCAAA | | 228 |
| IPO_03560 | IPO_03560_L | CACGATCATATATGGGTCCAGTT | 173 | 421 |
| | IPO_03560_R | GGTTCTTTTTGATCGTAGCCTTT | | 422 |
| IPO_03656 | IPO_03656_L | TCTGTTCCGAGTATCACCTTGTT | 156 | 423 |
| | IPO_03656_R | CATAGTATTCGCAGTCCAGATCC | | 424 |
| IPO_03659 | IPO_03659_L | GGATGTGGGGAGGTTTATTAGTC | 192 | 425 |
| | IPO_03659_R | TTCATCAACTCCTCAGGAATCAG | | 426 |
| IPO_03742 | IPO_03742_L | AAGATATGTGCCAGCTACCACTG | 206 | 427 |
| | IPO_03742_R | AGCATATACAGTCCCTCGTATGC | | 428 |
| IPO_03816 | IPO_03816_L | ATTTCAACGACCTGCATCAGA | 272 | 429 |
| | IPO_03816_R | CCACACCAGGTTCTTCTTGTTC | | 430 |
| IPO_04000 | IPO_04000_L | GTAAGGTCTGCAAGGACATCATC | 179 | 431 |
| | IPO_04000_R | GTGGCTGCGATAGAACTTGTAGT | | 432 |
| IPO_04001 | IPO_04001_L | GACCATCTTTCAGTGGTGGA | 110 | 433 |
| | IPO_04001_R | TAATGCCCTAAACTTTCCTGATG | | 434 |
| IPO_04002 | IPO_04002_L | TGTTTGATCTGAGCAAGTTTGTG | 250 | 435 |
| | IPO_04002_R | AGAAACTCATCCGCAAGGTC | | 436 |
| IPO_04003 | IPO_04003_L | GTAAGGAGAAGTGATGTCGGAAC | 283 | 437 |
| | IPO_04003_R | GTCCTTGTTCTTGAACTGGTACG | | 438 |
| IPO_04004 | IPO_04004_L | CTTGACGTCTGACAACCAAGTAG | 342 | 241 |
| | IPO_04004_R | ATAAGATAAACAGGTCGGCCTTC | | 242 |
| IPO_04067 | IPO_04067_L | CATGCCAAGGAACACATCAAG | 203 | 439 |
| | IPO_04067_R | AAGTATTTGTTGCCGTGGTACTC | | 440 |
| IPO_04353 | IPO_04353_L | AGGTACTCGGCTATCACAATCAC | 202 | 441 |
| | IPO_04353_R | ATGCATTCTCCATGTATTCCATC | | 442 |
| IPO_04521 | IPO_04521_L | TGAAGCACTGTCTATCAACCAGA | 222 | 229 |
| | IPO_04521_R | TTTGTCCTAGTCACAGCACTGAA | | 230 |
| IPO_04523 | IPO_04523_L | GACTTCGGCTATCTGGAGAAAAT | 217 | 231 |
| | IPO_04523_R | TCTTAGCAGGTTTAGGCTGAGTG | | 232 |
| IPO_04524 | IPO_04524_L | CATCTTCAAGGATGACTCTCTGG | 235 | 233 |
| | IPO_04524_R | GAAGAAGTGACCAGGCTGAATTT | | 234 |
| IPO_04525 | IPO_04525_L | ACTCAGTGACGAAGAGGTTGAAG | 250 | 235 |
| | IPO_04525_R | ACGAGTAGCTTCAATGGTGTCTT | | 236 |
| IPO_04526 | IPO_04526_L | ACACTATGCCTGCTGACTTGAA | 169 | 237 |
| | IPO_04526_R | AGGTGACTTCAACAATGTTAGGC | | 238 |
| IPO_04530 | IPO_04530_L | GAGACTCGGTTCAACAAGAAAAA | 207 | 443 |
| | IPO_04530_R | CTTAGACGCACTAGCGAAATACG | | 444 |

(a) sequences in bold correspond to part of the oligonucleotide spotted on the microarray

TABLE 3

| | | | |
|---|---|---|---|
| | | SEQ | |
| | | ID | |
| Oligo ID | Gene ID | NO: | Oligonucleotide sequence |

Race 3 biovar 2 specific genes clusters

| | | | | |
|---|---|---|---|---|
| 1 | IPO1609_0648 | IPO_00030 | 247 | GGCTCGAACAACTTGGAGAGATTTACGGAGGAGACGTGTCATTGCTCATGCCGAATTCACGCTACGCCGC |
| 1 | IPO1609_0647 | IPO_00031 | 248 | CCGGAGGAACCGCTGCGATGGACGCCATCACTGGAAAGTCTAGTGGCCGAACCGCGAAAGCGCGCGCATG |
| 2 | IPO1609_0644 | IPO_00043 | 249 | GTGACGATACCGCTGTTGCGTCTCTGTCGTTTGTTGTCCAAGAGGGCTTCAAACCAGATACGTTCTCCGC |
| 2 | IPO1609_0643 | IPO_00044 | 250 | GCGAACAATGTCACCGTCGCCTATGCAGAAGCGTTGCTTGTTGCCACGCCTGCCTCTCGTCTTGTTGATG |
| 2 | IPO1609_0641 | IPO_00045 | 251 | CAAGGTCTTTGACGAGATCGTTGGAAACATCAAGACCATTGGGCTAAAGAAGCCAATCACCGTCACGCCG |
| 2 | IPO1609_0642 | IPO_00045 | 252 | GACGAGAATTTCGTCAACCTTCTGCGAGCGGAATCTTTGGCGACACTTCCCAAATATCTGGCTGAGCGTG |
| 2 | IPO1609_0640 | IPO_00046* | 253 | CGCAACCGTGAGGGAGGTGCTCACCAACGAGAAATACATCGGCAACAACATCTATAACCGTGTGTCGTAC |
| 2 | IPO1609_0639 | IPO_00046/ | 254 | GCGCCTGGTTGTCGCGATAGTCGTCATCAGAGCGCAAGTGCTGCTGTTGCCACCGTCGGCGACGTTGGCG |
| | | 00047 | | |
| 3 | IPO1609_0625 | IPO_00232/ | 255 | GGTGCGACGTTCTTCTCGGCTCCAGATAAGGCGGTCCTGCTACTGTCGTTCAGATATCTCACAGACGACC |
| | | 00233 | | |
| 3 | IPO1609_0626 | IPO_00232/ | 256 | GGCTGTGAGTATGTTCTGAGGGTAGCCGCGCATCATCGTCCCGAACCACGTTCGTCAACTGGTAAGTTGG |
| | | 00233 | | |
| 3 | IPO1609_0624 | IPO_00233 | 257 | CCTGGTTGTGCTTGATAACCATCTGAACGTTGGAATGCAAGTGCTGGAAGACTCGCTATGCAACTGGCAG |
| 3 | IPO1609_0623 | IPO_00234 | 258 | CGGAACTAGGCACATACAAGCACGCCGTCGGAAGCCTACATTTGTACTCGAAGAACACGGACCAGGCGAC |
| 4 | IPO1609_0267 | IPO_00874/ | 259 | GCAGGATAAGAGCCGTGAGGTTCCTGTTCATCTGCGTAATGCACCAACAAAGCTCATGAAGGAACTCGGC |
| | | 00875 | | |
| 4 | IPO1609_0266 | IPO_00875 | 260 | GAGCGGTTCCTTGAAAGCTATGCTGGAGCCATCATCACTGATCCTGCTACGGCGATTGTGGAGCTTGTCG |
| 4 | IPO1609_0265 | IPO_00875/ | 261 | GTCTTCATCAACCACATTGAAGTTCACGACAAGCCAGCGGCAGACAACATCAGGAACATTGTGTTCGGCG |
| | | 00876 | | |
| 4 | IPO1609_0272 | IPO_00876 | 262 | CTGCCTATCATCCTGGATAGAGGGCGAAGCCGAACCTTGGAGCGCACAGTCAAGGAAGGCTTTGAACATG |
| 4 | IPO1609_0271 | IPO_00877 | 263 | CGGCTACAGGCAGTCTCAAAGAGACAGGTCTGAAGAAGACTATGCCATCTATTCGGTATGTGGCTGGTGC |
| 4 | IPO1609_0270 | IPO_00878 | 264 | CGTTGAACATGTGGATGGTAAGCACGGAGGTAAAGTCAGGCAGTTGATTAGACTGATCAAGGCATGGAAG |
| 4 | IPO1609_0269 | IPO_00878/ | 265 | CGCTGGTGCTGTGGTGGGCTCCCAAGTGAGTAAGAGCAGTGAAGTGAAGAATGTCACTGACTTGCAAGTG |
| | | 00879 | | |
| 4 | IPO1609_0268 | IP_00879 | 266 | CCAGCAATCCCACTAATGTTTGTGCTACTGGTCAGTCCTACGGCTTCAACTTCGCCAACAATCAATACAC |
| 4 | | IPO_00880* | | no representative oligonucleotide on the micro array |
| 4 | IPO1609_0659 | IPO_00881 | 267 | GGAGGTTTGGAGGCTCAGATCGGTGGTTTGTCCATGCAGTATCACAACCTGCTGTTGCTGCTGGCTGGTG |
| 4 | | IPO_00882* | | no representative oligonucleotide on the micro array |
| 4 | | IPO_00883* | | no representative oligonucleotide on the micro array |
| 4 | IPO1609_0151 | IPO_00883/ | 268 | GCTGTAGCACCGACTCTGACCGCCAATGACGAAGTTCTATTGAACTTCATTAGTGAATACGGTGATGGTG |
| | | 00884 | | |
| 5 | IPO160_0141 | IPO_01057/ | 269 | CGTCTTGTGCCTATAGCAATGAAATGCAGAAGGCTTGGCGGACAATATCAACTATGCCAGCTCGTGGTG |
| | | 01058 | | |
| 5 | IPO160_0135 | IPO_01058 | 270 | ATCATGGCTGAGATCCAGCGTATTGCCGCAACCGAGCTCATCGTTATCGACATGGGCGACAGGCTTGGCA |
| 6 | IPO1609_0124 | IPO_01130/ | 271 | GAGGTCAAGCTGGATCAAGCTGGAGTCGAAGTGGAGGAAGCGAAAGTCCACAAGTTCAGCAAGAACGATC |
| | | 01131 | | |
| 6 | IPO1609_0123 | IPO_01131 | 272 | CCACGATCAACTACTACCGTGTATCGACCAAGGATCAATCCATCGAATCCCAGCGTCAGAAGCTAGGCCC |
| 6 | IPO1609_0122 | IPO_01132 | 273 | CATCAACGACCCTTACTGTCCGAGTAACTGCCGATGGGCCACCAACAACGATCAGCAGAACAACAAGCGG |
| 7 | IPO1609_0159 | IPO_01258/ | 274 | GACCGACTGGAACGAGATGGCCAAGGCTCCGAGCAAGGTGGCAAAGATCGCGAAAGCAGTGGGACTCGAC |
| | | 01259 | | |
| 7 | IPO1609_0160 | IPO_01258/ | 275 | CTTCATGACGGTGTAGCCGTGACCCAGGTGTCTGACCTTGATGCGCGACTAAGCGCACACTTGATGCTCT |
| | | 01259 | | |
| 7 | IPO1609_0161 | IPO_01259 | 276 | GCCGGTGAAGATACGGGTCAGCGTCTCAAGCAGGCGCGTGAAATGCTCAAAGACAAACAGATCTTCGCGG |
| 7 | IPO1609_0386 | IPO_01260 | 277 | GCCACATCGAACTGTGGGCCGACGGCATCTCCCTGCCCTATGTCCTCTATGACCGGCTTTGCGAGATCGA |
| 8 | IPO1609_0167 | IPO_01311 | 278 | GGAGAACCTCGACAACCAGACCATCTACAAGATCCTAGTCAAGTGCATCAAGAAGGCGAAGGAGAGGCGG |
| 8 | IPO1609_0168 | IPO_01312 | 279 | GGCAGATGGACATGAATCTTCCTCTTGAATTTCACGTCCGCATTGAGGAGAC |
| 8 | IPO1609_0169 | IPO_01313* | 280 | GTGCCACTAGCTATTCGTGCGAGTCCCTGGCAGAGATATTCAGACGGCTACACCAGCAGGCTGGCATCGC |
| 8 | IPO1609_0170 | IPO_01314 | 281 | GCGCTGCCGCCGCTGGCTCGGGCGGCAATCGACCGCTACCTGGTTGAACGCGGGCTACCGATTACGCGCG |
| 9 | IPO1609_0216 | IPO_01877/ | 282 | GAGAAACATCCCGTCGCCATGCGCCGCATGTTGCTGGTTGCCGCAACCGTCACGCTGCTATCGCTGACTG |
| | | 01878 | | |
| 9 | | IPO_01878* | | no representative oligonucleotide on the micro array |
| 9 | IPO1609_0217 | IPO_01878/ | 283 | CGTTTCGTGCATACATCGACAGCCTTGACCACTACACTGACGCCCATGTGATTGTCCTTCCGTATGCGCC |
| | | 01879 | | |
| 9 | IPO1609_0218 | IPO_01878/ | 284 | GATATCTGGCAGGCATTTGCCAACCCAGACGAAGATCGCTTGCGCTACTTCAAGAAGGCCACTGATAAGG |
| | | 01879 | | |
| 10 | IPO1609_0244 | IPO_02090 | 285 | GACGTCAAGCTGCCTATCGGGCAGGTTTGCCCAGTGTCGTTGAACATCCTTATCAAGGGTGAGTCGGGCG |
| 10 | IPO1609_0245 | IPO_02091* | 286 | CTTGACCGATACGTCAAGGGCAGCCGACTGCCGACATATGCAGCGGCACTCATCCTGGCGAAAGAAGCGG |
| 10 | IPO1609_0246 | IPO_02092 | 287 | CGCAATGCATCCTCACCGAAGGTGTTGCTGACGCAACTGGCGGCGTCTCCGAACAGATCAAGGTTGGCCG |
| 10 | IPO1609_0247 | IPO_02092/ | 288 | CGTTCAGGAGGTCGTCATGGTCGATCCGAACGACAAGTACACGATGCAGTTGCCGCTGATGCGTTATCGC |
| | | 02093 | | |
| 10 | IPO1609_0248 | IPO_02093* | 289 | GGGCTCGTGCGTCAATGCTTTGGTTATGGCGCTGTTCGTTCTGCTGCTGACGCTGTTGGTCCCGGCGTGG |
| 10 | IPO1609_0249 | IPO_02093/ | 290 | GAGGCGATGGACGACGCTGAGGCTATCGAGTTCTTCGGCAGCCTGGATGAGCGTGATGAGGCGATTGAGG |
| | | 02094 | | |
| 10 | IPO1609_0250 | IPO_02094* | 291 | CCACAGAGCTGCAGCGGCTATCTGCTGTTGCAGCCGAGCGAATACTCGAACGTGATGGCTCTGTCCGGCG |
| 10 | IPO1609_0251 | IPO_02095 | 292 | GGGTGCTGCGATGGCGCAAACCACGACCAGCGGTATCGATTTCTCGTCCATGACGGGTGCCGTGAGCGCG |
| 10 | | IPO_02096* | | no representative oligonucleotide on the micro array |
| 10 | IPO1609_0252 | IPO_02097 | 293 | CGACAGTGTCTTCGTCGCTATCTTCACGGCGCTTTGGCAGATCTCAGAGGATCTGTTCATCGACTCGCTG |
| 10 | IPO1609_0253 | IPO_02098 | 294 | CGAGATTCCTGAAGCTGAGGTGCTGCGTTGGAATGAGCTGGTCGAGAACGACAGCCTGGTCATCTTGGAC |
| 10 | | IPO_02099* | | no representative oligonucleotide on the micro array |
| 10 | | IPO_02100* | | no representative oligonucleotide on the micro array |
| 10 | IPO1609_0257 | IPO_02100/ | 295 | CGCCGCACTGCATTGACCAAGCTCATTCGTCAGAAGCGATCTAACAACTTTCAGATTGCCAAAGAGATGG |
| | | 02101 | | |
| 10 | IPO1609_0258 | IPO_02100/ | 296 | GGAACAAATCAAGATGTTCAACTTCACGGCTGATGAACTTGGTTTCCATGCTGCTCCTGATGCACGTGCC |
| | | 02101 | | |
| 10 | IPO1609_0259 | IPO_02100/ | 297 | CTGTTTACCAACCACATAGCCTCACACACGGCTGCTGACGGATCAGACTTGGACGACTACTTGAGTCGTG |
| | | 02101 | | |
| 10 | | IPO_02101* | | no representative oligonucleotide on the micro array |
| 10 | IPO1609_0060 | IPO_02102 | 298 | GGTGGTCGAGGTGAGAAGTGTCTACGACAAACCTGTGAAAGCCGTTGAGAATCGAATATTGGCAATCAGG |
| 10 | IPO1609_0159 | IPO_02103* | | no representative oligonucleotide on the micro array |
| 10 | IPO1609_0061 | IPO_02103/ | 299 | CGTCATTCGTGAGAACCAGATCAAAGAGCTTCTGATTGCTTACAACAGCTACTTCATGGTGGCTGCTGCC |
| | | 02104 | | |
| 11 | IPO1609_0065 | IPO_02140 | 300 | GAAGTATGCTGGCCTGATCGGTCAGCTCAACTCAGCCGCTAGGGAAACGCTGATTTATCAGTCTCAGCCG |
| 11 | IPO1609_0066 | IPO_02141 | 301 | GCGTTACCGCATGGAGGCTACTGACGCAGCAAACCTCAAGAATCAGACCGTTACGTTCCAGATTAAAGTG |
| 11 | IPO1609_0067 | IPO_02141/ | 302 | CCGGATAGCCGCCATTGGCAGCACGGTCGGCAACCGGTTGTGGAGCCATGACGCGCCAACGCTGATGTTC |
| | | 02142 | | |
| 11 | IPO1609_0068 | IPO_02142 | 303 | GTCAATGGCACCTTGACGGTAGGCGACGCGGGAATCACGGGCGGCGCTGAAGACGAGAGCGTGGACGATT |
| 11 | IPO1609_0069 | IPO_02143 | 304 | GCCTTGTAGTCGGCGCGAGCAAGGTGCAGGTGCGCGTGCTGGACGAAGAGACGTTGGACAACATCAACCG |
| 11 | IPO1609_0070 | IPO_02144 | 305 | GCTTGGTCGTGAGCTTGTGGACTGCCAGTATTCCAAGACGCTATCCGGCCTGAAACTGAGCGAGATCGTG |
| 11 | IPO1609_0071 | IPO_02145 | 306 | GCGCTGTCTGGTGTTGCCTCAACGGATGGTGCCGTGCGTCGCAATCTGCTGCGCGAAGAAGCGTTGCGCG |
| 11 | IPO1609_0072 | IPO_02146 | 307 | CGGTGTTGGTGCCGCTGTCGGTGGCGGTCTGGGCTTAGCCTGGGAAGGTCTCAAAGACCTGCTGAAGCCA |
| 11 | IPO1609_0074 | IPO_02147 | 308 | CGCCGGCCGACATCGAAGCGAACATCGCAAGCGAGCATTACTTCACTGCCGAAGACGGCATGATGGCCGG |
| 11 | IPO1609_0076 | IPO_02148 | 309 | TTCCTGCTACATCAAGCGGCTGATTTCCATGTACCTGCATCGCTCCGATGGCAGCACCGACGACGCCTAT |
| 11 | IPO1609_0077 | IPO_02149 | 310 | CGTCGGTGGCCAAGTCCTGGAGTACACCGACGGCGATTTGTACTGGCGCGATCGCTACGAGTTCGACGCC |
| 11 | IPO1609_0078 | IPO_02150 | 311 | CCGCATCCGATGATCCGGTTCCTTCTGATGATCTGGCCATCATCGAAAGCGCACCGCGCCGCTTTGGCCG |
| 11 | IPO1609_0080 | IPO_02151 | 312 | CGGAAGACTGGAAGAAGATCGCCAAGCTAGTCACCAGTACCGGCAAATCCAACACCTACGAATGGCTGAG |
| 11 | IPO1609_0081 | IPO_02152 | 313 | GGTGAAGTGCTCGAAATCCTTTCGGTCGCACTCACCAACACACCCGCCCTGGATGGGCTAGAAGCCGTGG |
| 11 | IPO1609_0082 | IPO_02153 | 314 | CGCCGAGTGGTTCGCGCTCTACCGCGATGATGGCGCGATTGACGACTGGACGTTTGTGAGTGGCGTGCGG |
| 11 | IPO1609_0083 | IPO_02154 | 315 | CATGTTCCTTGTGGTGGCACTGCTGCTGGCGATCTTGTCAGTTTGGCTGTTCGACCGCTACGTGTGTCGG |
| 11 | IPO1609_0084 | IPO_02154/ | 316 | CGTGGTAGACGCCTACCCGGCTGTCGATGAAGATTTGCTGAGAACCTTGCCACCCGTGCTGCGTGCGGTG |
| | | 02155 | | |
| 11 | IPO1609_0085 | IPO_02155 | 317 | GCGCGTCAAGTGGGCCGTCACGTGCCGGACTTGGACAGCTTGGCCGTAGCTGAATGCCAAACGATTGCCG |
| 11 | IPO1609_0086 | IPO_02156 | 318 | GCAGACATGTTCTGCGAAGGATGGGTGTTTGCCGTGCGCGAGCTGGTGGGTGCCACCGTGCCGCCCGAAG |
| 11 | IPO1609_0087 | IPO_02157 | 319 | GACCGAAACCATTCTTGACACCGTGCGCGCCGGTAACGAGCTGACGATCACCGACCTGGGCAAGTTCGGC |
| 11 | IPO1609_0088 | IPO_02158 | 320 | TCACCTTGAGCACGTTCGACGGTCGCCAGCAGGTAGTCGTGTCCCAGGCCGAGAACATCGCTTTCGATGA |
| 11 | IPO1609_0089 | IPO_02159 | 321 | CCGCTGCCTGAAGTCGTCTGCCCGAACTGCCGCGTGCGGATGAGCCTGGATGTGGTGCTAGCCGATGACG |
| 11 | IPO1609_0090 | IPO_02159/ | 322 | GAAACAGCTAGCAGGCATCACCCAGGGACCTGTTGCAGCCGAACTGCTGGCGGGCTTGCGCCGAATCTGC |
| | | 02160 | | |
| 11 | IPO1609_0091 | IPO_02159/ | 323 | CGCTTCTTGGATGCCATCGCCAACGGCGCACCTTCAGCAGAGGCAGCCGCGTTTGGCGCCTGGATCGAAG |
| | | 02160 | | |
| 11 | IPO1609_0092 | IPO_02160 | 324 | CTTCGCAACACCGACAGTCTGCTCATCGTGGATGAGGCCGAAACGCTCACGCCGCACCAGCTCCACACGC |
| 11 | | IPO_02161* | | no representative oligonucleotide on the micro array |
| 11 | IPO1609_0093 | IPO_02162 | 325 | CGCTGTACATCGCCGCCTGGCGGAAGTGGACGGCTTCGCGGTCAGCTACGACCAAGTACGCAACTACCTG |
| 11 | IPO1609_0094 | IPO_02163 | 326 | CAAGCAACCGAAGATATCGACCTGGGCATCACGGAAGGCGCTCTAGTTCACGCCACGCAGACGGACGACG |
| 11 | | IPO_02164* | | no representative oligonucleotide on the micro array |
| 11 | IPO1609_0095 | IPO_02165 | 327 | GCCGACACGACGAAGCAAACACAGCACGCATACGAAACCGACCGCACGCCGCCGAAAGCCAGCTATCTGG |
| 11 | IPO1609_0096 | IPO_02166 | 328 | GGCGATCTTGGAATGTCAGACCGTGGCAAACAACACGCTGTTGAGCATCAACGGCATGAACAAGCGTTTC |
| 11 | | IPO_02167* | | no representative oligonucleotide on the micro array |
| 11 | IPO1609_0097 | IPO_02168* | 329 | GGACTGGGCCACTGGCAACAGGAAGCGGCCACGGTGCGGCCTGCGCTGAACCGCGCAACCATCGATAGCG |
| 11 | IPO1609_0098 | IPO_02168/ | 330 | GTCGCTAGCTGGATCAACCACGCCAAAGGCCAATACGAAATCTGCCGGGCTCGACTCGGCGCACTGATCG |
| | | 02169 | | |
| 11 | IPO1609_0099 | IPO_02168/ | 331 | GGAAGGTCTATCACGATCATCTCAGTGACCCTAACAAGAGGCCACCAGCGAACCTGGAACTTGTTGCTGC |
| | | 02169 | | |
| 12 | IPO1609_0028 | IPO_02922/ | 332 | CGCACTTACCTCGACGCCAGCATTGAGTCATTGGATCGCCACATTAAGCTGCTGACTAGCCTAATCGAAG |
| | | 02923 | | |
| 12 | IPO1609_0027 | IPO_02923 | 333 | CAAATTAAACACTACAAAGGGCTGGTAGTTTATGCGCCTCTAATCGCGCTATCTGTGGTCTTTGTGGCCG |
| 12 | IPO1609_0026 | IPO_02924 | 334 | GCGGTGCTTGCCTTGTCTGGCGCCAAACGCTTCCAGCACTTCATCAAGGTCGTTGCAGATTGGCAGGAGG |
| 12 | IPO1609_0025 | IPO_02924/ | 335 | ACGCGAATAAGGCATCGCTACCAGGTGACGTAAGCCGGGTATATAGAAATGCTTTAGACATGATCTGGAG |
| | | 02925 | | |
| 12 | IPO1609_0024 | IPO_02925* | 336 | CTATCGGTTCCGCTGCGCACATGGGCATGAAACGTCCCGCACCGGCGACTACGCCCTCCGTAGTCTGATC |
| 12 | | IPO_02926* | | no representative oligonucleotide on the micro array |
| 12 | IPO1609_0023 | IPO_02927 | 337 | CTTCGCTAAATAATAATGCTGAGGCTCATGATCTTCTATACTCGCTGCTTGCCCATGACAGTGAGGCGGC |
| 12 | IPO1609_0022 | IPO_02927/ | 338 | GACATTGCAGCATCCGTCTTGCGTGACATTGAAGTCGGCAAACACGGCTTAATGTCGGCTTCGGCGAGCG |
| | | 02928 | | |
| 12 | | IPO_02928* | | no representative oligonucleotide on the micro array |
| 12 | | IPO_02929* | | no representative oligonucleotide on the micro array |
| 12 | | IPO_02930* | | no representative oligonucleotide on the micro array |
| 12 | | IPO_02931* | | no representative oligonucleotide on the micro array |
| 12 | | IPO_02932* | | no representative oligonucleotide on the micro array |
| 12 | | IPO_02933* | | no representative oligonucleotide on the micro array |
| 12 | IPO1609_0020 | IPO_02934* | 339 | GTGCGCAAACAAGGCACGCGGCGAAGCGTCTCTGCGCAAAGACGGATTGGACGAACTGCGCCGCATCGCG |
| 12 | IPO1609_0019 | IPO_02934/ | 340 | GGCCGGAGACATCTTTGCGTTCCAGTTGGAGCAGTTTCCGGATCGGTACTTCTTTGGGCGAGTCGTGGAC |
| | | 02935 | | |
| 13 | IPO1609_0524 | IPO_03302 | 341 | CAGCGGACGAGCAACGATAGGTTGCTTCACGACGATCTCGGCGCGTGTATATCGGCAGTCAAGAAGTTCG |
| 13 | | IPO_03303* | | no representative oligonucleotide on the micro array |
| 13 | | IPO_03304* | | no representative oligonucleotide on the micro array |
| 13 | IPO1609_0522 | IPO_03304/ | 342 | CGTTGGGAAGACAAAGAGAACGTCTTTCTCTACGCATCAGGATTTCTTGCCGCAATGGATTTCCTCAGGC |
| | | 03305 | | |
| 13 | IPO1609_0523 | IPO_03304/ | 343 | CGCGGGCGTCGCTTGCCCTCGGTGGCGTCGGGATCTTCTATTCAAGAACTACGCCCTATTCGTGGCGGGC |
| | | 03305 | | |
| 13 | | IPO_03305* | | no representative oligonucleotide on the micro array |
| 13 | IPO1609_0521 | IPO_03306 | 344 | CAAGGCAATTTGATTGTGGAGGATCGTATTCAGGTTCACATGAGCTTGCGACTTCTGCATATTCCAACGG |
| 13 | IPO1609_0520 | IPO_03306/ | 345 | GTCGGGTTGTTGTACCTCTACGGCGAGCAAACCGGTCATCCATCAGCGGCTGGTGGCGCTCTACTGCTGT |
| | | 03307 | | |
| 14 | IPO1609_0457 | IPO_03656 | 346 | CGAGTATCACCTTGTTGAGGCTCGATCAGTTACTGAATGCCGCCTATGCCAACCTGTTGACCAAGATGCG |
| 14 | | IPO_03657* | | no representative oligonucleotide on the micro array |
| 14 | | IPO_03658* | | no representative oligonucleotide on the micro array |
| 14 | IPO1609_0456 | IPO_03659 | 347 | GATGGCATGGCGAACCATCACCTGATTCCTGAGGAGTTGATGAAACATCCTCGATACGCGGCGATGTTCC |
| 14 | | IPO_03660* | | no representative oligonucleotide on the micro array |
| 14 | IPO1609_0455 | IPO_03660/ | 348 | GGCTTGAAATGGTGGAGATTCAACTAGACAAGCATGGCCGCGCTGCCTTTCGTCTGAACATTGGTGTCGC |
| | | 03661 | | |
| 15 | IPO1609_0435 | IPO_03741/ | 349 | GCCACACAGCTTCTCGGCGGCTCAATGCGGTCGTGTGTCTCTTGAGATCGCTGACAGAACGCTCAGGGCG |
| | | 03742 | | |
| 15 | IPO1609_0434 | IPO_03742 | 350 | GTGCATACGAGGGACTGTATATGCTCTCGTGCATGGAAACACTCACAAAGGAACGCGCCGACCACTTGGC |
| 16 | IPO1609_0420 | IPO_03816 | 351 | CAATAGCCTTGCCAAGCACTGCAACATGAAGGCGGTCACGGATTTCAACGACCTGCATCAGAAGCAACTG |
| 16 | IPO1609_0418 | IPO_03816/ | 352 | GCGGAAGTGTGGGCAGAAGGAAGCTGTTGAGTATATCGCTGATTTGAATCGGTATACGACGCTTCGACCG |
| | | 03817 | | |
| 16 | IPO1609_0419 | IPO_03816/ | 353 | CGATCACTTCATCGAGCTATCAAATATTGGTGAAGAAGATCGTTCAGTGGAGGCAATGGCGGTTGGGATG |
| | | 03817 | | |
| 17 | IPO1609_0383 | IPO_03999/ | 354 | GTTGGTGAGCGAAGCGCAGATGCGTGCCGCTGGTGAAGCGATCTATGGTGAACGTAAAGTCGTTACGTGC |
| | | 04000 | | |
| 17 | IPO1609_0382 | IPO_04000 | 355 | CGTCGTGAACACCTCGGTTCGCAAGTACGACAAGAAGTACTACAAGTTCTATCGCAGCCACACCAAGAGC |
| 17 | IPO1609_0376 | IPO_04000/ | 356 | CGGCGTGATCGGCGTTGCGATCATCGGTATCTGCCTCGGTTTCAAGGCGATCGACCTGGGCAAGCGCGGC |
| | | 04001 | | |
| 17 | IPO1609_0377 | IPO_04000/ | 357 | GCGCTGGTGGCTCTCTTCTACGCGTTGGTGGCCATGATCGGTGCTATCAGCGCGCTCGTCTTCATTTGGG |
| | | 04001 | | |
| 17 | IPO1609_0378 | IPO_04000/ | 358 | GCCGCAATGGAGGTGTCGGCCATTGGTATTGCGGATATGGCTGCTGTTGTGGCGCGTGCCTCTACGCCGG |
| | | 04001 | | |
| 17 | IPO1609_0379 | IPO_04000/ | 359 | GGCAATTACGGTGTTCGCAGCATCGACGTGCCGTGCTCGTTGTTCCAGACCATCGGTCTGATCATTCTGG |
| | | 04001 | | |
| 17 | IPO1609_0380 | IPO_04000/ | 360 | GCTGCTATCTCGGCGTTGATCGCCAAGGTTGTCGGGCTGGCTACTTGGTTTGGGCAGCTCGCGATTGCCG |
| | | 04001 | | |
| 17 | IPO1609_0381 | IPO_04000/ | 361 | GATCTGATTGAGCGGTTGACGGAATTGGCGTTCGGCTACGCCGCTGAGATGCATTACCACCAGGAGAAGG |
| | | 04001 | | |
| 17 | IPO1609_0375 | IPO_04001 | 362 | CTGCTGCACTGCTGAGCAGTTTGCCGTGATCGGGATCGATGGCCCGACCATCTTTCAGTGGTGGAGCTGG |
| 17 | IPO1609_0374 | IPO_04002 | 363 | CTGCTTGTGATGGTCGTAATGGCGAGTCGGGTGGCGTACTGGGAGCGGCGTTTCTGGCAGTCGCTGGTCG |
| 17 | IPO1609_0373 | IPO_04003 | 364 | GGTGGTTATCAGCGCAAGGCGTACCAGTTCAAGAACAAGGACACCGGCGAGGTCGAGACGGTCACGCCGG |
| 17 | IPO1609_0372 | IPO_04003/ | 365 | ACAGCGACAAGCTCCAGTACGACTTTGGCAAGCATCCGCGAGTGGATGAGGTGACGGACGCCTATTTGGA |
| | | 04004 | | |
| 17 | IPO1609_0371 | IPO_04004 | 366 | GACGCAATACCGCAAGGGCGACCGCAAGATGGACAACGAAGCGTGCCTGCGCCTGGCGCAACTGCTCGAG |
| 18 | IPO1609_0264 | IPO_04521 | 367 | CGGCGTTCTACCAGCCAAGGAAGTGCTTTCCACTGCGAGATCTGTAGGCACATGGACATGGAGACACAAG |
| 18 | | IPO_04522* | | no representative oligonucleotide on the micro array |
| 18 | IPO1609_0263 | IPO_04523 | 368 | CGGCAGCACTCATGCCAGCGGGTTCATTGCCTCTCTACGTCCCAAGCTCAACTTCATTGTCACTGATACG |
| 18 | IPO1609_0262 | IPO_04524 | 369 | CGGCGTTCGTGAGGTACGTGGCTTCACTGGTGAGCAGCAAATTCAGCCTGGTCACTTCTTCATCCTCCAC |
| 18 | IPO1609_0261 | IPO_04525 | 370 | GTGGTGGATTCGGTCATCAAGAATGGCGAGATCAAGCAGGAAGGTGACAAACGCTTTGTTCGCATGGCCG |
| 18 | IPO1609_0260 | IPO_04526 | 371 | CCACGAACTGGATGTGGTGGTTGAGCAGTGCTACCAGATTAAGCCGTTCTACAGCGATACCGAACGCTTG |

Race 3 biovar 2 specific individual genes

| | | | | |
|---|---|---|---|---|
| 19 | IPO1609_0147 | IPO_01025/ | 372 | AGGCTATACGGACAGCACAGCCGCAAATTATGCCTGCCGACTGGGAGGTGAAAGAGGTTGGTGGCACGTT |
| | | 01026 | | |
| 20 | IPO1609_0146 | IPO_01030 | 373 | ACATGTTGTTTATCGGTGGATATGGCTGGGATGGCTCGAATCTGTATATTGAAATTAATACAGGAAAGGT |
| 21 | IPO1609_0185 | IPO_01362 | 374 | CAATTGGGTATACGTGATCTGTGGGTGCCTGATGGGATTCGCGTTCGGCTGGATTGCCATGCTCTGGCTC |
| 22 | IPO1609_0233 | IPO_02072/ | 375 | GCGGTAGCCGACTGGCACACGTATTTGGACGGCGTTGGCCGGGAAGATTTCCCTGACCAACCAGACGATC |
| | | 02073 | | |
| 22 | IPO1609_0234 | IPO_02072/ | 376 | CAGCCATTCTCTTCCAACTTTATTTCTACTGGATGGATCGCTATTACGTGTTTGCGCCGATGTGGAGCTC |
| | | 02073 | | |
| 23 | IPO1609_0111 | IPO_02448/ | 377 | CTTGAACCAAGCATAGAGCGCGTCGCATATCGCCAGGCCGTGTTCAAGCGTGACCTGATCATCGGTAAGC |
| | | 02449 | | |
| 24 | IPO1609_0563 | IPO_03105/ | 378 | GCCTATCGTCAACATGGTTTCCTATGTTGCTCAGAAGTCAGCGTGGAATATGCTCAATTTGAACGTGCCG |
| | | 03106 | | |
| 25 | IPO1609_0508 | IPO_03123 | 379 | CGCGTGACCAAACGCCAGCGCTTCCTGGCAGAGATGGAGAAGGTCTTG |
| 26 | IPO1609_0557 | IPO_03132 | 380 | CAAACATTGCCTTCACGGAAGATGAGCAGCATCTGTACAACCTGACGCTTGATGAGCTGGGCGACGAGTC |
| 27 | IPO1609_0420 | IPO_03433 | 381 | CAATAGCCTTGCCAAGCACTGCAACATGAAGGCGGTCACGGATTTCAACGACCTGCATCAGAAGCAACTG |
| 28 | IPO1609_0313 | IPO_04287 | 382 | GCCGGTTGCTGACGGAGATCGAGAGGCTGGCGGATGACTTTAGGGAACCGCTGATCAATGAAGTCGGCGA |
| 29 | PT04834A | IPO_04353 | 383 | CACAACCCTGCGCATTTCCGCGCCGAACTCTTGATGGAATACATGGAGAATGCATCCCCCTCAAAAAACG |
| 30 | IPO1609_0254 | IPO_04530 | 384 | GATATCGCGGACATAAGATGCTTCGTCGCCCTCGATGGCTCGTCGTATTTCGCTAGTGCGTCTAAGGCTG |
| 31 | IPO1609_0021 | IPO_04923 | 385 | GTCCAGACCTGTCTGTCACACTTGCCTACCAGCAGTCTTACAACGACTACGGATCGAATGTCGGCATCGG |
| 32 | IPO1609_0018 | IPO_04926 | 386 | GGTCGCCACATTCACATTGATGGCCCACTCGACGTATTGAACAAAGTTCCAGGCATTGATCATTGGTGGA |

TABLE 4

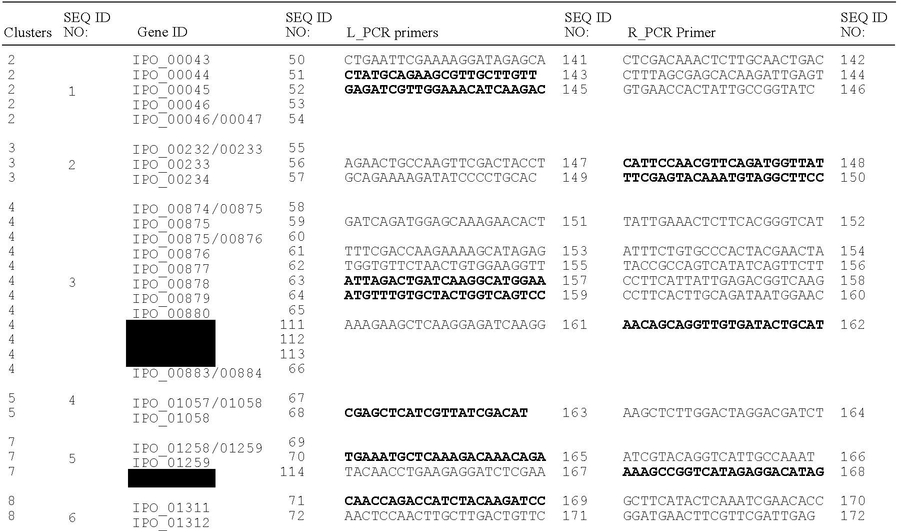

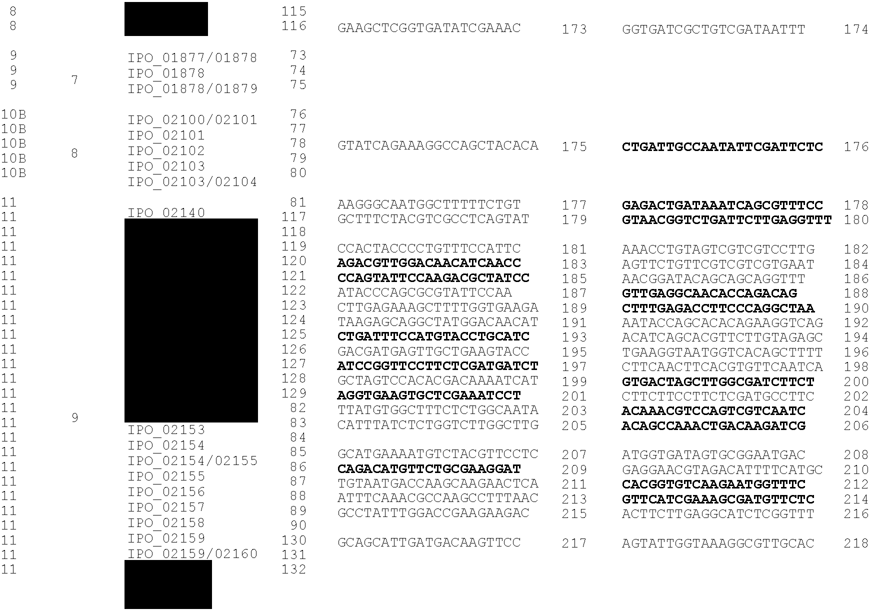

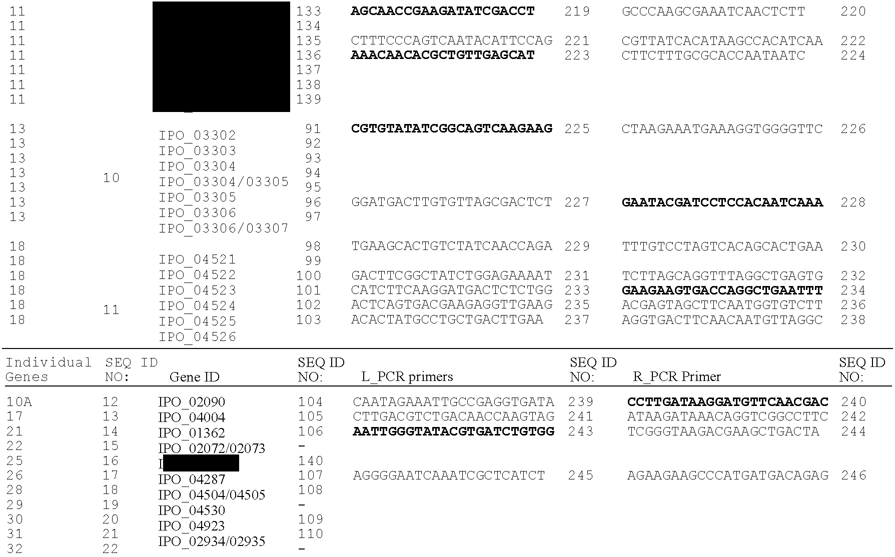

TABLE 5

| | GenBank Accession | |
|---|---|---|
| Gene ID | number | SEQ ID NO: |
| RRSL_00004 | NZ_AAKL01000174 | 23 |
| RRSL_00093 | NZ_AAKL01000099 | 24 |
| RRSL_00419 | NZ_AAKL01000074 | 25 |
| RRSL_00442 | NZ_AAKL01000066 | 26 |
| RRSL_00500 | NZ_AAKL01000026 | 27 |
| RRSL_00600 | NZ_AAKL01000073 | 28 |
| RRSL_00696 | NZ_AAKL01000059 | 29 |
| RRSL_00772 | NZ_AAKL01000058 | 30 |
| RRSL_01930 | NZ_AAKL01000033 | 31 |
| RRSL_01998 | NZ_AAKL01000044 | 32 |
| RRSL_02069 | NZ_AAKL01000022 | 33 |
| RRSL_02232 | NZ_AAKL01000025 | 34 |
| RRSL_02410 | NZ_AAKL01000024 | 35 |
| RRSL_02412 | NZ_AAKL01000024 | 36 |
| RRSL_02430 | NZ_AAKL01000024 | 37 |
| RRSL_03158 | NZ_AAKL01000027 | 38 |
| RRSL_03244 | NZ_AAKL01000017 | 39 |
| RRSL_03346 | NZ_AAKL01000012 | 40 |
| RRSL_03351 | NZ_AAKL01000012 | 41 |
| RRSL_03359 | NZ_AAKL01000012 | 42 |
| RRSL_03472 | NZ_AAKL01000008 | 43 |
| RRSL_03712 | NZ_AAKL01000013 | 44 |
| RRSL_03795 | NZ_AAKL01000009 | 45 |
| RRSL_03985 | NZ_AAKL01000007 | 46 |
| RRSL_04009 | NZ_AAKL01000007 | 47 |
| RRSL_04153 | NZ_AAKL01000004 | 48 |
| RRSL_04507 | NZ_AAKL01000002 | 49 |

### REFERENCES

Carmeille A, Prior P, Kodja H, Chiroleu F, Luisetti J, Besse P (2006) Evaluation of resistance to race 3, biovar 2 of Ralstonia solanacearum in tomato germplasm. J. Phytopathol. 154:398-402
Cook, D., and L. Sequeira. 1994. Strain differentiation of Pseudomonas solanacearum by molecular genetic methods, p. 77-94. In A. C. Hayward and G. L. Hartman (ed.), BActerial wilt—The disease and ist causative agent, Pseudomonas solanacearum. CAB International, Wallingford, UK.
Fegan, M., and P. Prior. (2005) How complex is the “Ralstonia solanacearum species complex”, p. 449-462. In C. Allen, P. Prior and C. Hayward (ed.), Bacterial Wilt: the Disease and the Ralstonia solanacearum species complex. APS Press, St. Paul, Minn., USA.
Fegan, M., and Prior, P. 2006. Diverse members of the Ralstonia solanacearum species complex cause bacterial wilts of banana. Australasian Plant Pathology 35, 2: 93-101.
Gabriel D W, Allen C, Schell M, Denny T P, Greenberg J T, Duan Y P, Flores-Cruz Z, Huang Q, Clifford J M, Presting G, González E T, Reddy J, Elphinstone J, Swanson J, Yao J, Mulholland V, Liu L, Farmerie W, Patnaikuni M, Balogh B, Norman D, Alvarez A, Castillo J A, Jones J, Saddler G, Walunas T, Zhukov A, Mikhailova N. (2006) Identification of open reading frames unique to a select agent: Ralstonia solanacearum race 3 biovar 2. 1: Mol Plant Microbe Interact. 2006 January; 19(1):69-79.
Guidot A, Prior P, Schoenfeld J, Carrera S, Genin S, Boucher C (2007) Genomic structure and phylogeny of the plant pathogen Ralstonia solanacearum inferred from gene distribution analysis. J Bacteriol. 189:377-87.
Occhialini A, Cunnac S, Reymond N, Genin S, Boucher C (2005) Genome-wide analysis of gene expression in Ralstonia solanacearum reveals that the hrpB gene acts as a regulatory switch controlling multiple virulence pathways. Mol Plant Microbe Interact. 18:938-49.
Prior P., Fegan M., 2005. Recent development in the phylogeny and classification of Ralstonia solanacearum. Acta Horticulturae. (ISHS) 695:127-136.
Reymond N, Charles H, Duret L, Calevro F, Beslon G, Fayard J M. (2004) ROSO: optimizing oligonucleotide probes for microarrays. Bioinformatics. 20:271-3
Salanoubat, M., S. Genin, F. Artiguenave, J. Gouzy, S. Mangenot, M. Arlat, A. Billault, P. Brother, J. C. Camus, L. Cattolico, M. Chandler, N. Choisne, C. Claudel-Renard, S. Cunnac, N. Demange, C. Gaspin, M. Lavie, A. Moisan, C. Robert, W. Saurin, T. Schiex, P. Siguier, P. Thébault, M. Whalen, P. Wincker, M. Levy, J. Weissenbach, and C. A. Boucher (2002) Genome sequence of the plant pathogen Ralstonia solanacearum. Nature 415:497-502.
Wicker E., Grassart L., Coranson-Beaudu R., Mian D., Guilbaud C., Fegan M. and Prior P. (2007). Ralstonia solanacearum strains in Martinique (French West Indies) exhibiting a new pathogenic potential. Applied and Environmental Microbiology. In press

1. A method for the detection of Ralstonia solanacearum race 3 biovar 2 strains in a medium, comprising the determination of the presence or the absence in a sample of the medium, of:
(i) at least one first nucleic acid target having a sequence selected from the group constituted of SEQ ID NO: 1-49, complementary sequences thereof, and homologous sequences thereof, or
(ii) at least one fragment of said first nucleic acid target, wherein said fragment is not constituted of or comprised in a sequence selected from the group constituted of SEQ ID NO: 111-140;
whereby, if said first nucleic acid target or fragment thereof is present in the sample, it is determined that Ralstonia solanacearum race 3 biovar 2 strain is present in the medium.

2. The method according to claim 1, wherein the medium is selected from the group constituted of a potato tissue, a tomato tissue, and a geranium tissue.

3. The method according to claim 1, wherein the medium is a potato tissue.

4. The method according to claim 3, wherein the potato tissue is the tuber.

5. The method according to claim 1, wherein the sample is obtained from the medium by a nucleic acid extraction.

6. The method according to claim 1, wherein the determination comprises at least one step of hybridization of the nucleic acid target or fragment thereof with a probe or a primer.

7. The method according to claim 6, wherein the probe or primer is a fragment of nucleic acid having a sequence selected from the group constituted of SEQ ID NO: 1-49, homologous sequences thereof, and complementary sequences thereof.

8. The method according to claim 6, wherein the probe or primer is selected from the group constituted of SEQ ID NO: 141-386.

9. The method according to claim 1, wherein the determination comprises at least one step of nucleic acid amplification.

10. The method according to claim 6, wherein the determination is implemented by a method selected from PCR and NASBA.

11. The method according to claim 6, wherein the determination is implemented by a method selected from Southern blotting, Northern blotting, dot blots, and nucleic acid micro or macro-array hybridization.

12. The method according to claim 1, comprising the determination of the presence or the absence in a sample of the medium, of:
(i) at least one second nucleic acid target having a sequence selected from the group constituted of SEQ ID NO: 111-140, complementary sequences thereof, and homologous sequences thereof, or
(ii) at least one fragment of said second target nucleic acid.

13. A nucleic acid having a sequence selected from the group constituted of SEQ ID NO: 1-22, SEQ ID NO: 50-110, SEQ ID NO: 111-140, SEQ ID NO: 141-246, and SEQ ID NO: 247-386 and their complementary sequences.

14. A nucleic acid micro or macro-array comprising a plurality of nucleic acid probes arranged onto a solid support, wherein the nucleic acid probes are fragments of nucleic acids having sequences selected from the group constituted of SEQ ID NO: 1-49, complementary sequences thereof, and homologous sequences thereof, provided that at least one of the nucleic acid probes is not a fragment of a nucleic acid having a sequence selected from the group consisting of SEQ ID NO: 111-140.

15. The nucleic acid micro or macro-array according to claim 14, wherein the nucleic acid probes comprise SEQ ID NO: 247-386.

16. A kit intended for the detection of Ralstonia solanacearum race 3 biovar 2 in a medium, comprising at least:
two primers suitable to amplify a portion of a nucleic acid having a sequence selected from the group constituted of SEQ ID NO: 1-49 and complementary sequences thereof, provided that said portion is not comprised in a nucleic acid having a sequence selected from the group consisting of SEQ ID NO: 111-140 and complementary sequences thereof;
optionally one detectable nucleic acid probe suitable to hybridize to said amplified portion.

17. (canceled)

18. The method according to claim 9, wherein the determination is implemented by a method selected from PCR and NASBA.
